SEQUENCE LISTING (6 sequences)

SEQ ID NO: 1 - DNA, 3480 bp, Homosapiens
  KCNH2, Kv11.1, HERG, HERG1, erg1
atgccggtgcggaggggccacgtcgcgccgcagaacaccttcctggacaccatcatccgcaagtttgagggccagagccgtaagttcatcatcgccaacgctcgggtggagaactgcgccgtcatctactgcaacgacggcttctgcgagctgtgcggctactcgcgggccgaggtgatgcagcgaccctgcacctgcgacttcctgcacgggccgcgcacgcagcgccgcgctgccgcgcagatcgcgcaggcactgctgggcgccgaggagcgcaaagtggaaatcgccttctaccggaaagatgggagctgcttcctatgtctggtggatgtggtgcccgtgaagaacgaggatggggctgtcatcatgttcatcctcaatttcgaggtggtgatggagaaggacatggtggggtccccggctcatgacaccaaccaccggggcccccccaccagctggctggccccaggccgcgccaagaccttccgcctgaagctgcccgcgctgctggcgctgacggcccgggagtcgtcggtgcggtcgggcggcgcgggcggcgcgggcgccccgggggccgtggtggtggacgtggacctgacgcccgcggcacccagcagcgagtcgctggccctggacgaagtgacagccatggacaaccacgtggcagggctcgggcccgcggaggagcggcgtgcgctggtgggtcccggctctccgccccgcagcgcgcccggccagctcccatcgccccgggcgcacagcctcaaccccgacgcctcgggctccagctgcagcctggcccggacgcgctcccgagaaagctgcgccagcgtgcgccgcgcctcgtcggccgacgacatcgaggccatgcgcgccggggtgctgcccccgccaccgcgccacgccagcaccggggccatgcacccactgcgcagcggcttgctcaactccacctcggactccgacctcgtgcgctaccgcaccattagcaagattccccaaatcaccctcaactttgtggacctcaagggcgaccccttcttggcttcgcccaccagtgaccgtgagatcatagcacctaagataaaggagcgaacccacaatgtcactgagaaggtcacccaggtcctgtccctgggcgccgacgtgctgcctgagtacaagctgcaggcaccgcgcatccaccgctggaccatcctgcattacagccccttcaaggccgtgtgggactggctcatcctgctgctggtcatctacacggctgtcttcacaccctactcggctgccttcctgctgaaggagacggaagaaggcccgcctgctaccgagtgtggctacgcctgccagccgctggctgtggtggacctcatcgtggacatcatgttcattgtggacatcctcatcaacttccgcaccacctacgtcaatgccaacgaggaggtggtcagccaccccggccgcatcgccgtccactacttcaagggctggttcctcatcgacatggtggccgccatccccttcgacctgctcatcttcggctctggctctgaggagctgatcgggctgctgaagactgcgcggctgctgcggctggtgcgcgtggcgcggaagctggatcgctactcagagtacggcgcggccgtgctgttcttgctcatgtgcacctttgcgctcatcgcgcactggctagcctgcatctggtacgccatcggcaacatggagcagccacacatggactcacgcatcggctggctgcacaacctgggcgaccagataggcaaaccctacaacagcagcggcctgggcggcccctccatcaaggacaagtatgtgacggcgctctacttcaccttcagcagcctcaccagtgtgggcttcggcaacgtctctcccaacaccaactcagagaagatcttctccatctgcgtcatgctcattggctccctcatgtatgctagcatcttcggcaacgtgtcggccatcatccagcggctgtactcgggcacagcccgctaccacacacagatgctgcgggtgcgggagttcatccgcttccaccagatccccaatcccctgcgccagcgcctcgaggagtacttccagcacgcctggtcctacaccaacggcatcgacatgaacgcggtgctgaagggcttccctgagtgcctgcaggctgacatctgcctgcacctgaaccgctcactgctgcagcactgcaaacccttccgaggggccaccaagggctgccttcgggccctggccatgaagttcaagaccacacatgcaccgccaggggacacactggtgcatgctggggacctgctcaccgccctgtacttcatctcccggggctccatcgagatcctgcggggcgacgtcgtcgtggccatcctggggaagaatgacatctttggggagcctctgaacctgtatgcaaggcctggcaagtcgaacggggatgtgcgggccctcacctactgtgacctacacaagatccatcgggacgacctgctggaggtgctggacatgtaccctgagttctccgaccacttctggtccagcctggagatcaccttcaacctgcgagataccaacatgatcccgggctcccccggcagtacggagttagagggtggcttcagtcggcaacgcaagcgcaagttgtccttccgcaggcgcacggacaaggacacggagcagccaggggaggtgtcggccttggggccgggccgggcgggggcagggccgagtagccggggccggccgggggggccgtggggggagagcccgtccagtggcccctccagccctgagagcagtgaggatgagggcccaggccgcagctccagccccctccgcctggtgcccttctccagccccaggccccccggagagccgccgggtggggagcccctgatggaggactgcgagaagagcagcgacacttgcaaccccctgtcaggcgccttctcaggagtgtccaacattttcagcttctggggggacagtcggggccgccagtaccaggagctccctcgatgccccgcccccacccccagcctcctcaacatccccctctccagcccgggtcggcggccccggggcgacgtggagagcaggctggatgccctccagcgccagctcaacaggctggagacccggctgagtgcagacatggccactgtcctgcagctgctacagaggcagatgacgctggtcccgcccgcctacagtgctgtgaccaccccggggcctggccccacttccacatccccgctgttgcccgtcagccccctccccaccctcaccttggactcgctttctcaggtttcccagttcatggcgtgtgaggagctgcccccgggggccccagagcttccccaagaaggccccacacgacgcctctccctaccgggccagctgggggccctcacctcccagcccctgcacagacacggctcggacccgggcagttag

SEQ ID NO: 2 - protein, 1159 aa, Homosapiens
  KCNH2, Kv11.1, HERG, HERG1, erg1
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

SEQ ID NO: 3 - DNA, 6051 bp, Homosapiens
  SCN5A, Nav1.5
atggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcgtatgtatcagaaaatataaaactaggcaatttgtcggctcttcgaactttcagagtcctgagagctctaaaaactatttcagttatcccagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtga

SEQ ID NO: 4 - protein, 2016 aa, Homosapiens
  SCN5A, Nav1.5
MANFLLPRGTSSFRRFTRESLAAIEKRMAEKQARGSTTLQESREGLPEEEAPRPQLDLQASKKLPDLYGNPPQELIGEPLEDLDPFYSTQKTFIVLNKGKTIFRFSATNALYVLSPFHPIRRAAVKILVHSLFNMLIMCTILTNCVFMAQHDPPPWTKYVEYTFTAIYTFESLVKILARGFCLHAFTFLRDPWNWLDFSVIIMAYVSENIKLGNLSALRTFRVLRALKTISVIPGLKTIVGALIQSVKKLADVMVLTVFCLSVFALIGLQLFMGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGKIYMIFFMLVIFLGSFYLVNLILAVVAMAYEEQNQATIAETEEKEKRFQEAMEMLKKEHEALTIRGVDTVSRSSLEMSPLAPVNSHERRSKRRKRMSSGTEECGEDRLPKSDSEDGPRAMNHLSLTRGLSRTSMKPRSSRGSIFTFRRRDLGSEADFADDENSTAGESESHHTSLLVPWPLRRTSAQGQPSPGTSAPGHALHGKKNSTVDCNGVVSLLGAGDPEATSPGSHLLRPVMLEHPPDTTTPSEEPGGPQMLTSQAPCVDGFEEPGARQRALSAVSVLTSALEELEESRHKCPPCWNRLAQRYLIWECCPLWMSIKQGVKLVVMDPFTDLTITMCIVLNTLFMALEHYNMTSEFEEMLQVGNLVFTGIFTAEMTFKIIALDPYYYFQQGWNIFDSIIVILSLMELGLSRMSNLSVLRSFRLLRVFKLAKSWPTLNTLIKIIGNSVGALGNLTLVLAIIVFIFAVVGMQLFGKNYSELRDSDSGLLPRWHMMDFFHAFLIIFRILCGEWIETMWDCMEVSGQSLCLLVFLLVMVIGNLVVLNLFLALLLSSFSADNLTAPDEDREMNNLQLALARIQRGLRFVKRTTWDFCCGLLRQRPQKPAALAAQGQLPSCIATPYSPPPPETEKVPPTRKETRFEEGEQPGQGTPGDPEPVCVPIAVAESDTDDQEEDEENSLGTEEESSKQQESQPVSGGPEAPPDSRTWSQVSATASSEAEASASQADWRQQWKAEPQAPGCGETPEDSCSEGSTADMTNTAELLEQIPDLGQDVKDPEDCFTEGCVRRCPCCAVDTTQAPGKVWWRLRKTCYHIVEHSWFETFIIFMILLSSGALAFEDIYLEERKTIKVLLEYADKMFTYVFVLEMLLKWVAYGFKKYFTNAWCWLDFLIVDVSLVSLVANTLGFAEMGPIKSLRTLRALRPLRALSRFEGMRVVVNALVGAIPSIMNVLLVCLIFWLIFSIMGVNLFAGKFGRCINQTEGDLPLNYTIVNNKSQCESLNLTGELYWTKVKVNFDNVGAGYLALLQVATFKGWMDIMYAAVDSRGYEEQPQWEYNLYMYIYFVIFIIFGSFFTLNLFIGVIIDNFNQQKKKLGGQDIFMTEEQKKYYNAMKKLGSKKPQKPIPRPLNKYQGFIFDIVTKQAFDVTIMFLICLNMVTMMVETDDQSPEKINILAKINLLFVAIFTGECIVKLAALRHYYFTNSWNIFDFVVVILSIVGTVLSDIIQKYFFSPTLFRVIRLARIGRILRLIRGAKGIRTLLFALMMSLPALFNIGLLLFLVMFIYSIFGMANFAYVKWEAGIDDMFNFQTFANSMLCLFQITTSAGWDGLLSPILNTGPPYCDPTLPNSNGSRGDCGSPAVGILFFTTYIIISFLIVVNMYIAIILENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 5 - DNA, 6417 bp, Homosapiens
  CACNA1C, Cav1.2
atggtcaatgagaatacgaggatgtacattccagaggaaaaccaccaaggttccaactatgggagcccacgccccgcccatgccaacatgaatgccaatgcggcagcggggctggcccctgagcacatccccaccccgggggctgccctgtcgtggcaggcggccatcgacgcagcccggcaggctaagctgatgggcagcgctggcaatgcgaccatctccacagtcagctccacgcagcggaagcggcagcaatatgggaaacccaagaagcagggcagcaccacggccacacgcccgccccgagccctgctctgcctgaccctgaagaaccccatccggagggcctgcatcagcattgtcgaatggaaaccatttgaaataattattttactgactatttttgccaattgtgtggccttagcgatctatattccctttccagaagatgattccaacgccaccaattccaacctggaacgagtggaatatctctttctcataatttttacggtggaagcgtttttaaaagtaatcgcctatggactcctctttcaccccaatgcctacctccgcaacggctggaacctactagattttataattgtggttgtggggctttttagtgcaattttagaacaagcaaccaaagcagatggggcaaacgctctcggagggaaaggggccggatttgatgtgaaggcgctgagggccttccgcgtgctgcgccccctgcggctggtgtccggagtcccaagtctccaggtggtcctgaattccatcatcaaggccatggtccccctgctgcacatcgccctgcttgtgctgtttgtcatcatcatctacgccatcatcggcttggagctcttcatggggaagatgcacaagacctgctacaaccaggagggcatagcagatgttccagcagaagatgacccttccccttgtgcgctggaaacgggccacgggcggcagtgccagaacggcacggtgtgcaagcccggctgggatggtcccaagcacggcatcaccaactttgacaactttgccttcgccatgctcacggtgttccagtgcatcaccatggagggctggacggacgtgctgtactgggtcaatgatgccgtaggaagggactggccctggatctattttgttacactaatcatcatagggtcattttttgtacttaacttggttctcggtgtgcttagcggagagttttccaaagagagggagaaggccaaggcccggggagatttccagaagctgcgggagaagcagcagctagaagaggatctcaaaggctacctggattggatcactcaggccgaagacatcgatcctgagaatgaggacgaaggcatggatgaggagaagccccgaaacatgagcatgcccaccagtgagaccgagtccgtcaacaccgaaaacgtggctggaggtgacatcgagggagaaaactgcggggccaggctggcccaccggatctccaagtcaaagttcagccgctactggcgccggtggaatcggttctgcagaaggaagtgccgcgccgcagtcaagtctaatgtcttctactggctggtgattttcctggtgttcctcaacacgctcaccattgcctctgagcactacaaccagcccaactggctcacagaagtccaagacacggcaaacaaggccctgctggccctgttcacggcagagatgctcctgaagatgtacagcctgggcctgcaggcctacttcgtgtccctcttcaaccgctttgactgcttcgtcgtgtgtggcggcatcctggagaccatcctggtggagaccaagatcatgtccccactgggcatctccgtgctcagatgcgtccggctgctgaggattttcaagatcacgaggtactggaactccttgagcaacctggtggcatccttgctgaactctgtgcgctccatcgcctccctgctccttctcctcttcctcttcatcatcatcttctccctcctggggatgcagctctttggaggaaagttcaactttgatgagatgcagacccggaggagcacattcgataacttcccccagtccctcctcactgtgtttcagatcctgaccggggaggactggaattcggtgatgtatgatgggatcatggcttatggcggcccctcttttccagggatgttagtctgtatttacttcatcatcctcttcatctgtggaaactatatcctactgaatgtgttcttggccattgctgtggacaacctggctgatgctgagagcctcacatctgcccaaaaggaggaggaagaggagaaggagagaaagaagctggccaggactgccagcccagagaagaaacaagagttggtggagaagccggcagtgggggaatccaaggaggagaagattgagctgaaatccatcacggctgacggagagtctccacccgccaccaagatcaacatggatgacctccagcccaatgaaaatgaggataagagcccctaccccaacccagaaactacaggagaagaggatgaggaggagccagagatgcctgtcggccctcgcccacgaccactctctgagcttcaccttaaggaaaaggcagtgcccatgccagaagccagcgcgtttttcatcttcagctctaacaacaggtttcgcctccagtgccaccgcattgtcaatgacacgatcttcaccaacctgatcctcttcttcattctgctcagcagcatttccctggctgctgaggacccggtccagcacacctccttcaggaaccatattctgttttattttgatattgtttttaccaccattttcaccattgaaattgctctgaagatgactgcttatggggctttcttgcacaagggttctttctgccggaactacttcaacatcctggacctgctggtggtcagcgtgtccctcatctcctttggcatccagtccagtgcaatcaatgtcgtgaagatcttgcgagtcctgcgagtactcaggcccctgagggccatcaacagggccaaggggctaaagcatgtggttcagtgtgtgtttgtcgccatccggaccatcgggaacatcgtgattgtcaccaccctgctgcagttcatgtttgcctgcatcggggtccagctcttcaagggaaagctgtacacctgttcagacagttccaagcagacagaggcggaatgcaagggcaactacatcacgtacaaagacggggaggttgaccaccccatcatccaaccccgcagctgggagaacagcaagtttgactttgacaatgttctggcagccatgatggccctcttcaccgtctccaccttcgaagggtggccagagctgctgtaccgctccatcgactcccacacggaagacaagggccccatctacaactaccgtgtggagatctccatcttcttcatcatctacatcatcatcatcgccttcttcatgatgaacatcttcgtgggcttcgtcatcgtcacctttcaggagcagggggagcaggagtacaagaactgtgagctggacaagaaccagcgacagtgcgtggaatacgccctcaaggcccggcccctgcggaggtacatccccaagaaccagcaccagtacaaagtgtggtacgtggtcaactccacctacttcgagtacctgatgttcgtcctcatcctgctcaacaccatctgcctggccatgcagcactacggccagagctgcctgttcaaaatcgccatgaacatcctcaacatgctcttcactggcctcttcaccgtggagatgatcctgaagctcattgccttcaaacccaagcactatttctgtgatgcatggaatacatttgacgccttgattgttgtgggtagcattgttgatatagcaatcaccgaggtaaacccagctgaacatacccaatgctctccctctatgaacgcagaggaaaactcccgcatctccatcaccttcttccgcctgttccgggtcatgcgtctggtgaagctgctgagccgtggggagggcatccggacgctgctgtggaccttcatcaagtccttccaggccctgccctatgtggccctcctgatcgtgatgctgttcttcatctacgcggtgatcgggatgcaggtgtttgggaaaattgccctgaatgataccacagagatcaaccggaacaacaactttcagaccttcccccaggccgtgctgctcctcttcaggtgtgccaccggggaggcctggcaggacatcatgctggcctgcatgccaggcaagaagtgtgccccagagtccgagcccagcaacagcacggagggtgaaacaccctgtggtagcagctttgctgtcttctacttcatcagcttctacatgctctgtgccttcctgatcatcaacctctttgtagctgtcatcatggacaactttgactacctgacaagggactggtccatccttggtccccaccacctggatgagtttaaaagaatctgggcagagtatgaccctgaagccaagggtcgtatcaaacacctggatgtggtgaccctcctccggcggattcagccgccactaggttttgggaagctgtgccctcaccgcgtggcttgcaaacgcctggtctccatgaacatgcctctgaacagcgacgggacagtcatgttcaatgccaccctgtttgccctggtcaggacggccctgaggatcaaaacagaagggaacctagaacaagccaatgaggagctgcgggcgatcatcaagaagatctggaagcggaccagcatgaagctgctggaccaggtggtgccccctgcaggtgatgatgaggtcaccgttggcaagttctacgccacgttcctgatccaggagtacttccggaagttcaagaagcgcaaagagcagggccttgtgggcaagccctcccagaggaacgcgctgtctctgcaggctggcttgcgcacactgcatgacatcgggcctgagatccgacgggccatctctggagatctcaccgctgaggaggagctggacaaggccatgaaggaggctgtgtccgctgcttctgaagatgacatcttcaggagggccggtggcctgttcggcaaccacgtcagctactaccaaagcgacggccggagcgccttcccccagaccttcaccactcagcgcccgctgcacatcaacaaggcgggcagcagccagggcgacactgagtcgccatcccacgagaagctggtggactccaccttcaccccgagcagctactcgtccaccggctccaacgccaacatcaacaacgccaacaacaccgccctgggtcgcctccctcgccccgccggctaccccagcacggtcagcactgtggagggccacgggccccccttgtcccctgccatccgggtgcaggaggtggcgtggaagctcagctccaacaggtgccactcccgggagagccaggcagccatggcgggtcaggaggagacgtctcaggatgagacctatgaagtgaagatgaaccatgacacggaggcctgcagtgagcccagcctgctctccacagagatgctctcctaccaggatgacgaaaatcggcaactgacgctcccagaggaggacaagagggacatccggcaatctccgaagaggggtttcctccgctctgcctcactaggtcgaagggcctccttccacctggaatgtctgaagcgacagaaggaccgagggggagacatctctcagaagacagtcctgcccttgcatctggttcatcatcaggcattggcagtggcaggcctgagccccctcctccagagaagccattcccctgcctcattccctaggccttttgccaccccaccagccacacctggcagccgaggctggcccccacagcccgtccccaccctgcggcttgagggggtcgagtccagtgagaaactcaacagcagcttcccatccatccactgcggctcctgggctgagaccacccccggtggcgggggcagcagcgccgcccggagagtccggcccgtctccctcatggtgcccagccaggctggggccccagggaggcagttccacggcagtgccagcagcctggtggaagcggtcttgatttcagaaggactggggcagtttgctcaagatcccaagttcatcgaggtcaccacccaggagctggccgacgcctgcgacatgaccatagaggagatggagagcgcggccgacaacatcctcagcgggggcgccccacagagccccaatggcgccctcttaccctttgtgaactgcagggacgcggggcaggaccgagccgggggcgaagaggacgcgggctgtgtgcgcgcgcggggtcgaccgagtgaggaggagctccaggacagcagggtctacgtcagcagcctgtag

SEQ ID NO: 6 - protein, 2138 aa, Homosapiens
  CACNA1C, Cav1.2
MVNENTRMYIPEENHQGSNYGSPRPAHANMNANAAAGLAPEHIPTPGAALSWQAAIDAARQAKLMGSAGNATISTVSSTQRKRQQYGKPKKQGSTTATRPPRALLCLTLKNPIRRACISIVEWKPFEIIILLTIFANCVALAIYIPFPEDDSNATNSNLERVEYLFLIIFTVEAFLKVIAYGLLFHPNAYLRNGWNLLDFIIVVVGLFSAILEQATKADGANALGGKGAGFDVKALRAFRVLRPLRLVSGVPSLQVVLNSIIKAMVPLLHIALLVLFVIIIYAIIGLELFMGKMHKTCYNQEGIADVPAEDDPSPCALETGHGRQCQNGTVCKPGWDGPKHGITNFDNFAFAMLTVFQCITMEGWTDVLYWVNDAVGRDWPWIYFVTLIIIGSFFVLNLVLGVLSGEFSKEREKAKARGDFQKLREKQQLEEDLKGYLDWITQAEDIDPENEDEGMDEEKPRNMSMPTSETESVNTENVAGGDIEGENCGARLAHRISKSKFSRYWRRWNRFCRRKCRAAVKSNVFYWLVIFLVFLNTLTIASEHYNQPNWLTEVQDTANKALLALFTAEMLLKMYSLGLQAYFVSLFNRFDCFVVCGGILETILVETKIMSPLGISVLRCVRLLRIFKITRYWNSLSNLVASLLNSVRSIASLLLLLFLFIIIFSLLGMQLFGGKFNFDEMQTRRSTFDNFPQSLLTVFQILTGEDWNSVMYDGIMAYGGPSFPGMLVCIYFIILFICGNYILLNVFLAIAVDNLADAESLTSAQKEEEEEKERKKLARTASPEKKQELVEKPAVGESKEEKIELKSITADGESPPATKINMDDLQPNENEDKSPYPNPETTGEEDEEEPEMPVGPRPRPLSELHLKEKAVPMPEASAFFIFSSNNRFRLQCHRIVNDTIFTNLILFFILLSSISLAAEDPVQHTSFRNHILFYFDIVFTTIFTIEIALKMTAYGAFLHKGSFCRNYFNILDLLVVSVSLISFGIQSSAINVVKILRVLRVLRPLRAINRAKGLKHVVQCVFVAIRTIGNIVIVTTLLQFMFACIGVQLFKGKLYTCSDSSKQTEAECKGNYITYKDGEVDHPIIQPRSWENSKFDFDNVLAAMMALFTVSTFEGWPELLYRSIDSHTEDKGPIYNYRVEISIFFIIYIIIIAFFMMNIFVGFVIVTFQEQGEQEYKNCELDKNQRQCVEYALKARPLRRYIPKNQHQYKVWYVVNSTYFEYLMFVLILLNTICLAMQHYGQSCLFKIAMNILNMLFTGLFTVEMILKLIAFKPKHYFCDAWNTFDALIVVGSIVDIAITEVNPAEHTQCSPSMNAEENSRISITFFRLFRVMRLVKLLSRGEGIRTLLWTFIKSFQALPYVALLIVMLFFIYAVIGMQVFGKIALNDTTEINRNNNFQTFPQAVLLLFRCATGEAWQDIMLACMPGKKCAPESEPSNSTEGETPCGSSFAVFYFISFYMLCAFLIINLFVAVIMDNFDYLTRDWSILGPHHLDEFKRIWAEYDPEAKGRIKHLDVVTLLRRIQPPLGFGKLCPHRVACKRLVSMNMPLNSDGTVMFNATLFALVRTALRIKTEGNLEQANEELRAIIKKIWKRTSMKLLDQVVPPAGDDEVTVGKFYATFLIQEYFRKFKKRKEQGLVGKPSQRNALSLQAGLRTLHDIGPEIRRAISGDLTAEEELDKAMKEAVSAASEDDIFRRAGGLFGNHVSYYQSDGRSAFPQTFTTQRPLHINKAGSSQGDTESPSHEKLVDSTFTPSSYSSTGSNANINNANNTALGRLPRPAGYPSTVSTVEGHGPPLSPAIRVQEVAWKLSSNRCHSRESQAAMAGQEETSQDETYEVKMNHDTEACSEPSLLSTEMLSYQDDENRQLTLPEEDKRDIRQSPKRGFLRSASLGRRASFHLECLKRQKDRGGDISQKTVLPLHLVHHQALAVAGLSPLLQRSHSPASFPRPFATPPATPGSRGWPPQPVPTLRLEGVESSEKLNSSFPSIHCGSWAETTPGGGGSSAARRVRPVSLMVPSQAGAPGRQFHGSASSLVEAVLISEGLGQFAQDPKFIEVTTQELADACDMTIEEMESAADNILSGGAPQSPNGALLPFVNCRDAGQDRAGGEEDAGCVRARGRPSEEELQDSRVYVSSL

(19) United States
(12) Patent Application Publication
(10) Pub. No.: US 2023/0349886 A1
(43) Pub. Date: Nov. 2, 2023

(54) SYSTEMS AND METHODS FOR PREDICTING CARDIOTOXICITY OF MOLECULAR PARAMETERS OF A COMPOUND BASED ON MACHINE LEARNING ALGORITHMS

(71) Applicant: UTI Limited Partnership, Calgary (CA)

(72) Inventors: Sergei NOSKOV, Calgary (CA); Soren WACKER, Calgary (CA); Henry DUFF, Calgary (CA); Jiqing GUO, Calgary (CA)

(21) Appl. No.: 17/715,796

(22) Filed: Apr. 7, 2022

Related U.S. Application Data
(63) Continuation of application No. 15/737,246, filed on Dec. 15, 2017, now abandoned.
(60) Provisional application No. 62/181,115, filed on Jun. 17, 2015.

(51) Int. Cl. G01N 33/50; G06N 20/00; G16B 15/00; G16B 35/00; G16B 40/00; G16C 20/60; G01N 33/68; G16B 20/00; G16C 20/30; G06N 20/20; G16H 50/00; G16B 15/30; G16B 40/20; G16B 35/20; G06N 5/01; G06N 7/01

(52) U.S. Cl. CPC G01N 33/5014; G01N 33/6872; G06N 5/01; G06N 7/01; G06N 20/00; G06N 20/20; G16B 15/00; G16B 15/30; G16B 20/00; G16B 35/00; G16B 35/20; G16B 40/00; G16B 40/20; G16C 20/30; G16C 20/60; G16H 50/00; G16C 20/70

(57) ABSTRACT

Systems and methods are provided for predicting cardiotoxicity of molecular parameters of a compound. A computer can provide as input to a machine learning algorithm the molecular parameters of the compound. The molecular parameters can include at least structural information about the compound. The machine learning algorithm can have been trained using respective molecular parameters of compounds known to have cardiotoxicity and of compounds known not to have cardiotoxicity. The computer can receive as output from the machine learning algorithm a representation of the predicted cardiotoxicity of each molecular parameter of at least a subset of the molecular parameters of the compound.

### CROSS REFERENCE TO RELATED APPLICATIONS

The present application is a continuation of 15/737,246, filed Dec. 15, 2017, which is a national stage filing under 35 U.S.C. § 371 of International Application No. PCT/CA2016/050705, filed on Jun. 16, 2016, which claims the benefit of priority of U.S. Provisional Application No. 62/181,115, filed Jun. 17, 2015, the content of each of which is hereby incorporated by reference in its entirety.

### SEQUENCE LISTING

This application incorporates by reference the Computer Readable Form (CRF) of a Sequence Listing in ASCII text format submitted via EFS-Web. The Sequence Listing text file submitted via EFS-Web, entitled 13449-018-999_SEQ_LISTING.txt, was created on Dec. 12, 2017, and is 68,019 bytes in size.

### TECHNICAL FIELD

This application generally relates to predicting cardiotoxicity of a compound.

### BACKGROUND

A given compound that is administered to a subject may be intended to interact with a desired target, e.g., a protein involved in a particular pathology that the compound is intended to treat, but potentially can interact with one or more unintended targets, e.g., proteins that are not involved in the particular pathology that the compound is intended to treat. Such interactions with unintended targets potentially can cause severe side effects. A prominent example is the hERG (human ether-a-go-go related gene) potassium channel, which is responsible for the repolarization of the cardiac action potential. In the late 1990s, numerous drugs had to be removed from the market because such drugs unintentionally blocked the hERG potassium channel, resulting in a prolongation of the QT-interval of the action potential and causing the subject to experience life threatening arrhythmia. Since then, each compound entering the market or assessed for clinical trials is assessed for safety with respect to the hERG potassium channel. Compounds that interfere with cardiac ion protein channels or other normal activity of the heart can be referred to as being “cardiotoxic” or as having “torsadogenic activity.” Cardiotoxic compounds can cause Torsades de Pointes.

Computer models for predicting the side effects of a compound with respect to the hERG potassium channel potentially can be helpful so as to sort out high risk compounds even before those compounds are synthesized. For example, receptor-based approaches utilize three-dimensional (3D) structural data available for intended and unintended targets, e.g., proteins. However, such approaches can be relatively expensive, which can make it prohibitive to use such approaches to analyze large datasets, e.g., large numbers of compounds. Additionally, such approaches can be limited to studies of molecules with available parameters (e.g., force-fields). Therefore, molecular simulations targeting protein-drug complexes are filling the niche for the “designer’s” approach to pre-clinical studies where the dataset is already curated and a relatively small sub-set of the potentially-toxic compounds identified. Other examples for receptor-based models use molecular docking, all-atom molecular dynamics, and Free Energy simulations.

Exemplary alternatives are ligand based models, which can be collectively categorized as structure-activity relationship (SAR) models, and which can be less expensive to use than are receptor-based approaches, and can be relatively computationally fast and reasonably accurate. In a SAR model, the chemical structure of a compound is known, such that the compound can be characterized using a set of parameters, for example, the compound’s solubility, the compound’s weight, or the number of rotatable bonds in the compound. Such so called “molecular parameters” then are used as input for machine learning algorithms to explore relationships within the data and to train models. Some molecular parameters are strongly correlated with one another. As one example, the number of rings in a compound, the number of atoms in a compound, and the molecular weight of the compound may be correlated with one another. Such correlations potentially can lead to high variance in the models, thus reducing the robustness of the model. The general recommendation to deal with covariant data is linearization, e.g., with principle component analysis (PCA) or feature selection. Alternatively, distance based methods like iso-mapping can be applied to learn the underlying structure of the data and train the model based on that structure. Another possibility is to use certain types of non-linear models that perform an internal feature selection. Finally, the selected set of features, which can include specific parameters, linear combinations or otherwise transformed collective coordinates, then can be fed into a machine learning algorithm.

Models to predict hERG affinities have been published for at least a decade. Some of them have been made publicly available. However, it appears that accuracies of some models can be overestimated due to a variety of reasons. For example, an apparently high correlation between assayed activity and in-silico predictions can arise from using a relatively limited training set and/or test set. For example, the generation of a dataset of active and inactive compounds is usually not a randomized and representative sample. Splitting a dataset up into training, test and validation set can therefore lead to artifacts. In machine learning a training set is used to train a model. If sufficient data is available, the remaining data is spilt up into a test set for the selection of the best model so as to avoid or inhibit over-fitting, and a validation set so as to estimate the off-sample accuracy/error, e.g., the prediction error in a sample that has not been used to build or select the model. If the data is not randomized, confounding variables can exist in the dataset which are not representative for the population of all data, which can lead to high off-sample accuracies in validation sets originating from the same sample as the training set.

Some studies measure the quality of the prediction based on the true positive rate, however this approach can overestimate the performance of the model. For example, based on a model classifying all compounds as “active,” then the true positive rate will be optimal (meaning one), but the resulting model cannot distinguish between the different classes (e.g., active or inactive). Therefore, other metrics must be used like the prediction accuracy (PA) or Kohen’s kappa (KK) or the F1 score. PA takes both classes into account and can lead to accurate estimations as long as both classes (active and inactive) are equally represented in the validated set. KK takes random correct classification into account and F1 is designed to account for unbalanced dataset similar to KK. Other metrics can be used to estimate the ranking quality of a model, such as the area under the receiver-operator-characteristic curve (AROC). For real number estimates metrics like the Pearson correlation coefficient (R) and the coefficient of determination (R2) or the root mean squared error (RMSE) are used. While r measures the coupling between two variables, R2 and RMSE measure the absolute agreement of two variables.

### SUMMARY

Embodiments of the present invention provide systems and methods for predicting cardiotoxicity of molecular parameters of a compound based on machine learning algorithms.

Under one aspect, a computer-implemented method of predicting cardiotoxicity of molecular parameters of a compound is provided. The method includes, by a computer, providing as input to a machine learning algorithm the molecular parameters of the compound. The molecular parameters can include at least structural information about the compound, and the machine learning algorithm can have been trained using respective molecular parameters of compounds known to have cardiotoxicity and of compounds known not to have cardiotoxicity. The method also can include, by the computer, receiving as output from the machine learning algorithm a representation of the predicted cardiotoxicity of each molecular parameter of at least a subset of the molecular parameters of the compound.

In some embodiments, the representation of the predicted cardiotoxicity includes, for each molecular parameter of at least the subset of the molecular parameters of the compound, a numerical value representing the predicted cardiotoxicity of that molecular parameter.

Some embodiments further include redesigning the compound so as not to include at least one of the molecular parameters of at least the subset. For example, the method can include, by the computer, providing as input to the machine learning algorithm the molecular parameters of the redesigned compound; and by the computer, receiving as output from the machine learning algorithm a representation of the predicted cardiotoxicity of each molecular parameter of at least a subset of the molecular parameters of the redesigned compound.

In some embodiments, the representation includes a value representative of a prediction that the molecular parameter of at least the subset will cause the compound to block two or more cardiac ion protein channels. For example, the two or more cardiac ion protein channels can be selected from the group consisting of: sodium ion channel proteins, calcium ion channel proteins, and potassium ion channel proteins. In some embodiments, the potassium ion channel protein hERG1, the sodium ion channel protein is hNaᵥ1.5, or the calcium channel protein is hCav1.2.

Some embodiments further include, by the computer, providing as input to the machine learning algorithm, respective molecular parameters of a plurality of compounds of which the previously recited compound is a member. Some embodiments further include, by the computer, receiving as output from the machine learning algorithm a representation of the predicted cardiotoxicity of each molecular parameter of at least a subset of the molecular parameters of each of the compounds of the plurality of compounds. Some embodiments further include, by the computer, selecting a compound of the plurality of compounds based on the predicted cardiotoxicity of each molecular parameter of at least a subset of the molecular parameters of each of the compounds of the plurality of compounds.

In some embodiments, the compounds known to have cardiotoxicity and the compounds known not to have cardiotoxicity are selected based on a statistical analysis of the molecular parameters of those compounds.

In some embodiments, the machine learning algorithm is selected from the group consisting of: a naive Bayes model, a naive Bayes bitvectors model, a decision tree model, a random forest model, a LogReg model, and a boosting model. In some embodiments, the boosting model includes the XGBoost algorithm.

In some embodiments, the molecular parameters further include one or more of physical information about the compound, and chemical information about the compound.

Under another aspect, a computer system for predicting cardiotoxicity of molecular parameters of a compound is provided. The computer system includes a processor; and at least one computer-readable medium. The medium can store the molecular parameters of the compound, the molecular parameters including at least structural information about the compound. The medium also can store a machine learning algorithm having been trained using respective molecular parameters of compounds known to have cardiotoxicity and of compounds known not to have cardiotoxicity. The medium also can store instructions for causing the processor to perform steps including: providing as input to the machine learning algorithm the molecular parameters of the compound; and receiving as output from the machine learning algorithm a representation of the predicted cardiotoxicity of each molecular parameter of at least a subset of the molecular parameters of the compound.

In some embodiments, the representation of the predicted cardiotoxicity includes, for each molecular parameter of at least a subset of the molecular parameters of the compound, a numerical value representing the predicted cardiotoxicity of that molecular parameter.

In some embodiments, the at least one computer-readable medium further stores instructions for causing the processor to redesign the compound so as not to include at least one of the molecular parameters of at least the subset.

In some embodiments, the at least one computer-readable medium further stores instructions for causing the processor to: provide as input to the machine learning algorithm the molecular parameters of the redesigned compound; and receive as output from the machine learning algorithm a representation of the predicted cardiotoxicity of each molecular parameter of at least a subset of the molecular parameters of the redesigned compound.

In some embodiments, the representation includes a value representative of a prediction that the molecular parameter of at least the subset will cause the compound to block two or more cardiac ion protein channels. In some embodiments, the two or more cardiac ion protein channels are selected from the group consisting of: sodium ion channel proteins, calcium ion channel proteins, and potassium ion channel proteins. In some embodiments, the potassium ion channel protein hERG1, the sodium ion channel protein is hNaᵥ1.5, or the calcium channel protein is hCav1.2.

In some embodiments, the at least one computer-readable medium further stores instructions for causing the processor to: provide as input to the machine learning algorithm respective molecular parameters of a plurality of compounds of which the previously recited compound is a member; receive as output from the machine learning algorithm a representation of the predicted cardiotoxicity of each molecular parameter of at least a subset of the molecular parameters of each of the compounds of the plurality of compounds; and select a compound of the plurality of compounds based on the predicted cardiotoxicity of each molecular parameter of at least a subset of the molecular parameters of each of the compounds of the plurality of compounds.

In some embodiments, the compounds known to have cardiotoxicity and the compounds known not to have cardiotoxicity are selected based on a statistical analysis of the molecular parameters of those compounds.

In some embodiments, the machine learning algorithm is selected from the group consisting of: a naive Bayes model, a naive Bayes bitvectors model, a decision tree model, a random forest model, a LogReg model, and a boosting model. In some embodiments, the boosting model includes the XGBoost algorithm.

In some embodiments, the molecular parameters are selected from the group consisting of: structural information about the compound, physical information about the compound, and chemical information about the compound.

Under another aspect, at least one computer-readable medium for use in predicting cardiotoxicity of molecular parameters of a compound is provided. The at least one computer-readable medium stores: the molecular parameters of the compound, the molecular parameters including at least structural information about the compound; a machine learning algorithm having been trained using respective molecular parameters of compounds known to have cardiotoxicity and of compounds known not to have cardiotoxicity; and instructions for causing a processor to perform steps including: providing as input to the machine learning algorithm the molecular parameters of the compound; and receiving as output from the machine learning algorithm a representation of the predicted cardiotoxicity of each molecular parameter of at least a subset of the molecular parameters of the compound.

### BRIEF DESCRIPTION OF THE FIGURES

FIG. 1A illustrates steps in an exemplary method of predicting cardiotoxicity of molecular parameters of a compound, according to some embodiments of the present invention.

FIG. 1B illustrates steps in an exemplary method of training a machine learning algorithm for predicting cardiotoxicity of molecular parameters of a compound, according to some embodiments of the present invention.

FIG. 2 illustrates an exemplary system for predicting cardiotoxicity of molecular parameters of a compound, according to some embodiments of the present invention.

FIG. 3A illustrates an exemplary probability distribution of mutual similarity among a plurality of compounds that are known to have cardiotoxicity and a plurality of compounds that are known not to have cardiotoxicity. Inset illustrates result of primary component analysis of such compounds.

FIG. 3B illustrates an exemplary probability distribution of mutual similarity among a subset of compounds that are known to have cardiotoxicity and a subset of compounds that are known not to have cardiotoxicity. Inset illustrates result of primary component analysis of such compounds.

FIGS. 4A-4B respectively illustrate exemplary IC50 and pIC50 values of an exemplary set of compounds, according to some embodiments of the present invention.

FIGS. 5A-5J illustrate ROC curves for an exemplary training set and test sets for exemplary machine learning algorithms, according to some embodiments of the present invention.

FIGS. 6A-6E illustrate exemplary performance measures of exemplary machine learning algorithms, according to some embodiments of the present invention.

FIGS. 7A-7C illustrate ROC curves for an exemplary training set, test set, and validation set for exemplary machine learning algorithms, according to some embodiments of the present invention.

FIG. 8 illustrates exemplary prediction accuracies for an exemplary training set, test set, and validation set for exemplary machine learning algorithms, according to some embodiments of the present invention.

FIG. 9 illustrates histograms showing exemplary predicted or actual numbers of active (1.0 on x-axis) and inactive (0.0 on x-axis) compounds in an exemplary test set with respect to different exemplary machine learning algorithms, according to some embodiments of the present invention. The histogram activities shows the actual distribution.

FIGS. 10A-10G illustrate exemplary performances of different exemplary machine learning algorithms with respect to an exemplary validation set, according to some embodiments of the present invention. Compounds with IC50 of less than or equal to 10 µM were considered “active.” The left-most panels indicate an exemplary probability to be active, the middle panels indicate an exemplary corresponding classification over the experimental pIC50 values, and the right-most panels are corresponding ROC curves.

FIG. 11 illustrates an exemplary heatmap of the mutual correlation coefficients of all features in an exemplary training set, according to some embodiments of the present invention.

FIGS. 12A-12H illustrate exemplary ROC curves for an exemplary training set and test set for exemplary machine learning algorithms using isomapping, according to some embodiments of the present invention.

FIGS. 13A-13E illustrate exemplary performance measures of exemplary machine learning algorithms using isomapping, according to some embodiments of the present invention.

FIGS. 14A-14C illustrate ROC curves for false positives for an exemplary training set, test set, and validation set for exemplary machine learning algorithms, according to some embodiments of the present invention.

FIG. 15 illustrates ROC curves for compounds in an exemplary training set for a NULL machine learning algorithm, according to some embodiments of the present invention.

FIGS. 16A-16D illustrate performance of an exemplary 3C model for assessment of torsadogenic potential for a blinded set of blockers, according to some embodiments of the present invention.

FIGS. 17A-17J illustrate probabilities to be active and ROC curves for an exemplary validation set for different machine learning algorithms, according to some embodiments of the present invention.

FIGS. 18A-18B respectively illustrate probabilities to be active and ROC curves for an exemplary validation set for a consensus among different machine learning algorithms, according to some embodiments of the present invention.

FIGS. 19A-19D illustrate probabilities to be active on hERG (yellow) with respect to antifungal activity (blue) for an exemplary set of compounds, according to some embodiments of the present invention.

FIG. 20 illustrates the pIC50 values of an exemplary set of compounds, according to some embodiments of the present invention.

FIG. 21A illustrates an example of the boosting model on a specific dimension of an input vector, according to some embodiments of the present invention.

FIG. 21B illustrates an example of a pair of an active compound (left) and inactive (right) where a change of a chemical group leads to a shift in the class probability.

FIG. 22 illustrates illustrate an AROC histogram of the molecular descriptors, according to some embodiments of the present invention.

FIGS. 23A-D illustrate ROC curves of the most predictive molecular descriptors (AROC>0.55) (FIG. 23A), normal features (FIG. 23B), 2D-pharmacophore features (FIG. 23C) and similarity based features (FIG. 23D), according to some embodiments of the present invention.

FIG. 24 illustrates steps in an exemplary method of selecting a model of molecular parameters of a compound that can be used for predicting cardiotoxicity of the compound, according to some embodiments of the present invention.

FIGS. 25A-D illustrate mean cross-validated R2 (Q2) from 10 fold cross-validation (FIG. 24A), the mean cross-validated AROC (cvAROC) from 10 fold cross-validation (FIG. 24C) and the corresponding box plots (FIGS. 24B and 24D), according to some embodiments of the present invention.

FIGS. 26A-D illustrate learning curves for different numbers of iterations (N) (feature set; 8, parameter set: 5). The error bars indicate the standard deviation of five repetitions with randomly selected training and test sets, according to some embodiments of the present invention.

FIG. 27A illustrates the correlation of fitted data with experimental data, the dashed line shows perfect correlation, the vertical and horizontal dashed lines show the cutoffs used for class and classification (>5 active), according to some embodiments of the present invention.

FIG. 27B illustrates the corresponding ROC curve using same class criterion as illustrated in FIG. 27A, according to some embodiments of the present invention. The dashed line shows the random distribution and the shaded area shows expected variance of random prediction.

FIGS. 28A-C illustrate model performance for test set 1, according to some embodiments of the present invention. FIG. 27A illustrates correlation with experimental data, the dashed line shows perfect correlation, horizontal and vertical dashed lines show cutoffs used for class and classification (>5 active). FIG. 27B illustrates ROC curve using same class criterion. The dashed line shows random distribution, and the shaded area shows expected variance of random prediction. FIG. 27C illustrates error over the distance to the training set for each compound.

FIG. 29A illustrates the approximated distribution of pIC50 values in training set and test sets, according to some embodiments of the present invention.

FIG. 29B illustrates the approximated distribution of the maximum similarities to compounds in the training set for all test sets, according to some embodiments of the present invention. For the training set the similarity to the next most similar compound is shown.

FIGS. 30A-C illustrate model performance for test set 2, according to some embodiments of the present invention.

FIGS. 31A-C illustrate model performance for test set 3, according to some embodiments of the present invention.

FIGS. 32A-C illustrate model performance for test set 4, according to some embodiments of the present invention.

FIGS. 33A-C illustrate model performance for combined test sets, according to some embodiments of the present invention.

FIGS. 34A and 34B illustrate the relationship, including AROC, R² and r, between the minimal distance to the training set (MDT) by combining all test sets, according to some embodiments of the present invention.

FIG. 35 illustrates the top 50 relative feature scores for the final XGBoost model, according to some embodiments of the present invention.

FIG. 36 illustrates the accumulated number of compounds per publications and separation in training and test sets (Publications ranked by number of compounds), according to some embodiments of the present invention.

FIG. 37 illustrates predicted activity data.

FIG. 38 illustrates exploratory data analysis for active compounds.

FIGS. 39-41 illustrate similarity heat maps.

FIG. 42 illustrates probability distribution of mutual similarity.

FIG. 43 illustrates interrelations of molecular properties.

FIG. 44 illustrates chemical group analysis.

FIG. 45 illustrates chemical moieties correlations.

FIG. 46 illustrates null models.

FIG. 47 illustrates logistic regression models.

FIG. 48 illustrates logistic regression models.

FIG. 49 illustrates naïve Bayes models.

FIG. 50 illustrates naïve Bayes models.

FIG. 51 illustrates naïve Bayes bitvectors.

FIG. 52 illustrates naïve Bayes bitvectors.

FIG. 53 illustrates decision tree models.

FIG. 54 illustrates decision tree models.

FIG. 55 illustrates random Forest models.

FIG. 56 illustrates random Forest models.

FIG. 57 illustrates boosting models.

FIG. 58 illustrates boosting models.

FIG. 59 illustrates prediction accuracy over number of features.

FIG. 60 illustrates model performance: training set.

FIG. 61 illustrates model performance: test.

FIG. 62 illustrates model performance: validation.

FIG. 63 illustrates accuracy comparison of various models.

FIG. 64 illustrates histograms of predicted classes: training set.

FIG. 65 illustrates histograms of predicted classes: test set.

FIG. 66 illustrates histograms of predicted classes: validation set.

FIG. 67 illustrates k-fold cross-validation for k = 132.

FIG. 68 illustrates R² correlation coefficients to experimentally available data used as test set for model validation.

FIG. 69 illustrates comparisons to available software-solutions for the same set (Experimental IC₅₀ to Schrodinger QPlogHERG).

FIG. 70 illustrates binary prediction of hERG versus hERG IC₅₀.

FIG. 71 illustrates exploratory data analysis for active compounds.

FIGS. 72-74 illustrate similarity heat maps.

FIG. 75 illustrates interrelations of molecular properties.

FIG. 76 illustrates probability distribution of mutual similarity.

FIG. 77 illustrates probability distribution of mutual similarity.

FIGS. 78-96 illustrate activity bar graphs.

FIGS. 97A-C illustrate activity bar graphs.

FIG. 98 illustrates chemical group analysis.

FIG. 99 illustrates null models.

FIGS. 100A and 100B illustrate logistic regression models.

FIGS. 101A and 101B illustrate naïve Bayes models.

FIGS. 102A and 102B illustrate decision tree models.

FIGS. 103A and 103B illustrate random Forest models.

FIGS. 104A and 104B illustrate boosting models.

FIG. 105 illustrates logistic regression model

FIGS. 106A-D illustrate number of features and prediction accuracy for various models.

FIG. 107 illustrates model performance: training data.

FIG. 108 illustrates boxplots grouped by activity for various models.

FIG. 109 illustrates histograms of predicted probabilities: training set.

FIG. 110 illustrates model performance: test data.

FIG. 111 illustrates boxplots grouped by activity for various models.

FIG. 112 illustrates histograms of predicted probabilities: testset.

FIG. 113 illustrates correlations for various models.

FIG. 114 illustrates model performance: validation.

FIG. 115 illustrates boxplots grouped by activity for various models.

FIG. 116 illustrates histograms of predicted probabilities for various models.

FIG. 117 illustrates k-fold cross-validation for k = 94.

FIGS. 118A and 118B illustrate exploratory data analysis for active compounds.

FIGS. 119-121 illustrate similarity heat maps.

FIG. 122 illustrates probability distribution of mutual similarity.

FIGS. 123-128 illustrate activity bar graphs.

FIG. 129 illustrates chemical group analysis.

FIG. 130 illustrates null models.

FIGS. 131A and 131B illustrate logistic regression models.

FIGS. 132A and 132B illustrate naïve Bayes models.

FIGS. 133A and 133B illustrate decision tree models.

FIGS. 134A and 134B illustrate random Forest models.

FIGS. 135A and 135B illustrate boosting models.

FIG. 136 illustrates logistic regression models.

FIG. 137 illustrates naïve Bayes models.

FIG. 138 illustrates decision tree models.

FIG. 139 illustrates random Forest models.

FIG. 140 illustrates boosting models.

FIG. 141 illustrates model performance: training data.

FIG. 142 illustrates boxplots grouped by activity for various models.

FIG. 143 illustrates histograms of predicted probabilities for various models.

FIG. 144 illustrates model performance: test data.

FIG. 145 illustrates boxplots grouped by activity for various models.

FIG. 146 illustrates histograms of predicted probabilities for various models.

FIG. 147 illustrates correlations for various models.

FIG. 148 illustrates k-fold cross-validation for k = 94.

### DETAILED DESCRIPTION

Embodiments of the present invention provide systems and methods for predicting cardiotoxicity of molecular parameters of a compound based on machine learning algorithms. For example, systems and methods are provided for predicting with improved accuracy, based on molecular parameters, whether a compound may block one or more, or even two or more, cardiac ion protein channels. For example, in some embodiments, activity of a given compound is predicted not only with respect to the hERG potassium channel, disclosed herein, but also with respect to one or more other cardiac ion protein channels, such as the hNaᵥ1.5 sodium channel, disclosed herein, and the hCaᵥ1.2 calcium channel, disclosed herein. As used herein, “active” compounds are considered to be compounds that have an IC50 value of lower than 10 µM with respect to blocking a cardiac ion protein channel, including but not limited to hERG, hNaᵥ1.5, or hCav1.2. The present systems and methods can facilitate accurate and rapid pre-clinical and pre-synthetic screening programs for pipelines of compounds. In comparison, other types of computational analysis can be so computationally time consuming as to preclude performing such analysis on practically useful numbers of compounds.

It should be appreciated the assessment of cardiotoxicity has become important for the approval of new compounds as pharmaceutical drugs. Demand for computational assessments of cardiotoxicity also is likely to increase. For many targets (including desired and unintended targets), extensive assay libraries are publicly available and are stored in databases such as ChEMBL (available online at www.ebi.ac.uk/chembl/ and operated by the European Molecular Biology Laboratory-European Bioinformatics Institute). Currently, there appears to be no crystal structure available for hERG. Therefore, structure based drug design has been done with homology models. Available models of hERG typically include the pore region of the potassium channel and in some cases the voltage sensing domains. hERG, however, has been studied extensively and a large amount of ligand affinities has been collected and is provided in databases which are publicly accessible. In the case of hERG, it has been suggested that molecules do not only bind to the inner cavity, but also target a binding site at the voltage sensors (NS1643). It is also likely that residues in the binding site adopt different conformations for different ligands. By modeling docking with just a single structure potentially can neglect compounds that bind to a different conformation or a different binding site. Additionally, different stereoisomers can have different affinities. However, the present systems and methods need not necessarily depend on the particular structure of the target, nor on the conformation of the compound.

In some embodiments, “activity” can include a binary definition, e.g., a definition of a compound, as a whole, being either active or inactive. Additionally, the present systems and methods can provide finer binning, ranges of pIC50, or raw pIC50 values, and per-group decomposition with statistical weights corresponding to risk factors associated with the functional group or other molecular parameter. Probabilities for a compound to be active can be output. For example, the present systems and methods can correlate molecular parameters with experimental data, so that a user can be provided with an estimation about the affinity. Some implementations can use a linear regression model that directly predicts pIC50s. Other embodiments readily can be envisions based on the teachings herein.

### 6.1 Cardiac Ion Protein Channels

### 6.1.1 Human Ether-a-Go-Go Related Gene 1 (hERG1) Channel

Cardiotoxicity is a leading cause of attrition in clinical studies and post-marketing withdrawal. The human Ether-a-go-go Related Gene 1 (hERG1) K⁺ ion channel is implicated in cardiotoxicity, and the U.S. Food and Drug Administration (FDA) requires that candidate drugs be screened for activity against the hERG1 channel. Recent investigations suggest that non-hERG cardiac ion channels are also implicated in cardiotoxicity. Therefore, screening of candidate drugs for activity against cardiac ion channels, including hERG1, is recommended.

The hERG1 ion channel (also referred to as KCNH2 or Kv11.1) is a key element for the rapid component of the delayed rectified potassium currents (I_{Kr}) in cardiac myocytes, required for the normal repolarization phase of the cardiac action potential (Curran et al., 1995, “A Molecular Basis for Cardiac-Arrhythmia; HERG Mutations Cause Long QT Syndrome,” Cell, 80, 795-803; Tseng, 2001, “I(Kr): The hERG Channel,” J. Mol. Cell. Cardiol., 33, 835-49; Vandenberg et al., 2001, “HERG Kþ Channels: Friend and Foe,” Trends. Pharm. Sci. 22, 240-246). Loss of function mutations in hERG1 cause increased duration of ventricular repolarization, which leads to prolongation of the time interval between Q and T waves of the body surface electrocardiogram (long QT syndrome-LQTS) (Vandenberg et al., 2001; Splawski et al., 2000, “Spectrum of Mutations in Long-QT Syndrome Genes KVLQT1, HERG, SCN5A, KCNE1, and KCNE2,” Circulation, 102, 1178-1185; Witchel et al., 2000, “Familial and Acquired Long QT Syndrome and the Cardiac Rapid Delayed Rectifier Potassium Current, Clin. Exp. Pharmacol. Physiol., 27, 753-766). LQTS leads to serious cardiovascular disorders, such as tachyarrhythmia and sudden cardiac death.

Diverse types of organic compounds used both in common cardiac and noncardiac medications, such as antibiotics, antihistamines, and antibacterials, can reduce the repolarizing current I_{Kr} (i.e., with binding to the central cavity of the pore domain of hERG1) and lead to ventricular arrhythmia (Lees-Miller et al., 2000, “Novel Gain-of-Function Mechanism in Kþ Channel-Related Long-QT Syndrome: Altered Gating and Selectivity in the HERG1 N629D Mutant,” Circ. Res., 86, 507-513; Mitcheson et al., 2005, “Structural Determinants for High-affinity Block of hERG Potassium Channels,” Novartis Found. Symp. 266, 136-150; Lees-Miller et al., 2000, “Molecular Determinant of High-Affinity Dofetilide Binding to HERG1 Expressed in Xenopus Oocytes: Involvement of S6 Sites,” Mol. Pharmacol., 57, 367-374). Therefore, several approved drugs (i.e., terfenadine, cisapride, astemizole, and grepafloxin) have been withdrawn from the market, whereas several drugs, such as thioridazine, haloperidol, sertindole, and pimozide, are restricted in their use because of their effects on QT interval prolongation (Du et al., 2009, “Interactions between hERG Potassium Channel and Blockers,” Curr. Top. Med. Chem., 9, 330-338; Sanguinetti et al., 2006, “hERG Potassium Channels and Cardiac Arrhythmia,” Nature, 440, 463-469).

Accordingly, in some embodiments of the systems and methods disclosed herein, the cardiac ion protein channel is the Human Ether-a-go-go Related Gene 1 (hERG1) channel. The DNA and amino acid sequences for hERG1 are provided as SEQ ID NO: 1 and SEQ ID NO: 2, respectively. Without being limited by any theory, in one aspect of the disclosure, the blocking of the central pore cavity or channel of hERG by a drug is a predictor of the cardiotoxicity of the drug. Undesired drug blockade of K⁺ ion flux in hERG1 can lead to long QT syndrome, eventually inducing fibrillation and arrhythmia. hERG1 blockade is a significant problem experienced during the course of many drug discovery programs.

### 6.1.2 Human Naᵥ1.5 Voltage Gated Sodium Channel:

The Naᵥ1.5 voltage gated sodium channel (VGSC) is responsible for initiating the myocardial action potential and blocking Naᵥ1.5 through either mutations or its interactions with small molecule drugs or toxins have been associated with a wide range of cardiac diseases. These diseases include long QT syndrome 3 (LQT3), Brugada syndrome 1 (BRGDA1) and sudden infant death syndrome (SIDS).

Accordingly, in other embodiments of the systems and methods disclosed herein, the cardiac ion protein channel is the hNaᵥ1.5 voltage gated sodium channel. The DNA and amino acid sequences for hNaᵥ1.5 are provided as SEQ ID NO: 3 and SEQ ID NO: 4, respectively.

Without being limited by any theory, in one aspect of the disclosure, the blocking of the central pore cavity or channel of hNaᵥ1.5 by a drug is a predictor of the cardiotoxicity of the drug. Undesired drug blockade of Na⁺ ion flux in hNaᵥ1.5 can lead to long QT syndrome, eventually inducing fibrillation and arrhythmia. Blockage of hNaᵥ1.5 is a significant problem experienced during the course of many drug discovery programs. For example, ranolazine is understood to block only the slowly inactivating component of the sodium current.

### 6.1.3 Human Caᵥ1.2 Voltage Gated Calcium Channel:

The Caᵥ1.2 voltage gated calcium channel is also responsible for mediating the entry of calcium ions into excitable cells and blocking Caᵥ1.2 through either mutations or its interactions with small molecule drugs or toxins have been associated with a wide range of cardiac diseases. These diseases include long QT syndrome 3 (LQT3); Brugada syndrome 1 (BRGDA1); inherited neuronal ion channelopathies such as described in Catterall et al., “Inherited neuronal channelopathies: New windows on complex neurological diseases,” J. Neurosci. 28(46): 11768-11777 (2008),” the entire contents of which are incorporated by reference herein; and atrial fibrillation, which can have a genetic component, such as described in Christophersen et al., “Genetics of atrial fibrillation: From families to genomes,” J Hum. Genet. 2015 May 21, doi: 10.1038/jhg.2015.44 (epub ahead of print), the entire contents of which are incorporated by reference herein.

Accordingly, in still other embodiments of the systems and methods disclosed herein, the cardiac ion protein channel is the hCaᵥ1.2 voltage gated calcium channel. The DNA and amino acid sequences for hCaᵥ1.2 are provided as SEQ ID NO: 5 and SEQ ID NO: 6, respectively.

Without being limited by any theory, in one aspect of the disclosure, the blocking of the central pore cavity or channel of hCaᵥ1.2 by a drug is a predictor of the cardiotoxicity of the drug. Undesired drug blockade of Ca⁺² ion flux in hCaᵥ1.2 can lead to long QT syndrome, eventually inducing fibrillation and arrhythmia. Blockage of hCaᵥ1.2 is a significant problem experienced during the course of many drug discovery programs.

### 6.2 Compounds

In some embodiments of the systems and methods disclosed herein, the compound is selected from a list of compounds that have failed in clinical trials, or were halted in clinical trials due to cardiotoxicity. Such compounds could benefit from a structural prediction of the molecular parameter or subset of molecular parameters that may be responsible for blocking two or more of the cardiac ion protein channels disclosed herein.

Accordingly, in some embodiments, the compound is selected from Table 1, below:

TABLE 1

Cardiac Hazardous Drugs

| Category of Drug | Drug |
|---|---|
| Calcium channel blockers | Prenylamine (TdP reported; withdrawn) |
| | Bepridil (TdP reported; withdrawn) |
| | Terodiline (TdP reported; withdrawn) |
| Psychiatric drugs | Thioridazine (TdP reported) |
| | Chlorpromazine (TdP reported) |
| | Haloperidol (TdP reported) |
| | Droperidol (TdP reported) |
| | Amitriptyline |
| | Nortriptyline |
| | Imipramine (TdP reported) |
| | Desipramine (TdP reported) |
| | Clomapramine |
| | Maprotiline (TdP reported) |
| | Doxepin (TdP reported) |
| | Lithium (TdP reported) |
| | Chloral hydrate |
| | Sertindole (TdP reported; withdrawn in the UK) |
| | Pimozide (TdP reported) |
| | Ziprasidone |
| Antihistamines | Terfenadine (TdP reported; withdrawn in the USA) |
| | Astemizole (TdP reported) |
| | Diphenhydramine (TdP reported) |
| | Hydroxyzine |
| | Ebastine |
| | Loratadine |
| | Mizolastine |
| Antimicrobial and antimalarial drugs | Erythromycin (TdP reported) |
| | Clarithromycin (TdP reported) |
| | Ketoconazole |
| | Pentamidine (TdP reported) |
| | Quinine |
| | Chloroquine (TdP reported) |
| | Halofantrine (TdP reported) |
| | Amantadine (TdP reported) |
| | Sparfloxacin |
| | Grepafloxacin (TdP reported; withdrawn) |
| | Pentavalent antimonial meglumine |
| Serotonin agonists/antagonists | Ketanserin (TdP reported) |
| | Cisapride (TdP reported; withdrawn) |
| Immunosuppressant | Tacrolimus (TdP reported) |
| Antidiuretic hormone | Vasopressin (TdP reported) |
| Other agents | Adenosine |
| | Organophosphates |
| | Probucol (TdP reported) |
| | Papaverine (TdP reported) |
| | Cocaine |

In other embodiments, the compound is an anticancer agent, such as an anthracycline, mitoxantrone, cyclophosphamide, fluorouracil, capecitabine and trastuzumab. In some embodiments, the compound is an immunomodulating drug, such as interferon-alpha-2, interleukin-2, infliximab and etanercept. In some embodiments, the compound is an antidiabetic drug, such as rosiglitazone, pioglitazone and troglitazone. In some embodiments, the compound is an antimigraine drug, such as ergotamine and methysergide. In some embodiments, the compound is an appetite suppressant, such as fenfulramine, dexfenfluramine and phentermine. In some embodiments, the compound is a tricyclic antidepressants. In some embodiments, the compound is an antipsychotic drug, such as clozapine. In some embodiments, the compound is an antiparkinsonian drug, such as pergolide and cabergoline. In some embodiments, the compound is a glucocorticoid. In some embodiments, the compound is an antifungal drugs such as itraconazole and amphotericin B. In some embodiments, the compound is an NSAID, including selective cyclo-oxygenase (COX)-2 inhibitors.

In still other embodiments, the compound is an antihistamine, an antiarrhythmic, an antianginal, an antipsychotic, an anticholinergic, an antitussive, an antibiotic, an antispasmodic, a calcium antagonist, an inotrope, an ACE inhibitor, an antihypertensive, a beta-blocker, an antiepileptic, a gastroprokinetic agent, an alpha1-blocker, an antidepressant, an aldosterone antagonist, an opiate, an anesthetic, an antiviral, a PDE inhibitor, an antifungal, a serotonin antagonist, an antiestrogen, or a diuretic.

In still other embodiments, the compound is an active ingredient in a natural product.

In still other embodiments, the compound is a toxin or environmental pollutant.

In still other embodiments, the compound is an antiviral agent. For example, in some embodiments, the compound is selected from the group consisting of a protease inhibitor, an integrase inhibitor, a chemokine inhibitor, a nucleoside or nucleotide reverse transcriptase inhibitor, a non-nucleoside reverse transcriptase inhibitor, and an entry inhibitor.

In still other embodiments, the compound is capable of inhibiting hepatitis C virus (HCV) infection. For example, in some embodiments, the compound is an inhibitor of HCV NS3/4A serine protease. In some embodiments, the compound is an inhibitor of HCV NS5B RNA dependent RNA polymerase. In some embodiments, the compound is an inhibitor of HCV NS5A monomer protein.

In still other embodiments, the compounds is selected from the group consisting of Abacavir, Aciclovir, Acyclovir, Adefovir, Amantadine, Amprenavir, Ampligen, Arbidol, Atazanavir, Balavir, Boceprevirertet, Cidofovir, Darunavir, Delavirdine, Didanosine. Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Interferon type III, Interferon type II, Interferon type I, Interferon, Lamivudine, Lopinavir, Loviride, Maraviroc, Moroxydine, Methisazone, Nelfinavir, Nevirapine, Nexavir, Oseltamivir (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza), and Zidovudine.

### 6.3 Systems and Methods

In some embodiments, the present systems and methods encompass preprocessing of data regarding compounds available for use in preparing training sets, test sets, and validation sets respectively for use in training, testing, or validating a machine learning algorithm. For example, higher accuracies have been observed in validation set that was generated from the same population of compounds as the training set, but lower accuracies have been observed for independent validation sets that were not generated from the same population as the data set. The apparently exaggerated accuracy in the circumstance where the validation set was generated from the same population of compounds as the training set is believed to arise from confounding, e.g., correlation with third variables. Confounding typically is reduced via randomization or stratification of the population of compounds. However, to stratify a dataset of compounds can be relatively difficult for high dimensional data and even may remove the actual information that distinguishes active from inactive compounds. Regarding randomization, the samples of active an inactive compounds in a database such as ChEMBL can be biased by the nature of experiments which were performed in preparing the database. For example, experiments can be designed to find and report active compounds and can focus on derivatives that share high structural similarity. Randomization can reduce such a bias, but can sacrifice instances that can be valuable for the model building process. Accordingly, without randomization or stratification, the reported errors in validation sets should be interpreted with special care. Therefore, it is recommended to at least one alternative validation set that originates from a different population than the training and test sets. Additionally, as provided in greater detail herein, the present systems and methods, the compounds used to prepare the training set, test set, and validation set can be selected based on a statistical analysis of the molecular parameters of those compounds.

Note that although compounds can be considered “active” based on their activity, the data in databases such as ChEMBL can include different assays in different cell lines, and therefore the apparent activity of a compound can be based, in part, upon the particular assay or cell line used, rather than being based on the interaction between the compound and the target. Accordingly, it can be desirable to include in the data set only compounds for which activity was assessed using a single specific assay, because the IC50 depends on the environment in the cell (e.g., ion concentration, pH, and the like). However, such a strategy may exclude too many compounds, thus yielding a training set, test set, and validation set that are too small to accurately train and assess a machine learning algorithm. In some embodiments, the machine-learning based systems and methods provided herein include creating training and validation sets based on one or more single-source cell lines that overexpress hERG and NaV1.5 channels. Additionally, potential variability of data from various cell lines can be accounted for based on literature and data mining on available data from clinical trials and reported torsadogenic activity for large panels of compounds as an additional training descriptor correlated to molecular parameters such as can be used herein.

The present systems and methods are believed to be useful for targets with or without structural model, and for which binding assays have been performed for a hundred compounds or more, or on the order of hundreds of compounds (or more). The present systems and methods are believed to facilitate the process of drug optimization and can serve as a warning system for structures which are likely to reveal interactions between a compound and an unintended target, particularly, a cardiac ion protein channel. Therefore, the present systems and methods can support both positive design (optimization of affinity against target) and negative design (increase of specificity for target). An exemplary advantage of providing predictions of cardiotoxicity based on molecular parameters is the relatively short computational time, thus facilitating rapid screening of a large number of compounds.

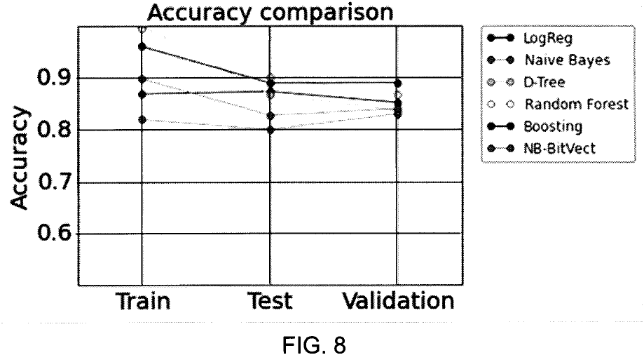

Drug induced QT prolongation is known to be a multi-channel phenomenon in which hERG blockage contributes, noting that there are FDA approved drugs that block hERG but do not prolong the QT interval. The present systems and methods optionally encompass multi-target approaches to predict the occurrence of drug induced QT prolongation more accurately by predicting a compound’s interactions with multiple ion channels. In one nonlimiting example, a polynomial regression models based machine learning algorithm received as input various molecular parameters (e.g., solubility, lipophilicity, molecular weight, number of specific atoms, molecular fingerprints and other molecular and structural properties, such as distances between atoms and groups of atoms or groups with distinct functions such as hydrogen donors or aceptors) for compounds in an exemplary dataset of established blockers of hERG1, Caᵥ1.2 and Naᵥ1.5 channels with reported dysrhythmic activity and electrophysiological data. In one nonlimiting example, described in greater detail below in the “Examples” section, the Pearson correlation coefficients in the validation set between experimental and predicted pIC50 of hERG/Naᵥ1.5/Caᵥ2.1 model are 0.78/0.6/0.51 respectively (with saquinavir as clear outlier in all datasets) with blinded predictive power in torsadogenic activity of ~70% for identification of true-positives (torsadogenic) and 49% of true-negatives (non-torsadogenic). Therefore, the preliminary model described in greater detail below with reference to FIG. 8 can provide enhanced accuracy relative to single-channel based predictive platforms. For example, various models have been generated to predict hERG blockade with prediction accuracies above 80% for relatively small and curated data sets. The models can be distributed independently and optionally can be combined into a multi-target prediction system, that allows one to optimize drugs with respect to various targets simultaneously. Optionally, structure-based results, e.g., from molecular docking, in principle can be integrated into the model design, as soon as structural models of the targets are available. For example, the binding affinities to different conformational states of hERG1 channel are known to correlate strongly with the blocker’s efficacy. The analysis of different molecular parameters, e.g., molecular group decomposition, from the present systems and methods can aid results of receptor-drug modeling, thus facilitating identification of potentially dangerous moieties (e.g., chemical groups) in the assessed groups of the compounds. Characteristic features of large data sets of compounds, which can have varying chemical scaffolds, then can be developed or identified.

In one nonlimiting example, several thousands of compounds can be estimated on a single CPU in a few minutes and the approach can be easily run in parallel. Optionally, based on the predicted cardiotoxicity of certain molecular parameters, a compound can be redesigned.

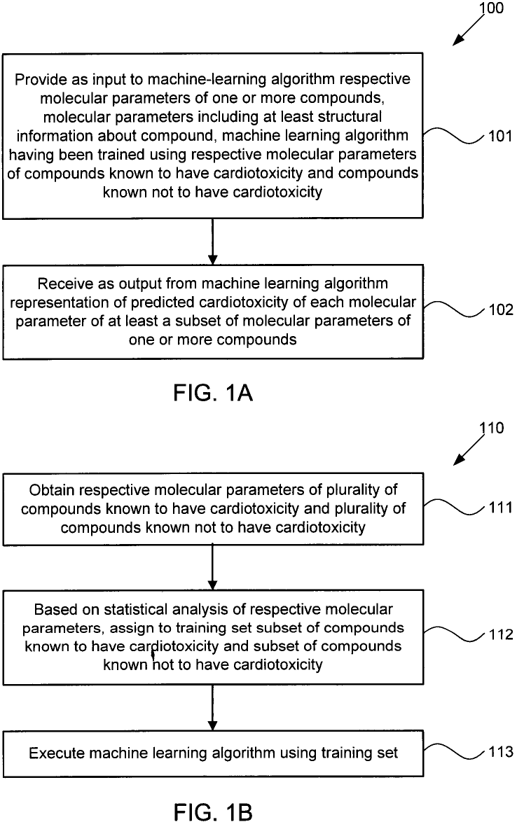

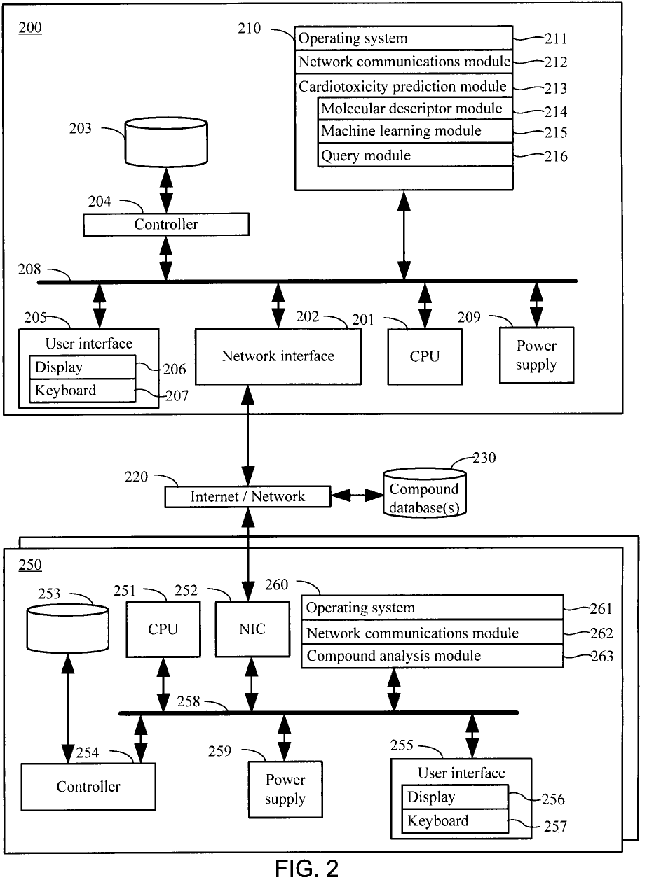

FIG. 1A illustrates steps in an exemplary method of predicting cardiotoxicity of molecular parameters of a compound, according to some embodiments of the present invention. Method 100 illustrated in FIG. 1A includes providing as input to a machine learning algorithm respective molecular parameters of one or more compound (step 101). In some embodiments, the molecular parameters can include at least structural information about the compound(s) (step 101). By “structural information” it is meant information regarding the presence and relative arrangement of atoms within the compound, e.g., within different portions of the compound. Other exemplary molecular parameters include, but are not limited to, physical information about the compound(s) and chemical information about the compound(s). Physical information about the compound(s) can include, but is not limited to, molecular weight, number of atoms and rings, and molecular volume. Chemical information about the compound(s) can include, but is not limited to, polarity, and the number of certain chemical groups. Other examples of physical and chemical information about the compound(s) are provided elsewhere herein. In one example, a suitably programmed computer such as described below with reference to FIG. 2 suitably can obtain the molecular parameters via a user interface, via the network, or from a local or remote computer-readable medium, such as the ChEMBL database. The computer can store the molecular parameters in any suitable computer-readable medium. In one nonlimiting example, the computer obtains the molecular parameters for each compound in the form of a SMILES (simplified molecular-input line-entry system) file such known in the art.

In some embodiments, the machine learning algorithm has been trained using respective molecular parameters of compounds known to have cardiotoxicity and of compounds known not to have cardiotoxicity (step 101). An exemplary method of training such a machine learning algorithm is provided below with reference to FIG. 1B. Exemplary machine learning algorithms include, but are not limited to, a naive Bayes model, a naive Bayes bitvectors model, a decision tree model, a random forest model, a LogReg model, and a boosting model. In some embodiments, the boosting model includes the XGBoost algorithm. In one nonlimiting example, the machine learning algorithm is stored on the same computer-readable medium as are the molecular parameters. In another nonlimiting example, the machine learning algorithm is stored in a different computer-readable medium than are the molecular parameters. The machine learning algorithm can receive as input the molecular parameters of the compound from the computer in any suitable manner. For example, in some embodiments, the same computer can obtain the molecular parameters and also can execute the machine learning algorithm, providing the molecular parameters to that algorithm. In other embodiments, a first computer can obtain the molecular parameters and can transmit the molecular parameters via any suitable wired or wireless communication channel to a second computer that can execute the machine learning algorithm, receiving the molecular parameters as input.

Method 100 illustrated in FIG. 1A further includes receiving as output from the machine learning algorithm a representation of the predicted cardiotoxicity of each molecular parameter of at least a subset of the molecular parameters of the one or more compounds (102). The machine learning algorithm can provide the output to the computer in any suitable manner. For example, in some embodiments, the same computer can execute the machine learning algorithm and receive the output from that algorithm. In other embodiments, a first computer can execute the machine learning algorithm and can transmit the output via any suitable wired or wireless communication channel to a second computer that can receive the output. The computer can store the output in any suitable computer-readable medium. In one nonlimiting example, the machine learning algorithm is stored on the same computer-readable medium as is the output. In another nonlimiting example, the machine learning algorithm is stored in a different computer-readable medium than is the output.

In one nonlimiting example, the representation of the predicted cardiotoxicity includes, for each molecular parameter of at least the subset of the molecular parameters of the compound, a numerical value representing the predicted cardiotoxicity of that molecular parameter. Table 2 illustrates an exemplary output including such a representation. In the examples provided in Table 2, it can be seen that “fr_piperidine” is associated with the greatest risk of cardiotoxicity, “TPSA” is associated with the next highest risk of cardiotoxicity, and so on.

TABLE 2

Exemplary Output

| Molecular Parameter (meaning) | Value |
|---|---|
| fr_piperidine (number of piperidine groups) | 0.73 |
| TPSA (topological surface area) | 0.71 |
| fr_halogen (number of halogens) | 0.67 |
| fNHAcc (fraction of hydrogen acceptors) | 0.67 |
| fNHDon (fraction of hydrogen donors) | 0.67 |
| LogP (logarithm of the partition coefficient) | 0.64 |
| NOCount (number of nitrogens and oxygens) | 0.64 |

In some embodiments, the representation provided as output in step 102 includes a value representative of a prediction that the molecular parameter of at least the subset will cause the compound to block two or more cardiac ion protein channels. In some embodiments, the two or more cardiac ion protein channels include two or more of sodium ion channel proteins, calcium ion channel proteins, and potassium ion channel proteins. Illustratively, the potassium ion channel protein can be hERG1, the sodium ion channel protein can be hNaᵥ1.5, and the calcium channel protein is hCav1.2. As noted elsewhere herein, the present systems and methods optionally can predict the cardiotoxicity of molecular parameters with respect to multiple targets. For example, the output provided in step 102 can include a plurality of predictions that the molecular descriptor will cause the compound to block a corresponding plurality of cardiac ion protein channels, e.g., two or more of hERG1, hNaᵥ1.5, and hCav1.2. In one example, the information from relative blockade of 3 major cardiac currents can be used as a safety assessment score generated from Rudy model of cardiac currents generating torsadogenicity metrics.

Optionally, method 100 illustrated in FIG. 1A further can include redesigning the compound so as not to include at least one of the molecular parameters of at least the subset. For example, based on the output of step 102, the computer can identify one or more molecular parameters that are predicted to be relatively cardiotoxic, and can modify such molecular parameter(s) so as to provide a compound having reduced predicted cardiotoxicity. For example, the computer can obtain the molecular parameters of one of the original compounds of step 101, and can “redesign” the compound by appropriately adjusting the value of one or more of such molecular parameters that are predicted to be relatively cardiotoxic. The computer then can execute steps 101 and 102 of method 100 based on the redesigned compound. For example, the computer can provide as input to the machine learning algorithm the molecular parameters of the redesigned compound in a manner analogous to that described above with reference to step 101; and can receive as output from the machine learning algorithm a representation of the predicted cardiotoxicity of each molecular parameter of at least a subset of the molecular parameters of the redesigned compound in a manner analogous to that described above with reference to step 102. Optionally, such compound redesign and re-analysis can be repeated any suitable number of times. Optionally, following one or more such redesign steps, the compound can be synthesized in a laboratory and evaluated for effectiveness with respect to the desired target, as well as for cardiotoxicity.

Additionally, note that method 100 optionally can be executed for any desired number of compounds. For example, method 100 further can include, by the computer, providing as input to the machine learning algorithm, respective molecular parameters of a plurality of compounds (of which the compound described above is a member), and can receive as output from the machine learning algorithm a representation of the predicted cardiotoxicity of each molecular parameter of at least a subset of the molecular parameters of each of the compounds of the plurality of compounds. Additionally, method 100 optionally includes, by the computer, selecting a compound of the plurality of compounds based on the predicted cardiotoxicity of each molecular parameter of at least a subset of the molecular parameters of each of the compounds of the plurality of compounds.

The machine learning algorithm used in method 100 can be trained using any suitable technique. In some embodiments, the compounds known to have cardiotoxicity and the compounds known not to have cardiotoxicity, upon which the machine learning algorithm is trained, can be selected based on a statistical analysis of the molecular parameters of those compounds. For example, FIG. 1B illustrates steps in an exemplary method of training a machine learning algorithm for predicting cardiotoxicity of molecular parameters of a compound, according to some embodiments of the present invention.

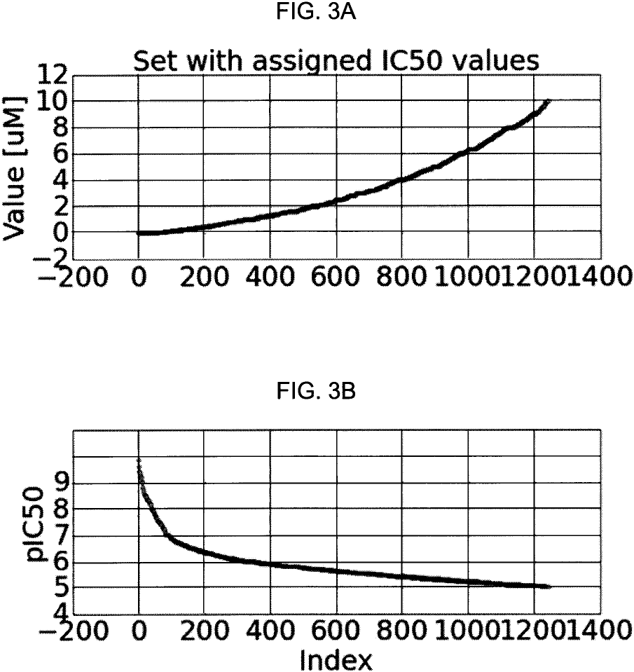

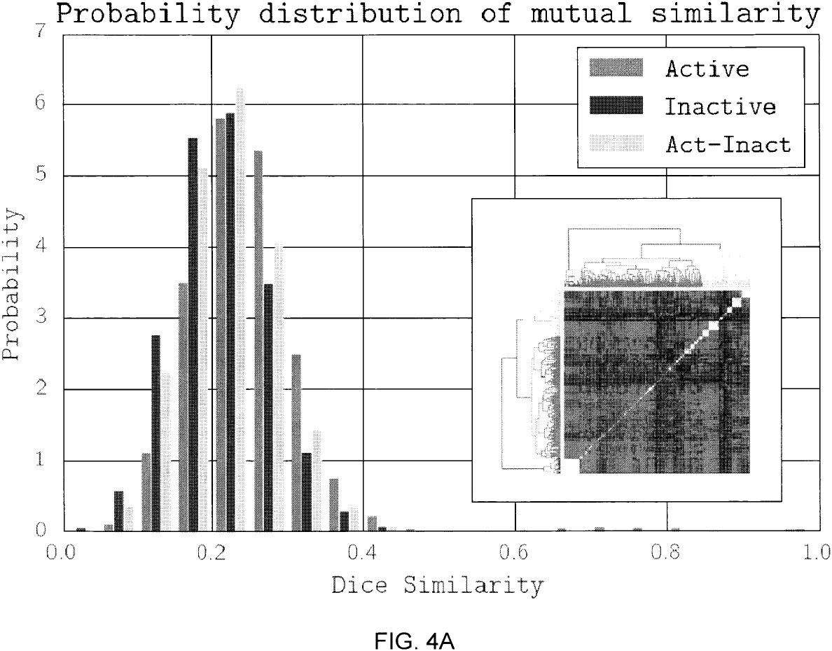

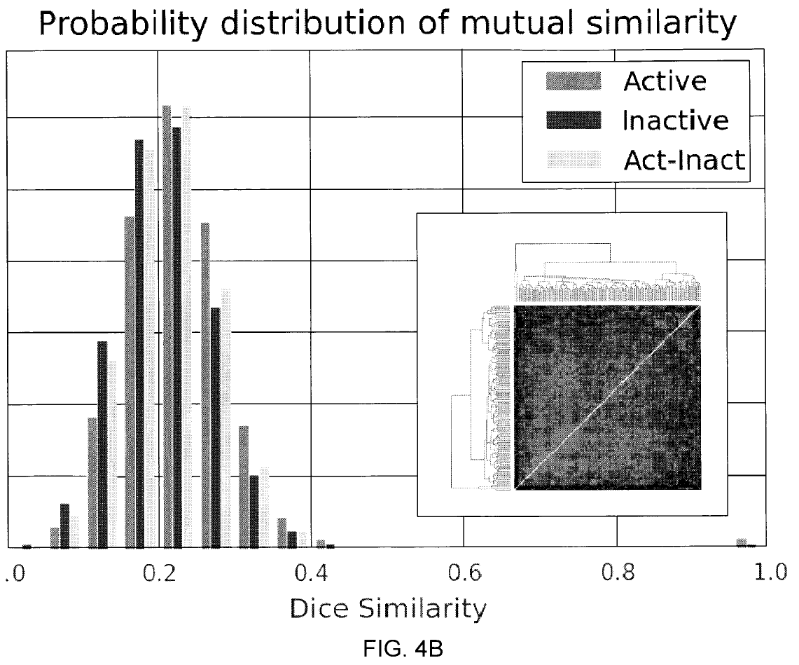

Method 110 illustrated in FIG. 1B includes obtaining respective molecular parameters of a plurality of compounds known to have cardiotoxicity and a plurality of compounds known not to have cardiotoxicity (step 111). In one example, a suitably programmed computer such as described below with reference to FIG. 2 suitably can obtain the molecular parameters via a user interface, via the network, or from a local or remote computer-readable medium. The computer can store the molecular parameters in any suitable computer-readable medium. In one nonlimiting example, the computer obtains the molecular parameters for each compound in the form of a SMILES (simplified molecular-input line-entry system) file such known in the art. In some embodiments, the molecular parameters can be obtained from a publically accessible compound database such as described below with reference to FIG. 2. The compounds for which molecular parameters are obtained in step 111 can have a distribution of activities, e.g., can have a distribution of IC50′s or (dimensionless) pIC50′s such as respectively illustrated in FIGS. 3A-3B, wherein “active” compounds can be considered those with an IC50 of less than 10 µM.

Method 110 illustrated in FIG. 1B further includes, based on a statistical analysis of the respective molecular parameters, assigning to a training set a subset of compounds known to have cardiotoxicity and a subset of compounds known not to have cardiotoxicity (step 112). As one example, principal component analysis (PCA) can be used so as to identify, and to reduce or eliminate, mutual similarity among a plurality of compounds known to have cardiotoxicity and a plurality of compounds known not to have cardiotoxicity, but selecting only a subset of each such plurality. For example, FIG. 3A illustrates an exemplary probability distribution of mutual similarity among a plurality of compounds that are known to have cardiotoxicity (“active”), a plurality of compounds that are known not to have cardiotoxicity (“inactive”), and similarity between active and inactive compounds (“act-inact”). Compounds can be considered to have molecular parameters that are similar to one another based upon such compounds having a dice similarity of greater than 0.15. It can be seen in FIG. 3A that the pluralities of compounds include a relatively wide range of molecular parameters. The inset to FIG. 3A illustrates the result of PCA of such compounds. The yellow boxes that appear along the diagonal correspond to clusters of compounds that have similar molecular parameters as one another. Such clusters represent redundancy within the pluralities of compounds, e.g., sets of compounds that have similar molecular parameters as one another and thus potentially can skew the training of the machine learning algorithm.

PCA or other suitable technique can be used so as to curate the pluralities of compounds, e.g., so as to assign to a training set a subset of compounds known to have cardiotoxicity or to a subset of compounds known not to have cardiotoxicity. For example, PCA can be used to generate a linear independent set of input features. The features under consideration can be standardized, e.g., by converting the features to Z-score = (x-MEAN)/STD, where x is the value of a specific feature and MEAN and STD respectively are the average and standard deviation of all values of that feature in the dataset. The PCA is applied to the standardized values. The output of the PCA can include linear independent linear combinations of features, in other words, collective coordinates that describe the largest variances in the dataset. The PCA vectors are useful to reduce the number of independent features used to train models.

For example, FIG. 3B illustrates an exemplary probability distribution of mutual similarity among a subset of compounds that are known to have cardiotoxicity (“active”), a subset of compounds that are known not to have cardiotoxicity (“inactive”), and similarity between active and inactive compounds (“act-inact”), wherein the subsets were selected using PCA. It can be seen in FIG. 3B that the pluralities of compounds again include a relatively wide range of molecular parameters. The inset to FIG. 3B illustrates the result of PCA of such compounds, in which it can be seen that substantially no yellow boxes appear along the diagonal that would correspond to clusters of compounds that have similar molecular parameters as one another, as they did in the inset to FIG. 3B. Accordingly, in some embodiments, the statistical analysis of step 112 of method 110 provides that the subsets of compounds known to have cardiotoxicity or known not to have cardiotoxicity include substantially no clusters representing redundancy within the pluralities of compounds, that otherwise potentially can skew the training of the machine learning algorithm.

Referring again to FIG. 1B, method 110 further includes executing a machine learning algorithm using the training set. Exemplary machine learning algorithms include, but are not limited to, a naive Bayes model, a naive Bayes bitvectors model, a decision tree model, a random forest model, a LogReg model, and a boosting model. In some embodiments, the boosting model includes the XGBoost algorithm. In one nonlimiting example, the machine learning algorithm is stored on the same computer-readable medium as are the molecular parameters. In another nonlimiting example, the machine learning algorithm is stored in a different computer-readable medium than are the molecular parameters. The machine learning algorithm can receive as input the molecular parameters of the compound from the computer in any suitable manner. For example, in some embodiments, the same computer can obtain the molecular parameters and also can execute the machine learning algorithm, providing the molecular parameters to that algorithm. In other embodiments, a first computer can obtain the molecular parameters and can transmit the molecular parameters via any suitable wired or wireless communication channel to a second computer that can execute the machine learning algorithm, receiving the molecular parameters as input. The resulting trained machine learning algorithm can be used, for example, in any suitable method for predicting cardiotoxicity of molecular parameters of a compound, including but not limited to method 100 described above with reference to FIG. 1A.

It should be noted that methods 100 and 110 can be executed using any suitable combination of hardware and software. For example, FIG. 2 illustrates an exemplary system for predicting cardiotoxicity of molecular parameters of a compound, according to some embodiments of the present invention. The computer-based architecture illustrated in FIG. 2 includes system 200 that is configured to implement one or both of methods 100 and 110; one or more compound databases 230 that are configured to store molecular parameters for compounds known to be cardiotoxic or known not to be cardiotoxic, such as ChEMBL, that are configured to communicate with system 200 via the Internet or other network 220; and a plurality of remote clients 250 that are configured to communicate with system 200 via the Internet or other network 220, are configured to receive user queries requesting predictions of cardiotoxicity of molecular parameters of one or more compounds, to submit such queries to system 200, to receive the results of such queries from system 200, and to output the results of such queries to the user. Alternatively, information within one or more of remote data sources 230 can be converted to local storage within system 200. It will be appreciated that remote compound databases 230 can be operated by an independent entity and need not necessarily be considered to be part of the present invention; accordingly, the architectural details of such data sources 230 are omitted from FIG. 2 for simplicity.

As illustrated in FIG. 2, system 200 includes one or more processing units (CPU’s) 201 (e.g., processing means), a network or other communications interface (NIC) 202 (e.g., networking means), one or more non-volatile, non-transitory, computer readable memory devices or media such as magnetic disk storage or persistent devices 203 (e.g., memory means or storage means) optionally accessed by one or more controllers 204, a user interface 205 including a display 206 and a keyboard 207 or other suitable device for accepting user input, a memory 210 (e.g., memory means or storage means), one or more communication busses 208 for interconnecting the aforementioned components, and a power supply 209 for powering the aforementioned components. Data in memory 210 can be seamlessly shared with non-volatile memory 203 using known computing techniques such as caching. Memory 210 or memory 203 can include mass storage that is remotely located with respect to the central processing unit(s) 201. In other words, some data stored in memory 210 or memory 203 can be hosted on computers that are external to system 200 but that can be electronically accessed by system 200 over an Internet, intranet, or other form of network or electronic cable using network interface 202. In one illustrative embodiment, system 200 is a personal computer. Of course, the present methods equivalently can be performed using commercially available or custom hardware with dozens or more processors connected in parallel, at even greater speed.

Memory 203 can store one or more databases that store molecular parameters of one or more compounds. Preferably, such database(s) respond appropriately to queries from various modules that can be stored within memory 210, such as described further below. Memory 210 preferably stores an operating system 211 that is configured to handle various basic system services and to perform hardware dependent tasks, and a network communications module 212 that is configured to connect system 200 to various other computers such as remote curated data sources 230 and to clients 250 via one or more communication networks 120, such as the Internet, other wide area networks, local area networks (e.g., a local wired or wireless network can connect the system 200 to the remote client 250), metropolitan area networks, and so on.

Memory 210 also can store a cardiotoxicity prediction module 213 that includes a plurality of modules configured to cause processing unit 201 to execute the various steps of one or both of methods 100 and 110. For example, cardiotoxicity prediction module 213 can include a molecular descriptor module 214 configured to cause processing unit 201 to obtain molecular descriptors for one or more compounds from data source 230, from memory 203, or from a remote client 250, such as described above with reference to step 101 of method 100 or step 111 of method 110. In some embodiments, the molecular descriptors include at least structural information about the one or more compounds. As noted herein, in one illustrative embodiment the molecular descriptors are in the form of SMILES files, although other suitable formats can be used. Molecular descriptor module 214 also can be configured to cause processing unit 201 to store molecular descriptors within a database in memory 203, such as described above with reference to step 101 of method 100 or step 111 of method 110. Molecular descriptor module 214 also can be configured to cause processing unit 201 to assign to a training set, based on a statistical analysis of respective molecular parameters, a subset of compounds known to have cardiotoxicity and a subset of compounds known not to have cardiotoxicity, such as described above with reference to step 112 of method 110.

Cardiotoxicity prediction module 213 illustrated in FIG. 2 also includes a machine learning module 215 configured to cause processing unit 201 to train a machine learning algorithm in a manner such as described above with reference to step 113 of method 110, or to provide as input to a machine learning algorithm the respective molecular parameters of one or more compounds, where the machine learning algorithm has been trained using respective molecular parameters of compounds known to have cardiotoxicity and compounds known not to have cardiotoxicity, in a manner such as described above with reference to step 101 of method 100. For example, machine learning module 215 can include instructions for causing processing unit 201 to input into the trained machine learning algorithm the molecular descriptors of one or more compounds. In some embodiments, processing unit 201 also executes such a machine learning algorithm.

In one nonlimiting example, molecular descriptor module 214 need not necessarily require a user to input or define specific molecular parameters or machine learning algorithms to be used, and instead automatically can obtain and train different machine learning algorithms so as to generate best guess/scoring. In one nonlimiting example, molecular parameters can include any of the following molecular parameters available in RDKit:

fr_C_O_noCOO, PEOE_VSA3, Chi4v, fr_Ar_COO, fr_SH, Chi4n, SMR_VSA10, fr_para_hydroxylation, fr_barbitur, fr_Ar_NH, fr_halogen, fr_dihydropyridine, fr_priamide, SlogP_VSA4, fr_guanido, MinPartialCharge, fr_furan, fr_morpholine, fr_nitroso, SlogP_VSA6, fr_COO2, fr_amidine, SMR_VSA7, fr_benzodiazepine, ExactMolWt, fr_Imine, MolWt, fr_hdrzine, fr_urea, NumAromaticRings, fr_quatN, NumSaturatedHeterocycles, NumAliphaticHeterocycles, fr_benzene, fr_phos_acid, fr_sulfone, VSA_EState10, fr_aniline, fr_N_O, fr_sulfonamd, fr_thiazole, TPSA, SMR_VSA5, PEOE_VSA14, PEOE_VSA13, PEOE_VSA12, PEOE_VSA11, PEOE_VSA10, BalabanJ, fr_lactone, fr_Al_COO, EState_VSA10, EStat_VSA11, HeavyAtomMolWt, fr_nitro_arom, Chi0, Chi1, NumAliphaticRings, MolLogP, fr_nitro, fr_Al_OH, fr_azo, NumAliphaticCarbocycles, fr_C_O, fr_ether, fr_phenol_noOrthoHbond, fr_alkyl_halide, NumValenceElectrons, fr_aryl_methyl, fr_Ndealkylation2, MinEStateIndex, fr_term_acetylene, HallKierAlpha, fr_C_S, fr_thiocyan, fr_ketone_Topliss, VSA_EState4, VSA_EState5, VSA_EState6, VSA_EState7, NumHDonors, VSA_EState2, EState_VSA9, fr_HOCCN, fr_phos_ester, MaxAbsEStateIndex, SlogP_VSA12, VSA_EState9, SlogP_VSA10, SlogP_VSA11, fr_COO, NHOHCount, fr_unbrch_alkane, NumSaturatedRings, MaxPartialCharge, fr_methoxy, fr_thiophene, SlogP_VSA8, SlogP_VSA9, MinAbsPartialCharge, SlogP_VSA5, NumAromaticCarbocycles, SlogP_VSA7, SlogP_VSA1, SlogP_VSA2, SlogP_VSA3, NumRadicalElectrons, fr_NH2, fr_piperzine, fr_nitrile, NumHeteroatoms, fr_NH1, fr_NH0, BertzCT, LabuteASA, fr _amide, Chi3n, fr_imidazole, SMR_VSA3, SMR_VSA2, SMR_VSA1, Chi3v, SMR_VSA6, EState_VSA8, SMR_VSA4, EState_VSA6, EState_VSA7, EState_VSA4, SMR_VSA8, EState_VSA2, EState VSA3, fr_Ndealkylation1, EState_VSA1, fr_ketone, Kappa3, Chi0n, fr_diazo, Kappa2, fr_Ar_N, fr_Nhpyrrole, fr_ester, SMR_VSA9, VSA_EState1, fr_prisulfonamd, fr_oxime, EState_VSA5, VSA_EState3, fr_isocyan, Chi2n, Chi2v, HeavyAtomCount, fr_azide, NumHAcceptors, fr_lactam, fr_allylic_oxid, VSA_EState8, fr_oxazole, fr_piperdine, fr_Ar_OH, fr_sulfide, fr_alkyl_carbamate, NOCount, PEOE_VSA9, PEOE_VSA8, PEOE_VSA7, PEOE_VSA6, PEOE_VSAS, PEOE_VSA4, MaxEStateIndex, PEOE_VSA2, PEOE_VSA1, NumSaturatedCarbocycles, fr_imide, FractionCSP3, Chi1v, fr_Al_OH_noTert, fr_epoxide, fr_hdrzone, fr_isothiocyan, NumAromaticHeterocycles, fr_bicyclic, Kappal, MinAbsEStateIndex, fr_phenol, MoIMR, Chi1n, fr_aldehyde, fr_pyridine, fr_tetrazole, RingCount, fr_nitro_arom_nonortho, Chi0v, fr_ArN, NumRotatableBonds, or MaxAbsPartialCharge.

In one nonlimiting example, any of such molecular parameters can be calculated based on a SMILES file, e.g., a SMILES string such as ‘CCC’. Molecular parameter module 214 can build a molecule object which is then standardized. Afterwards, the molecular parameters are calculated. For each machine learning algorithm, molecular parameters that do not carry any information can be removed.

In some embodiments, molecular parameters include chemical features with topological (2D) distances between them that produce 2D pharmacophore or 2D fingerprint features. For example, 2D pharmacophore features include the feature definitions from Gobbi and Poppinger (Gobbi and Poppinger 1998) as implemented in RDKit. In some embodiments, the compounds are converted to 2D fingerprints represented as bit-vectors. Each element of the bitvectors serves as a feature for the machine learning algorithm, while keeping bits that were activated at least 100 times.

Cardiotoxicity prediction module 213 illustrated in FIG. 2 also includes a query module 216 configured to cause processing unit 201 to receive a query term identifying one or more compounds for which cardiotoxicity is to be predicted in a manner such as described above with reference to step 101 of FIG. 1A. In some embodiments, query module 216 causes processing unit 201 to cause display 206 to display a graphical user interface (GUI) that allows the user to readily define the query term. For example, the GUI can include a list of compounds that are available for analysis and a mechanism configured to permit the user to select from the list, e.g., by presenting check boxes or radio buttons adjacent the compounds that the user can select, or by allowing the user to highlight within the list the compounds of interest, using keyboard 207 or other suitable user interface device coupled to system 200. The GUI also can be configured to facilitate the user’s selection of a particular operation to be perform on the selected compounds, such as by allowing the user to redesign compounds by identifying one or more molecular parameters to be altered. For example, the GUI can present the user with output representing the predicted cardiotoxicity of each molecular descriptor of at least a subset of the molecular descriptors of a compound, and the GUI can permit the user to adjust one or more of such molecular descriptors and to run a new prediction in a manner such as described above with reference to method 100. Additionally, as noted below, query module 216 can cause processing unit 201 to accept query terms that are defined remotely, e.g., at remote client 250.

Query module 216 also causes processing unit 201 to provide as input to the trained machine learning algorithm the molecular descriptors of the one or more compounds, in a manner such as described above with reference to step 101 of method 100. Based on the machine learning algorithm’s response, query module 216 causes processing unit 201 to generate an output that represents the predicted cardiotoxicity of each molecular descriptor of at least a subset of the molecular descriptors of the compound, in a manner such as described above with reference to step 102 of method 100. Exemplary suitable outputs are described herein, and others readily can be envisioned. For example, query module 216 can cause processing unit 201 to cause display 206 to display, for each molecular descriptor of at least a subset of molecular descriptors of the compound, a numerical value representing the representing the predicted cardiotoxicity of that molecular parameter. Alternatively, query module 216 can cause processing unit 201 to generate a signal for transmission via a suitable communication channel to remote client 250. Query module 216 further can cause processing unit 201 to cause such an output to be stored in memory 203, to be printed on an associated printer (not illustrated), or otherwise provided to the user. Exemplary outputs are described in greater detail below with reference to Example 2.

Optionally, system 200 is connected via a network such as the Internet 220 to one or more remote clients 250, which permit users who are remote from system 200 to submit and receive the results of queries to system 200. Typically, remote client 250 can include one or more processing units (CPUs) 251; a network or other communications interface (NIC) 252; one or more magnetic disk storage and/or persistent storage devices 253 that are accessed by one or more controllers 254; a user interface 255 including a display 256 and a keyboard 257 or other suitable device configured to accept user input; a memory 260; one or more communication busses 258 for interconnecting the aforementioned components; and a power supply 259 for powering the aforementioned components. In some embodiments, data in memory 260 can be seamlessly shared with non-volatile memory 253 using known computing techniques such as caching.

The memory 260 preferably stores an operating system 261 configured to handle various basic system services and to perform hardware dependent tasks; and a network communication module 262 that is configured to connect remote client 250 to other computers such as system 200. The memory 260 preferably also stores compound analysis module 263 that is configured to cause processing unit 251 to receive user input defining query terms in a manner analogous to query module 216 of system 200, and to transmit such query terms to query module 216 for use in predicting cardiotoxicity of molecular parameters of a compound. Compound analysis module 263 can cause processing unit 251 to receive a response from query module 216 based on the query terms, and to output such response in a manner analogous to that described above, e.g., can cause display 256 to display a representation of the predicted cardiotoxicity of each molecular parameter of at least a subset of the molecular parameters of the compound.

Note that memories 203 and 210 of system 200 and memories 253 and 260 of remote client 250 can include any suitable internal or external memory device, such as FLASH, RAM, ROM, EPROM, EEPROM, or a magnetic or optical disk or tape.

Accordingly, embodiments of the present invention provide a computer system for predicting cardiotoxicity of molecular parameters of a compound. The computer system can include a processor (e.g., processing unit 201 of system 200 or processing unit 251 of remote client 250), and at least one computer-readable medium (e.g., memory 203, memory 210, memory 253, memory 260, compound database(s) 230, or any suitable combination thereof). The memory can store the molecular parameters of the compound, the molecular parameters including at least structural information about the compound. The memory also can store a machine learning algorithm having been trained using respective molecular parameters of compounds known to have cardiotoxicity and of compounds known not to have cardiotoxicity (e.g., machine learning module 215). The memory also can include instructions for causing the processor to perform a step including providing as input to the machine learning algorithm the molecular parameters of the compound (e.g., molecular descriptor module 214, query module 216, compound analysis module 263, or any suitable combination thereof). The memory also can include instructions for causing the processor to receive as output from the machine learning algorithm a representation of the predicted cardiotoxicity of each molecular parameter of at least a subset of the molecular parameters of the compound (e.g., query module 216, compound analysis module 263, or any suitable combination thereof).

In some embodiments, the representation of the predicted cardiotoxicity includes, for each molecular parameter of at least a subset of the molecular parameters of the compound, a numerical value representing the predicted cardiotoxicity of that molecular parameter. In some embodiments, the at least one computer-readable medium further stores instructions for causing the processor to redesign the compound so as not to include at least one of the molecular parameters of at least the subset. In some embodiments, the at least one computer-readable medium further storing instructions for causing the processor to provide as input to the machine learning algorithm the molecular parameters of the redesigned compound; and receive as output from the machine learning algorithm a representation of the predicted cardiotoxicity of each molecular parameter of at least a subset of the molecular parameters of the redesigned compound.

In some embodiments, the representation includes a value representative of a prediction that the molecular parameter of at least the subset will cause the compound to block two or more cardiac ion protein channels. In some embodiments, the two or more cardiac ion protein channels are selected from the group consisting of: sodium ion channel proteins, calcium ion channel proteins, and potassium ion channel proteins. In some embodiments, the potassium ion channel protein is hERG1, the sodium ion channel protein is hNaᵥ1.5, or the calcium channel protein is hCav1.2.

In some embodiments, the at least one computer-readable medium further stores instructions for causing the processor to provide as input to the machine learning algorithm respective molecular parameters of a plurality of compounds of which the previously recited compound is a member; receive as output from the machine learning algorithm a representation of the predicted cardiotoxicity of each molecular parameter of at least a subset of the molecular parameters of each of the compounds of the plurality of compounds; and select a compound of the plurality of compounds based on the predicted cardiotoxicity of each molecular parameter of at least a subset of the molecular parameters of each of the compounds of the plurality of compounds.

In some embodiments, the compounds known to have cardiotoxicity and the compounds known not to have cardiotoxicity are selected based on a statistical analysis of the molecular parameters of those compounds.

In some embodiments, the machine learning algorithm is selected from the group consisting of: a naive Bayes model, a naive Bayes bitvectors model, a decision tree model, a random forest model, a LogReg model, and a boosting model. In some embodiments, the boosting model includes the XGBoost algorithm. In some embodiments, the molecular parameters are selected from the group consisting of: structural information about the compound, physical information about the compound, and chemical information about the compound.

Embodiments of the present invention further provide at least one computer-readable medium for use in predicting cardiotoxicity of molecular parameters of a compound (e.g., any suitable combination of memory 203, memory 211, compound database(s) 230, memory 253, and memory 260. The at least one computer-readable medium stores the molecular parameters of the compound, the molecular parameters including at least structural information about the compound. The at least one computer-readable medium further stores a machine learning algorithm having been trained using respective molecular parameters of compounds known to have cardiotoxicity and of compounds known not to have cardiotoxicity. The at least one computer-readable medium further stores instructions for causing a processor (e.g., processing unit 201 or processing unit 251, or any suitable combination thereof) to perform steps including: providing as input to the machine learning algorithm the molecular parameters of the compound; and receiving as output from the machine learning algorithm a representation of the predicted cardiotoxicity of each molecular parameter of at least a subset of the molecular parameters of the compound.

### 6.4 Examples

The following examples are intended to be purely exemplary, and not limiting of the present invention.

### 6.4.1 Example 1

In a first example, the present systems and methods were implemented in the Python 2.7 programming language (available from the Python Software Foundation at www.python.org) and the IPython notebook web application (available from the IPython development team at ipython.org). The scikit-learn machine learning in Python library was used for the machine learning algorithms (available from the scikit-learn Project at scikit-learn.org). The calculation of the molecular parameters was done using RDkit Open-Source Cheminformatics Software (available at www.rdkit.org). For the molecular fingerprints, a bitlength of 1024 bits and a depth of 2 was used. Compounds included those listed in Kramer et al., “MICE models: Superior to the HERG model in predicting Torsade de Pointes,” Scientific Reports 3: 2100, pages 1-7 (2013) and in the Supplementary Material thereto, the entire contents of which are incorporated by reference herein, exemplary compounds of which are described below in Table 5. Similarities among compounds were calculated based on fingerprint comparison using the Dice similarity score. A molecular fingerprint can be expressed as a bitvector (a vector with only 0 and 1 as components) that is based on the structure of the molecule. The fingerprints can be encoded descriptions of the molecular topology (e.g., atom types and connectivity). There may not be a straightforward connection between the molecular fingerprint and molecular parameters such as used herein, unless the bits of the fingerprint happen to represent specific structural components or other molecular parameters of the compound.

Compound structures and bioassays were taken from the ChEMBL database. Entries for the ChEMBL target ID: CHEMBL240 with the assay description ‘Inhibition of human ERG’ and bioactivity type ‘IC50’ were included. As actives served all compounds with values below 10000 nM. The values were converted to dimensionless pIC50 values. Inactive compounds were compounds were the assay description contained ‘Not Active’. No decoys structures were included. Duplicates and ambiguously labeled compounds were removed from the dataset. Compounds assayed recently were saved for final validations and removed from the dataset. These compounds are referred to as second validation set V2. The final dataset contained 1083 active and 910 inactive compounds. The dataset was randomly split up in train, test, and validation sets. The training set contained 60% of the active compounds and 60% of the inactive compounds. 182 of each active and inactive compounds served as training set. The remaining 434 compounds were defined as the first validation set V1.

The training set was used to train several machine learning algorithms. First, NULL-model based machine learning algorithms were built based on a single molecular parameter. For each molecular parameter the compounds were ranked according to the parameter values. Then, the area under the receiver-operator-characteristic curve (AROC) values were calculated using the roc_score function in scikit-learn. The parameters were sorted according to the AROC values in ascending order and feed successively into the model building algorithms, except for the naive Bayes BitVector (NBBV) model, where the molecular fingerprints were used exclusively as input. The standard parameters for other machine learning algorithms were used as implemented in the scikit-learn Python library except for the following options. For the decision tree machine learning algorithm and the random forest machine learning algorithm, max_features was set to the number of input features. For each set of features, the following machine learning algorithms were executed and applied to the test set: logistic regression (LR), naive Bayes (NB), decision tree (DT), random forest (RF), boosting (BO), and XGBoost. The machine learning algorithm with the highest accuracy was selected for further evaluation. The selected machine learning algorithm then was applied to the validation set V1 and the second validation set V2.

The second validation set V2 contained detailed IC50 values for hERG. Compounds with pIC50 values above 5 (corresponding to IC50 of 10 µM) were labeled as active and with pIC50 values below 5 were labeled as inactive. The quality of the prediction was evaluated in terms of prediction accuracy (AC), true-positive rate (TPR), false-positive rate (FPR), true-negative rate (TPN), false-negative rate (FPN), Kohen’s Kappa (KK), the F1 score (F1), the AROC, the correlation of the predicted class probability to be active with the pIC50 values, sensitivity, and specificity. Sensitivity can be expressed as TP/(TP+FN) and specificity can be expressed as TN/(FP+TN), where TP is the number of true positives, FP is the number of false positives, TN is the number of true negatives, and FN is the number of false negatives. Such performance metrics are well known in the art.

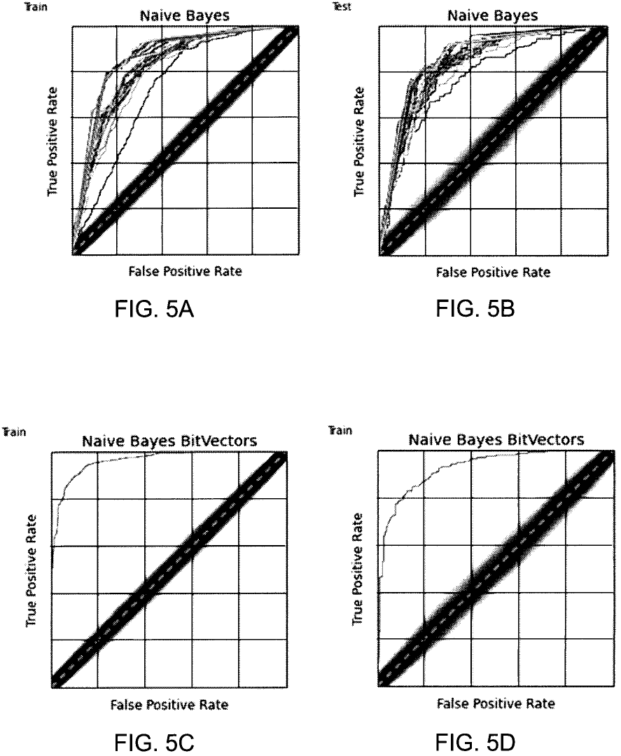

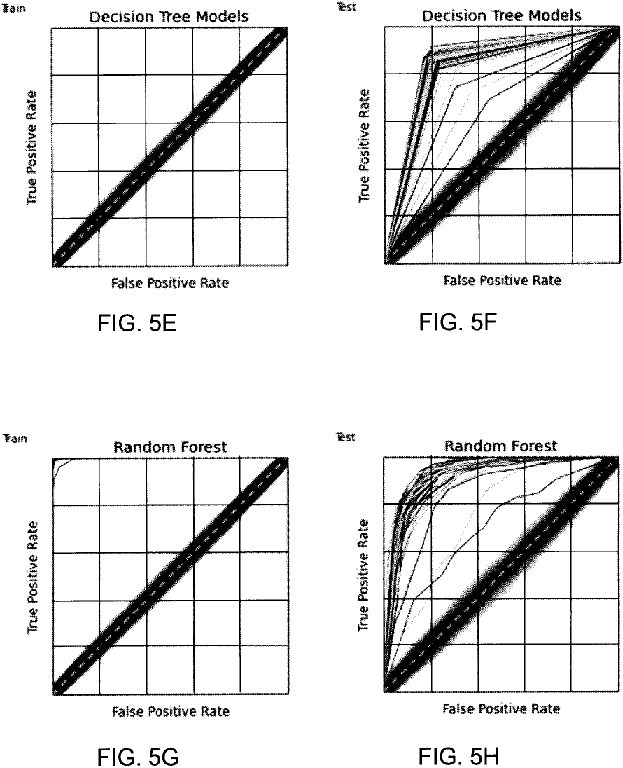

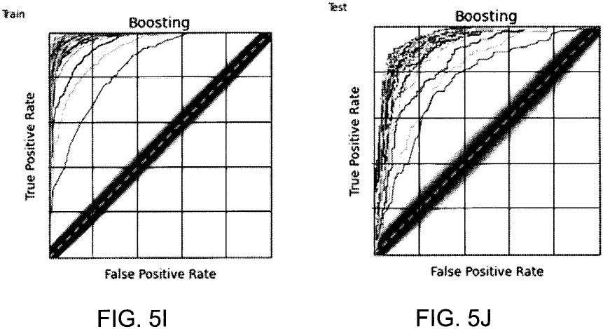

FIGS. 5A-5J illustrate ROC curves for an exemplary training set and test sets for exemplary machine learning algorithms, according to some embodiments of the present invention. More specifically, FIG. 5A illustrates ROC curves for a naive Bayes machine learning algorithm for the training set of Example 1, and FIG. 5B illustrates ROC curves for that naive Bayes machine learning algorithm for the test set of Example 1. FIG. 5C illustrates ROC curves for a naive Bayes bitvectors machine learning algorithm for the training set of Example 1, and FIG. 5D illustrates ROC curves for that naive Bayes bitvectors machine learning algorithm for the test set of Example 1. FIG. 5E illustrates ROC curves for a decision tree machine learning algorithm for the training set of Example 1, and FIG. 5F illustrates ROC curves for that decision tree machine learning algorithm for the test set of Example 1. FIG. 5G illustrates ROC curves for a random forest machine learning algorithm for the training set of Example 1, and FIG. 5H illustrates ROC curves for that random forest machine learning algorithm for the test set of Example 1. FIG. 5I illustrates ROC curves for a boosting machine learning algorithm for the training set of Example 1, and FIG. 5J illustrates ROC curves for that boosting machine learning algorithm for the test set of Example 1. Based on FIGS. 5A-5I, it can be understood that based on a given set of “actives” and “inactives,” the ROC curve can express how well the two groups are separated from each other in respect to a continuous number, e.g., a predicted probability. A random number generator would be expected to produce a line along the diagonal of an ROC plot, indicating a mixture of active and inactive compounds. A perfect separation between active and inactive compounds would be expected to produce a line that extends from the lower left corner to the upper left corner to the upper right corner. Thus, based on FIGS. 5A-5I, it can be seen that the class probability leads to a significant separation of active and inactive compounds in the analyzed dataset.

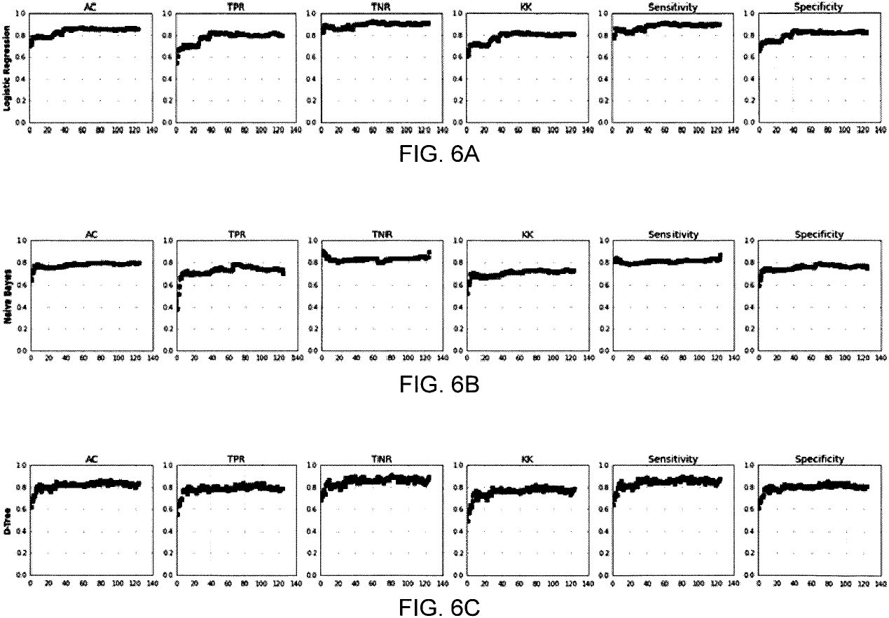

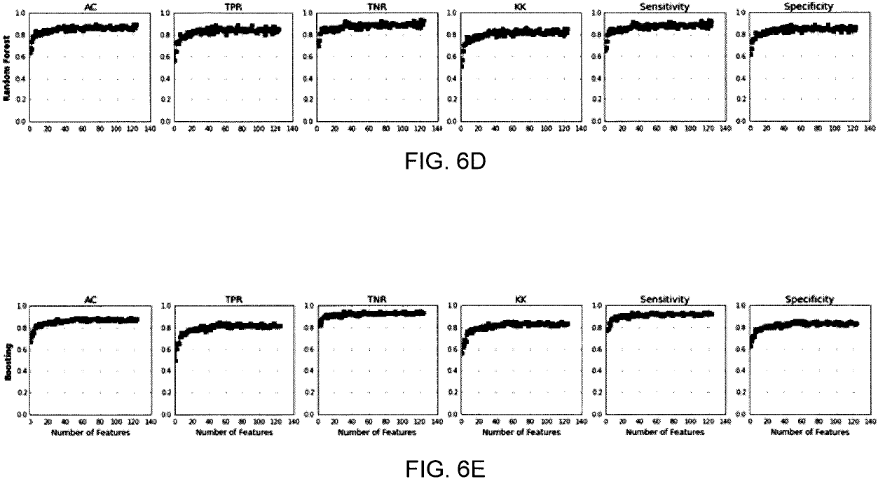

FIGS. 6A-6E illustrate exemplary performance measures of exemplary machine learning algorithms, according to some embodiments of the present invention. More specifically, FIGS. 6A-6E illustrate prediction accuracy (AC), true-positive rate (TPR), true-negative rate (TNR), Kohen’s Kappa (KK), sensitivity, and specificity for the following respective machine learning algorithms using the training set of Example 1: logistic regression, naive Bayes, decision tree, random forest, and boosting. Table 3 lists different performance measures of these machine learning algorithms (MLAs), ordered by prediction accuracy (AC), for the validation set of Example 1. The quality of a classification can be measured by different metrics that provide information about different aspects of the classification. For example, the area under the ROC curve can be based on the class probability that underlies a classification (assigning a compound to a predicted class), whereas AC, sensitivity, and specificity are based on a classification. For example, a low AC combined with a high AROC can indicate that a different cutoff can be used for the classification.

TABLE 3

Performance Measures for Machine Learning Algorithms

| MLA | AROC | AC | Sensitivity | Specificity | True Pos. | True Neg. | False Pos. | False Neg. |
|---|---|---|---|---|---|---|---|---|
| Boosting | 0.935192 | 0.889780 | 0.951724 | 0.864407 | 138 | 306 | 48 | 7 |
| Random Forest | 0.921038 | 0.867735 | 0.852941 | 0.875380 | 145 | 288 | 41 | 25 |
| LogReg | 0.897145 | 0.851703 | 0.858974 | 0.848397 | 134 | 291 | 52 | 22 |
| NB-BitVect | 0.920042 | 0.839679 | 0.831250 | 0.843658 | 133 | 286 | 53 | 27 |
| D-Tree | 0.821018 | 0.835671 | 0.788889 | 0.862069 | 142 | 275 | 44 | 38 |
| Naive Bayes | 0.861675 | 0.829659 | 0.817610 | 0.835294 | 130 | 284 | 56 | 29 |

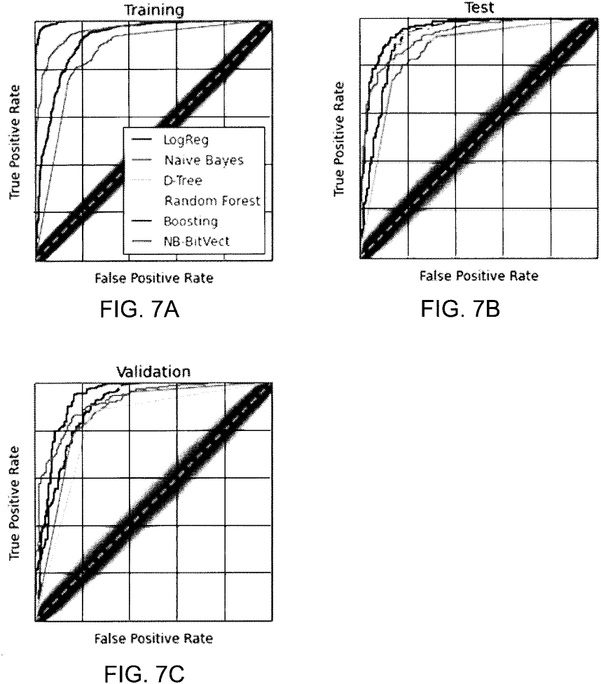

FIGS. 7A-7C illustrate ROC curves for an exemplary training set, test set, and validation set for exemplary machine learning algorithms, according to some embodiments of the present invention. More specifically, FIG. 7A illustrates respective ROC curves for the following machine learning algorithms for the training set of Example 1: logistic regression (LogReg), naive Bayes, decision tree (D-Tree), random forest, boosting, and naive Bayes bitvector (NB-BitVect). FIG. 7B illustrates respective ROC curves for those same machine learning algorithms for the test set of Example 1. FIG. 7C illustrates respective ROC curves for those same machine learning algorithms for the validation set of Example 1.

FIG. 8 illustrates exemplary prediction accuracies for an exemplary training set, test set, and validation set for exemplary machine learning algorithms, according to some embodiments of the present invention. More specifically, FIG. 8 illustrates the respective accuracies of the following machine learning algorithms for the training set, test set, and validation set of Example 1: logistic regression (LogReg), naive Bayes, decision tree (D-Tree), random forest, boosting, and naive Bayes bitvector (NB-BitVect). Based on FIG. 8, it can be understood that different models perform well in the validation set. The graphs compare the quality of the fit and off-sample performances (test and validation) of different models.

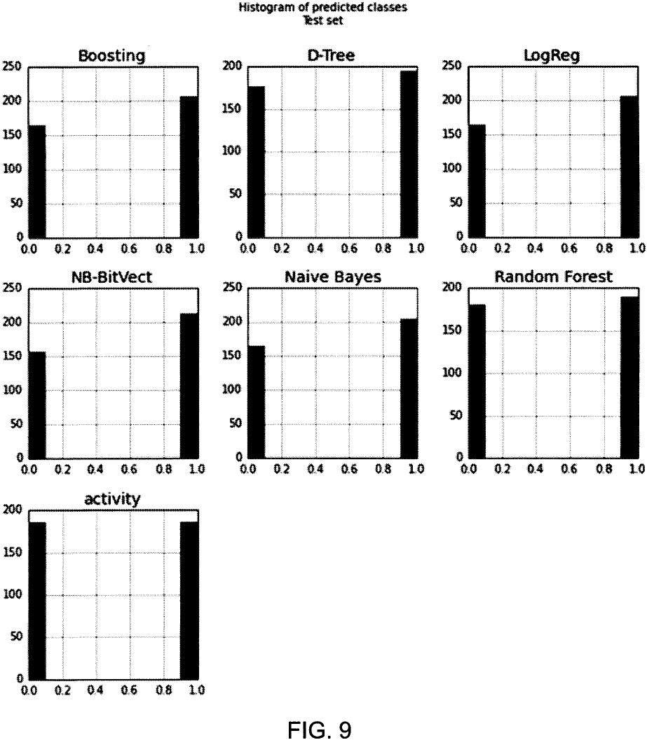

FIG. 9 illustrates histograms showing exemplary predicted or actual numbers of active (1.0 on x-axis) and inactive (0.0 on x-axis) compounds in an exemplary test set with respect to different exemplary machine learning algorithms, according to some embodiments of the present invention. The histogram activities shows the actual distribution. More specifically, FIG. 9 illustrates histograms showing exemplary predicted numbers of active and inactive compounds for the test set of Example 1 for the following machine learning algorithms: boosting, decision tree (D-Tree), logistic regression (LogReg), naive Bayes bitvector (NB-BitVect), naive Bayes, and random forest. Additionally, the lower left plot of FIG. 9 shows the actual activities of the compounds for the test set of Example 1. Similar conclusions as from FIGS. 7 plus the cutoff that has been applied (0.5) leads to classifications with accuracies of above 0.8 in the validation set. FIG. 9 shows the raw number of compounds respectively classified as actives and inactives as compared to the actual number of actives and inactives in the dataset (‘activity’). Random forest and decision tree can be seen to predict the correct numbers.

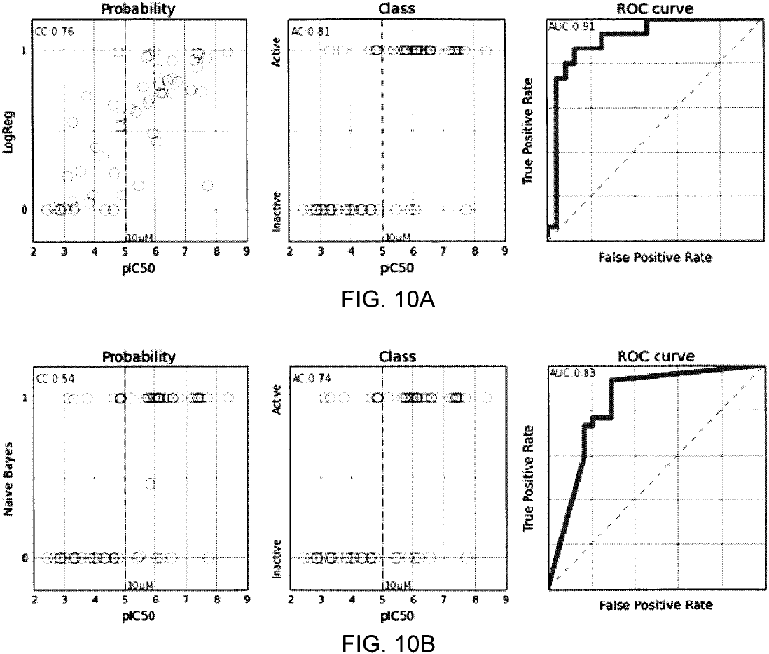

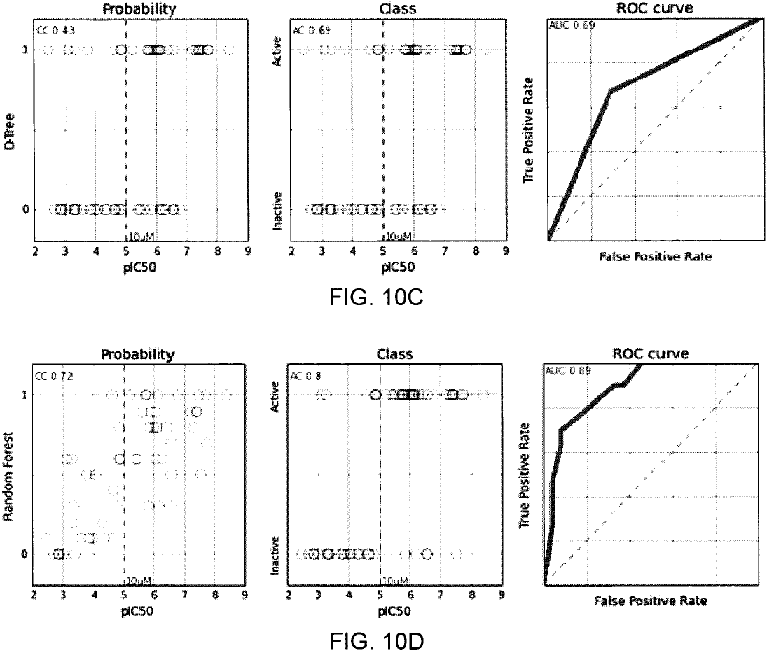

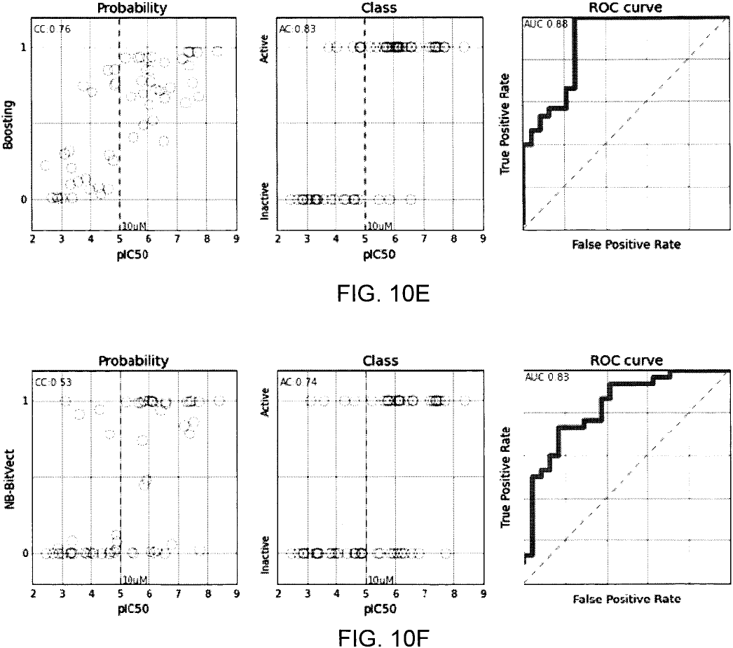

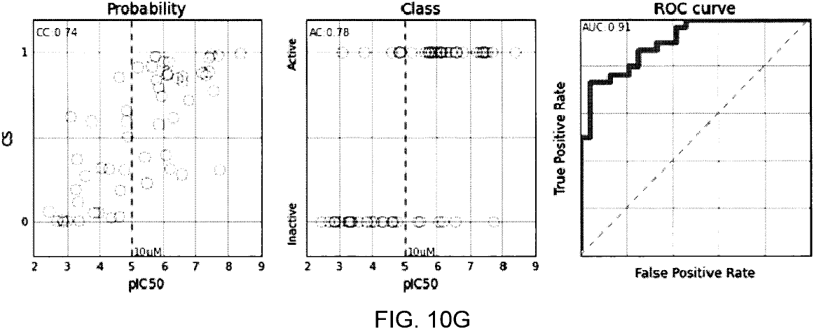

FIGS. 10A-10G illustrate exemplary performances of different exemplary machine learning algorithms with respect to an exemplary validation set, according to some embodiments of the present invention. Compounds with IC50 of less than or equal to 10 µM were considered “active.” The left-most panels indicate an exemplary probability to be active, the middle panels indicate an exemplary corresponding classification over the experimental pIC50 values, and the right-most panels are corresponding ROC curves. FIG. 10A provides such information for the logistic regression (LogReg) machine learning algorithm. FIG. 10B provides such information for the naive Bayes machine learning algorithm. FIG. 10C provides such information for the decision tree (D-Tree) machine learning algorithm. FIG. 10D provides such information for the random forest machine learning algorithm. FIG. 10E provides such information for the boosting machine learning algorithm. FIG. 10F provides such information for the naive Bayes bitvector (NB-BitVect) machine learning algorithm. FIG. 10F provides such information for the Consensus Scoring (CS) machine learning algorithm, which aims to gain more robust and more accurate results for off-sample instances (compounds that have not been used in training and test sets).

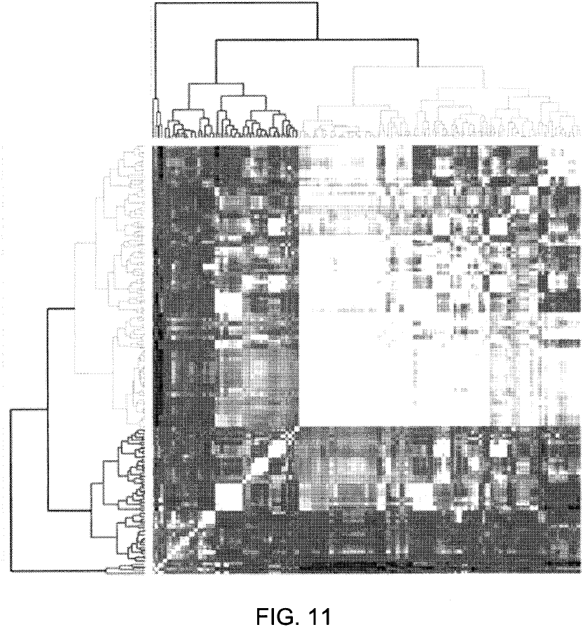

FIG. 11 illustrates an exemplary heatmap of the mutual correlation coefficients of all features in an exemplary training set, more specifically, the training set of Example 1, according to some embodiments of the present invention. From FIG. 11, it can be seen that some of the molecular parameters of the training set are strongly correlated with each other and a feature selection or linearization may be useful to be applied.

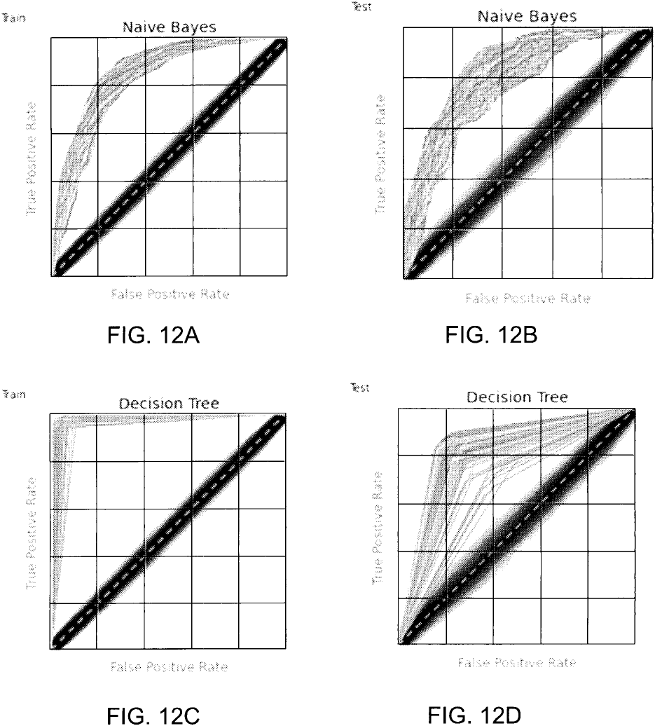

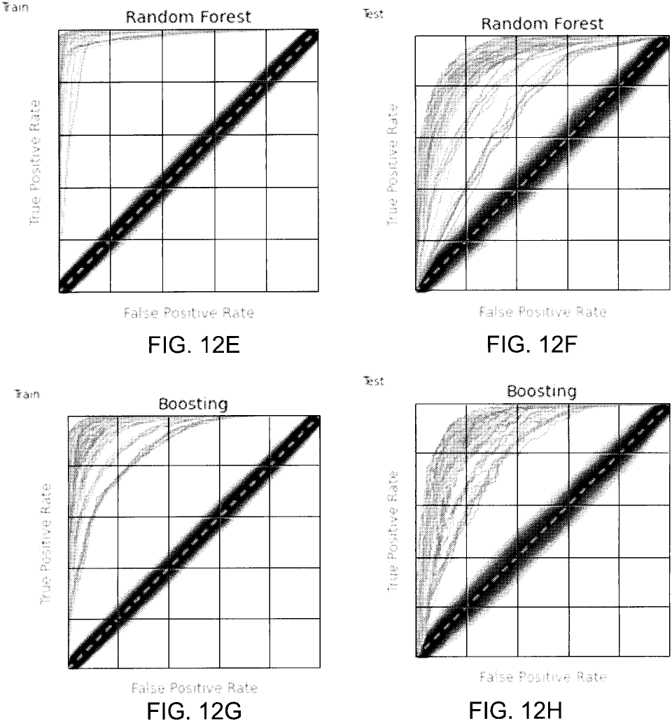

FIGS. 12A-12H illustrate exemplary ROC curves for an exemplary training set and test set for exemplary machine learning algorithms using isomapping, according to some embodiments of the present invention. Isomapping is a distance based method that learns and simplifies the structure of the input data. Isomapping aims to conserve the distances of instances in a high dimensional space by using a smaller number of dimensions. The isomap vectors can be used an input for machine learning. For example,, FIG. 12A illustrates ROC curves for a naive Bayes machine learning algorithm using isomapping to modify the training set of Example 1, and FIG. 12B illustrates ROC curves for that naive Bayes machine learning algorithm using isomapping to modify the test set of Example 1. FIG. 12C illustrates ROC curves for a decision tree machine learning algorithm using isomapping to modify the training set of Example 1, and FIG. 12D illustrates ROC curves for that decision tree machine learning algorithm using isomapping to modify the test set of Example 1. FIG. 12E illustrates ROC curves for a random forest machine learning algorithm using isomapping to modify the training set of Example 1, and FIG. 12F illustrates ROC curves for that random forest machine learning algorithm using isomapping to modify the test set of Example 1. FIG. 12G illustrates ROC curves for a boosting machine learning algorithm using isomapping to modify the training set of Example 1, and FIG. 12H illustrates ROC curves for that boosting machine learning algorithm using isomapping to modify the test set of Example 1. The plots visualize the fit of the training data and the performance in the test set. With more features used as input better fits are and predictions are possible in general.

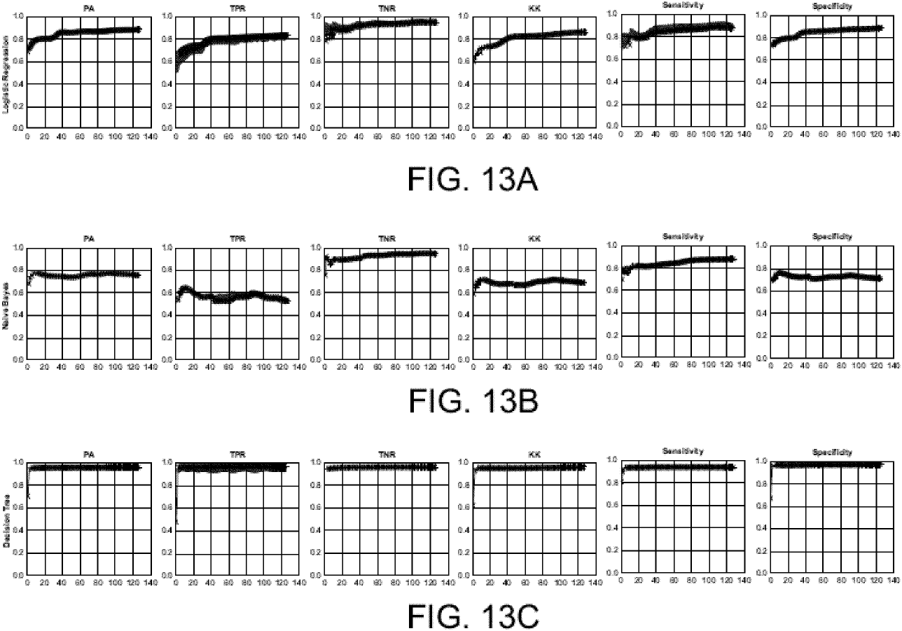

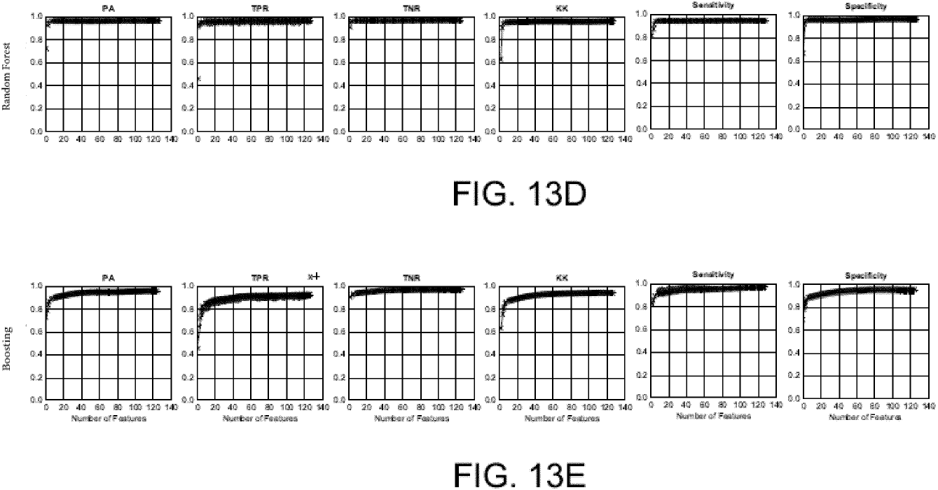

FIGS. 13A-13E illustrate exemplary performance measures of exemplary machine learning algorithms using isomapping, according to some embodiments of the present invention. The blue background spans minimum and maximum, mean (black x) and median (white +). More specifically, FIGS. 13A-13E illustrate prediction accuracy (AC), true-positive rate (TPR), true-negative rate (TNR), Kohen’s Kappa (KK), sensitivity, and specificity for the following respective machine learning algorithms using isomapping to modify the training set of Example 1: logistic regression, naive Bayes, decision tree, random forest, and boosting. From these plots, it can be understood that isomapping can lead to similar predictions as using the raw features, but potentially with enhanced accuracy. Table 4 lists different performance measures of these machine learning algorithms (MLAs) using isomapping, ordered by prediction accuracy (AC), for the validation set of Example 1 modified using isomapping.

TABLE 4

Performance Measures of Machine Learning Algorithms Using Isomapping

| Family | AROC | AC | Sensitivity | Specificity | True Pos. | True Neg. | False Pos. | False Neg. |
|---|---|---|---|---|---|---|---|---|
| RandomForest | 0.894588 | 0.849123 | 0.806867 | 0.878338 | 188 | 296 | 41 | 45 |
| LogReg | 0.885547 | 0.833333 | 0.835000 | 0.832432 | 167 | 308 | 62 | 33 |
| Boosting | 0.915462 | 0.833333 | 0.799107 | 0.855491 | 179 | 296 | 50 | 45 |
| D-Tree | 0.802181 | 0.796491 | 0.738397 | 0.837838 | 175 | 279 | 54 | 62 |
| NaiveBayes | 0.833491 | 0.794737 | 0.791667 | 0.796296 | 152 | 301 | 77 | 40 |

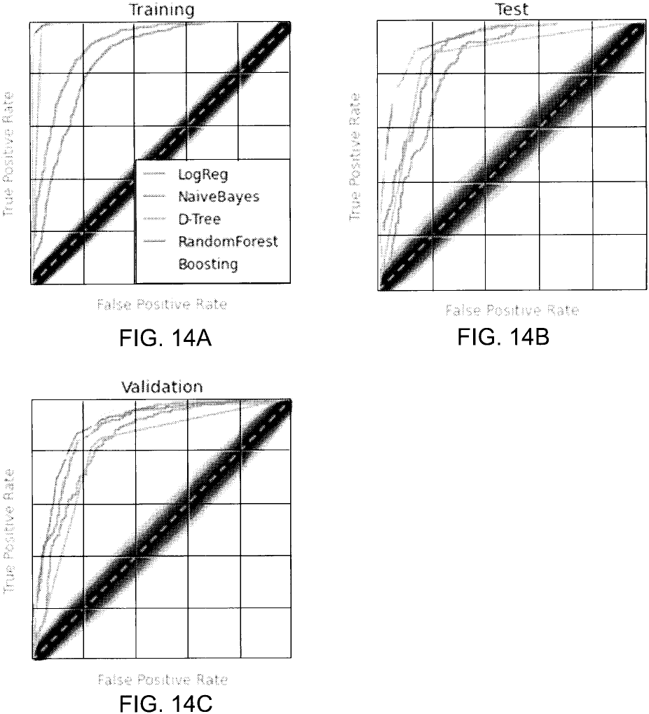

FIGS. 14A-14C illustrate ROC curves for false positives for an exemplary training set, test set, and validation set for exemplary machine learning algorithms, according to some embodiments of the present invention, without using isomapping. More specifically, FIG. 14A illustrates respective ROC curves for false positives for the following machine learning algorithms for the training set of Example 1: logistic regression (LogReg), naive Bayes, decision tree (D-Tree), random forest, boosting, and naive Bayes bitvector (NB-BitVect). FIG. 14B illustrates respective ROC curves for false positives for those same machine learning algorithms for the test set of Example 1. FIG. 14C illustrates respective ROC curves for false positives for those same machine learning algorithms for the validation set of Example 1.

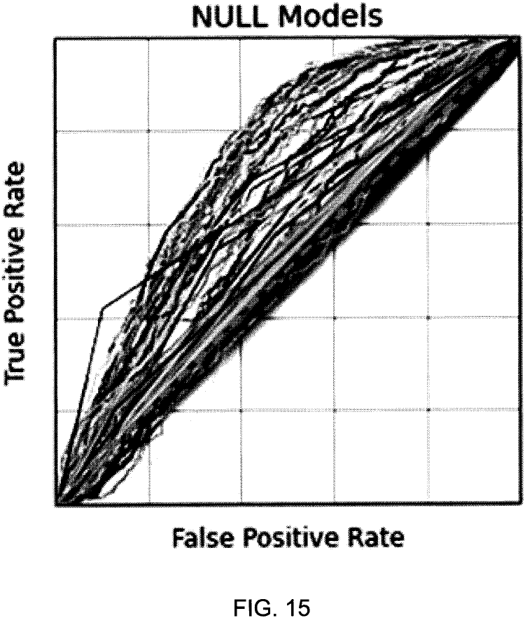

FIG. 15 illustrates ROC curves for compounds in an exemplary training set for a NULL machine learning algorithm, according to some embodiments of the present invention. More specifically, FIG. 15 illustrates ROC curves that were generated by sorting compounds according to the values of individual molecular parameters, such as LogP, molecular weight, and the like, in ascending order (descending when AROC was negative). From these plots, it can be understood that single molecular parameters can have predictive power. Models that are built on a plurality of such molecular parameters (e.g., machine learning algorithms that are trained on a plurality of such molecular parameters) thus can have improved performance relative to those that are built on or trained on a single one of such molecular parameters.

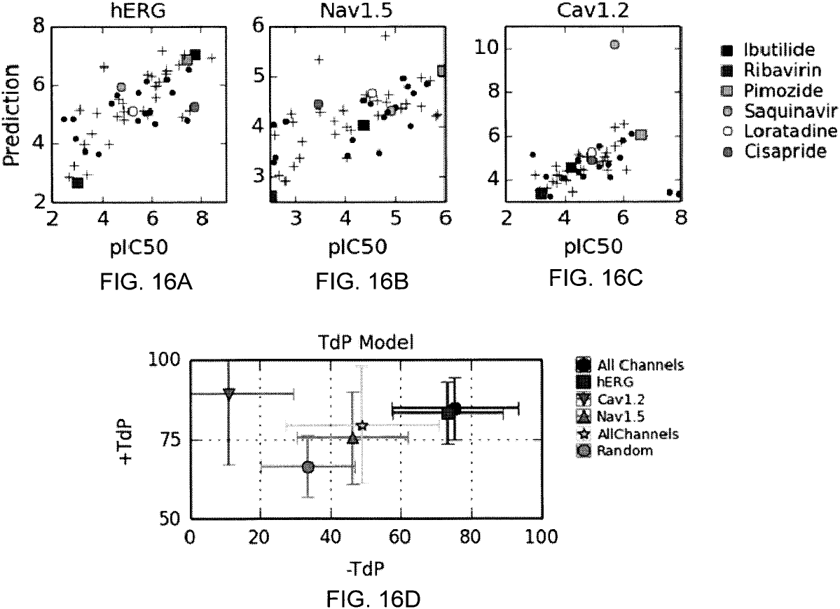

FIGS. 16A-16D illustrate performance of an exemplary 3C model for assessment of torsadogenic potential for a blinded set of blockers, according to some embodiments of the present invention. FIGS. 16A-16C respectively illustrate scatter plots of experimental and predicted pIC50 values for (A) hERG1, (B), Naᵥ1.5, and (C) Caᵥ1.2 for the training set (+,□) and validation set (◦,•) of Example 1. An exemplary selection of compounds is highlighted. Experimental data (IC50 values for hERG1 and Naᵥ1.5 converted to pIC50) for the training set and validation set were adapted from Kramer et al., “MICE models: Superior to the HERG model in predicting Torsade de Pointes,” Scientific Reports 3: 2100, pages 1-7 (2013) and in the Supplementary Material thereto, the entire contents of which are incorporated by reference herein. FIG. 16D illustrates exemplary performance of logistic regression models in terms of true positive rate (+TdP) and true negative rate (-TdP). Evaluation was based on 9 random selections of training sets for +TdP and 16 random selections of training sets for -TdP. The error bars in FIG. 16D indicate the standard deviations. Random-generation predictor set is shown for comparison. Y-axis displays percentage for true predictions of torsadogenic blockers and X-axis for “neutral” or -TdP blockers. From these plots, it can be understood that using hERG in combination with other channels, e.g., NaV and CaV channels, can lead to significantly improved predictions of cardiotoxicity.

### 6.4.2 Example 2

Using the software packages described above, standard evocations and modules were instantiated using the following code:

‘Set working directory’
import os,sys
PATH=“/home/swacker/Documents/Notebooks/Modeling/016-hER G-model-
publication”
os.chdir(“%(PATH)s/301-Validation” %vars())
sys.path.append(“%(PATH)s/lib” %vars())
from modeling import *
import pickle
%pylab inline
plt.style.use(‘ggplot’)
#Set seeds for random number generator.
np.random.seed(12345)
random.seed(12345)
#Options
SaveFigOpt={‘prefix’:‘hERG-Validation-’,‘path’:‘./figures’}
#PlotDemo()

Additionally, the interactive namespace was populated from numpy and matplotlib.

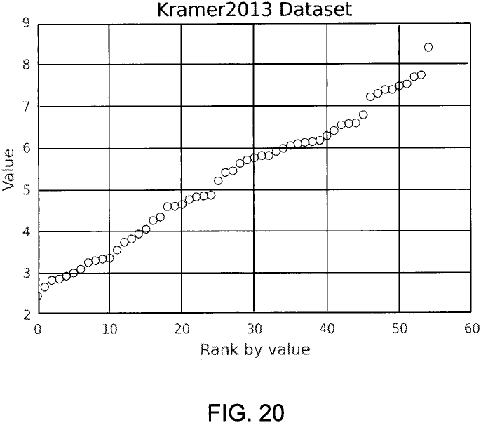

The compounds to be analyzed (which also can be referred to as ligands) were loaded from respective SMILES (smi) file, that contains SMILES codes and unique IDs for each compound. For example, the SMILES (.smi) files were read and converted to a pandas (python module) DataFrame instance which is a table like object. Then molecular parameters for those compounds were calculated and included into a table such as partially reproduced in Table 5. These molecular parameters were later used by the machine learning algorithms to classify the compounds. This example uses the same dataset of compounds to validate the hERG machine learning algorithms as was used in Example 1, e.g., compounds from Kramer et al., “MICE models: Superior to the HERG model in predicting Torsade de Pointes,” Scientific Reports 3: 2100, pages 1-7 (2013) and in the Supplementary Material thereto, the entire contents of which are incorporated by reference herein. The pIC50 values of the compounds used in this Example are shown in FIG. 20. Additionally, machine learning algorithms such as described above in Example 1 were loaded.

TABLE 5

Compounds

| | |
|---|---|
| | smiles |
| ID | |
| amiodarone | CCCCc1c(C(=O)c2cc(I)c(OCCN(CC)CC)c(I)c2)c2cccc... |
| astemizole | COc1ccc(CCN2CCC(CC2)Nc2nc3ccccc3n2Cc2ccc(F)cc2... |
| bepridil | CC(C)COCC(CN(Cc1ccccc1)c1ccccc1)N1CCCC1 |
| ceftriaxone | CO/N=C(\C(=O)N[C@H]1[C@H]2SCC(=C(N2C1=O)C(=O)O... |
| chlorpromazine | CN(C)CCCN1c2ccccc2Sc2ccc(Cl)cc12 |
| cilostazol | O=C1CCc2cc(OCCCCc3nnnn3C3CCCCC3)ccc2N1 |
| cisapride | COC1CN(CCCOc2ccc(F)cc2)CCC1NC(=O)c1cc(Cl)c(N)c... |
| clozapine | CN1CCN(CC1)C1=Nc2cc(Cl)ccc2Nc2ccccc12 |
| dasatinib | Cc1nc(Nc2ncc(s2)C(=O)Nc2c(C)cccc2Cl)cc(n1)N1CC... |
| diazepam | CN1c2ccc(Cl)cc2C(=NCC1=O)c1ccccc1 |
| diltiazem | COc1ccc(cc1)[C@@H]1Sc2ccccc2N(CCN(C)C)C(=O)[C@... |
| disopyramide | CC(C)N(CCC(C(=O)N)(c1ccccc1)c1ccccn1)C(C)C |
| dofetilide | CN(CCOc1ccc(NS(=O)(=O)C)cc1)CCc1ccc(NS(=O)(=O)... |
| donepezil | COc1cc2c(cc1OC)C(=O)C(CC1CCN(Cc3ccccc3)CC1)C2 |
| droperidol | Fc1ccc(cc1)C(=O)CCCN1CCC(=CC1)n1c(=O)[nH]c2ccc... |
| duloxetine | CNCC[C@H](Oc1c2ccccc2ccc1)c1cccs1 |
| flecainide | FC(F)(F)COc1ccc(OCC(F)(F)F)c(cl)C(=O)NCC1CCCCN1 |
| halofantrine | CCCCN(CCCC)CCC(O)c1cc2c(Cl)cc(Cl)cc2c2cc(ccc12... |
| haloperidol | OC1(CCN(CCCC(=O)c2ccc(F)cc2)CC1)clccc(C1)cc1 |
| ibutilide | CCCCCCCN(CC)CCCC(O)c1ccc(NS(=O)(=O)C)cc1 |
| lamivudine | Nc1nc(=O)n(cc1)[C@@H]1CS[C@H](CO)O1 |
| loratadine | CCOC(=O)N1CCC(=C2c3ccc(C1)cc3CCc3cccnc23)CC1 |
| methadone | CCC(=O)C(CC(C)N(C)C)(c1ccccc1)c1ccccc1 |
| metronidazole | Cc1ncc(n1CCO)[N+](=O)[O-] |
| mibefradil | COCC(=0)O[C@]1(CCN(C)CCCc2nc3ccccc3[nH]2)CCc2c... |
| mitoxantrone | OCCNCCNc1ccc(NCCNCCO)c2c1C(=O)c1c(O)ccc(O)c1C2=O |
| moxifloxacin | COc1c2n(cc(C(=O)O)c(=O)c2cc(F)clNIC[C@@H]2CCCN... |
| nifedipine | COC(=O)C1=C(C)NC(=C(Clclccccc1[N+](=0)[0-])C(=... |
| nilotinib | Cc1cn(cn1)c1cc(NC(=O)c2ccc(C)c(Nc3nccc(n3)c3cc... |
| nitrendipine | CCOC(=O)C1=C(C)NC(=C(C1c1cccc(c1)[N+](=O)[O-])... |
| paliperidone | Cc1c(CCN2CCC(CC2)c2noc3cc(F)ccc23)c(=O)n2CCC[C... |
| paroxetine | Fc1ccc(cc1)[C@@H]1CCNC[C@H]1COc1ccc20COc2c1 |
| pentobarbital | CCCC(C)C1(CC)C(=0)NC(=0)NC1=0 |
| phenytoin | O=C1NC(=O)C(N1)(c1ccccc1)c1ccccc1 |
| pimozide | Fc1ccc(cc1)C(CCCN1CCC(CC1)n1c(=O)[nH]c2ccccc12... |
| piperacillin | CCN1CCN(C(=O)N[C@@H](C(=O)N[C@H]2[C@H]3SC(C)(C... |
| procainamide | CCN(CC)CCNC(=O)c1ccc(N)cc1 |
| quinidine | COc1ccc2nccc([C@H](O)[C@H]3C[C@@H]4CCN3C[C@@H]... |
| raltegravir | Cn1c(=O)c(O)c(nc1C(C)(C)NC(=O)c1nnc(C)o1)C(=O)... |
| ribavirin | NC(=O)c1nn(cn1)[C@@H]1O[C@H](CO)[C@@H](O)[C@H]1O |
| risperidone | Cc1c(CCN2CCC(CC2)c2noc3cc(F)ccc23)c(=O)n2CCCCc2n1 |
| saquinavir | CC(C)(C)NC(=O)[C@@H]1C[@@H]2CCCC[C@@H]2CN1C[C... |
| sertindole | Fc1ccc(cc1)n1cc(C2CCN(CCN3CCNC3=O)CC2)c2cc(C1)... |
| sitagliptin | N[C@@H](CC(=O)N1CCn2c(C1)nnc2C(F)(F)F)Cc1cc(F)... |
| solifenacin | OC(=O)CCC(=O)O.O=C(O[C@H]1CN2CC[C@H]1CC2)N1CCc... |
| sotalol | CC(C)NCC(O)c1ccc(NS(=O)(=O)C)cc1 |
| sparfloxacin | C[C@H]1CN(C[C@@H](C)N1)clc(F)c(N)c2c(=O)c(cn(C... |
| sunitinib | CCN(CC)CCNC(=O)c1c(C)[nH]c(/C=C/2\C(=O)Nc3ccc(... |
| telbivudine | Cc1cn([C@@H]2C[C@@H](O)[C@H](CO)02)c(=O)[nH]c1=O |
| terfenadine | CC(C)(C)c1ccc(cc1)C(O)CCCN1CCC(CC1)C(O)(c1cccc... |
| terodiline | CC(CC(c1ccccc1)c1ccccc1)NC(C)(C)C |
| thioridazine | CSc1ccc2Sc3ccccc3N(CCC3CCCCN3C)c2c1 |
| verapamil | COc1ccc(CCN(C)CCCC(C#N)(C(C)C)c2ccc(OC)c(OC)c2... |
| voriconazole | C[C@@H](c1ncncc1F)[C@](O)(Cn1cncn1)c1ccc(F)cc1F |

The following code was used to draw certain of the above compounds based on the SMILES files for the compounds, the drawings being reproduced further below:
Draw.MolsToGridImage([Chem.MolFromSmiles(x) for x in ligands.smiles.head(9)],subImgSize=(200,200))
#Example of input molecules

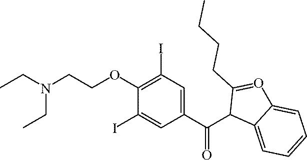

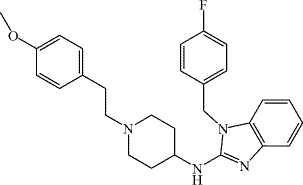 [SMILES: COc1ccc(CCN2CCC(Nc3nc4ccccc4n3Cc3ccc(F)cc3)CC2)cc1]

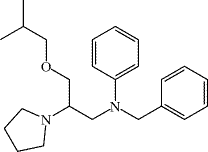 [SMILES: CC(C)COCC(CN(Cc1ccccc1)c1ccccc1)N1CCCC1]

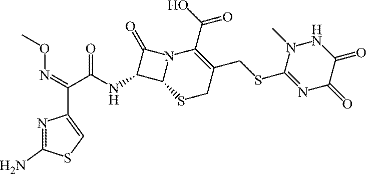 [SMILES: CO/N=C(\C(=O)N[C@@H]1C(=O)N2C(C(=O)O)=C(CSc3nc(=O)c(=O)[nH]n3C)CS[C@H]12)c1csc(N)n1]

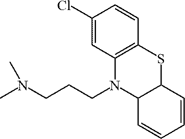 [SMILES: CN(C)CCCN1c2cc(Cl)ccc2SC2C=CC=CC21]

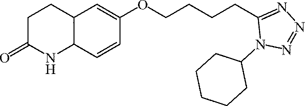 [SMILES: O=C1CCC2C=C(OCCCCc3nnnn3C3CCCCC3)C=CC2N1]

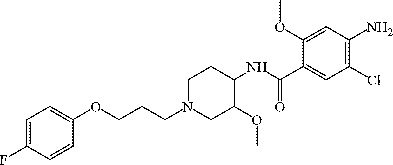 [SMILES: COc1cc(N)c(Cl)cc1C(=O)NC1CCN(CCCOc2ccc(F)cc2)CC1OC]

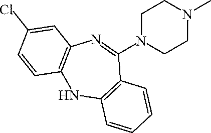 [SMILES: CN1CCN(C2=Nc3cc(Cl)ccc3Nc3ccccc32)CC1]

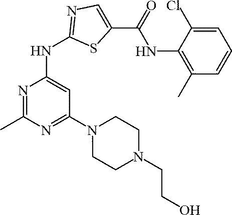 [SMILES: Cc1nc(Nc2ncc(C(=O)Nc3c(C)cccc3Cl)s2)cc(N2CCN(CCO)CC2)n1]

The following code was used to add the molecular parameters present in rdkit to the information about the molecules:
ligands = AddMolProp(ligands) #Adds all molecular properties present in rdkit to the dataframe
ligands

Exemplary molecular parameters for the compounds are listed in Table 6. Table 7 below lists the meaning of the molecular parameter of Table 6.

TABLE 6

Molecular Parameters of Compounds

| | BalabanJ | BertzCT | Chi0 | Chi0n | Chi0v | Chil | Chi1n | Chilv | Chi2n |
|---|---|---|---|---|---|---|---|---|---|
| amiodarone | 2.459198 | 2163.558389 | 47.651818 | 43.927887 | 19.242884 | 26.262485 | 22.319406 | 9.976904 | 5.171468 |
| astemizole | 1.861688 | 2571.670485 | 50.615919 | 47.575067 | 16.575067 | 28.931928 | 24.530512 | 9.083299 | 6.057630 |
| bepridil | 2.863355 | 1987.027393 | 48.790011 | 47.302675 | 13.302675 | 26.545994 | 23.999889 | 6.999889 | 4.371434 |
| ceftriaxone | 1.898858 | 2119.096445 | 41.471809 | 34.660192 | 19.109681 | 24.384442 | 17.363786 | 11.116173 | 6.025366 |
| chlorpromazine | 2.520936 | 1301.719185 | 31.290011 | 29.180640 | 11.753065 | 17.756666 | 14.938871 | 6.633332 | 3.665933 |
| cilostazol | 1.917910 | 1873.786547 | 42.601930 | 40.052565 | 13.052565 | 23.619750 | 20.527620 | 7.080406 | 4.561713 |
| cisapride | 2.475056 | 2070.346935 | 48.358925 | 44.230563 | 15.986492 | 26.813614 | 22.240115 | 8.276439 | 5.183575 |
| clozapine | 2.285315 | 1523.096995 | 32.444711 | 30.166819 | 11.922748 | 18.789358 | 15.622264 | 6.553015 | 4.275050 |
| dasatinib | 1.946002 | 2151.032424 | 45.927839 | 41.733205 | 17.305630 | 26.293525 | 21.135863 | 9.527649 | 5.530042 |
| diazepam | 2.655631 | 1225.664766 | 25.367361 | 22.680640 | 10.436569 | 14.945988 | 11.761140 | 5.639105 | 3.625365 |
| diltiazem | 2.744517 | 1880.410499 | 43.358925 | 39.935669 | 14.752165 | 24.246300 | 20.224634 | 8.041131 | 4.833327 |
| disopyramide | 3.821721 | 1711.359219 | 43.290011 | 41.249889 | 12.249889 | 23.643417 | 20.690192 | 6.295765 | 4.271793 |
| dofetilide | 2.910683 | 2056.422982 | 44.867361 | 40.699379 | 15.332372 | 24.434022 | 20.110773 | 9.596472 | 4.422186 |
| donepezil | 2.116532 | 1993.804310 | 44.997117 | 42.671958 | 13.671958 | 24.957790 | 21.941441 | 7.441441 | 5.112317 |
| droperidol | 2.046545 | 2071.553169 | 38.969655 | 35.536102 | 13.536102 | 22.320938 | 18.333299 | 7.386085 | 4.964535 |
| duloxetine | 2.529909 | 1505.995164 | 31.074468 | 29.263710 | 11.080207 | 17.961843 | 14.960924 | 6.330207 | 3.517165 |
| flecainide | 3.028043 | 1470.686824 | 38.610366 | 32.886959 | 12.886959 | 20.770397 | 16.443369 | 6.548941 | 4.287988 |
| halofantrine | 2.872749 | 2292.227419 | 50.367361 | 45.745284 | 17.257142 | 27.456171 | 23.228104 | 9.075785 | 5.750668 |
| haloperidol | 2.438774 | 1659.918667 | 38.790011 | 35.519639 | 13.275568 | 21.688222 | 18.115281 | 7.084998 | 4.559095 |
| ibutilide | 4.569370 | 1934.844519 | 50.825909 | 48.527420 | 13.343917 | 26.474121 | 23.924045 | 7.508646 | 3.664905 |
| lamivudine | 2.718996 | 790.685105 | 20.292529 | 17.974634 | 7.791131 | 11.633463 | 8.869061 | 4.382882 | 2.291056 |
| loratadine | 2.354789 | 1837.215359 | 39.074468 | 36.088888 | 13.844817 | 22.095818 | 18.669389 | 7.547353 | 4.915244 |
| methadone | 3.922638 | 1554.294606 | 40.082904 | 38.355462 | 11.355462 | 21.871579 | 19.374945 | 5.874945 | 4.013141 |
| metronidazole | 3.786642 | 565.809712 | 16.800965 | 14.566386 | 5.566386 | 9.269891 | 7.069162 | 2.660913 | 1.677206 |
| mibefradil | 2.446721 | 2757.141897 | 58.858925 | 55.444350 | 17.444350 | 32.246300 | 28.222064 | 9.274851 | 6.392256 |
| mitoxantrone | 2.616413 | 1926.239559 | 47.220732 | 43.238344 | 15.238344 | 26.857584 | 21.435447 | 8.013599 | 5.335868 |
| moxifloxacin | 2.126377 | 2036.708106 | 41.419767 | 37.852598 | 13.852598 | 23.367967 | 19.603919 | 7.748457 | 5.736015 |
| nifedipine | 3.542999 | 1387.455084 | 33.997117 | 29.843917 | 11.843917 | 18.996465 | 14.957927 | 6.010714 | 4.193773 |
| nilotinib | 1.817836 | 2684.484557 | 46.494235 | 40.672637 | 18.672637 | 27.780300 | 21.019600 | 10.125173 | 7.024390 |
| nitrendipine | 3.509243 | 1552.217689 | 36.497117 | 32.343917 | 12.343917 | 20.246465 | 16.207927 | 6.260714 | 4.295835 |
| paliperidone | 1.885838 | 2273.366075 | 45.333981 | 41.891564 | 14.891564 | 25.556472 | 21.654638 | 8.246390 | 5.835615 |
| paroxetine | 2.126411 | 1555.535991 | 34.126874 | 31.549923 | 11.549923 | 19.597583 | 16.308154 | 6.360941 | 4.253346 |
| pentobarbital | 4.566551 | 863.046326 | 27.886751 | 25.619172 | 7.619172 | 14.547435 | 12.651227 | 3.756800 | 2.616486 |
| phenytoin | 2.680103 | 1056.051291 | 23.737604 | 21.210924 | 9.210924 | 14.237604 | 10.947103 | 5.052675 | 3.521744 |
| pimozide | 1.936628 | 2552.884108 | 49.201706 | 45.505818 | 16.505818 | 28.052988 | 23.568157 | 9.120943 | 6.199393 |
| piperacillin | 2.049230 | 2196.376048 | 49.729168 | 44.002054 | 17.818551 | 27.836775 | 22.296681 | 9.810502 | 6.490111 |
| procainamide | 3.926665 | 1020.584426 | 30.773503 | 29.249889 | 8.249889 | 16.523503 | 14.387406 | 4.045765 | 2.423187 |
| quinidine | 2.333505 | 1755.594917 | 37.549524 | 35.710924 | 11.710924 | 21.284470 | 18.388655 | 6.480406 | 4.615148 |
| raltegravir | 2.540893 | 1977.469546 | 41.350488 | 36.102487 | 15.102487 | 23.737065 | 18.126077 | 7.823402 | 5.511179 |
| ribavirin | 2.795727 | 811.253563 | 22.629392 | 19.830096 | 7.830096 | 13.120157 | 9.635558 | 4.016386 | 2.749015 |
| risperidone | 1.877562 | 2247.335286 | 44.626874 | 41.483315 | 14.483315 | 24.995812 | 21.542266 | 8.042266 | 5.631491 |
| saquinavir | 2.218440 | 3808.545837 | 78.770620 | 73.724523 | 23.724523 | 43.477580 | 37.348219 | 12.703902 | 8.759409 |
| sertindole | 1.943936 | 2218.779424 | 44.469655 | 40.953032 | 15.708961 | 25.282263 | 21.238977 | 8.669728 | 5.758410 |
| sitagliptin | 2.363138 | 1469.777562 | 33.842417 | 27.912103 | 12.912103 | 18.994700 | 14.194906 | 6.800479 | 4.821909 |
| solifenacin | 0.000001 | 2378.340278 | 52.936275 | 48.843917 | 16.843917 | 29.640800 | 24.837006 | 9.020510 | 5.988649 |
| sotalol | 4.107052 | 1132.642695 | 30.825909 | 28.527420 | 9.343917 | 16.512796 | 13.897652 | 5.535040 | 2.691298 |
| sparfloxacin | 2.444261 | 1826.122489 | 39.290011 | 35.269528 | 13.269528 | 22.094200 | 18.002687 | 7.252798 | 5.454221 |
| sunitinib | 2.541752 | 1978.641171 | 44.392305 | 40.983315 | 13.983315 | 24.588887 | 20.701332 | 7.359692 | 5.088142 |
| telbivudine | 3.068619 | 1020.454709 | 24.585422 | 21.935669 | 7.935669 | 13.715178 | 10.856489 | 4.092779 | 2.814269 |
| terfenadine | 2.262548 | 2620.997880 | 60.383869 | 58.263710 | 17.263710 | 33.474950 | 29.645565 | 9.329069 | 6.556131 |
| terodiline | 3.586369 | 1429.532669 | 38.505553 | 37.447214 | 10.447214 | 21.005553 | 18.894427 | 5.447214 | 3.670820 |
| thioridazine | 2.265835 | 1798.898434 | 39.997117 | 38.210924 | 13.843917 | 22.441117 | 19.658137 | 8.291131 | 4.506744 |
| verapamil | 3.554557 | 2362.551183 | 57.041087 | 54.027420 | 16.027420 | 30.765344 | 27.027420 | 8.027420 | 5.395565 |
| voriconazole | 2.785883 | 1440.468997 | 30.041087 | 25.778210 | 11.778210 | 17.716175 | 13.141780 | 6.233532 | 4.367560 |

TABLE 6

(cont.) Molecular Parameters of Compounds

| | fr_ sulfide | fr_ sulfonamd | fr_ sulfone | fr_term_ acetylene | fr_ tetrazole | fr_thiocyan | fr_thiophene | fr_unbrch_ alkane | fr_ urea |
|---|---|---|---|---|---|---|---|---|---|
| amiodarone | 0 | 0 | 0 | 0 | 0 | 0 | 0 | 0 | 0 |
| astemizole | 0 | 0 | 0 | 0 | 0 | 0 | 0 | 0 | 0 |
| bepridil | 0 | 0 | 0 | 0 | 0 | 0 | 0 | 0 | 0 |
| ceftriaxone | 2 | 0 | 0 | 0 | 0 | 1 | 0 | 0 | 0 |
| chlorpromazine | 0 | 0 | 0 | 0 | 0 | 0 | 0 | 0 | 0 |
| cilostazol | 0 | 0 | 0 | 0 | 1 | 0 | 0 | 0 | 0 |
| cisapride | 0 | 0 | 0 | 0 | 0 | 0 | 0 | 0 | 0 |
| clozapine | 0 | 0 | 0 | 0 | 0 | 0 | 0 | 0 | 0 |
| dasatinib | 0 | 0 | 0 | 0 | 0 | 1 | 0 | 0 | 0 |
| diazepam | 0 | 0 | 0 | 0 | 0 | 0 | 0 | 0 | 0 |
| diltiazem | 1 | 0 | 0 | 0 | 0 | 0 | 0 | 0 | 0 |
| disopyramide | 0 | 0 | 0 | 0 | 0 | 0 | 0 | 0 | 0 |
| dofetilide | 0 | 2 | 0 | 0 | 0 | 0 | 0 | 0 | 0 |
| donepezil | 0 | 0 | 0 | 0 | 0 | 0 | 0 | 0 | 0 |
| droperidol | 0 | 0 | 0 | 0 | 0 | 0 | 0 | 0 | 0 |
| duloxetine | 0 | 0 | 0 | 0 | 0 | 0 | 0 | 1 | 0 |
| flecainide | 0 | 0 | 0 | 0 | 0 | 0 | 0 | 0 | 0 |
| halofantrine | 0 | 0 | 0 | 0 | 0 | 0 | 0 | 0 | 0 |
| haloperidol | 0 | 0 | 0 | 0 | 0 | 0 | 0 | 0 | 0 |
| ibutilide | 0 | 1 | 0 | 0 | 0 | 0 | 0 | 0 | 0 |
| lamivudine | 1 | 0 | 0 | 0 | 0 | 0 | 0 | 0 | 0 |
| loratadine | 0 | 0 | 0 | 0 | 0 | 0 | 0 | 0 | 0 |
| methadone | 0 | 0 | 0 | 0 | 0 | 0 | 0 | 0 | 0 |
| metronidazole | 0 | 0 | 0 | 0 | 0 | 0 | 0 | 0 | 0 |
| mibefradil | 0 | 0 | 0 | 0 | 0 | 0 | 0 | 0 | 0 |
| mitoxantrone | 0 | 0 | 0 | 0 | 0 | 0 | 0 | 0 | 0 |
| moxifloxacin | 0 | 0 | 0 | 0 | 0 | 0 | 0 | 0 | 0 |
| nifedipine | 0 | 0 | 0 | 0 | 0 | 0 | 0 | 0 | 0 |
| nilotinib | 0 | 0 | 0 | 0 | 0 | 0 | 0 | 0 | 0 |
| nitrendipine | 0 | 0 | 0 | 0 | 0 | 0 | 0 | 0 | 0 |
| paliperidone | 0 | 0 | 0 | 0 | 0 | 0 | 0 | 0 | 0 |
| paroxetine | 0 | 0 | 0 | 0 | 0 | 0 | 0 | 0 | 0 |
| pentobarbital | 0 | 0 | 0 | 0 | 0 | 0 | 0 | 0 | 0 |
| phenytoin | 0 | 0 | 0 | 0 | 0 | 0 | 0 | 0 | 0 |
| pimozide | 0 | 0 | 0 | 0 | 0 | 0 | 0 | 0 | 0 |
| piperacillin | 1 | 0 | 0 | 0 | 0 | 0 | 0 | 0 | 0 |
| procainamide | 0 | 0 | 0 | 0 | 0 | 0 | 0 | 0 | 0 |
| quinidine | 0 | 0 | 0 | 0 | 0 | 0 | 0 | 0 | 0 |
| raltegravir | 0 | 0 | 0 | 0 | 0 | 0 | 0 | 0 | 0 |
| ribavirin | 0 | 0 | 0 | 0 | 0 | 0 | 0 | 0 | 0 |
| risperidone | 0 | 0 | 0 | 0 | 0 | 0 | 0 | 0 | 0 |
| saquinavir | 0 | 0 | 0 | 0 | 0 | 0 | 0 | 0 | 0 |
| sertindole | 0 | 0 | 0 | 0 | 0 | 0 | 0 | 0 | 0 |
| sitagliptin | 0 | 0 | 0 | 0 | 0 | 0 | 0 | 0 | 0 |
| solifenacin | 0 | 0 | 0 | 0 | 0 | 0 | 0 | 0 | 0 |
| sotalol | 0 | 1 | 0 | 0 | 0 | 0 | 0 | 0 | 0 |
| sparfloxacin | 0 | 0 | 0 | 0 | 0 | 0 | 0 | 0 | 0 |
| sunitinib | 0 | 0 | 0 | 0 | 0 | 0 | 0 | 0 | 0 |
| telbivudine | 0 | 0 | 0 | 0 | 0 | 0 | 0 | 0 | 0 |
| terfenadine | 0 | 0 | 0 | 0 | 0 | 0 | 0 | 0 | 0 |
| terodiline | 0 | 0 | 0 | 0 | 0 | 0 | 0 | 0 | 0 |
| thioridazine | 1 | 0 | 0 | 0 | 0 | 0 | 0 | 0 | 0 |
| verapamil | 0 | 0 | 0 | 0 | 0 | 0 | 0 | 0 | 0 |
| voriconazole | 0 | 0 | 0 | 0 | 0 | 0 | 0 | 0 | 0 |

TABLE 7

Molecular Parameters of Table 6

| | |
|---|---|
| Molecular Parameter | Meaning |
| BalabanJ | Calculate Balaban’s J value for a molecule such as described in Chem. Phys. Lett. vol 89, 399-404, (1982) |
| BertzCT | A topological index meant to quantify “complexity” of molecules such as described in J. Am. Chem. Soc., vol 103, 3599-601 (1981) |
| Chi0 | Average valency connectivity index. |
| Chi0n | Connectivity descriptor such as described in Rev. Comp. Chem. Vol. 2, 367-422, (1991) |
| Chi0v | Connectivity descriptor such as described in Rev. Comp. Chem. Vol. 2, 367-422, (1991) |
| Chi1 | Connectivity descriptor such as described in Rev. Comp. Chem. Vol. 2, 367-422, (1991) |
| Chi1n | Connectivity descriptor such as described in Rev. Comp. Chem. Vol. 2, 367-422, (1991) |
| Chi1v | Connectivity descriptor such as described in Rev. Comp. Chem. Vol. 2, 367-422, (1991) |
| Chi2n | Connectivity descriptor such as described in Rev. Comp. Chem. Vol. 2, 367-422, (1991) |
| fr_sulfide | Number of sulfide groups |
| fr_sulfonamd | Number of sulfonamide groups |
| fr_sulfone | Number of sulfone groups |
| fr_term_acetylene | Number of terminal acetylenes |
| fr_tetrazole | Number of tetrazole groups |
| fr_thiocyan | Number of thiocyanates |
| fr_thiophene | Number of thiophene rings |
| fr_unbrch_alkane | Number of unbranched alkanes of at least 4 members (excludes halogenated alkanes) |
| fr_urea | Number of urea groups |

Table 8 lists additional molecular parameters that may or may not appear in Table 6, and also or alternatively can be used. Certain information in Table 8 adapted from Wicker et al., “Will it crystallize? Predicting crystallinity of molecular materials,” CrystEngComm 17: 1927-1934 and supporting information, DOI: 10.1039/C4CE01912A (2014), the entire contents of which are incorporated by reference herein.

TABLE 8

Additional Molecular Parameters

| | | |
|---|---|---|
| Molecular Parameter | Meaning | Source |
| NumAromaticRings | Number of aromatic rings | |
| SMR_VSA7 | MOE MR VSA descriptors | |
| SlogP_VSA | MOE logP VSA descriptors | |
| MolWt, HeavyAtomMolWt, NumRadicalElectrons, NumValenceElectrons, HeavyAtomCount, NumHeteroatoms, NumRotatableBonds, RingCount | Self-explanatory | Implementation can be found in open source RDKit version 2012.12.1 descriptor module |
| Chi0v, Chilv, Chi2v, Chi3v, Chi4v, ChiNv, HallKierAlpha, Kappa1, Kappa2, Kappa3 | | Rev. Comp. Chem. vol 2, 367-422, (1991) |
| Chi0n, Chi1n, Chi2n, Chi3n, Chi4n, ChiNn | | Similar to Hall Kier ChiXv, but uses nVal instead of valence |
| Ipc | | J. Chem. Phys., vol 67, 4517-33 (1977) |
| LabuteASA, PEOE-VSA1 -PEOE-VSA14, SMR-VSA1 -SMR-VSA10, SlogP-VSA1 -SlogP-VSA12 | | J. Mol. Graph. Mod., vol 18, 464-77 (2000) |
| TPSA | | J. Med. Chem., vol 43, 3714-7, (2000) |
| MolLogP, MolMR | | J. Chem. Inform. Comput. Sci., vol 39, 868-73 (1999) |
| EState-VSA1 - EState-VSA11, VSA-EState1 - VSA-EState 10 | | MOE-type descriptors using electrotopological state indices and surface area contributions developed at RD from J. Chem. Inform. Comput. Sci., vol 31, 76-81 (1991) |
| NOCount | Number of Nitrogen and Oxygen atoms | |
| NumHAcceptors Number of Hydrogen Bond Acceptors | NumHAcceptors Number of Hydrogen Bond Acceptors | |
| NumHDonors Number of Hydrogen Bond Donors | NumHDonors Number of Hydrogen Bond Donors | |
| fr-Al-COO | Number of aliphatic carboxylic acids | |
| fr-Al-OH | Number of aliphatic hydroxyl groups | |
| fr-Al-OH-noTert | Number of aliphatic hydroxyl groups excluding tert-OH | |
| fr-ArN | Number of N functional groups attached to aromatics | |
| fr-Ar-COO | Number of Aromatic carboxylic acids | |
| fr-Ar-N | Number of aromatic nitrogens | |
| fr-Ar-NH | Number of aromatic amines | |
| fr-Ar-OH | Number of aromatic hydroxyl groups | |
| fr-COO | Number of carboxylic acids | |
| fr-CO02 | Number of carboxylic acids | |
| fr-C-O | Number of carbonyl | |
| fr-C-O-noCOO | Number of carbonyl O, excluding COOH | |
| fr-C-S | Number of thiocarbonyl | |
| fr-HOCCN | Number of C(OH)CCN-Ctert-alkyl or C(OH)CCNcyclic | |
| fr-Imine | Number of Imines | |
| fr-NH0 | Number of Tertiary amines | |
| fr-NH1 | Number of Secondary amines | |
| fr-NH2 | Number of Primary amines | |
| fr-N-O | Number of hydroxylamine groups | |
| fr-Ndealkylation1 | Number of XCCNR groups | |
| fr-Ndealkylation2 | Number of tert-alicyclic amines (no heteroatoms, not quinine-like bridged N) | |
| fr-Nhpyrrole | Number of H-pyrrole nitrogens | |
| fr-SH | Number of thiol groups | |
| fr-aldehyde | Number of aldehydes | |
| fr-alkyl-carbamate | Number of alkyl carbamates | |
| fr-alkyl-halide | Number of alkyl halides | |
| fr-allylic-oxid | Number of allylic oxidation sites excluding steroid dienone | |
| fr-amide | Number of amides | |
| fr-amidine | Number of amidine groups | |
| fr-aniline | Number of anilines | |
| fr-aryl-methyl | Number of aryl methyl sites for hydroxylation | |
| fr-azide | Number of azide groups | |
| fr-azo | Number of azo groups | |
| fr-barbitur | Number of barbiturate groups | |
| fr-benzene | Number of benzene rings | |
| fr-benzodiazepine | Number of benzodiazepines with no additional fused rings | |
| fr-bicyclic | Number of bicyclic rings | |
| fr-diazo | Number of diazo groups | |
| fr-dihydropyridine | Number of dihydropyridines | |
| fr-epoxide | Number of epoxide rings | |
| fr-ester | Number of esters | |
| fr-ether | Number of ether oxygens (including phenoxy) | |
| fr-furan | Number of furan rings | |
| fr-guanido | Number of guanidine groups | |
| fr-halogen | Number of halogens | |
| fr-hdrzine | Number of hydrazine groups | |
| fr-hdrzone | Number of hydrazone groups | |
| fr-imidazole | Number of imidazole rings | |
| fr-imide | Number of imide groups | |
| fr-isocyan | Number of isocyanates | |
| fr-isothiocyan | Number of isothiocyanates | |
| fr-ketone | Number of ketones | |
| fr-ketone-Topliss | Number of ketones excluding diaryl, a,b-unsat. | |
| fr-lactam | Number of beta lactams | |
| fr-lactone | Number of cyclic esters (lactones) | |
| fr-methoxy | Number of methoxy groups —OCH₃ | |
| fr-morpholine | Number of morpholine rings | |
| fr-nitrile | Number of nitriles | |
| fr-nitro | Number of nitro groups | |
| fr-nitro-arom | Number of nitro benzene ring substituents | |
| fr-nitro-arom-nonortho | Number of non-ortho nitro benzene ring substituents | |
| fr-nitroso | Number of nitroso groups, excluding NO₂ | |
| fr-oxazole | Number of oxazole rings | |
| fr-oxime | Number of oxime groups | |
| fr-para-hydroxylation | Number of para-hydroxylation sites | |
| fr-phenol | Number of phenols | |
| fr-phenol-noOrthoHbond | Number of phenolic OH excluding ortho intramolecular Hbond substituents | |
| fr-phos-acid | Number of phosphoric acid groups | |
| fr-phos-ester | Number of phosphoric ester groups | |
| fr-piperdine | Number of piperdine rings | |
| fr-piperzine | Number of piperzine rings | |
| fr-priamide | Number of primary amides | |
| fr-prisulfonamd | Number of primary sulfonamides | |
| fr-pyridine | Number of pyridine rings | |
| fr-quatN | Number of quarternary nitrogens | |
| fr-sulfide | Number of thioether | |
| fr-sulfonamd | Number of sulfonamides | |
| fr-sulfone | Number of sulfone groups | |
| fr-term-acetylene | Number of terminal acetylenes | |
| fr-tetrazole | Number of tetrazole rings | |
| fr-thiazole | Number of thiazole rings | |
| fr-thiocyan | Number of thiocyanates | |
| fr-thiophene | Number of thiophene rings | |
| fr-unbrch-alkane | Number of unbranched alkanes of at least 4 members (excludes halogenated alkanes) | |
| fr-urea | Number of urea groups | |

The following code was used to load the following machine algorithms (models): boosting (BO), decision tree (DT), logistic regression (LR), naive bayes (LB), and random forest (RF):

models = LoadModels(‘../201-Model-AllFeatures/out/*.p’) #Loads models models #Contains vector of features required by the model, a unique ID of the model, an info string and the acctual model.

#all this information is actually stored in the model as attributes model.ID, model.type, model.X, model.info.
../201-Model-AllFeatures/out/hERG-Model-AllFeatures-BO-model.p
../201-Model-AllFeatures/out/hERG-Model-AllFeatures-DT-model.p
../201-Model-AllFeatures/out/hERG-Model-AllFeatures-LR-model.p
../201-Model-AllFeatures/out/hERG-Model-AllFeatures-NB-model.p
../201-Model-AllFeatures/out/hERG-Model-AllFeatures-RF-model.p

The models need a DataFrame with the columns listed in the attribute X. The function AddMolProp() adds all molecular parameters present in the RDKit python package. The current molecular parameters used in RDKit are provided elsewhere herein.

Applying each of the machine learning algorithms (models) to the dataframe (molecular parameters of compounds such as listed in Table 5) outputs a prediction containing the class probability to be active, the predicted classification, and the compound ID in the index and as a separate column. The output also can include an indication of which model was used, a unique model-ID (e.g., 6c61f5e5-5378-4bbe-835b-05f0cddb4742 for the boosting algorithm), and an information string, e.g. the target(s) for which the machine learning algorithm was trained. Table 9 lists exemplary outputs of the boosting machine learning algorithm. The function ScoreModels() applies all models to the prepared DataFrame and returns a DataFrame with the classification and the corresponding class probabilities such as shown in Table 9.

TABLE 9

Outputs for Boosting Machine Learning Algorithm

| | Probability | Classification | Model | Model-ID | Target |
|---|---|---|---|---|---|
| ID | | | | | |
| amiodarone | 0.735695 | 1 | Boosting | 6c61f5e5-5378-4bbe-835b-05f0cddb4742 | hERG |
| astemizole | 0.971209 | 1 | Boosting | 6c61f5e5-5378-4bbe-835b-05f0cddb4742 | hERG |
| bepridil | 0.888848 | 1 | Boosting | 6c61f5e5-5378-4bbe-835b-05f0cddb4742 | hERG |
| ceftriaxone | 0.018208 | 0 | Boosting | 6c61f5e5-5378-4bbe-835b-05f0cddb4742 | hERG |
| chlorpromazine | 0.717121 | 1 | Boosting | 6c61f5e5-5378-4bbe-835b-05f0cddb4742 | hERG |
| cilostazol | 0.561805 | 1 | Boosting | 6c61f5e5-5378-4bbe-835b-05f0cddb4742 | hERG |
| cisapride | 0.969072 | 1 | Boosting | 6c61f5e5-5378-4bbe-835b-05f0cddb4742 | hERG |
| clozapine | 0.905098 | 1 | Boosting | 6c61f5e5-5378-4bbe-835b-05f0cddb4742 | hERG |
| dasatinib | 0.873862 | 1 | Boosting | 6c61f5e5-5378-4bbe-835b-05f0cddb4742 | hERG |
| diazepam | 0.074113 | 0 | Boosting | 6c61f5e5-5378-4bbe-835b-05f0cddb4742 | hERG |
| diltiazem | 0.904612 | 1 | Boosting | 6c61f5e5-5378-4bbe-835b-05f0cddb4742 | hERG |
| disopyramide | 0.817667 | 1 | Boosting | 6c61f5e5-5378-4bbe-835b-05f0cddb4742 | hERG |
| dofetilide | 0.864117 | 1 | Boosting | 6c61f5e5-5378-4bbe-835b-05f0cddb4742 | hERG |
| donepezil | 0.943334 | 1 | Boosting | 6c61f5e5-5378-4bbe-835b-05f0cddb4742 | hERG |
| droperidol | 0.906458 | 1 | Boosting | 6c61f5e5-5378-4bbe-835b-05f0cddb4742 | hERG |
| duloxetine | 0.787037 | 1 | Boosting | 6c61f5e5-5378-4bbe-835b-05f0cddb4742 | hERG |
| flecainide | 0.739089 | 1 | Boosting | 6c61f5e5-5378-4bbe-835b-05f0cddb4742 | hERG |
| halofantrine | 0.829434 | 1 | Boosting | 6c61f5e5-5378-4bbe-835b-05f0cddb4742 | hERG |
| haloperidol | 0.916841 | 1 | Boosting | 6c61f5e5-5378-4bbe-835b-05f0cddb4742 | hERG |
| ibutilide | 0.309506 | 0 | Boosting | 6c61f5e5-5378-4bbe-835b-05f0cddb4742 | hERG |
| lamivudine | 0.019748 | 0 | Boosting | 6c61f5e5-5378-4bbe-835b-05f0cddb4742 | hERG |
| loratadine | 0.898572 | 1 | Boosting | 6c61f5e5-5378-4bbe-835b-05f0cddb4742 | hERG |
| methadone | 0.584744 | 1 | Boosting | 6c61f5e5-5378-4bbe-835b-05f0cddb4742 | hERG |
| metronidazole | 0.012415 | 0 | Boosting | 6c61f5e5-5378-4bbe-835b-05f0cddb4742 | hERG |
| mibefradil | 0.915556 | 1 | Boosting | 6c61f5e5-5378-4bbe-835b-05f0cddb4742 | hERG |
| mitoxantrone | 0.218479 | 0 | Boosting | 6c61f5e5-5378-4bbe-835b-05f0cddb4742 | hERG |
| moxifloxacin | 0.595103 | 1 | Boosting | 6c61f5e5-5378-4bbe-835b-05f0cddb4742 | hERG |
| nifedipine | 0.048603 | 0 | Boosting | 6c61f5e5-5378-4bbe-835b-05f0cddb4742 | hERG |
| nilotinib | 0.962630 | 1 | Boosting | 6c61f5e5-5378-4bbe-835b-05f0cddb4742 | hERG |
| nitrendipine | 0.122214 | 0 | Boosting | 6c61f5e5-5378-4bbe-835b-05f0cddb4742 | hERG |
| paliperidone | 0.916460 | 1 | Boosting | 6c61f5e5-5378-4bbe-835b-05f0cddb4742 | hERG |
| paroxetine | 0.934697 | 1 | Boosting | 6c61f5e5-5378-4bbe-835b-05f0cddb4742 | hERG |
| pentobarbital | 0.015339 | 0 | Boosting | 6c61f5e5-5378-4bbe-835b-05f0cddb4742 | hERG |
| phenytoin | 0.051538 | 0 | Boosting | 6c61f5e5-5378-4bbe-835b-05f0cddb4742 | hERG |
| pimozide | 0.971281 | 1 | Boosting | 6c61f5e5-5378-4bbe-835b-05f0cddb4742 | hERG |
| piperacillin | 0.080560 | 0 | Boosting | 6c61f5e5-5378-4bbe-835b-05f0cddb4742 | hERG |
| procainamide | 0.122428 | 0 | Boosting | 6c61f5e5-5378-4bbe-835b-05f0cddb4742 | hERG |
| quinidine | 0.802696 | 1 | Boosting | 6c61f5e5-5378-4bbe-835b-05f0cddb4742 | hERG |
| raltegravir | 0.262385 | 0 | Boosting | 6c61f5e5-5378-4bbe-835b-05f0cddb4742 | hERG |
| ribavirin | 0.032124 | 0 | Boosting | 6c61f5e5-5378-4bbe-835b-05f0cddb4742 | hERG |
| risperidone | 0.955991 | 1 | Boosting | 6c61f5e5-5378-4bbe-835b-05f0cddb4742 | hERG |
| saquinavir | 0.130612 | 0 | Boosting | 6c61f5e5-5378-4bbe-835b-05f0cddb4742 | hERG |
| sertindole | 0.968285 | 1 | Boosting | 6c61f5e5-5378-4bbe-835b-05f0cddb4742 | hERG |
| sitagliptin | 0.740898 | 1 | Boosting | 6c61f5e5-5378-4bbe-835b-05f0cddb4742 | hERG |
| solifenacin | 0.365867 | 0 | Boosting | 6c61f5e5-5378-4bbe-835b-05f0cddb4742 | hERG |
| sotalol | 0.042638 | 0 | Boosting | 6c61f5e5-5378-4bbe-835b-05f0cddb4742 | hERG |
| sparfloxacin | 0.306478 | 0 | Boosting | 6c61f5e5-5378-4bbe-835b-05f0cddb4742 | hERG |
| sunitinib | 0.817265 | 1 | Boosting | 6c61f5e5-5378-4bbe-835b-05f0cddb4742 | hERG |
| telbivudine | 0.028096 | 0 | Boosting | 6c61f5e5-5378-4bbe-835b-05f0cddb4742 | hERG |
| terfenadine | 0.635287 | 1 | Boosting | 6c61f5e5-5378-4bbe-835b-05f0cddb4742 | hERG |
| terodiline | 0.863722 | 1 | Boosting | 6c61f5e5-5378-4bbe-835b-05f0cddb4742 | hERG |
| thioridazine | 0.827654 | 1 | Boosting | 6c61f5e5-5378-4bbe-835b-05f0cddb4742 | hERG |
| verapamil | 0.681325 | 1 | Boosting | 6c61f5e5-5378-4bbe-835b-05f0cddb4742 | hERG |
| voriconazole | 0.103862 | 0 | Boosting | 6c61f5e5-5378-4bbe-835b-05f0cddb4742 | hERG |

Such predictions were validated by comparing the classification and the class probabilities from the models with actual experimental data. Various metrics were to assess different aspects of the quality of the predictions. The following code was used to load experimental data that was in the form of a CSV file, although it should be understood that any other suitable file format can be used:

EXPDATA=pd.io.parsers.read_csv(“../data/Nature-Brown2013-Activities.csv”)
#Read csv file with experimental data
EXPDATA.head()

Exemplary experimental data for certain compounds is listed in Table 10.

TABLE 10

Exemplary Experimental Data

| Experimental Parameter/ID | amiodarone | astemizole | bepridil | ceftriaxone | chlorpromazine |
|---|---|---|---|---|---|
| TdP Risk | 1 | 1 | 1 | 0 | 1 |
| HERG-IC50 | 0.86 | 0.004 | 0.16 | 445.7 | 1.5 |
| HERG-pIC50 | 6.065502 | 8.39794 | 6.79588 | 3.350957 | 5.823909 |
| HERG-IC50-SEM | 0.12 | 0.001 | 0.02 | 146.9 | 0.1 |
| HERG-maxinhib | 81.8 | 83.6 | 86.4 | 7.5 | 96.7 |
| HERG-maxinhib-SEM | 2.7 | 4.4 | 5.3 | 2.1 | 0.7 |
| CAV1p2-IC50 | 1.9 | 1.1 | 1 | 153.8 | 3.4 |
| CAV1p2-pIC50 | 5.721246 | 5.958607 | 6 | 3.813044 | 5.468521 |
| CAV1P2-IC50-SEM | 0.2 | 0.1 | 0.2 | 23.9 | 0.3 |
| CAV1p2-maxinhib | 57.3 | 96.8 | 95.7 | 38.8 | 99.2 |
| CAV1p2-maxinhib-SEM | 3.4 | 0.1 | 1.3 | 4.2 | 0.8 |
| NAV1p5-IC50 | 15.9 | 3 | 2.3 | 555.9 | 3 |
| NAV1p5-pIC50 | 4.798603 | 5.522879 | 5.638272 | 3.255003 | 5.522879 |
| NAV1p5-IC50-SEM | 2.1 | 0.2 | 0.3 | 159.6 | 0.4 |
| NAV1p5-maxinhib | 86 | 90.5 | 100 | 14.3 | 97.1 |
| µM | 9.4 | 3.6 | 0 | 4.2 | 2 |
| Free drug (µM) | 0.0008 | 0.0003 | 0.035 | 23.17 | 0.038 |
| pFD | 9.09691 | 9.522879 | 7.455932 | 4.635074 | 7.420216 |

The pIC50 of each compound was calculated based on data such as shown in Table 10 using the following code, and the calculated pIC50s are shown in Table 11. For example, in order to compare the classification, experimental values are loaded from a comma separated values (cvs) file which are provided as pIC50s.
EXPDATA[‘pIC50’]=IC50_to_pIC50(EXPDATA[‘HERG-IC50’]) #It is better to work with pIC50 values instead of IC50
EXPDATA.index = EXPDATA[‘ID’]
pIC50s=EXPDATA[[‘ID’,‘pIC50’]]
pIC50s #The index must contain the unique compound ID as workaround for the bug in pd.DataFrame.join()

TABLE 11

pIC50s

| ID | pIC50 |
|---|---|
| amiodarone | 6.065502 |
| astemizole | 8.397940 |
| bepridil | 6.795880 |
| ceftriaxone | 3.350957 |
| chlorpromazine | 5.823909 |
| cilostazol | 4.860121 |
| cisapride | 7.698970 |
| clozapine | 5.638272 |
| dasatinib | 4.610834 |
| diazepam | 4.274088 |
| diltiazem | 4.879426 |
| disopyramide | 4.841638 |
| dofetilide | 7.522879 |
| donepezil | 6.154902 |
| droperidol | 7.221849 |
| duloxetine | 5.420216 |
| flecainide | 5.823909 |
| halofantrine | 6.420216 |
| haloperidol | 7.397940 |
| ibutilide | 7.744727 |
| lamivudine | 2.687400 |
| linezolid | 2.940361 |
| loratadine | 5.214670 |
| methadone | 5.455932 |
| metronidazole | 2.872830 |
| mibefradil | 5.769551 |
| mitoxantrone | 3.268089 |
| moxifloxacin | 4.064493 |
| nifedipine | 4.356547 |
| nilotinib | 6.000000 |
| nitrendipine | 4.609065 |
| paliperidone | 6.107905 |
| paroxetine | 5.721246 |
| pentobarbital | 2.843481 |
| phenytoin | 3.832683 |
| pimozide | 7.397940 |
| piperacillin | 2.467870 |
| procainamide | 3.564793 |
| quinidine | 6.142668 |
| raltegravir | 3.106349 |
| ribavirin | 3.014574 |
| risperidone | 6.585027 |
| saquinavir | 4.772113 |
| sertindole | 7.481486 |
| sitagliptin | 3.757707 |
| solifenacin | 6.552842 |
| sotalol | 3.953115 |
| sparfloxacin | 4.655608 |
| sunitinib | 5.920819 |
| telbivudine | 3.373968 |
| terfenadine | 7.301030 |
| terodiline | 6.187087 |
| thioridazine | 6.301030 |
| verapamil | 6.602060 |
| voriconazole | 3.309007 |

The validation and evaluation of the quality of the prediction can be performed by analyzing the correlation of the class probabilities with the experimental values, the receiver-operator-characteristic (ROC) curve. The class probabilities can be used to classify the compounds. In some embodiments, a class probability of less than 0.5 is labeled as ‘inactive’ against a given target and larger than or equal to 0.5 as ‘active.’ Furthermore, multiple metrics can used to measure the quality of the classification.

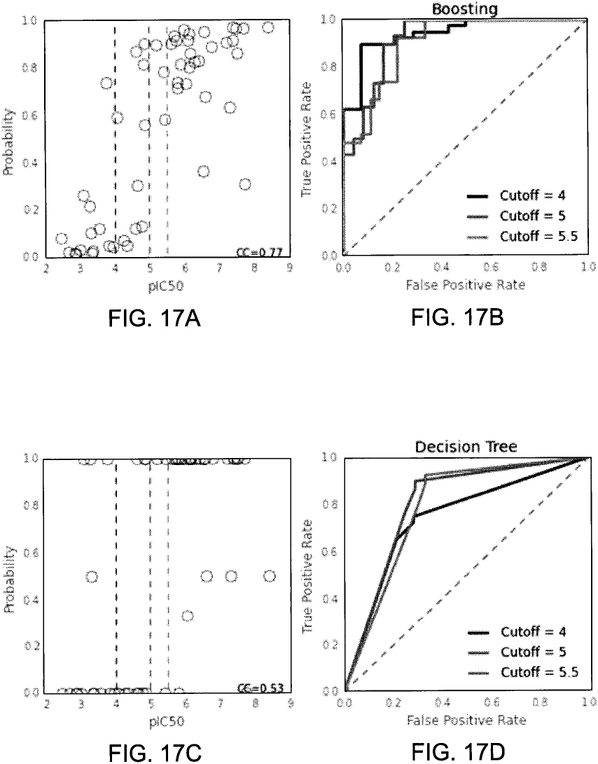

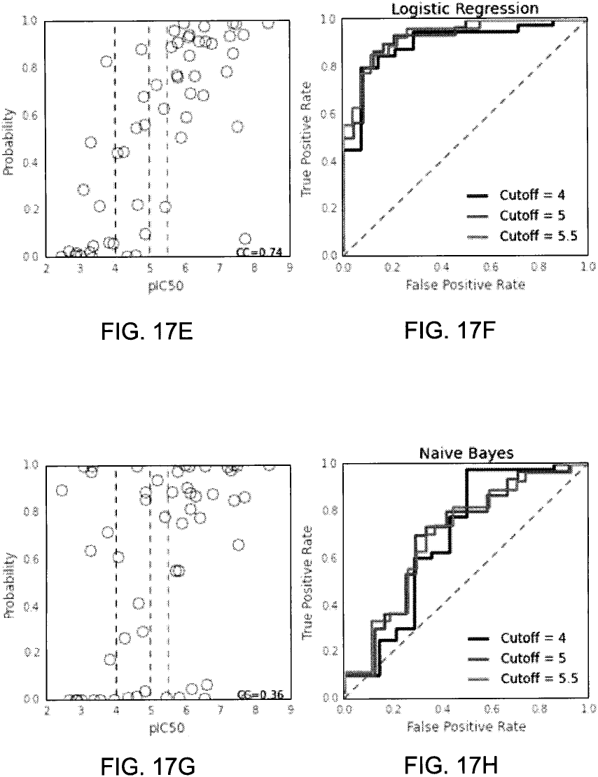

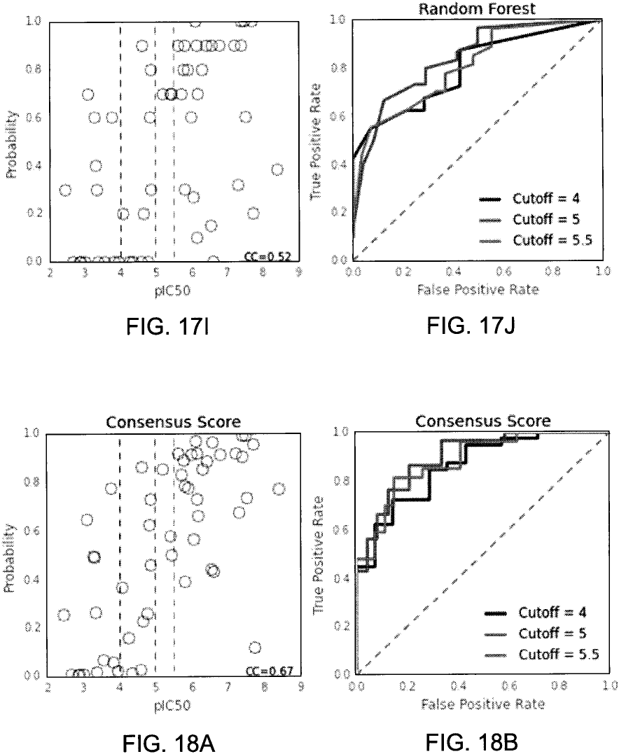

An example of an analysis is shown in FIGS. 17A-17J. The scatter plots (left plots in FIGS. 17A-17J) show the class probability to be active over the experimentally validated pIC50 values. The value for the Pearson correlation coefficient is shown in the figure (CC). The graphs (left) show receiver operator characteristic curves according to different cutoffs applied to the experimental data, that defines ‘active’ and ‘inactive’ compounds. For the model building a cutoff of 5 (pIC50) has been used. For the classification always a cutoff of 0.5 (class probability) has been applied so far. For example, a compound with the class probability 0.8 can be classified as ‘active’. The final dataframe contains the predictive power of the models according to the individual cutoffs and different metrics:

The following code was used to validate the predictions:

out=[]
cutoffs=[4,5,5.5]
for pred in predictions:
name = pred[‘Model’][0]
result=Validate_prediction(pIC50s,pred,title=name,cutoffs=cutoffs)
result[‘Model’]=pred[‘Model’].values[0]
out.append(result)
pd.concat(out).sort(‘PA’,ascending=False)
(prop.get_family(), self.defaultFamily[fontext]))

FIGS. 17A-17J illustrate probabilities to be active and ROC curves for an exemplary validation set for different machine learning algorithms, according to some embodiments of the present invention. More specifically, FIG. 17A illustrates probabilities to be active using the boosting machine learning algorithm, and FIG. 17B illustrates ROC curves for the boosting machine learning algorithm for different cutoffs. FIG. 17C illustrates probabilities to be active using the decision tree machine learning algorithm, and FIG. 17D illustrates ROC curves for the decision tree machine learning algorithm for different cutoffs. FIG. 17E illustrates probabilities to be active using the logistic regression machine learning algorithm, and FIG. 17F illustrates ROC curves for the logistic regression machine learning algorithm for different cutoffs. FIG. 17G illustrates probabilities to be active using the native Bayes machine learning algorithm, and FIG. 17H illustrates ROC curves for the native Bayes machine learning algorithm for different cutoffs. FIG. 17I illustrates probabilities to be active using the random forest machine learning algorithm, and FIG. 17H illustrates ROC curves for the random forest machine learning algorithm for different cutoffs. The scatter plots (FIGS. 17A, 17C, 17E, 17G, and 17I) show the class probability to be active over the experimentally validated pIC50 values. The value for the pearson correlation coefficient is shown in the figure (CC). The ROC graphs (FIGS. 17B, 17D, 17F, 17H, and 17J) show receiver operator characteristic curves according to different cutoffs applied to the experimental data, that defines ‘active’ and ‘inactive’ compounds. For the model building a cutoff of 5.5 has been used. For the classification always a cutoff of 0.5 has been applied so far. In one example, a compound with the class probability 0.8 is classified as ‘active’. In other examples, cutoffs of 3, 4, and 5 can be used.

Table 12 includes performance measurements of the various machine learning algorithms (MLAs) for different cutoffs.

TABLE 12

Performance Measurements

| Performance Measurement /MLA | Meaning | Logistic Regression | Boosting | Boosting | Logistic Regression | Logistic Regression |
|---|---|---|---|---|---|---|
| F1 | | 0.844444 | 0.764706 | 0.818182 | 0.833333 | 0.742857 |
| FN | Number of false negatives | 2 | 7 | 2 | 1 | 8 |
| FNR | False negative rate | 0.066667 | 0.175 | 0.066667 | 0.037037 | 0.2 |
| FP | Number of false positives | 5 | 1 | 6 | 7 | 1 |
| FPR | False positive rate | 0.208333 | 0.071429 | 0.25 | 0.259259 | 0.071429 |
| KK | Kohen’s kappa | 0.827869 | 0.816638 | 0.803279 | 0.802469 | 0.793718 |
| AC | Prediction accuracy | 0.87037 | 0.851852 | 0.851852 | 0.851852 | 0.833333 |
| Precision | | 0.791667 | 0.928571 | 0.75 | 0.740741 | 0.928571 |
| Sensitivity | | 0.904762 | 0.65 | 0.9 | 0.952381 | 0.619048 |
| Specificity | | 0.848485 | 0.970588 | 0.823529 | 0.787879 | 0.969697 |
| TN | Number of true negatives | 28 | 33 | 28 | 26 | 32 |
| TNR | True negative rate | 0.933333 | 0.825 | 0.933333 | 0.962963 | 0.8 |
| TP | Number of true positives | 19 | 13 | 18 | 20 | 13 |
| TPR | True positive rate | 0.791667 | 0.928571 | 0.75 | 0.740741 | 0.928571 |
| CC | Correlation coefficient | 0.74 | 0.77 | 0.77 | 0.74 | 0.74 |
| Cutoff | | 5 | 4 | 5 | 5.5 | 4 |
| AROC | Area under the receiver-operator curve | 0.925 | 0.944643 | 0.922222 | 0.932785 | 0.901786 |
| F1 | | 0.765957 | 0.734694 | 0.730769 | 0.72 | 0.636364 |
| FN | Number of false negatives | 2 | 7 | 6 | 8 | 6 |
| FNR | False negative rate | 0.074074 | 0.233333 | 0.222222 | 0.266667 | 0.2 |
| FP | Number of false positives | 9 | 6 | 8 | 6 | 10 |
| FPR | False positive rate | 0.333333 | 0.25 | 0.296296 | 0.25 | 0.416667 |
| KK | Kohen’s kappa | 0.728395 | 0.680328 | 0.654321 | 0.655738 | 0.606557 |
| AC | Prediction accuracy | 0.796296 | 0.759259 | 0.740741 | 0.740741 | 0.703704 |
| Precision | | 0.666667 | 0.75 | 0.703704 | 0.75 | 0.583333 |
| Sensitivity | | 0.9 | 0.72 | 0.76 | 0.692308 | 0.7 |
| Specificity | | 0.735294 | 0.793103 | 0.724138 | 0.785714 | 0.705882 |
| TN | Number of true negatives | 25 | 23 | 21 | 22 | 24 |
| TNR | True negative rate | 0.925926 | 0.766667 | 0.777778 | 0.733333 | 0.8 |
| TP | Number of true positives | 18 | 18 | 19 | 18 | 14 |
| TPR | True positive rate | 0.666667 | 0.75 | 0.703704 | 0.75 | 0.583333 |
| CC | Correlation coefficient | 0.77 | 0.53 | 0.53 | 0.52 | 0.36 |
| Cutoff | | 5.5 | 5 | 5.5 | 5 | 5 |
| AROC | Area under the receiver-operator curve | 0.903978 | 0.802778 | 0.788066 | 0.839583 | 0.697222 |
| F1 | | 0.564103 | 0.638298 | 0.679245 | 0.470588 | 0.55 |
| FN | Number of false negatives | 14 | 5 | 8 | 12 | 15 |
| FNR | False negative rate | 0.35 | 0.185185 | 0.296296 | 0.3 | 0.375 |
| FP | Number of false positives | 3 | 12 | 9 | 6 | 3 |
| FPR | False positive rate | 0.214286 | 0.444444 | 0.333333 | 0.428571 | 0.214286 |
| KK | Kohen’s kappa | 0.610357 | 0.580247 | 0.580247 | 0.587436 | 0.587436 |
| AC | Prediction accuracy | 0.685185 | 0.685185 | 0.685185 | 0.666667 | 0.666667 |
| Precision | | 0.785714 | 0.555556 | 0.666667 | 0.571429 | 0.785714 |
| Sensitivity | | 0.44 | 0.75 | 0.692308 | 0.4 | 0.423077 |
| Specificity | | 0.896552 | 0.647059 | 0.678571 | 0.823529 | 0.892857 |
| TN | Number of true negatives | 26 | 22 | 19 | 28 | 25 |
| TNR | True negative rate | 0.65 | 0.814815 | 0.703704 | 0.7 | 0.625 |
| TP | Number of true positives | 11 | 15 | 18 | 8 | 11 |
| TPR | True positive rate | 0.785714 | 0.555556 | 0.666667 | 0.571429 | 0.785714 |
| CC | Correlation coefficient | 0.53 | 0.36 | 0.52 | 0.36 | 0.52 |
| Cutoff | | 4 | 5.5 | 5.5 | 4 | 4 |
| AROC | Area under the receiver-operator curve | 0.74375 | 0.695473 | 0.806584 | 0.6875 | 0.799107 |

The predictions of the different models can be combined to obtain a consensus-score CS which can be superior over individual scores in many applications. In this example, the CS is simply the average of the class probabilities, which was calculated using the following code:

CS = pd.concat(predictions).groupby(‘ID’).mean()
CS[‘Classification’]=Classify(CS[‘Probability’],0.5)
def
Validate_prediction(pIC50s,probabilities,cutoffs=[5.5],inverse=False,pIC50colNa
me=‘pIC50’,title=None):
‘′’

This function validates the prediction according to a set of cutoffs used for defining ‘active’ and ‘inactive’ compounds.

pIC50s and prediction are pandas.DataFrames containing the values and the
compounds-IDs
‘′’
import pandas as pd
from sklearn.metrics import roc_curve,confusion_matrix,roc_auc_score
Colors = get_colors(len(cutoffs)
results=[]
plt.figure().set_size_inches((8,4))
#Convert cutoffs to iterable list in case a float/int cutoff has been used as input
#if not isiterable(cutoffs): cutoffs = [cutoffs]
for i,cutoff in enumerate(cutoffs):
color = Colors.next()
data = Join(probabilities,pIC50s)#.dropna()
data[‘Class’] = Classify(data[pIC50colName],cutoff)
auroc = roc_auc_score(data[‘Class’],data[‘Probability’])
plt.subplot(121)
if title: plt.title(title)
if i == 0: CC = PlotCor(data[pIC50colName],data[‘Probability’])
plt.vlines(cutoff,-0.2,1.2,linestyle=‘--’,color=color)
plt.xlabel(‘pIC50’)
plt.ylabel(‘Probability’)
plt.subplot(122)
if title: plt.title(title)
PlotROC(*roc_curve(data[‘Class’],data[‘Probability’],pos_label=1),\
linewidth=2.5,\
label=‘Cutoff = ’+str(cutoff),\
color=color)
tmp = EvalConfusionMatrix(
confusion _matrix(data[‘Classification’],data[‘Class’]))
tmp[‘CC’]=CC
tmp [‘Cutoff]=cutoff
tmp[‘AROC’]=auroc
results.append(tmp)
#PlotRandomROC(data[‘Class’],400)
if len(cutoffs) > 1: plt.legend(loc=4)
plt.tight_layout()
plt.show()
return pd.concat(results).sort(‘PA’,ascending=False)

FIGS. 18A-18B respectively illustrate probabilities to be active and ROC curves for an exemplary validation set for a consensus among different machine learning algorithms, namely a consensus prepared using the above code, according to some embodiments of the present invention.

Table 13 includes performance measurements of a consensus the various machine learning algorithm for different cutoffs, prepared using the above code.

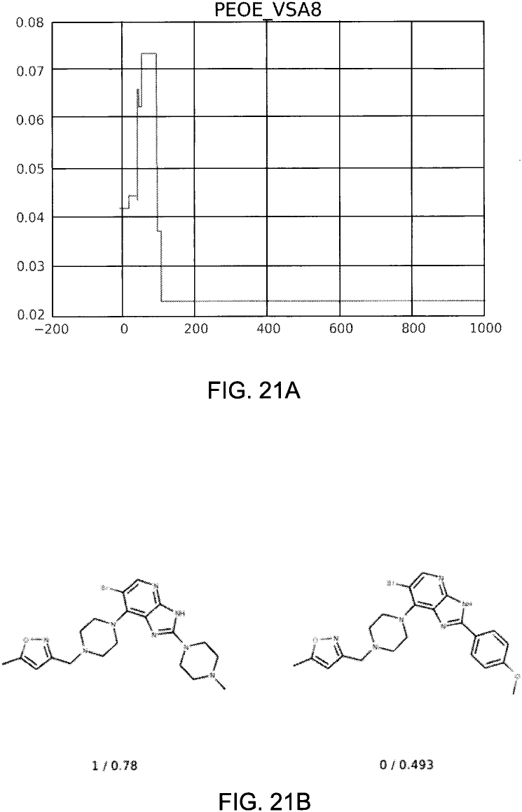

This example also shows how a model (trained machine learning algorithm) can be visualized and interpreted. In general, the interpretation of the models is not straightforward because many input parameters are used which are not necessarily linearly independent. An example of the boosting model on a specific dimension of the input vector is shown in FIG. 21A. However, similar input structures can be compared with small structural changes that lead to a comparably large shift in the class probability. FIG. 21B shows an example of a pair of an active compound (left) and inactive (right) where a change of a chemical group lead to a shift of 0.287 in the class probability. The comparison of the structures shows that an exchange of a aromatic ring with a carboxyl group has been replaced by a an aliphatic ring with two nitrogens. Reasoning how this structural change causes a change in the activity is beyond the scope of the current models. However, this analysis identifies pairs of compounds that can be studied with structure based methods like docking or molecular dynamics simulations.

TABLE 13

Performance Measurements for Consensus

| Performance Measurement | Meaning | Decision Tree | Naive Bayes | Random Forest |
|---|---|---|---|---|
| F1 | | 0.808511 | 0.76 | 0.648649 |
| FN | Number of false negatives | 4 | 4 | 11 |
| FNR | False negative rate | 0.133333 | 0.148148 | 0.275 |
| FP | Number of false positives | 5 | 8 | 2 |
| FPR | False positive rate | 0.208333 | 0.296296 | 0.142857 |
| KK | Kohen’s kappa | 0.778689 | 0.703704 | 0.702037 |
| AC | Prediction accuracy | 0.833333 | 0.777778 | 0.759259 |
| Precision | | 0.791667 | 0.703704 | 0.857143 |
| Sensitivity | | 0.826087 | 0.826087 | 0.521739 |
| Specificity | | 0.83871 | 0.741935 | 0.935484 |
| TN | Number of true negatives | 26 | 23 | 29 |
| TNR | True negative rate | 0.866667 | 0.851852 | 0.725 |
| TP | Number of true positives | 19 | 19 | 12 |
| TPR | True positive rate | 0.791667 | 0.703704 | 0.857143 |
| CC | Correlation coefficient | 0.67 | 0.67 | 0.67 |
| Cutoff | | 5 | 5.5 | 4 |
| AROC | Area under the receiver-operator curve | 0.9 | 0.884774 | 0.864286 |

### 6.4.3 Example 3

Some embodiments of the present systems and method s provide for “rehabilitation” or “redesign” of compounds that are predicted to include molecular parameters that are likely to be cardiotoxic, e.g., are likely to block the hERG1 channel or two or more of the channels disclosed herein. As used herein, “rehabilitation” can mean reducing one or more side effects (e.g., decreasing a hERG1 blocking affinity) while maintaining efficient binding to the desired target. In one non-limiting example, azole-based antifungal drugs can be at least partially rehabilitated while at least partially retaining their efficacy. For example, the systems and methods provided herein can be used to perform one or more of the following steps:
1) Predict the likelihood that a compound will block one or more, two or more, or all three of hERG, Nₐᵥ or Cₐᵥ ion protein channels,
2) Identify one or more therapeutic effect sites responsible for the desired target activity;
3) Re-design one or more molecular parameters, e.g., compound moieties, predicted to be responsible for blocking one or more, two or more, or all three of hERG, Nₐᵥ or Cₐᵥ ion protein channels, and that also are predicted to dispensable (e.g., not necessary) for binding to the desired target,
4) Perform differential analysis for on- and off- target interactions and perform fragment-based drug modification.

For example, the present systems and methods can be used to predict interactions between compounds and one or more, two or more, or all three of hERG, Caᵥ1.2 and Naᵥ1.5 channels. As provided elsewhere herein, such systems and methods can facilitate rapid and accurate evaluation of drug candidates, significantly reducing potential risks in drug development, e.g., based on molecular parameters of those compounds (e.g., solubility, lipophilicity, molecular weight, number of specific atoms, molecular fingerprints and other molecular and structural properties). Such molecular parameters can serve as variables used for supervised learning to against experimental activity data (e.g., ChEMBL database) to create predictive models. The Pearson correlation coefficients in the validation set between experimental and predicted pIC50 of hERG/Naᵥ1.5/Caᵥ2.1 model are 0.78/0.6/0.51 respectively (with saquinavir as clear outlier in all datasets) with blinded predictive power in torsadogenic activity of ~70% for identification of true-positives (torsadogenic) and 49% of true-negatives (non-torsadogenic). Therefore, the preliminary model shown in FIGS. 16A-16D, discussed elsewhere herein, already supersedes single-channel based predictive platforms.

The introduction of azoles and derivatives revolutionized the treatment of fungal and trypanosomosis infections. For example, miconazole, an imidazole antifungal agent, is directly associated with acquired QT prolongation and ventricular arrhythmias. For further details, see Kikuchi et al., “Blockade of HERG cardiac K+ current by antifungal drug miconazole,” British Journal of Pharmacology 144: 840-848 (2005), the entire contents of which are incorporated by reference herein. This is one of many examples of antifungal agents that are in clinical use despite apparent risks to patients. Our preliminary data from the previous grant on hERG1 blockade supported by the Heart and Stroke Foundation (Alberta, NWT) demonstrate that it possible to have compounds based on the structure of miconazole with substantially reduces effects on APs in neonatal mice cardiomyocytes. Some of these compounds have greater or comparable antifungal activity than miconazole itself, thus substantially reducing potential risks to patients. Azole derivatives are often prescribed in cases of the systemic fungal infection with IV delivery route posing substantial risks because of hERG, Naᵥ and likely Caᵥ blockade.

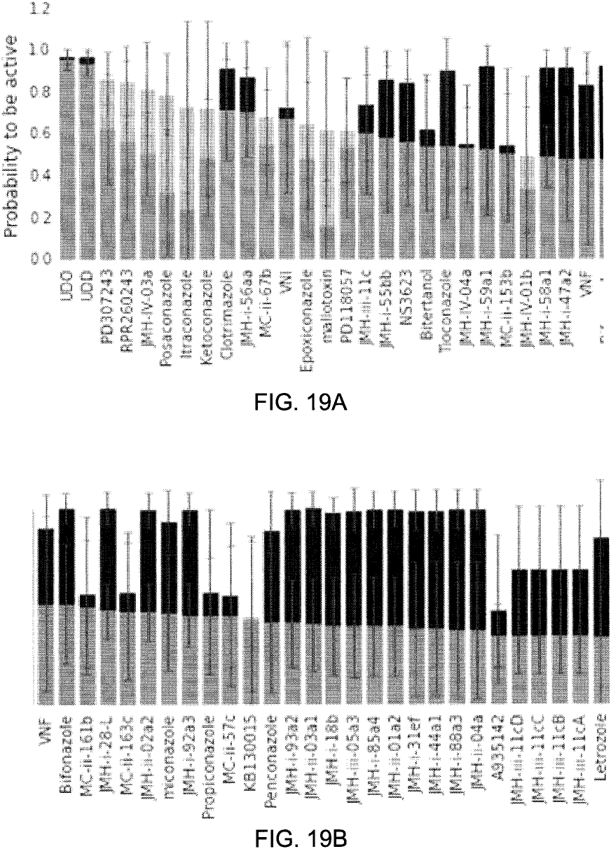

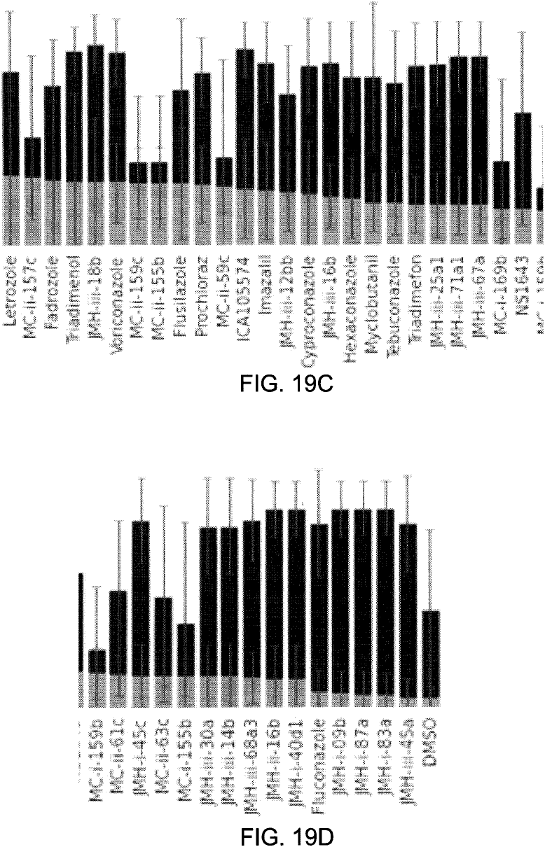

For example, FIGS. 19A-19D illustrate probabilities to be active on hERG (yellow) with respect to antifungal activity (blue) for an exemplary set of compounds, according to some embodiments of the present invention. FIGS. 19A-19D were prepared using steps analogous to those described above in Examples 1 and 2, but for an exemplary set of antifungal compounds.

With respect to the rehabilitation of established compounds that display unwanted interactions with cardiac targets, the compound can be as a starting material and different molecular parameters that are predicted to be cardiotoxic, including but not limited to structural features, can be blocked, cleaved or otherwise altered and the resulting modified compound then re-analyzed as provided herein so as to predict cardiotoxicity. Optionally, the compound or the modified compound, or both, can be chemically synthesized and assayed, e.g., tested for desired activity, such as antifungal activity.

### 6.4.4 Example 4

Appendix A attached hereto is incorporated by reference herein and forms part of the present disclosure, and relates to an example for predicting cardiotoxicity with respect to the hERG channel, using as input the same test set as described above with reference to Examples 1 and 2. More specifically, Appendix A includes hERG python code, with relevant inputs, example of SMILES input for a training set, and comparison to two other programs -Schrodinger Inc. and web-based server for QSAR hERG prediction. In this example, developer’s bits of explicit coding are omitted, and outputs are provided.

### 6.4.5 Example 5

Appendix B attached hereto is incorporated by reference herein and forms part of the present disclosure, and relates to an example for predicting cardiotoxicity with respect to the Nav1.5 channel, using as input the same test set as described above with reference to Examples 1 and 2. More specifically, Appendix B includes Nav1.5 python code, with relevant inputs, and example of SMILES input for a training set. In this example, developer’s bits of explicit coding are omitted, and outputs are provided.

### 6.4.6 Example 6

Appendix C attached hereto is incorporated by reference herein and forms part of the present disclosure, and relates to an example for predicting cardiotoxicity with respect to the Cav1.2 channel, using as input the same test set as described above with reference to Examples 1 and 2. More specifically, Appendix C includes Cav1.2 python code, with relevant inputs, and example of SMILES input for a training set. In this example, developer’s bits of explicit coding are omitted, and outputs are provided.

### 6.4.7 Example 7

The following example relates to a quantitative structure activity relationship (QSAR) model for the voltage gated potassium channel, known as hERG. The model is based on the XGBoost algorithm and trained on publicly available data from the ChEMBL database. The model performs well on compounds that are similar to the training set with a coefficient of determination of up to R²=0.8 and allows to quantitatively estimate the potential of novel chemical scaffolds to block hERG. The example employs a boosting tree algorithm for the machine learning algorithm to build the QSAR model. The purpose of the model is to quantitatively estimate the potential of novel chemical scaffolds in respect to hERG. In alternative embodiments, the methods presented below are applicable to other supervised learning tasks.

We used the currently most advanced publicly available boosting tree algorithms, which is known as extreme gradient boosting (XGBoost) to build a predictive model based on the content of the ChEMBL database. XGBoost is a parallel tree learning algorithm to build boosting tree classification, regression and ranking models (Chen et al., 2016, “XGBoost: A Scalable Tree Boosting System,” arXiv:1603.02754 [cs.LG], available at https://arxiv.org/abs/1603.02754). In recent years, ensemble methods such as random forest have been successfully applied to both classification and regression problems. Ensemble tree models use tree-like structures to classify instances or fit arbitrary functions. The fit is done based on attributes of the instances called features. An instance can be a chemical compound and the features may be molecular descriptors such as the weight, number of hydrogen bond donors. Each tree consists of a number of branches where the dataset is split corresponding to a chosen feature and a split value. The number of splits is often set to a smaller number what limits the ability of a single tree to fit a function accurately. But if many trees (ensembles) are combined very accurate classifiers can be designed. In boosting algorithms each tree aims to fit the instances better that are missed by the previous trees.

Our model is based on open source software and trained on publicly available data from ChEMBL database (Gaulton et al., 2012, “ChEMBL: a large-scale bioactivity database for drug discovery,” Nucleic Acids Res., 40, D1100-7). We used the open source toolkit for cheminformatics RDKit (Landrum, “RDKit: Open-source cheminformatics” available at http://www.rdkit.org) to handle chemical structures, reactions and transformations and to calculate molecular descriptors and similarities.

The ChEMBL database (December 2015) was queried for bioactivities for the target ‘CHEMBL240’ the potassium voltage-gated channel subfamily H member 2 (hERG) using the python chembl-webresource-client. The ChEMBL database at the date of evaluation contained single protein bioactivities for 6018 different targets. hERG ranked 54 for the number of available bioactivities and 1st when the query was restricted to IC50 values. Assay descriptions that were included were: ‘Inhibition of human ERG’, ‘Binding affinity to human ERG’, ‘Inhibition of human ERG at 10 uM’ and ‘Inhibition of human ERG channel’. Furthermore, the query was restricted to the bioactivity type ‘IC50’, the assay type ‘B’, the operator ‘=’ and the target confidence ‘9’. Then the dataset was restricted to assays that contain at least 6 activities for different compounds in order to increase the consistency of experimental data in the training set. Only items with a specified IC50 value were selected.

Duplicate compounds in the dataset were identified based on compound similarities. In case the same value was reported multiple times the items were grouped. If multiple items with different IC50 values remained the record with the minimal IC50 value was selected after checking the values in the original publications. In some cases the reported IC50 values was transcribed wrongly into ChEMBL. In this cases the values were corrected. In the following the four different test sets are described.

From the curated ChEMBL dataset that contained 699 compounds 100 compounds were selected randomly and saved as first external test set (Test1). This set contained experimental values obtained by the same 40 assay-IDs as the training set. Test set 2 (Test2) contained 55 compounds with IC50 values measured with the same protocol and were reported by Kramer et al. (Kramer et al., 2013, “MICE models: superior to the HERG model in predicting Torsade de Pointes,” Sci Rep., 3, 2100). Smiles codes for the compounds were generated with “molconvert” from ChemAxon’s MarvinSketch. The records belonging to assays that contain between 2 and 5 compounds were defined as test set 3 (Test3), in total 155 compounds. The remaining 73 entries were defined as test set 4 (Test4). Every record in Test4 had a unique ChEMBL assay ID. Compounds with a pIC50 > 5 were defined as ‘active’ compounds.

### 6.4.7.1 Feature Generation and Feature Sets

The smiles codes for the compounds were obtained from the ChEMBL database. The codes were used to generate RDKit molecule objects (RDKit version: 2015.03.1) and standardized using molvs (version 0.03) package. Hydrogen atoms were added. The resulting structures were used to calculate all molecular descriptors that were implemented in RDKit.

### 6.4.7.2 Predictive Features

All molecular descriptors with zero variance were removed. The remaining features were filtered, so that no two features had a mutual absolute pearson correlation above 0.99 based on data in the training set. The final set contained 150 descriptors. As mentioned above we used a cutoff of pIC50 > 5 to define ‘active’ compounds. Based on this assignment we analysed the predictive power of individual the molecular descriptors using the receiver-operator-characteristic (ROC) and the corresponding area under the ROC (AROC) for each descriptor. The molecular descriptors with an AROC > 0.55 are referred to as ‘predictive features’ comprising 55 features.

### 6.4.7.3 Normal Features

The predictive features were subjected to a test for normality. Features that met the criterion for normality were selected. The ‘normal features’ are a subset of the predictive features. More information can be found in the supplementary information. The normal features comprised 48 features.

### 6.4.7.4 Similarity Based Features

The compound similarities were calculated with RDkit. We used morgan fingerprints (nBits=1024, radius=2) and the Tanimoto similarity score of the standardized molecules. For each compound the four most similar compounds in the training set were identified. The similarities as well as the pIC50 values of the corresponding compounds were added to the database as features. The similarity of the most similar compounds is named ‘sim0’, the second best ‘sim1’ and so on. The corresponding pIC50 values ‘value0’, ‘value1’, and so on. These features were based on the K-nearest neighbors algorithm and the observation that similar compounds have higher probability to have similar pIC50 values and used to predict the activity of a compound based on the activity of the most similar compounds, wherein the eight features are referred to as similarity based features.

### 6.4.7.5 2D Pharmacophore Features

A set of chemical features with topological (2D) distances between them produce 2D pharmacophore features. We used the feature definitions from Gobbi and Poppinger (Gobbi et al., 1998, “Genetic optimization of combinatorial libraries,” Biotechnol. Bioeng., 61, 47-54) as implemented in RDKit. The compounds in the training set were converted to 2D fingerprints represented as bit-vectors. Each element of the bitvectors served as a feature for the machine learning algorithm, while keeping bits that were activated at least 100 times.

### 6.4.7.6 Feature Sets

Different sets of features were evaluated. The following list indicates each set and assigns a number to serve as reference: 1: PredictiveFeatures, 2: NormalFeatures, 3: SimilarityFeatures, 4: Pharm2DFeatures, 5: PredictiveFeatures + SimilarityFeatures, 6: PredictiveFeatures+Pharm2DFeatures, 7: PredictiveFeatures + Pharm2DFeatures + SimilarityFeatures, 8: NormalFeatures + Pharm2DFeatures.

### 6.4.7.7 Model Selection

For use XGBoost algorithm, a stepwise protocol was used. First, cross-validation was used to identify the most dominant features and parameters. Learning curves were generated to characterize the dependency of the model performance in respect to the size of the dataset as well as to identify the optimal number of iterations for the generation of the final model.

Since XGBoost can be quite sensitive to the parameters used to fit the model, we performed a grid search to find a combination of parameters and features with best cross-validated performance. The parameters and corresponding values for the grid search were: ‘colsample_bytree’: 0.3/0.5/0.8/0.9, ‘subsample’: 0.2/0.5/0.8/0.9, ‘max_depth’: 3/4/5/6/8 and ‘eta’: 0.001/0.01/0.05/0.1. The dataset was shuffled and afterwards divided into 10 mutually exclusive groups. For each fold, a model was trained using compounds from 9 groups and the remaining group was used for validation. The RMSE for training and validation set was monitored. Once the validation error did not increase within 1000 iterations, the fitting was stopped. The results of the best iteration were stored for the validation set. The stored data was used to calculate the cross-validated coefficient of determination Q2, AROC, and other metrics. This procedure was repeated for each combination of features and parameters as set forth above.

For the selected parameters and features, learning curves were generated. The learning curve monitors the on- and off-sample performance over the size of the training set. The learning curves were generated by averaging the performance of 20 repetitions. Random samples of the complete training served as validation set and fractions of the remaining compound were used to fit the model. In some embodiments, the size of the validation set was 10% of the complete training set. The number of regression trees was controlled for each curve and varied between 200 and 1,500.

The final model was trained using the complete training set and a predefined number of iterations. The final model trained using 900 iterations, which was slightly higher than the optimal number identified with the learning curves. This was done to compensate for the slightly higher number of training samples when using the full dataset. The parameters for the final model were: colsample_bytree: 0.9, eta: 0.01, max_depth: 3, subsample: 0.2.

### 6.4.7.8 Y-randomization

The Y-randomization test prevents high model performance due to chance correlation. We used y-randomization, i.e. randomly shuffle the target variable and repeat the model selection and fitting procedure. In some embodiments, Y-randomization was used to eliminate chance correlation among the generated model. In some embodiments, no Y-randomization was used.

A stepwise protocol was used to select the model parameters and features. 10-fold cross-validation was used to identify the most dominant feature sets (F1-8) and hyper-parameters. The optimal number of iterations was estimated based the shape of learning curves. Afterwards, the model was applied to pre-defined test sets to estimate the model’s ability to generalize and to estimate the off-sample performance. The method of model selection

### 6.4.7.9 Predictive Power of Individual Features

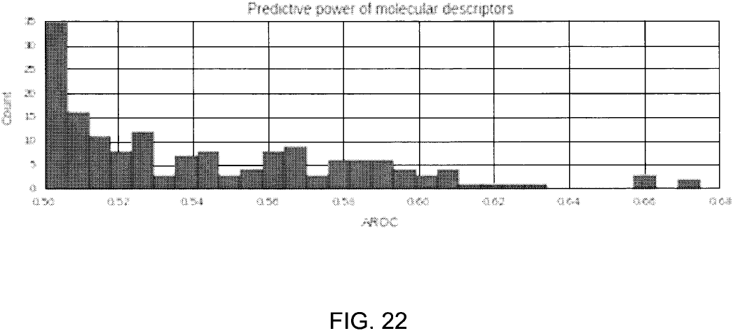

We performed a ranking of all features by calculating the area under the ROC-curve (AROC). We applied defined positive class as compounds with pIC50 > 5 otherwise the compounds belong to the negative class. The sign of the feature was changed when the AROC was negative, ensuring a ranking according to the actual discriminative power. A histogram of the AROC values for the molecular descriptors is shown in FIG. 22. Only five descriptors generated a curve with an AROC above 0.65. The highest value (0.67) was obtained by the molecular LogP value (MolLogP), wherein in some embodiments, a valid model has at least have an AROC of 0.67. We analyzed the predictive power of the remaining features by calculating the area under the ROC curves (AROC).

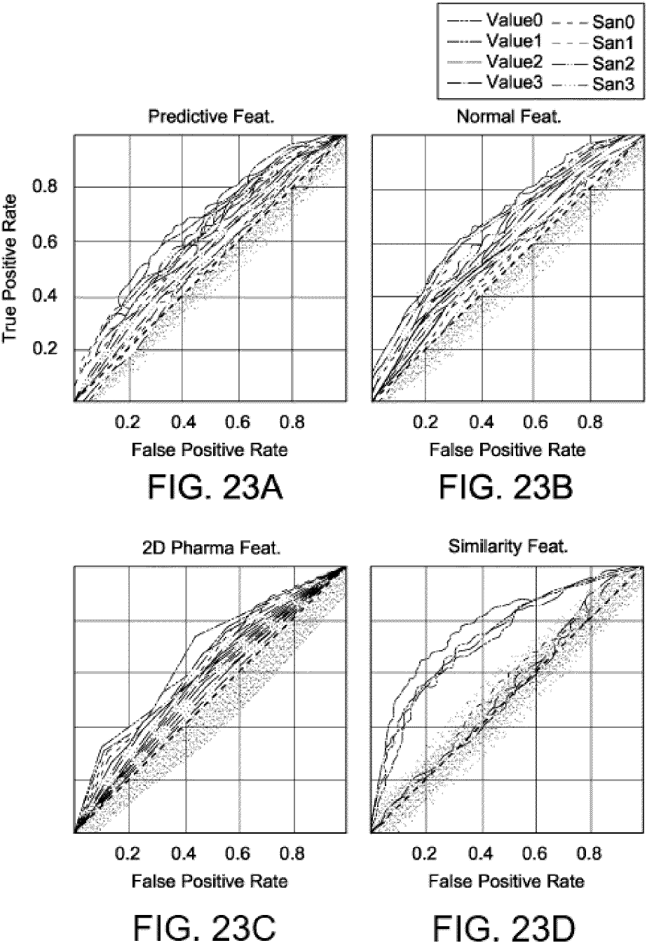

The ROC curves of all groups of features are shown in FIGS. 23A-D The most predictive molecular descriptors was the molecular logP value. The valueX features, i.e prediction based on the pIC50 values of the most similar compounds, generate a highly significant ROC-curve. In contrast the simX features are essentially random predictions. This is expected since the maximal similarity to the training set does not carry information about activity per se. The simX features are still meaningful, since these feature can serve as weighting factors and contribute to a better prediction for models that are limited to the similarity based features.

### 6.4.7.10 Model Selection

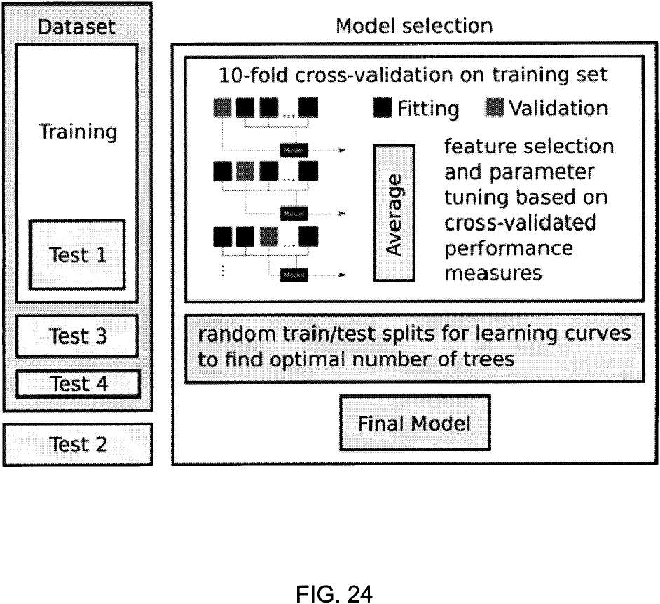

Steps of the method of selecting a model for predicting cardiotoxicity of a compound are shown in FIG. 24, according to some embodiments of the present invention.

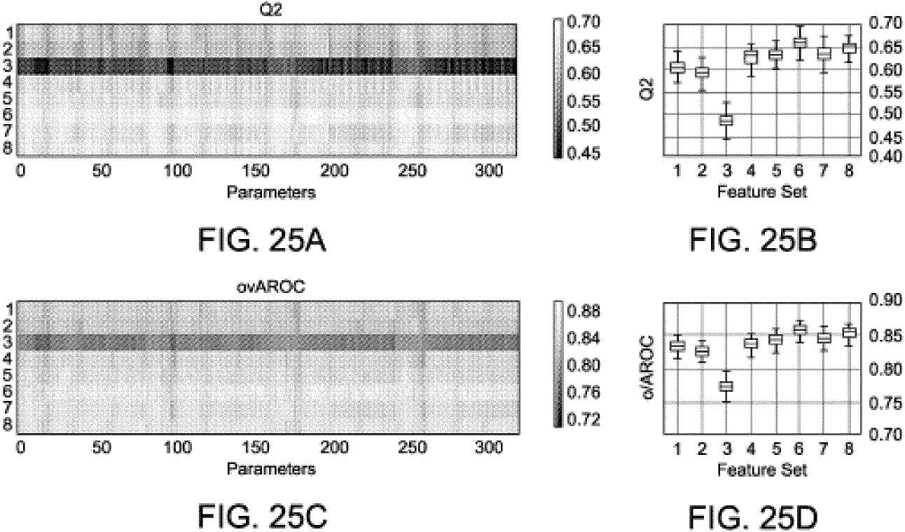

We tested 320 different sets of parameters times 8 different sets of features. The best set of features according to both the mean cross-validated R2 (Q2) score and cross-validated AROC (cvAROC) was feature set 6, the combination of the predictive molecular descriptors and the 2D pharmacophore features. This combination performed best for a broad range of parameters on both metrics (FIGS. 25A-D). The median Q2 of feature set 6 was 0.66 followed by feature set 8 with 0.65. The worst set was feature set 3 with 0.49. The maximum value was 0.67 by feature set 6. From feature sets F1-F4 the 2D-pharmacophore features (F4) did perform best.

The individual similarity based features (value0-4) showed the highest AROC values without resulting in over-all improvement. The maximum AROC using only the similarity based features was 0.8 compared to 0.78 gained by the feature ‘value0’ only. The median Q2 values was 0.49 with a standard deviation of 0.018. Together, with the predictive features (F5) the median Q2 value increased to 0.63 compared to 0.61 using only the predictive features (F1), while adding them to the predictive features and the pharmacophore features decreased the performance (F7).

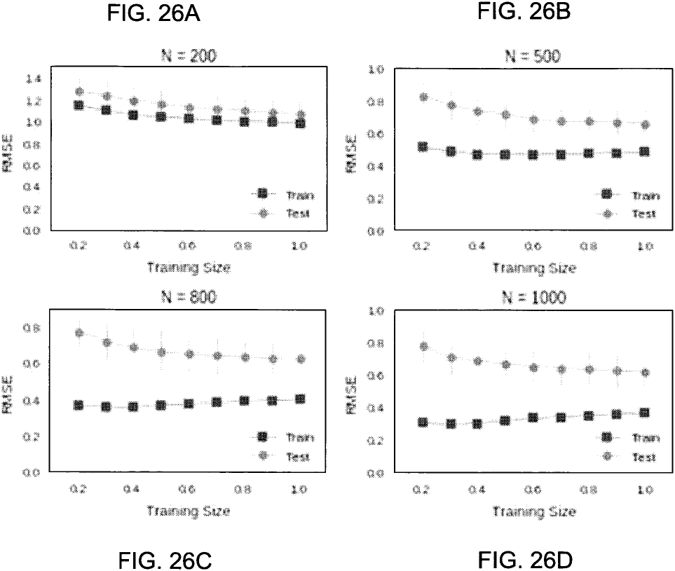

As shown in FIGS. 26A-D train and test error had similar values for less iterations. For more iterations both curve decreased to lower values due to a better fit of the training data. For all curves the validation curve had a negative slope, indicating that the predictive power increased with the size of the training set.

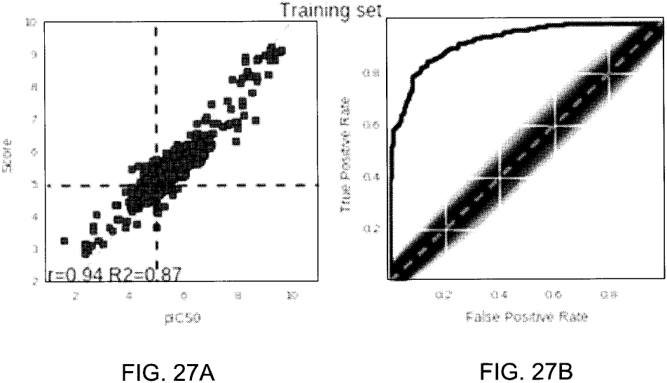

The off-sample error stopped decreasing at around 800 iterations. For this set of parameters and the dataset at hand, the optimal number of iterations was around 800. Based on this results we fitted the final model using 900 iterations taking into account the slightly bigger size of the training set when using all compounds. FIGS. 27A-B shows the fitted training data and the corresponding ROC curve. The RMSE was around 0.5 units and the AROC was 0.90.

### 6.4.7.11 Model Evaluation

After identification of the best features and parameters we trained the model using the complete training set of around 700 compounds. Afterwards, we evaluated the off-sample performance based on 4 different test sets. The sets differed in their origin, size and composition. Test set 1 (Test1) was a randomly chosen subset removed from the training set before modeling. Test set 2 (Test2) comprised 55 compounds with experimental data from the same protocol (Kramer et al., 2013, “MICE models: superior to the HERG model in predicting Torsade de Pointes,” Sci Rep., 3, 2100). Initially, test sets 3 (Test3) and 4 (Test4) were not dedicated as test sets because, especially for Test4, the number of assays and therefore the expected inconsistency of experimental factors is high. However, these sets are still valuable to probe the limitations of our model and useful to define a reasonable applicability domain.

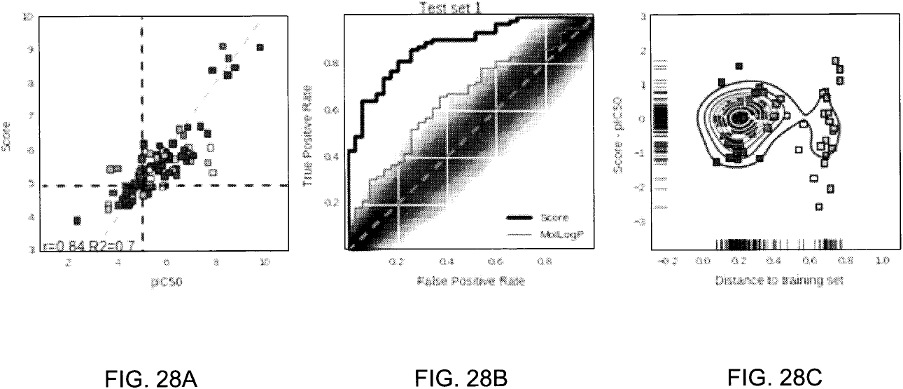

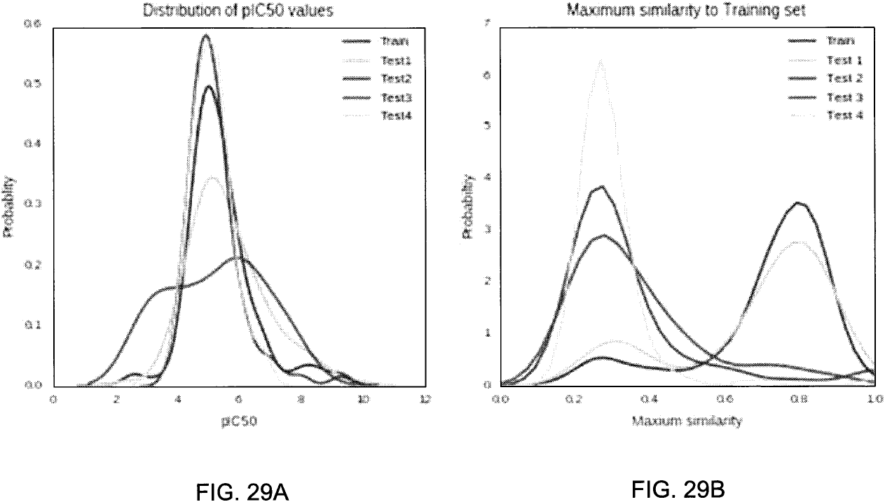

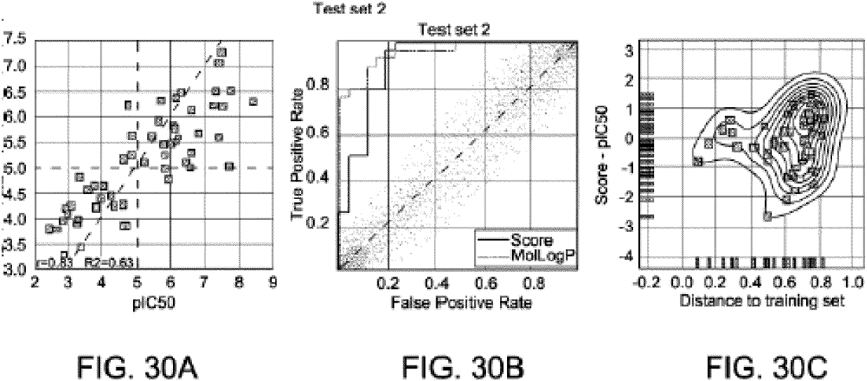

For Test1 the final model demonstrated reasonable performance to estimate a compound’s pIC50 value. The correlation between the predicted (score) and the experimental pIC50 values is 0.84 the R² score is 0.7, as shown in FIGS. 28A-C. The relative ranking performance was quantified with the AROC which was 0.88. The root mean squared error (RMSE) was 0.7. The similarity between the compounds in Test1 and the training set was high compared to the other test sets FIG. 29B. Although, as shown in FIGS. 30A-C, with 0.91 the AROC for to Test2 higher as for Test1. Also the RMSE was higher (0.95).

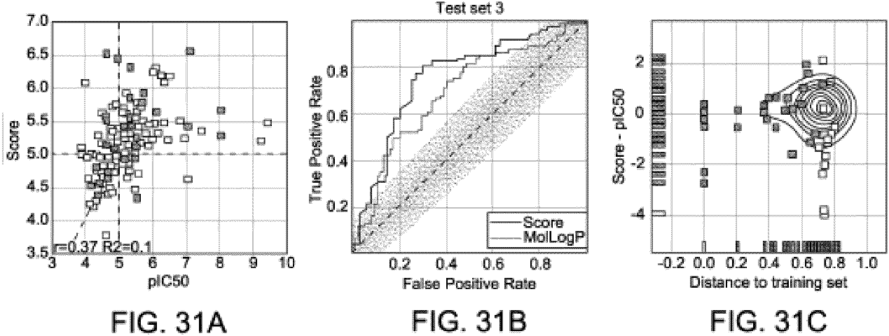

For Test3 the AROC was 0.76, as shown in FIGS. 31A-C. Interestingly, in two cases compounds that were structurally identical to compounds in the training set showed significant deviances from the experimental data. This was because the separation of the experimental data in training set and test sets was done using assay identification numbers in the ChEMBL database. Within each set duplicates were removed, but not duplicates that appear in different sets. The disagreement of the predicted value with the experimental value demonstrates how different reported pIC50 values from different assays can be. The related compounds are CHEMBL41, CHEMBL549. CHEMBL41 is also known as Prozac it is identical to CHEMBL1201082 which is the corresponding salt. CHEMBL549 is also known as Cilatopram.

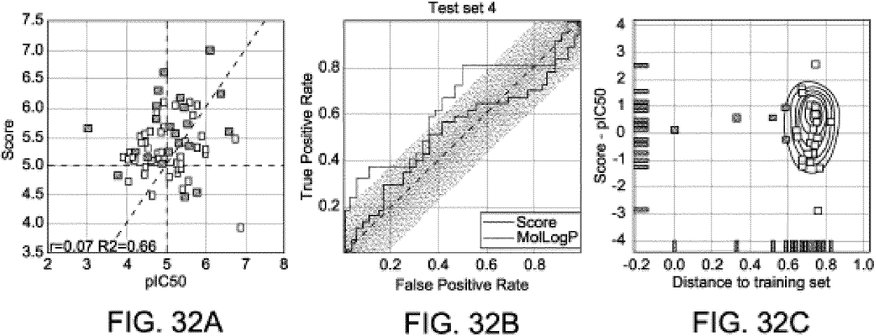

Finally, for Test 4 the AROC dropped to 0.52 which can be considered a random prediction. As shown in FIGS. 32A-C, the ROC curve was within the area that is expected for random predictions. Both the average distance to the training set and the experimental diversity was highest for Test4. Also the range of pIC50 values that the test set spans was lower. This made it challenging to predict the pIC50 values. For the majority of compounds, a smaller distance to the training set went along with lower RMSE values.

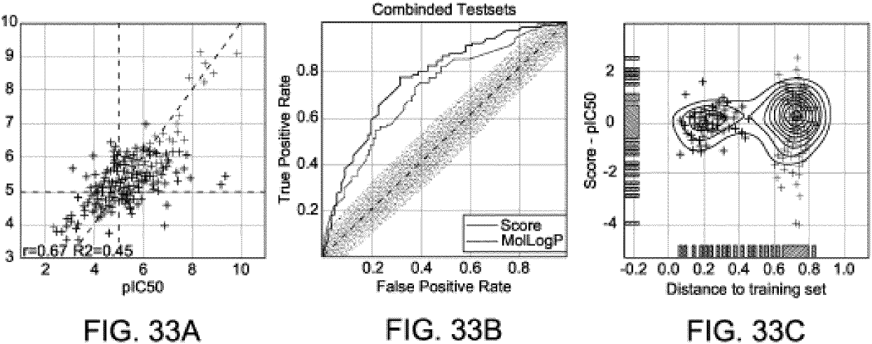

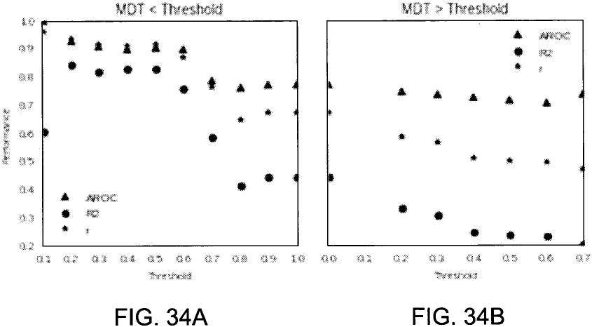

We investigated the relationship between the RMSE and the minimal distance to the training set (MDT) by combining all test sets, as shown in FIGS. 33A-C. Then performed a threshold analysis by neglecting all compounds with MDT above/below the threshold value and analysed the performance on the remaining compounds. For this analysis, we removed all compounds with MDT=0. The results for AROC, R² and r are shown in FIGS. 34A-B. We observed a clear drop of the performance when considering compound with a MDT larger than 0.6. The AROC dropped to values 0.8, the R² went down to 0.4. When considering compounds with MDT larger than the threshold, all values decreased even further. When exclusively considering the performance for compounds with MDT > 0.6, the AROC stayed above 0.7, which is a significant result.

### 6.4.7.12 Model Interpretation

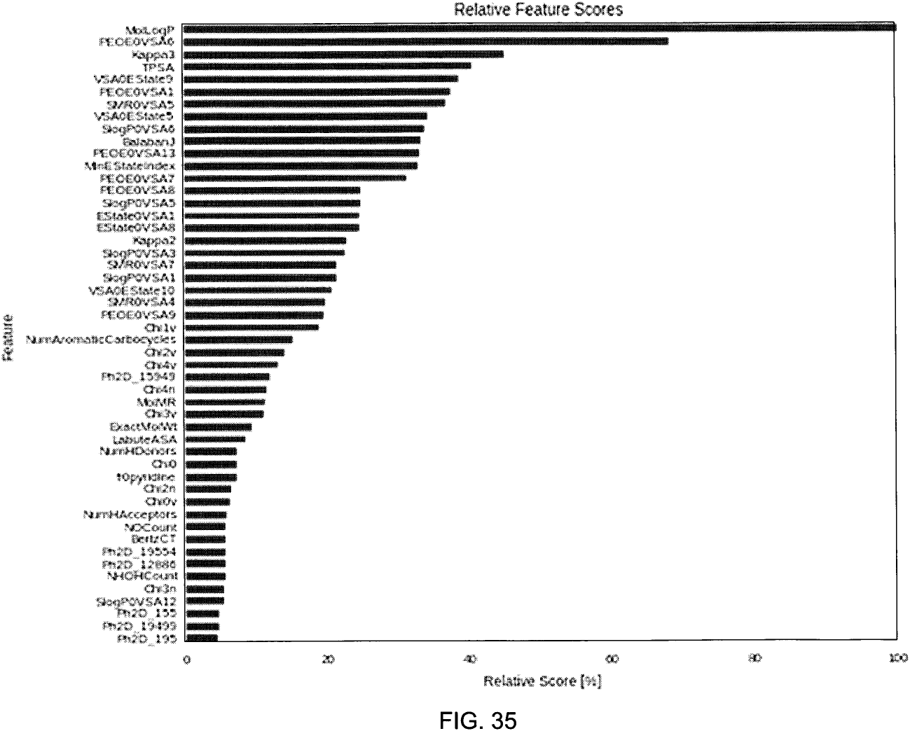

The frequencies of the features used in the final model were evaluated with the fscore function implemented in XGBoost. The feature that was most frequently used by the model was the molecular logP value, followed by MOE like descriptor (PEOE _VSA6), Kappa3 and the topological polar surface area. The top ranks were dominated by molecular descriptors. 2D-Pharmacophore features had lower scores. FIG. 35 shows that the model frequently uses a variety of features. The model allows estimating the pIC50 value within a range of around one pIC50 units for compounds that are similar to the training set.

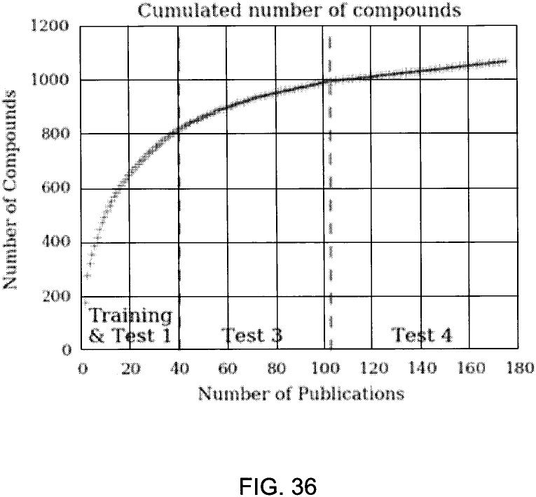

The distribution of pIC50 values in the training set had a maximum at 5 which is used as the classification cutoff to define active and inactive classes. This means low and highly active compounds were overrepresented in Test3 compared the other sets, which made it easier for the model to distinguish both classes, as shown in FIGS. 29A-B and FIG. 36. The per compound similarity between Test3 and the training set was rather low compared to Test1. Test4 was less predictive, because the set had a comparably narrow range of pIC50 values in addition to the compounds being very dissimilar to the training set. As shown in FIG. 29A, Test4 contained almost no compounds with a maximum similarity above 0.5. The distributions of both the pI50 values as well as the maximum similarity of Test1 was most similar to training set. This was expected because both sets were drawn randomly from the same pool of compounds, while exhibiting some differences as well, which are based on by random fluctuations and the limited size of 700 compounds in total. The distribution of pIC50 values of Test 3 and 4 is very similar to each other, as illustrated in FIG. 29A.

We found AROC values around 0.9 and R² above 0.8 for compounds that were similar to the training set (MDT < 0.5). For subsets of less similar compounds, the performance dropped. For the least similar subset (MDT > 0.7), the result was still better than random, while the best performance was observed for compounds that were similar to the compounds in the training set.

The performance on the least similar subset was still better than the performance on Test4, indicating that the diversity among the experimental methods used for compounds in Test4 reduces the predictive power of the model. In Test4, every compound had a different ChEMBL assay ID. The model performed well on Test2 with the majority of compounds in Test2 having an MDT above 0.6, which is based on the compounds in Test2 sharing the same experimental conditions, thus displaying a uniform distribution of pIC50 values.

For Test1, a simple random selection of 100 compounds, the R² value was 0.7 and the correlation coefficient was 0.84. The differences in performance shows that it is important to take the test set composition into account when estimating the off-sample performance in addition to capturing time dependence, which was not considered in this current model.

None of the compounds in the test sets was used during the training and model selection process, except for two compounds that were present in Test3. These compounds had different experimental values in both sets and therefore, did result in an overestimation of the performance of the model, while being representative of the diversity of the data. In some cases, different pIC50 were measured for different isoforms.

The present model was based on 0D, 1D and 2D descriptors without taking into account the conformation of a compound. An improvement of the model would include capturing effects that depend on 3D conformations like stereoisomerism.

Even for the training set the model had an RMSE above 0.4 pIC50 units and at least 0.51 for the test sets. If pIC50 values for a group of new compounds is known, the data of these compounds can easily be included in the training set to retrain the model. Since structurally closely related compounds are more likely to have similar activities, the model is able to estimate the affinities of such derivative compounds fairly accurate. By generating thousands of derivatives of a single compound, the model can be used to rank and prioritize these derivatives.

Besides fscore as “feature importance,” the addition to features with low scores significantly increased the performance of the model. As shown in FIGS. 26A-D, incorporating the 2D-pharmacophore features boost model performance, while none of these features had a relative score above 10% (FIG. 35). In some cases, the values provided by the fscore function resulted in a more pronounced boost of performance in comparison to the other model parameters.

After the tuning process, the final model was trained on the complete dataset using in total 120 features to ensure all available information was used to build the model. The gradient boosting regression tree algorithm performs internal feature evaluation and only uses features that are important to improve the fit. Using a small learning rate and randomly chosen subsamples reduced the risk for overfitting, since for every step the algorithm evaluated a different sample of the dataset.

The learning curve allows visualizing how the model behaviour changes with varying the size of the training set. The learning curve is also used to analyze whether the model suffers from high bias or variance, and to decide whether the inclusion of more data would improve the model. If the learning curve indicates that the regression model suffers from high variance in the dataset, adding more reliable features and consistent data likely improves the model. Addition of 3D features will likely boost the performance of the model, since for some compounds we observed a steady decrease of the validation RMSE when increasing the training set size.

Comparing curves with different number of trees allowed us to determine when the model started to overfit the training data. Overfitting occurs, when the error in the test set stopped decreasing and eventually started increasing with more iterations indicating that the model generalizes. One of the advantages of tree based models is their robustness against overfitting. The error-bars in the plots were the calculated using standard deviations, expecting lower RMSE values for the training set (Train) as compared to the validation/test sets (Test). Feature selection and parameter tuning was done as described in detail above.

Different stereoisomers were not distinguishable in our modelling approach. Most compounds come as mixtures between all isomers and the effective IC50 is an average dominated by the stereoisomer with the lowest IC50 value. Stereoisomerism is not captured in the less than 3D descriptor space of RDKit molecular descriptors or 2D-pharmacophore features. An option for the model includes to add 3D features, which is motivated by the fact that, for example, terfenadine and its metabolite fexofenadine undergo a change in their 3D equilibrium structure due to the formation of an intramolecular hydrogen bond, which prevents hERG blocking. Addition of 3D features would require identifying a finite number of relevant 3D conformers among infinitely many possible confirmations of a particular compound. The model of this example was based on molecular properties that do not depend on the 3D conformation of neither the ligand nor a receptor, providing a baseline for structure based virtual screening of compounds against hERG.

### Alternative Embodiments

It should be understood that the examples and code sections provided above are intended to be purely exemplary and not limiting of the present invention.

Additionally, it should be noted that the systems and methods can be implemented on various types of data processor environments (e.g., on one or more data processors) which execute instructions (e.g., software instructions) to perform operations disclosed herein. Non-limiting examples include implementation on a single general purpose computer or workstation, or on a networked system, or in a client-server configuration, or in an application service provider configuration. For example, the methods and systems described herein can be implemented on many different types of processing devices by program code comprising program instructions that are executable by the device processing subsystem. The software program instructions can include source code, object code, machine code, or any other stored data that is operable to cause a processing system to perform the methods and operations described herein. Other implementations can also be used, however, such as firmware or even appropriately designed hardware configured to carry out the methods and systems described herein. For example, a computer can be programmed with instructions to perform the various steps of one or both of the flowcharts shown in FIGS. 1A-1B.

It is further noted that the systems and methods can include data signals conveyed via networks (e.g., local area network, wide area network, internet, combinations thereof, etc.), fiber optic medium, carrier waves, wireless networks, etc. for communication with one or more data processing devices. The data signals can carry any or all of the data disclosed herein that is provided to or from a device.

The systems’ and methods’ data (e.g., associations, mappings, data input, data output, intermediate data results, final data results, etc.) can be stored and implemented in one or more different types of computer-implemented data stores, such as different types of storage devices and programming constructs (e.g., RAM, ROM, Flash memory, flat files, databases, programming data structures, programming variables, IF-THEN (or similar type) statement constructs, etc.). It is noted that data structures describe formats for use in organizing and storing data in databases, programs, memory, or other computer-readable media for use by a computer program.

The systems and methods further can be provided on many different types of computer-readable storage media including computer storage mechanisms (e.g., non-transitory media, such as CD-ROM, diskette, RAM, flash memory, computer’s hard drive, etc.) that contain instructions (e.g., software) for use in execution by a processor to perform the methods’ operations and implement the systems described herein.

Moreover, the computer components, software modules, functions, data stores and data structures described herein can be connected directly or indirectly to each other in order to allow the flow of data needed for their operations. It is also noted that a module or processor includes but is not limited to a unit of code that performs a software operation, and can be implemented for example as a subroutine unit of code, or as a software function unit of code, or as an object (as in an object-oriented paradigm), or as an applet, or in a computer script language, or as another type of computer code. The software components and/or functionality can be located on a single computer or distributed across multiple computers depending upon the situation at hand.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Finally, as used in the description herein and throughout the claims that follow, the meanings of “and” and “or” include both the conjunctive and disjunctive and can be used interchangeably unless the context expressly dictates otherwise; the phrase “exclusive or” can be used to indicate situation where only the disjunctive meaning can apply.

### Other Alternative Embodiments

While various illustrative embodiments of the invention are described above, it will be apparent to one skilled in the art that various changes and modifications may be made therein without departing from the invention. The appended claims are intended to cover all such changes and modifications that fall within the true spirit and scope of the invention.

### APPENDIX A

[image]

### hERG Activity Prediction

Can we use molecular structures, related properties and similiarities to distinguish between hERG active and inactive compounds?
desired accuracy +90%

RDkit : Convert smiles codes into molecular descriptors Out[16]:

| | CSP3 | LogP | MW | NAr omRing | NG ACC | NH At | NH Don | NO Count | NRings |
|---|---|---|---|---|---|---|---|---|---|
| 0 | 1.00 | 1.41630 | 44.062600 | 0 | 0 | 3 | 0 | 0 | 0 |
| 1 | 0.75 | 0.77407 | 85.052764 | 0 | 2 | 6 | 1 | 2 | 1 |
| 2 | 1.00 | -1.07150 | 48.021129 | 0 | 2 | 3 | 2 | 2 | 0 |

| | NRotB | fr_sulfide | fr_sulfonam d | fr_sulfone | fr_term_acetylene | fr_tetrazole | fr_thiazole | fr_thiocyan | fr_thiophene | |
|---|---|---|---|---|---|---|---|---|---|---|
| 0 | 0 | 0 | 0 | 0 | 0 | 0 | 0 | 0 | 0 | 0 |
| 1 | 0 | 0 | 0 | 0 | 0 | 0 | 0 | 0 | 0 | 0 |
| 2 | 0 | 0 | 0 | 0 | 0 | 0 | 0 | 0 | 0 | 0 |

3 rows × 96 columns

In :

### Data Acquisition

In :

# REC
REC_ID=‘CHEMBL240’
# Get compound record...

In :

‘Get set of active compound IDs from chEMBL database’
REC_bioact = pd.DataFrame(targets.bioactivities(REC_ID))[
[‘parent_cmpd_chemblid’,\
‘value’,\
‘units’,\
‘activity_comment’,\
‘bioactivity_type’,\
‘assay_description’,\
‘name_in_reference’,\
‘reference’]]
print ‘Number of entries before filtering:’,len(REC_bioact) “‘Compounds with IC50
activities (actives and inactives)”’
REC_bioact=REC_bioact[\
(REC_bioact.value != ‘Unspecified’) &\
(REC_bioact.bioactivity_type == ‘IC50’) &\
(REC_bioact.reference !=‘Unspecified’) &\
(REC_bioact.assay_description == ‘Inhibition of human ERG’) ]
‘FILTER maximum activity 20000nM’
REC_bioact=REC_bioact[(REC_bioact.value.astype(float) <=10000)]
‘FILTER activity not exactly 10000nM, because most likely an experimental artifact’
REC_bioact=REC_bioact[(REC_bioact.value.astype(float) !=10000)]
REC_bioact=REC_bioact[(REC_bioact.value.astype(float) !=20000)]
REC_bioact=REC_bioact[(REC_bioact.value.astype(float) !=30000)]
REC_bioact=REC_bioact[(REC_bioact.value.astype(float) !=25000)]
‘FILTER for duplicates’
REC_bioact=REC_bioact.drop_duplicates(subset=‘parent_cmpd_chemblid’)
‘Convert value to float and calculate pIC50 = -log10(X/nM)’
REC_bioact.value=REC_bioact.value.astype(float)
REC_bioact[‘logval’]=-1*np.log10(REC_bioact.value/1000000000.)
‘Add activity column’
REC_bioact[‘activity’]=[ act(i) for i in REC_bioact.value ]
REC_bioact=REC_bioact.sort(‘value’)
print ‘Number of entries after filtering: ’,len(REC_bioact)
REC_bioact.hist()

Number of entries before filtering: 14056
Number of entries after filtering: 416

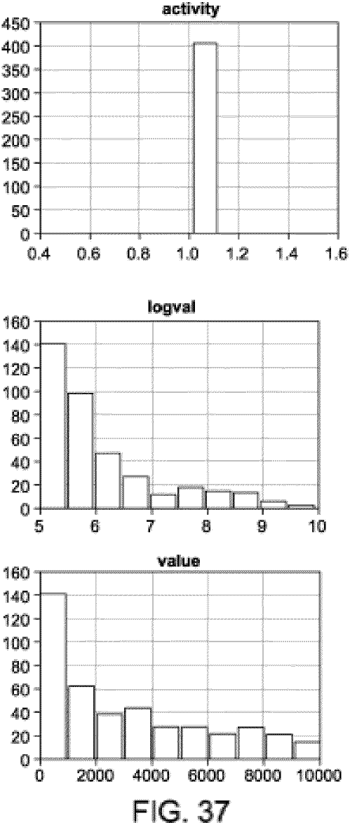

Out: (see FIG. 37)

The data reveals several plateaus e.g. at 10000 nM and 30000 nM. These might be experimental artifacts. The data spans six orders of magnitude. In this figures compounds without a value (the inactive set) is omitted.

In :

‘Get SMILES structures’
print “Database connection:”, compounds.status()
‘Workaround a problem with compounds.get() returning empty list with certain IDs is
the querry’
Database connection: True

Found structures of 415 compounds

Defining decoys In [31]: Found structures of 485 compounds

Build training, test and validation set In [32]:

act=REC_data[REC_data.act
Found 144 actives and 341 inactive compounds. 485 CHEMBL168366 1
CHEMBL2334620 1
CHEMBL19593b 1
CHEMBL49537 1
CHEMBL226140 1
dtype: int64 In [33]:
n_a+n_a_t:N_a], inact_sample[n_i+n_i_t:N_i]])
print “Training set contains ”, len(train), “ compounds”, len(train[train.activity==1]),
len(train[train.activity==0])
print “Test set contains ”, len(test), “ compounds”, len(test[test.activity==1]),
len(test[test.activity==0])
print “Validation set contains ”, len(valid), “ compounds” , len(valid[valid.activity==1]) ,
len(valid[valid.activity==0])
print “Alltogether: ”, len(train)+len(test)+len(valid)
print “Checking overlap (should be 0!)”
print ‘b in A’,len(train[train.chemblId.isin( test.chemblId )]) print ‘a in B’,len(test
[test.chemblId.isin( train.chemblId )]) Training set contains 290 compounds 86 204
Test set contains 96 compounds 28 68
Validation set contains 99 compounds 30 69
Alltogether: 485
Checking overlap (should be 0!) b in A 0
a in B 0

Exploratory Data Analysis

In : (see FIG. 38)

0.43 9400.0

In :

‘Drawing compounds from the active set’
Draw.MolsToGridlmage([Chem.MolFromSmiles(x) for x in
act.smiles.head(9)],sublmgSize=(200,200))

Out:

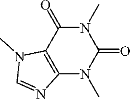

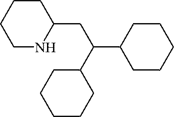 [SMILES: C1CCC(C(CC2CCCCN2)C2CCCCC2)CC1]

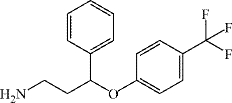 [SMILES: NCCC(Oc1ccc(C(F)(F)F)cc1)c1ccccc1]

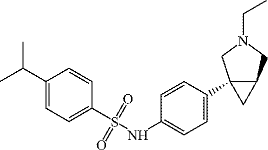 [SMILES: CCN1C[C@H]2C[C@@]2(c2ccc(NS(=O)(=O)c3ccc(C(C)C)cc3)cc2)C1]

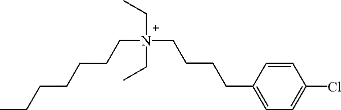 [SMILES: CCCCCCC[N+](CC)(CC)CCCCc1ccc(Cl)cc1]

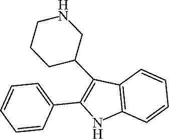 [SMILES: c1ccc(-c2[nH]c3ccccc3c2C2CCCNC2)cc1]

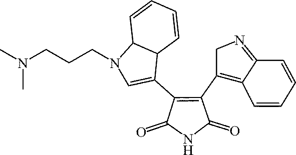 [SMILES: CN(C)CCCN1C=C(C2=C(C3=c4ccccc4=NC3)C(=O)NC2=O)C2C=CC=CC21]

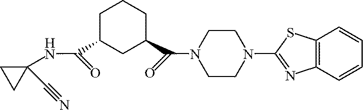 [SMILES: N#CC1(NC(=O)[C@@H]2CCC[C@@H](C(=O)N3CCN(c4nc5ccccc5s4)CC3)C2)CC1]

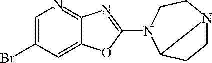 [SMILES: Brc1cnc2nc(N3CCN4CCC43)oc2c1]

In :

‘Drawing compounds from the inactive set’
Draw. MolsToGridImage([Chem.MolFromSmiles(x) for x in
inact.smiles.head(9)],sublmgSize=(200,200))

Out:

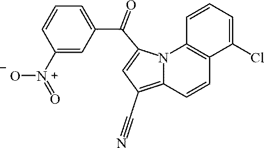 [SMILES: N#Cc1cc(C(=O)c2cccc([N+](=O)[O-])c2)n2c1ccc1c(Cl)cccc12]

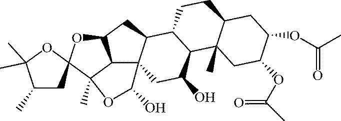 [SMILES: CC(=O)O[C@H]1C[C@@H]2CC[C@@H]3[C@H]([C@@H](O)C[C@]45[C@H](O)O[C@]6(C)[C@H]4[C@H](C[C@@H]35)O[C@]63C[C@H](C)C(C)(C)O3)[C@@]2(C)C[C@H]1OC(C)=O]

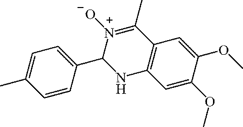

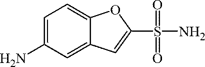 [SMILES: Nc1ccc2oc(S(N)(=O)=O)cc2c1]

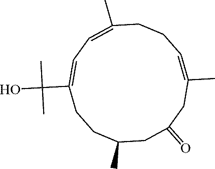 [SMILES: C/C1=C/C=C(/C(C)(C)O)CC[C@H](C)CC(=O)C/C(C)=C\CC1]

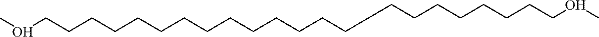

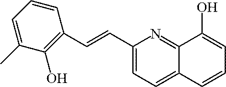 [SMILES: Cc1cccc(/C=C/c2ccc3cccc(O)c3n2)c1O]

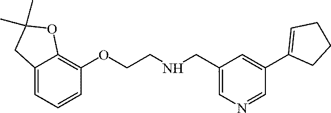 [SMILES: CC1(C)Cc2cccc(OCCNCc3cncc(C4=CCCC4)c3)c2O1]

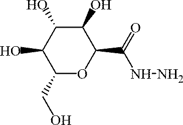 [SMILES: NNC(=O)[C@H]1O[C@H](CO)[C@@H](O)[C@H](O)[C@H]1O]

### Similarity

In : (see FIG. 39)

In :

hh = heatmap(Similarities(act_fps,inact_fps)) plt.savefig(“heatmap-similarities-
act_inact.png”,dpi=300) hh = heatmap(Similarities(inact_fps,inact_fps))
plt.savefig(“heatmap-similarities-inact.png”,dpi=300) (see FIGS. 40 and 41)

### Heatmap of the Active and Inactive Sample, Based on Dice Similarity Score

S Dice =2p+2p+q+d
P number of positive in both
q number of positive in A but not B
d number of positive in B but not A
Note that aggreements are weighted twice

Dissimilarty is black and similarity is white. The maps shows there are some clusters of similar ligands in the active set. Less so in the inactives set. There are no high similarities between active and inactive samples.

In : (see FIG. 42)

The histogram show the probability of mutual similarities. The distribution for the inactive set is shifted to lower values, indicating a higher diversity as compared to the active sample. Due to the diversity filtering the similarity distributions are very similar.

### Molecular Properties Output

---Actives---
---Inactives---
Out:

| | mean | std |
|---|---|---|
| CSP3 | 0.056972 | -0.066808 |
| LogP | 0.588021 | -0.461807 |
| MW | 8.023136 | -35.742153 |
| NAromRing | 0.056544 | -0.393138 |
| NHAt | 0.344505 | -2.609633 |
| NOCount | -0.797652 | -0.777007 |
| NRings | 0.187984 | -0.466200 |
| NRotB | -0.042408 | -1.372971 |
| TPSA | -21.675017 | -16.322524 |
| fNHAcc | -0.023556 | -0.008093 |
| fNHDon | -0.025944 | -0.024799 |

We see some general tendencies. For all properties the deviation is smaller in the active set. Interesting is that the active compounds are bound to positive logP values. Both, the fraction of hydrogen donors and acceptors seems to be smaller. So the topological polar surface area, which makes sense because this area depends on the donors and acceptors. Furthermore, there seems to be at least one aromatic ring in the actives.

### Molecular Properties: Interrelations

In : (see FIG. 43)

The interrelations of the properties in the active (red) and the inactive (blue) samples look very similar. However, we have to keep in mind that the inactives are random decoys and no proven inactives. The plots show that the actives occupy a subspace of the inactive compounds. Every region occupied by actives also has some inactives. Notably, the actives are bound to positive values in the logP values, the molecular weight (MW). The fraction of hydrogen donors (fNHDon) is lower. (see FIG. 44)

### Chemical Groups Analysis

The analysis of the mean of the counts of chemical groups shows that the chemical moieties correlate strongly in both sets active and inactive. A high number of a particular moiety in one group usually means a higher number in the other group too. For all groups the means are within the mutual standard deviations. Therefore, we cannot conclude that the occurrence of a certain moiety has special explanatory power, in the first place. However, we should look at the p-values. Also interesting would be to look at combinations.

In :

fr_C_O -4.0670 +0.0001
fr_COO2 -5.6422 +0.0000
fr_COO -5.6422 +0.0000
fr_Al_COO -5.2128 +0.0000
fr_piperdine +3.8616 +0.0002
fr_NH0 +3.7160 +0.0003

In : (see FIG. 45)

### NULL Models

In :

‘“Set X_names=features to include the chemical groups.
Set X_names=properties to only use the other molecular descriptors. ’”
print features
print properties X_names = features y_name = ‘activity’
print len(X_names), pow(2,len(X_names))
[‘CSP3’, ‘LogP’, ‘MW, ’NAromRing’, ‘NHAt’, ‘NOCount’, ‘NRings’, ‘NRotB’, ‘TPSA’,
‘fNHAcc’, ‘fNHDon’, ‘fr_C_O’, ‘fr_COO2’, ‘fr_COO’, ‘fr_Al_COO’, ‘fr_piperdine’,
‘fr_NH0’] [‘CSP3’, ‘LogP’, ‘MW, ‘NAromRing’, ‘NHAt’, ‘NOCount’, ‘NRings’, ‘NRotB’,
‘TPSA’, ‘fNHAcc’, ‘fNHDon’]
17 131072

In :

### Generation of Null Models

(see FIG. 46)

| | X | auroc |
|---|---|---|
| 10 | fNHDon | 0.645862 |
| 16 | fr_NH0 | 0.637654 |
| 8 | TPSA | 0.631840 |
| 15 | fr_piperdine | 0.630557 |
| 11 | fr_C_O | 0.596472 |
| 9 | fNHAcc | 0.596187 |
| 0 | CSP3 | 0.595218 |
| 1 | LogP | 0.589746 |
| 12 | fr_COO2 | 0.578431 |
| 13 | fr_COO | 0.578431 |
| 14 | fr_Al_COO | 0.568627 |
| 6 | NRings | 0.555147 |
| 5 | NOCount | 0.551841 |
| 7 | NRotB | 0.545913 |
| 2 | MW | 0.542921 |
| 4 | NHAt | 0.536594 |
| 3 | NAromRing | 0.510944 |

The shape of the ROCs shows that the variables are relatively weak predictors.

### Model Building

### Logistic Regression

In : (see FIGS. 47 and 48)

### Naive Bayes

“Naive Bayes methods are a set of supervised learning algorithms based on applying Bayes’ theorem with the “naive” assumption of independence between every pair of features. Naive Bayes learners and classifiers can be extremely fast compared to more sophisticated methods. [...] [Although] naive Bayes is known as a decent classifier, it is known to be a bad estimator, so the probability outputs from predict_proba are not to be taken too seriously.” scikit-learn 0.15.2 documentation In [55]: (see FIGS. 49 and 50)

### Naive Bayes on Bit-Vectors

A new approach which works with Morgan fingerprints and no molecular features. Could be a good candidate for consensus scoring.

In : (see FIGS. 51 and 52)

### Decision Tree Module

In : (see FIGS. 53 and 54)

### Random Forest Module

In : (see FIGS. 55 and 56)

### Boosting Module

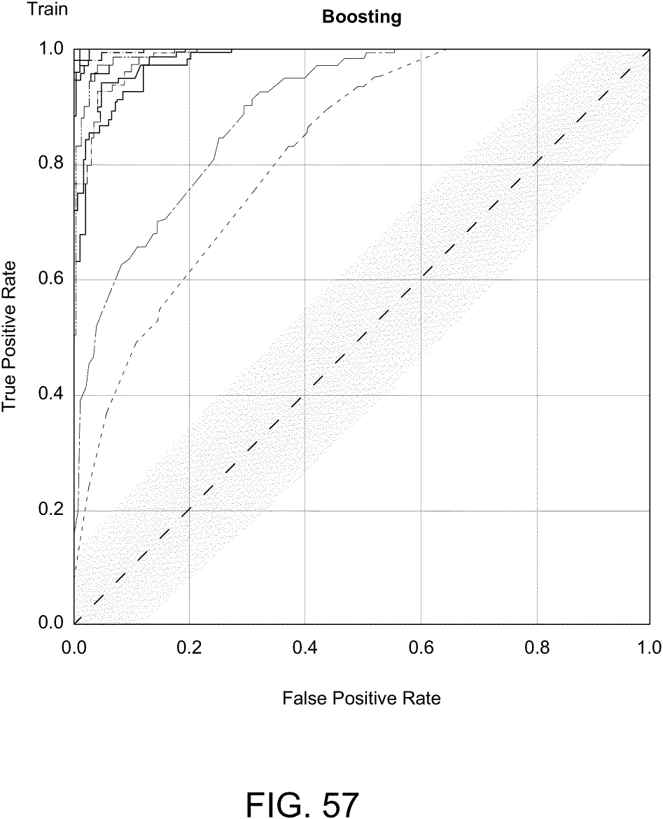

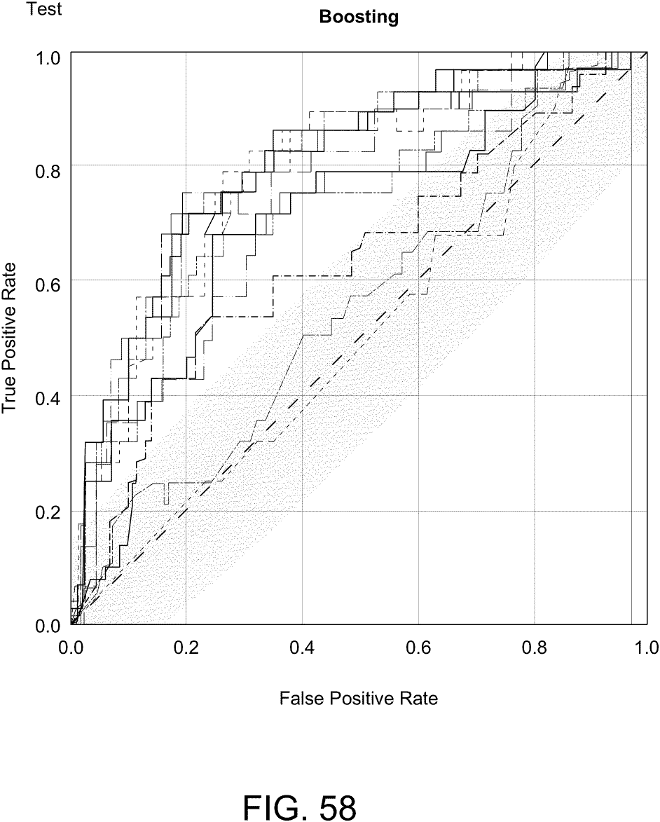

In : (see FIGS. 57 and 58)

### Model Analysis

### Prediction Accuracy Over Number of Features

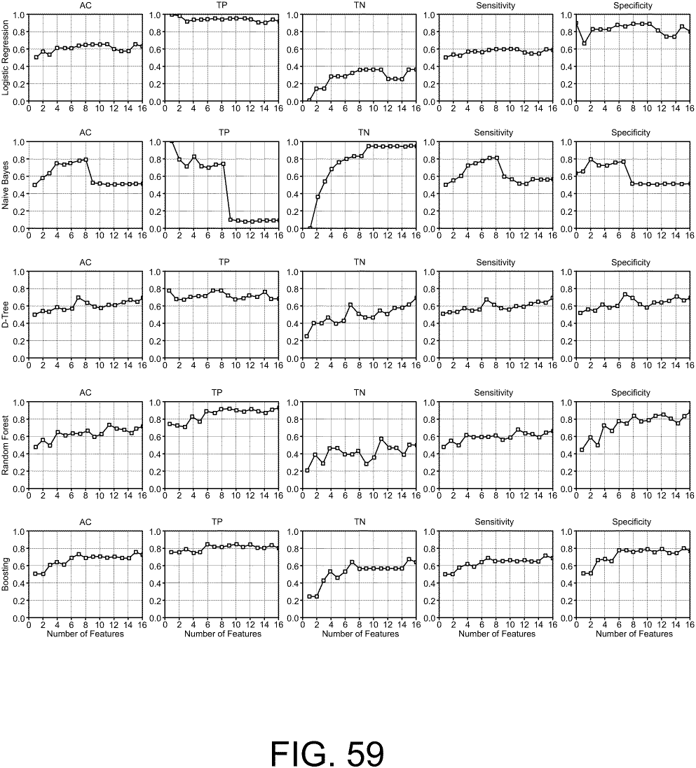

In : (see FIG. 59)

We would expect an initially increasing accuracy which drops and decreases with a certain model complexity. The drop indicate over-fitting. We see that holds for the maximum accuracy, but not for the mean or the median. The accuracies are averaged over the number of features used in the model. Maybe, the average accuracy should not be used to do this check.

### Model Selection

The models are selected according to the the highest out-of-sample accuracy (or the lowest out-of-sample error).

In :

feat=‘AC’ N_X=14
SelectModelScheme = 1
if SelectModelScheme == 0:
LR_model=LR_models_test[LR_models_test[‘N_X’]==N_X].sort(feat,ascending=Fa
Ise).model.values[0]
NB_model=NB_models_test[NB_models_test[‘N_X’]==N_X].sort(feat,ascending=F
alse).model.values[0]
DT_model=DT_models_test[DT_models_test[‘N_X’]==N_X].sort(feat,ascending=F
alse).model.values[0]
RF_model=RF_models_test[RF_models_test[‘N_X’]==N_X].sort(feat,ascending=F
alse).model.values[0]
BO_model=BO_models_test[BO_models_test[‘N_X’]==N_X].sort(feat,ascending=F
alse).model.values[0]
else:
LR_model=LR_models_test.sort(feat,ascending=False).model.values[0]
NB_model=NB_models_test.sort(feat,ascending=False).model.values[0]
DT_model=DT_models_test.sort(feat,ascending=False).model.values[0]
RF_model=RF_models_test.sort(feat,ascending=False).model.values[0]
BO_model=BO_models_test.sort(feat,ascending=False).model.values[0]
NBBV_model=NBBV_models_test.sort(feat,ascending=False).model.values[0]
BestModels_byAC=[]
for i,model in zip([‘LogReg’,‘Naive Bayes’,‘D-Tree’,‘Random
Forest’,‘Boosting’,‘NB_BitVect’],\
[LR_model,NB_model,DT_model,RF_model,BO_model,NBBV_model]):
model.label=i
BestModels_byAC.append({‘model’:model,‘Family’:i})
BestModels_byAC=pd.DataFrame(BestModels_byAC)

In :

print “Family Features #Features\n---------------------”
for i in BestModels_byAC.model:
print i.label, i.X , len(i.X)
Family Features #Features
---------------------
LogReg [‘fNHDon’, ‘fr_NHO’, ‘TPSA’, ‘fr_piperdine’, ‘fr_C_O’, ‘fNHAcc’, ‘CSP3’, ‘LogP’,
‘fr-COO2’] 9
Naive Bayes [‘fNHDon’, ‘fr_NHO’, ‘TPSA’, ‘fr_piperdine’, ‘fr_C_O’, ‘fNHAcc’, ‘CSP3’,
‘LogP’] 8
D-Tree [‘fNHDon’, ‘fr_NHO’, ‘TPSA’, ‘fr_piperdine’, ‘fr_C_O’, ‘fNHAcc’, ‘CSP3’] 7
Random Forest [‘fNHDon’, ‘fr_NHO’, ‘TPSA’, ‘fr_piperdine’, ‘fr_C_O’, ‘fNHAcc’, ‘CSP3’,
‘LogP’, ‘fr_COO2’, ‘fr_COO’, ‘fr_Al_COO’] 11
Boosting [‘fNHDon’, ‘fr_NHO’, ‘TPSA’, ‘fr_piperdine’, ‘fr_C_O’, ‘fNHAcc’, ‘CSP3’, ‘LogP’,
‘fr_COO2’, ‘fr_COO’, ‘fr_Al_COO’, ‘NRings’, ‘NOCount’, ‘NRotB’, ‘MW] 15
NB_BitVect [‘FP’] 1

### Model Performance

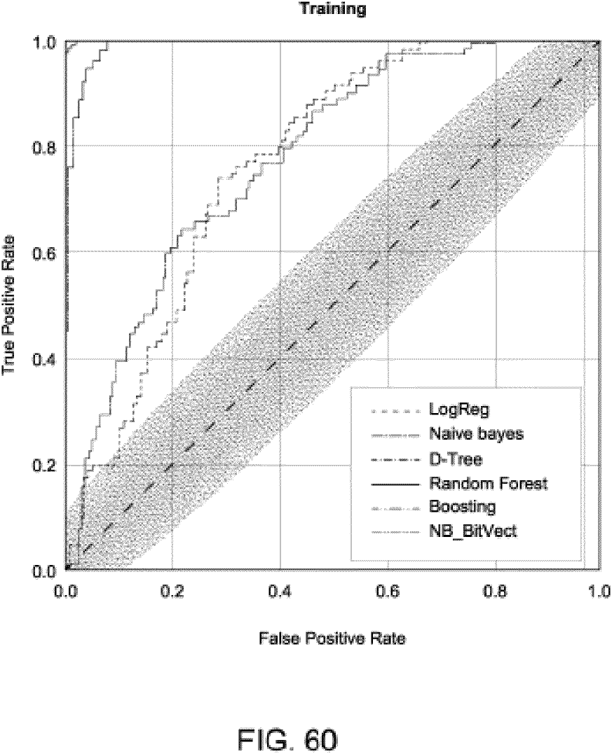

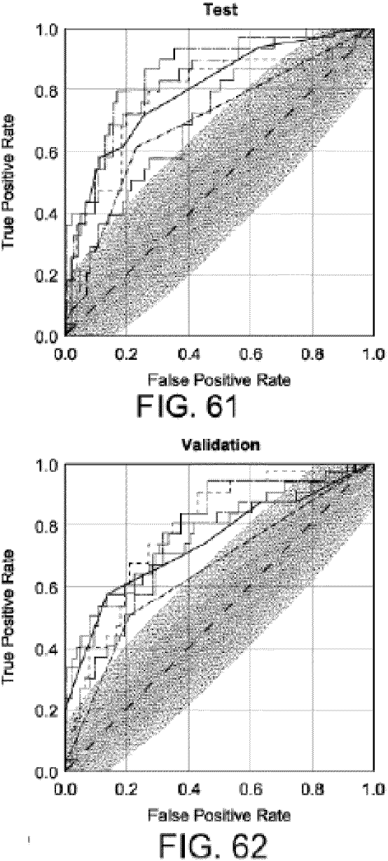

Training set (see FIGS. 60-62)

In :

A1=A[[‘Family’,‘auroc’,‘AC’,‘Sensitivity’,‘Specificity’,‘TP’,‘TN’,‘FP’,‘FN’]].sort(‘AC’,ascendi
ng=False)
print A1

| | Family | auroc | | AC Sensitivity | Specificity TP | \ |
|---|---|---|---|---|---|---|
| 2 | D-Tree | 1.000000 | 1.000000 | 1.000000 | 1.000000 | 1.000000 |
| 4 | Boosting | 0.999715 | 0.985921 | 0.977163 | 0.995006 | 0.995098 |
| 3 | Random Forest | 0.994899 | 0.982558 | 0.966292 | 1.000000 | 1.000000 |
| 5 | NB_BitVect | 0.989569 | 0.947332 | 0.952909 | 0.941892 | 0.941176 |
| 1 | Naive Bayes | 0.771546 | 0.682798 | 0.679785 | 0.685913 | 0.691176 |
| 0 | LogReg | 0.763053 | 0.577348 | 0.547751 | 0.703448 | 0.887255 |

| | FN | FP | FN |
|---|---|---|---|
| 2 | 1.000000 | 0.000000 | 0.000000 |
| 4 | 0.976744 | 0.004902 | 0.023256 |
| 3 | 0.965116 | 0.000000 | 0.034884 |
| 5 | 0.953488 | 0.058824 | 0.046512 |
| 1 | 0.674419 | 0.308824 | 0.325581 |
| 0 | 0.267442 | 0.112745 | 0.732558 |

In :

B[[‘Family’,‘auroc’,‘AC’,‘Sensitivity’,‘Specificity’,‘TP’,‘TN’,‘FP’,‘FN’]].sort(‘AC’,ascending=False)

Out[65]:

| | Family | auroc | AC | Sensitivity | Specificity | TP | TN | FP | FN |
|---|---|---|---|---|---|---|---|---|---|
| 1 | Naive Bayes | 0.849790 | 0.778361 | 0.804598 | 0.756286 | 0.735294 | 0.821429 | 0.264706 | 0.178571 |
| 4 | Boosting | 0.815126 | 0.758403 | 0.722826 | 0.807500 | 0.838235 | 0.678571 | 0.161765 | 0.321429 |
| 3 | Random Forest | 0.786765 | 0.726891 | 0.673077 | 0.829268 | 0.882353 | 0.571429 | 0.117647 | 0.428571 |
| 2 | D-Tree | 0.685924 | 0.685924 | 0.660617 | 0.720698 | 0.764706 | 0.607143 | 0.235294 | 0.392857 |
| 0 | LogReg | 0.775210 | 0.656513 | 0.597898 | 0.890052 | 0.955882 | 0.357143 | 0.044118 | 0.642857 |
| 5 | NB_BitVect | 0.675420 | 0.618697 | 0.585997 | 0.691525 | 0.808824 | 0.428571 | 0.191176 | 0.571429 |

In :

C[[‘Family’,‘AC’,‘Sensitivity’,‘Specificity’,‘TP’,‘TN’,‘FP’,‘FN’]].sort(‘AC’,ascending=False)

Out[66]

| | Family | AC | Sensitivity | Specificity | TP | TN | FP | FN |
|---|---|---|---|---|---|---|---|---|
| 1 | Naive Bayes | 0.714493 | 0.722892 | 0.706704 | 0.695652 | 0.733333 | 0.304348 | 0.266667 |
| 5 | NB_BitVect | 0.708696 | 0.654506 | 0.821429 | 0.884058 | 0.533333 | 0.115942 | 0.466667 |
| 4 | Boosting | 0.682609 | 0.627530 | 0.821429 | 0.898551 | 0.466667 | 0.101449 | 0.533333 |
| 3 | Random Forest | 0.673188 | 0.617042 | 0.832869 | 0.913043 | 0.433333 | 0.086957 | 0.566667 |
| 2 | D-Tree | 0.641304 | 0.610169 | 0.696970 | 0.782609 | 0.500000 | 0.217391 | 0.500000 |
| 0 | LogReg | 0.554348 | 0.530612 | 0.741935 | 0.942029 | 0.166667 | 0.057971 | 0.833333 |

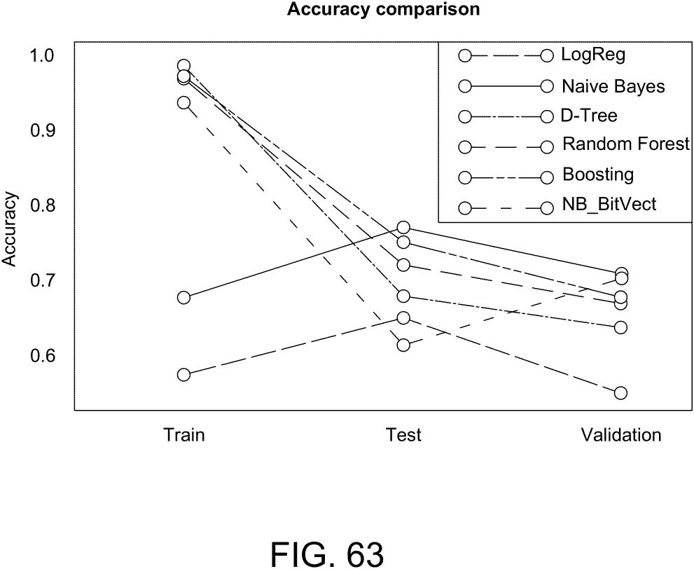

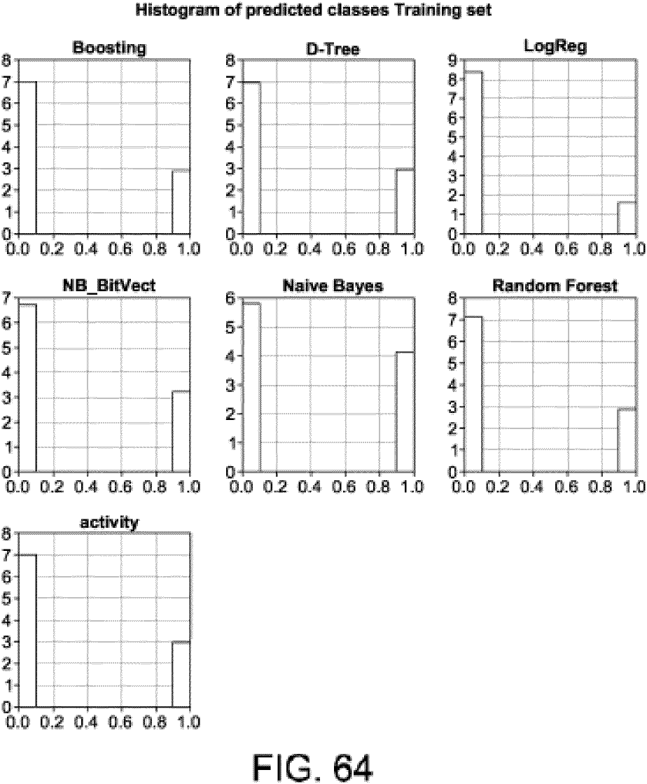

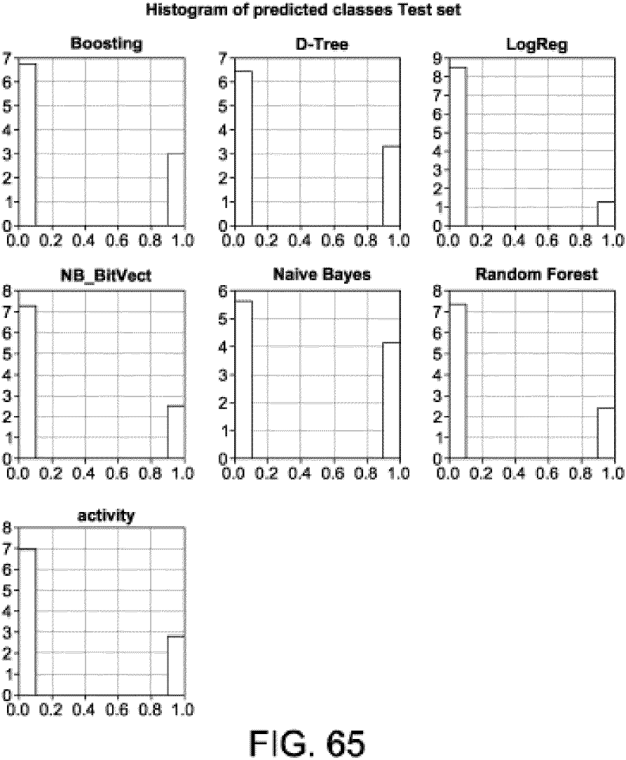

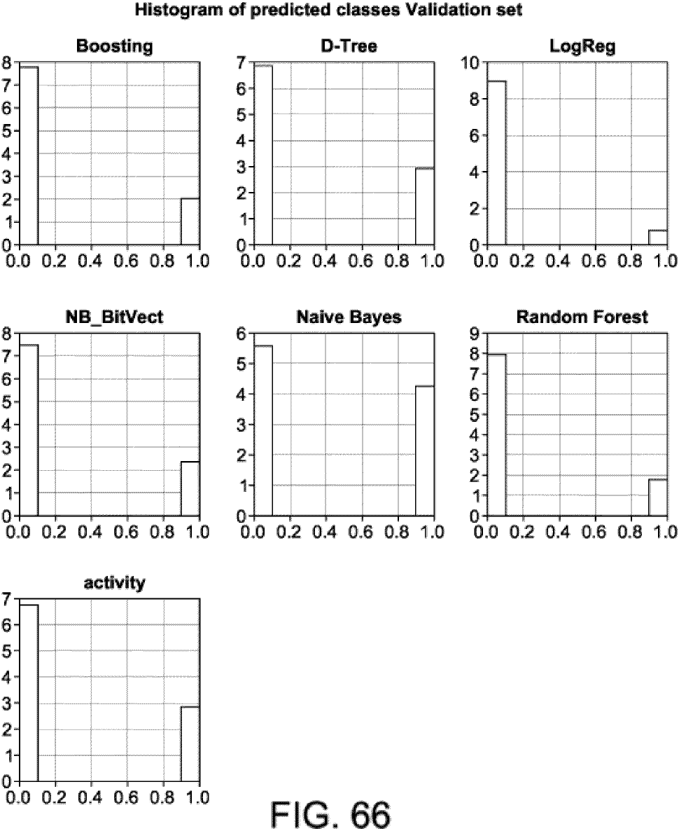

In : (see FIG. 63)
In : (see FIGS. 64 and 65)
In : (see FIG. 66)

### Result Interpretation

The Random Forest and the Boosting models usually have the highest prediction accuracies (AC). The false negative rate (FN) is very high in all models. The ROCs show that the calculated class probabilites the models can be used to rank a list of compounds. Now, it remains to be seen, whether the models can predict hERG active and inactive compounds.

### Quantitative Prediction

A try to fit the pIC50 values in a linear regression model. Cross-validated with k-means crossvalidation.

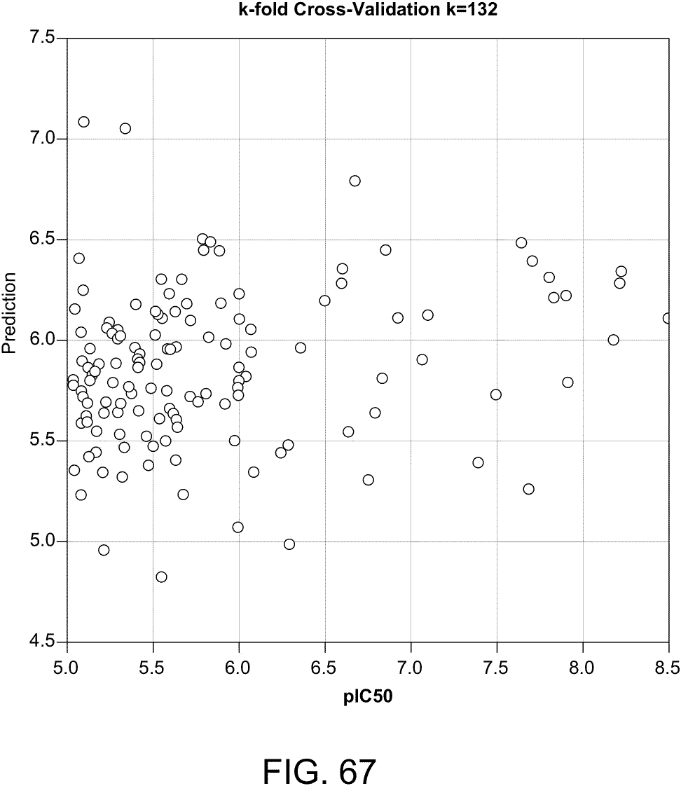

In : (see FIG. 67)

Application of Machine Learning Algorithms for Test-Set of Blockers published by Barakat et al, Toxicology Letters, 2014, doi:10.1016/j.toxlet.2014.08.007.

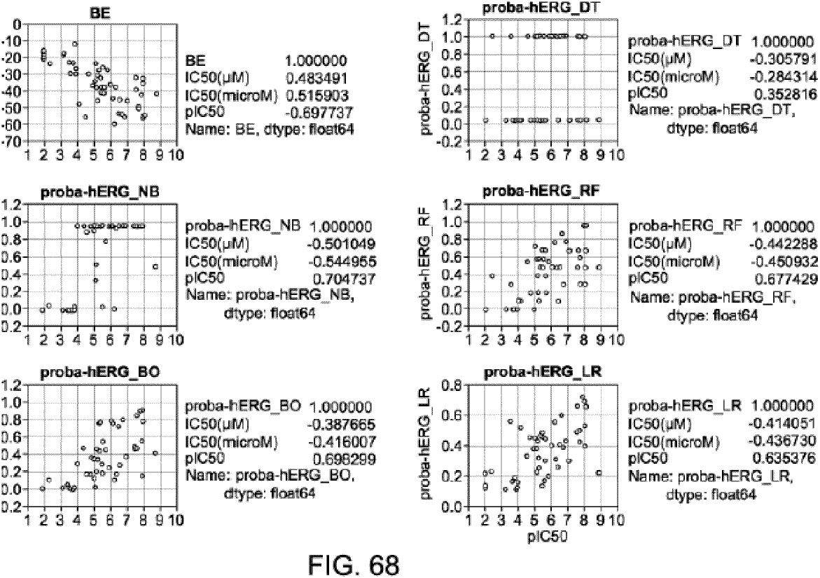

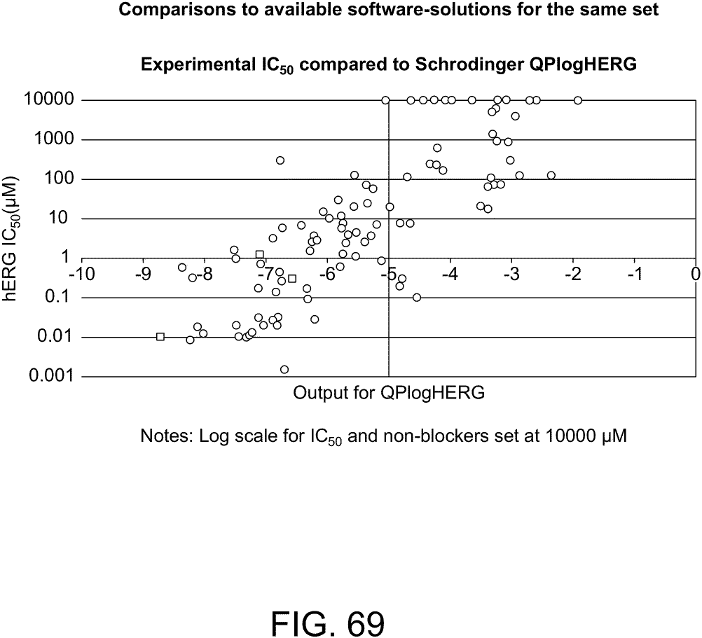

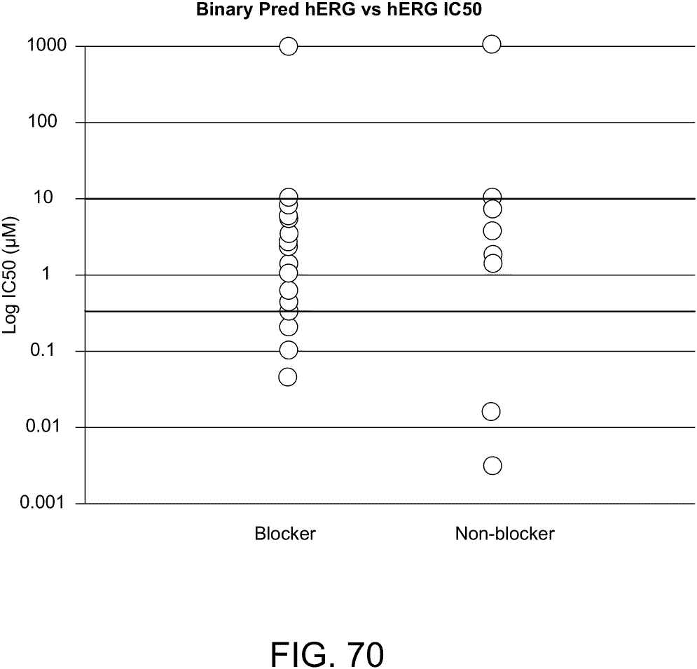

pIC50 outputs shows R² correlation coefficients to experimentally available data from Barakat et al used as a test set for model validation. (see FIGS. 68-70)

### Comparisons to Available Software-Solutions for the Same Set

89 compounds . QSAR platform from http://labmol.farmacia.ufg.br/predherg/.

Relevant citation: Braga, R. C.; Alves, V. M.; Silva, M. F. B.; Muratov, E.; Fourches, D.; Tropsha, A.; Andrade, C. H. Tuning hERG out: Antitarget QSAR Models for Drug Development. Curr. Top. Med. Chem. 2014, 14, 1399-1415.

Example of input files for SMILES structures for the training set (Brown training set).

The proper reference: Kramer J, Obejero-Paz CA, Myatt G, Kuryshev YA, Bruening-Wright A, et al. (2013) MICE Models: Superior to the HERG Model in Predicting Torsade de Pointes. Nature Scientific Reports 3: Artn 2100.

CCCCc1c(C(=O)c2cc(I)c(OCCN(CC)CC)c(I)c2)c2ccccc2o1 amiodarone
COclccc(CCN2CCC(CC2)Nc2nc3ccccc3n2Cc2ccc(F)cc2)ccl astemizole
CC(C)COCC(CN(CC1ccccc1)c1ccccc1)N1CCCC1 bepridil
CO/N=C(\C(=O)N[C@H]1[C@H]2SCC(=C(N2C1=O)C(=O)O)CSc1nc(=O)c(=O)[nH]n
1C) /c1csc (N)n1 ceftriaxone
CN(C)CCCN1c2cccec28c2cecec(C1)cc12 chlorpromazine
O=C1CCC2cc(OCCCCc3nnnn3C3CCCCC3)ccc2N1 cilostazol
COC1CN(CCCOc2ccc(F)cc2)CCC1NC(=O)c1cc(C1)c(N)cc1OC cisapride
CN1CCN(CC1)C1=Nc2cc(C1)ccc2Nc2ccccc12 clozapine
Cc1nc(Nc2ncc(s2)C(=O)Nc2c(C)cccc2C1)cc(n1)N1CCN(CCO)CC1 dasatinib
CN1c2ccc(C1)cc2C(=NCC1=O)c1ccccc1 diazepam
COc1ccc (cc1) [C@@H] 1Sc2ccccc2N (CCN(C) C) C (=O) [C@@H] 1OC (=O) C
diltiazem
CC(C)N(CCC(C(=O)N)(c1ccccc1)c1ccccn1)C(C)C disopyramide
CN(CCOc1ccc (NS(=O)(=O)C)cc1)CCc1ccc(NS(=O)(=O)C)cc1 dofetilide
COc1cc2c(cc1OC)C(=O)C(CC1CCN(Cc3ccccc3)CC1)C2 donepezil
Fc1ccc(cc1)C(=O)CCCN1CCC(=CC1)n1c(=O)[nH]c2ccccc12 droperidol
CNCC[C@H](Oc1c2ccccc2ccc1)c1cccs1 duloxetine
FC(F)(F)COc1ccc(OCC(F)(F)F)c(c1)C(=O)NCC1CCCCN1 flecainide
CCCCN(CCCC)CCC(O)c1cc2c(C1)cc(C1)cc2c2cc(ccc12)C(F)(F)F
halofantrine
OC1(CCN(CCCC(=O)c2ccc(F)cc2)CC1)c1ccc(C1)cc1 haloperidol
CCCCCCCN(CC)CCCC(O)c1ccc(NS(=O)(=O)C)cc1 ibutilide
Nc1nc(=O)n(cc1)[C@@H]1CS[C@H](CO)O1 lamivudine
CCOC(=O)N1CCC(=C2c3ccc(C1)cc3CCc3cccnc23)CC1 loratadine
CCC(=O)C(CC(C)N(C)C)(c1ccccc1)c1ccccc1 methadone
Cclncc (n1CCO) [N+] (=O) [O-] metronidazole
COCC(=O)O[C@]1(CCN(C)CCCc2nc3ccccc3[nH]2)CCc2cc(F)ccc2[C@@H]1C(C)C
mibefradil
OCCNCCNc1ccc(NCCNCCO)c2c1C(=O)c1c(O)ccc(O)c1C2=O mitoxantrone
COc1c2n(cc(C(=O)O)c(=O)c2cc(F)c1N1C[C@@H]2CCCN[C@@H]2C1)C1CC1
moxifloxacin
COC(=O)C1=C(C)NC(=C(C1c1ccccc1[N+](=O)[O-])C(=O)OC)C nifedipine
CC1cn(cn1)c1cc(NC(=O)c2ccc(C)c(Nc3nccc(n3)c3cccnc3)c2)cc(c1)C(F
) (F) F nilotinib
CCOC(=O)C1=C(C)NC(=C(C1c1cccc(c1)[N+](=O)[O-])C(=O)OC)C
nitrendipine
Cc1(CCN2CCC(CC2)c2noc3cc(F)ccc23)c(=O)n2CCC[C@@H](O)c2n1
paliperidone
Fc1ccc (cc1) [C@@H] 1CCNC[C@H] 1COc1ccc2OCOc2c1 paroxetine
CCCC(C)C1(CC)C(=O)NC(=O)NC1=O pentobarbital
O=C1NC(=O)C(N1)(c1ccccc1)c1ccccc1 phenytoin
Fc1ccc(cc1)C(CCCN1CCC(CC1)n1c(=O)[nH]c2ccccc12)c1ccc(F)cc1
pimozide
CCN1CCN(C(=O)N[C@@H](C(=O)N[C@H]2[C@H]3SC(C)(C)[C@@H](N3C2=O)C(=O)O
)c2ccccc2)C(=O)C1=O piperacillin
CCN(CC)CCNC(=O)c1ccc(N)cc1 procainamide
COc1ccc2nccc(@H](O)[C@H]3C[C@@H]4CCN3C[C@@H]4C=C)c2c1
quinidine
Cn1c(=O)c(O)c(nc1C(C)(C)NC(=O)c1nnc(C)o1)C(=O)NCc1ccc(F)cc1
raltegravir
NC(=O)c1nn(cn1)[C@@H]1O[C@H](CO)[C@@H](O)[C@H]1O ribavirin
Cc1c(CCN2CCC (CC2) c2noc3cc(F)ccc23)c(=O)n2CCCCc2n1 risperidone
CC(C)(C)NC(=O)[CCCH]1C[CCCH]2CCCC[CCCH]2CN1C[CCCH](O)[C@H](CC1c
cccc1)NC(=O)[C@H](CC(=O)N)NC(=O)c1ccc2ccccc2n1 saquinavir
Fc1ccc(cc1)n1cc(C2CCN(CCN3CCNC3=0)CC2)c2cc(C1)ccc12 sertindole
N[C@@H](CC(=O)N1CCn2c(C1)nnc2C(F)(F)F)Cc1cc(F)c(F)cc1F
sitagliptin
OC(=O)CCC(=O)O.O=C(O[C@H]1CN2CC[C@H]1CC2)N1CCc2ccccc2[C@@H]1c1c
cccc1 solifenacin
CC(C)NCC(O)c1ccc(NS(=O)(=O)C)ccl1 sotalol
C[C@H]1CN(C[C@@H](C)N1)c1c(F)c(N)c2c(=O)c(cn(C3CC3)c2c1F)C(=O)O
sparfloxacin
CCN(CC)CCNC=O)c1c(C)[nH]c(/C=C/2\C(=O)Nc3ccc(F)cc23)c1C
sunitinib
Cc1cn([C@@H]2C[C@@H](O)[C@H](CO)O2)c(=O)[nH]c1=O telbivudine
CC(C)(C)c1ccc(cc1)C(O)CCCN1CCC(CC1)C(O)(c1ccccc1)c1ccccc1
terfenadine
CC(CC(c1ccccc1)c1ccccc1)NC(C)(C)C erodiline
CSclccc2Sc3ccccc3N(CCC3CCCCN3C)c2cl thioridazine
COc1ccc(CCN(C)CCCC(C#N)(C(C)C)c2ccc(OC)c(OC)c2)cc1OC verapamil
C[C@@H](c1ncncc1F)[C@](O)(Cn1cncn1)c1cc(F)cc1F voriconazole

Set of activity parameters for Brown dataset (see reference above for definitions) used for TdP+ prediction training and machine learning algorithms

| | Ris | IC50 | pIC50 | IC50- | maxInhi | HERG- max SEM | CA IC50 | pIC50 | IC50- | CAV1 P 2- hib | 2- Max SEM | 5-IC50 | pIC50 | NAV SEM | NAV1 P 5- hib | uM | Fre (uM) | pFD |
|---|---|---|---|---|---|---|---|---|---|---|---|---|---|---|---|---|---|---|
| amiodaron | 1 | 0.86 | 6.0655 549 | 0.12 | 81.8 | 2.7 | 1.9 | 5.7212 399 | 0.2 | 57.3 | 3.4 | 15.9 | 4.7986 876 | 2.1 | 86 | 9.4 | 0.00 08 | 9.096 1001 |
| astemizole | 1 | 0.00 | 8.3979 009 | 0.00 | 83.6 | 4.4 | 1.1 | 5.9586 315 | 0.1 | 96.8 | 0.1 | 3 | 5.5228 745 | 0.2 | 90.5 | 3.6 | 0.00 03 | 9.522 7874 |
| bepridil | 1 | 0.16 | 6.7958 017 | 0.02 | 86.4 | 5.3 | 1 | 6 | 0.2 | 95.7 | 1.3 | 2.3 | 5.6382 164 | 0.3 | 100 | 0 | 0.03 5 | 7.455 3195 |
| ceftriaxone | 0 | 445. | 3.3509 366 | 146. | 7.5 | 2.1 | 153. 8 | 3.8130 665 | 23.9 | 38.8 | 4.2 | 555. | 3.2550 326 | 159. | 14.3 | 4.2 | 23.1 7 | 4.635 7396 |
| chlorprom ne | 1 | 1.5 | 5.8239 741 | 0.1 | 96.7 | 0.7 | 3.4 | 5.4685 083 | 0.3 | 99.2 | 0.8 | 3 | 5.5228 745 | 0.4 | 97.1 | 2 | 0.03 8 | 7.420 ⁻1640 |
| cilostazol | 1 | 13.8 | 4.8601 914 | 1 | 65.9 | 3.2 | 91.2 | 4.0400 162 | 24.9 | 24.6 | 6.2 | 93.7 | 4.0282 409 | 22.2 | 22.9 | 0.9 | 0.12 8 | 6.892 ⁻900 |
| cisapride | 1 | 0.02 | 7.6989 004 | 0.00 | 82.1 | 3.2 | 11.8 | 4.9281 993 | 1.3 | 42.8 | 3.8 | 337 | 3.4723 099 | 94.6 | 22.9 | 6 | 0.00 3 | 8.522 7874 |
| clozapine | 1 | 2.3 | 5.6382 164 | 0.3 | 97.7 | 1.5 | 3.6 | 5.4436 499 | 0.4 | 73.5 | 3.2 | 15.1 | 4.8210 053 | 2.7 | 91.8 | 2 | 0.07 1 | 7.148 ⁻4165 |
| dasatinib | 0 | 24.5 | 4.6108 916 | 1.4 | 86.6 | 1.2 | 81.1 | 4.0909 146 | 6.9 | 52.2 | 4 | 76.3 | 4.1174 462 | 10.8 | 58.9 | 6.5 | 0.04 1 | 1.387 ⁻1614 |
| diazepam | 0 | 53.2 | 4.2740 368 | 5.8 | 66.4 | 4.8 | 30.5 | 4.5157 161 | 4.5 | 49.5 | 3.1 | 306. | 3.5137 239 | 37.6 | 24.3 | 3.1 | 0.02 9 | 7.537 0200 |
| diltiazem | 0 | 13.2 | 4.8794 069 | 0.7 | 92 | 1.8 | 0.76 | 6.1191 408 | 0.08 | 91 | 4.3 | 22.4 | 4.6497 982 | 2.2 | 100 | 0 | 0.12 2 | 6.913 4016 |
| disopyrami | 1 | 14.4 | 4.8416 508 | 2 | 86.3 | 2.8 | 103.7 | 2.9843 902 | 96.2 | 22.1 | 1.3 | 168. | 3.7736 913 | 15.4 | 68.9 | 2.7 | 0.74 2 | 6.129 9609 |
| dofetilide | 1 | 0.03 | 7.5228 745 | 0.00 | 99.5 | 0.2 | 26.7 | 4.5734 739 | 4.3 | 54.2 | 1.1 | 162. | 3.7902 985 | 23.6 | 14.7 | 2.6 | 0.00 2 | 9.698 7000 |
| donepezil | 0 | 0.7 | 6.1549 96 | 0.04 | 98.3 | 0.4 | 34.3 | 4.4647 88 | 9.6 | 55.7 | 7.3 | 38.5 | 4.4145 271 | 6.4 | 48.9 | 5.4 | 0.00 3 | 8.522 7874 |
| droperidol | 1 | 0.06 | 7.2218 75 | 0.00 | 95 | 0.5 | 7.6 | 5.1191 408 | 0.7 | 83.4 | 2.6 | 22.7 | 4.6439 143 | 4.5 | 59 | 11.1 | 0.01 6 | 7.795 8001 |
| duloxetine | 0 | 3.8 | 5.4202 403 | 0.4 | 96.3 | 1.2 | 2.8 | 5.5528 969 | 0.31 | 99.8 | 0.5 | 5.1 | 5.2924 824 | 0.43 | 100 | 0 | 0.01 6 | 7.795 8001 |
| flecainide | 1 | 1.5 | 5.8239 741 | 0.07 | 94.9 | 0.5 | 27.1 | 4.5670 709 | 3.2 | 76.4 | 3.4 | 6.2 | 5.2076 311 | 0.6 | 98 | 2 | 0.75 3 | 6.123 0502 |
| halofantrin | 1 | 0.38 | 6.4202 403 | 0.02 | 77.9 | 4 | 1.9 | 5.7212 399 | 0.2 | 87 | 5.4 | 331. | 3.4799 672 | 80.9 | 8.3 | 1.8 | 0.17 2 | 6.764 7155 |
| haloperidol | 1 | 0.04 | 7.3979 009 | 0.00 | 89.2 | 1.8 | 1.3 | 5.8860 648 | 0.1 | 94 | 1.7 | 4.3 | 5.3665 544 | 0.2 | 96.6 | 1.5 | 0.00 4 | 9.397 4000 |
| | | | 7.7447 | | | | | 4.2041 | | | | | 4.3716 | | | | | 6.853 |
| ibutilide | 1 | 0.01 | 495 | 0.00 | 98.2 | 1.4 | 62.5 | 983 | 8.1 | 85.2 | 4.4 | 42.5 | 07 | 5.4 | 87.9 | 2.3 | 0.14 | 7196 |
| lamivudine | 0 | 205 | 2.6873 561 | 379. | 11.2 | 3.2 | 54.2 | 4.2660 714 | 6.8 | 81.3 | 3.1 | 1571. | 2.8037 251 | 272. | 14.3 | 3.1 | 19.5 4 | 4.709 7544 |
| linezolid | 0 | 1147. | 2.9403 862 | 163. | 19.3 | 3.9 | 105. 4 | 3.9771 389 | 12.7 | 70.3 | 3.2 | 2644. | 2.5776 429 | 416. | 9.6 | 0.9 | 59.1 1 | 4.228 3904 |
| loratadine | 0 | 6.1 | 5.2146 165 | 0.4 | 94.8 | 1.8 | 11.4 | 4.9430 149 | 1.2 | 93.2 | 1.5 | 28.9 | 4.5391 157 | 3.1 | 92.8 | 7.2 | 0.00 04 | 9.397 4000 a |
| methadone | 1 | 3.5 | 5.455931 956 | 0.3 | 91.6 | 1.7 | 37.4 | 4.427128 398 | 5.8 | 96.9 | 4.2 | 31.8 | 4.497572 88 | 1.4 | 100 | 0 | 0.50 7 | 6.2949 92041 |
| metronidazol e | 0 | 1340.2 | 2.872830 386 | 216.5 | 16.2 | 4.5 | 177. 9 | 3.749824 052 | 67.3 | 57.4 | 10.8 | 2073.2 | 2.683358 8 | 301.8 | 12 | 2 | 187 | 3.7281 58394 |
| mibefradil | 0 | 1.7 | 5.769551 079 | 0.1 | 95.2 | 0.8 | 0.51 | 6.292429 824 | 0.04 | 92.1 | 0.7 | 5.6 | 5.251811 973 | 0.5 | 100 | 0 | 0.01 2 | 7.9208 18754 |
| mitoxantrone | 0 | 539.4 | 3.268089 058 | 83.6 | 14.8 | 2.2 | 22.5 | 4.647817 482 | 4.1 | 53.4 | 4.3 | 93.5 | 4.029188 389 | 27.8 | 50.8 | 9.8 | 0.22 5 | 6.6478 17482 |
| moxifloxacin | 1 | 86.2 | 4.064492 734 | 10.5 | 75.9 | 4.8 | 173 | 3.761953 897 | 26.3 | 53.7 | 3.8 | 1112 | 2.953895 213 | 383.9 | 17.4 | 5.6 | 10.9 6 | 4.9601 89446 |
| nifedipine | 0 | 44 | 4.356547 324 | 6.1 | 65.8 | 3.7 | 0.01 2 | 7.920818 754 | 0.001 | 88.3 | 1.3 | 88.5 | 4.053056 729 | 11.5 | 54.1 | 3.2 | 0.00 8 | 8.0969 10013 |
| nilotinib | 1 | 1 | 6 | 0.1 | 68.2 | 4.7 | 17.5 | 4.756961 951 | 2.7 | 32.4 | 4.9 | 13.3 | 4.876148 359 | 2.3 | 35.5 | 6.2 | 0.17 2 | 6.7644 71553 |
| nitrendipine | 0 | 24.6 | 4.609064 893 | 2.8 | 68.3 | 1.3 | 0.02 5 | 7.602059 991 | 0.002 | 74.6 | 0.5 | 21.6 | 4.665546 249 | 1.9 | 86.3 | 3.8 | 0.00 3 | 8.5228 78745 |
| paliperidone | 1 | 0.78 | 6.107905 397 | 0.05 | 95 | 0.5 | 193. 9 | 3.712422 191 | 22.2 | 33.1 | 3.9 | 109 | 3.962573 502 | 11.5 | 51.2 | 4.1 | 0.06 9 | 7.1611 50909 |
| paroxetine | 1 | 1.9 | 5.721246 399 | 0.1 | 99.5 | 0.2 | 3.9 | 5.408935 393 | 0.2 | 98.5 | 0.2 | 9.8 | 5.008773 924 | 1.6 | 93.7 | 4.2 | 0.01 4 | 7.8538 71964 |
| pentobarbital | 0 | 1433.9 | 2.843481 135 | 128.5 | 39.5 | 2.6 | 299 | 3.524328 812 | 48.2 | 82.1 | 5.9 | 2686 | 2.570893 992 | 776.1 | 9 | 4.9 | 5.17 1 | 5.2864 25462 |
| phenytoin | 0 | 147 | 3.832682 665 | 13.7 | 16.4 | 1.7 | 21.9 | 4.659555 885 | 3.1 | 91.7 | 3.3 | 72.4 | 4.140261 434 | 14.7 | 59.3 | 11.1 | 4.36 | 5.3605 13511 |
| pimozide | 1 | 0.04 | 7.397940 009 | 0.01 | 91.3 | 3.6 | 0.24 | 6.619788 758 | 0.02 | 88.1 | 3.9 | 1.1 | 5.958607 315 | 0.2 | 97.5 | 0.7 | 0.00 05 | 9.3010 29996 |
| piperacillin | 0 | 3405.1 | 2.467870 129 | 1041.1 | 3.9 | 2.9 | 1226 | 2.911509 53 | 346.6 | 15.9 | 6 | 2433.8 | 2.613715 113 | 561.2 | 9.7 | 3 | 1378 | 2.8607 50782 |
| procainamide | 1 | 272.4 | 3.564792 897 | 36.1 | 50.7 | 7.4 | 389. 5 | 3.409492 538 | 59.5 | 44.3 | 2.3 | 746.6 | 3.126912 014 | 114.3 | 29.3 | 5.1 | 54.1 8 | 4.2661 60999 |
| quinidine | 1 | 0.72 | 6.142667 504 | 0.08 | 100 | 0 | 6.4 | 5.193820 026 | 0.7 | 58 | 4.4 | 14.6 | 4.835647 144 | 1 | 97.5 | 2.4 | 3.23 7 | 5.4898 57301 |
| raltegravir | 0 | 782.8 | 3.106349 183 | 93.8 | 22.6 | 2.7 | 246. 7 | 3.607830 851 | 32.8 | 54.5 | 4.8 | 824.2 | 3.083967 39 | 95 | 27.6 | 2.9 | 7 | 5.1549 0196 |
| ribavirin | 0 | 967 | 3.014573 526 | 114.5 | 21.4 | 3.1 | 622. 5 | 3.205860 644 | 68.6 | 31.9 | 3.4 | 2997.5 | 2.523240 808 | 802.5 | 6.9 | 2 | 27.8 8 | 4.5547 07231 |
| | | | 6.585026 | | | | | 4.465973 | | | | | 4.362510 | | | | 0.00 | 8.6989 |
| risperidone | 1 | 0.26 | 652 | 0.02 | 79.7 | 2.5 | 34.2 | 894 | 3.2 | 70.4 | 3.8 | 43.4 | 271 | 8.3 | 73.9 | 7.2 | 2 | 70004 |
| saquinavir | 0 | 16.9 | 4.772113 295 | 0.8 | 73.1 | 3.6 | 1.9 | 5.721246 399 | 0.3 | 90.5 | 1.2 | 12.1 | 4.917214 63 | 1 | 89.9 | 4.8 | 0.13 | 6.8860 56648 |
| sertindole | 1 | 0.033 | 7.481486 06 | 0.001 | 94.3 | 0.6 | 6.3 | 5.200659 451 | 0.5 | 91.5 | 1.8 | 6.9 | 5.161150 909 | 1.2 | 95.5 | 4.4 | 0.00 2 | 8.6989 70004 |
| sitagliptin | 0 | 174.7 | 3.757707 095 | 15 | 35.6 | 2.8 | 147. 1 | 3.832387 327 | 22.3 | 35.8 | 2.4 | 1220.8 | 2.913355 479 | 270.1 | 18.3 | 3.4 | 0.44 2 | 6.3545 77731 |
| solifenacin | 1 | 0.28 | 6.552841 969 | 0.05 | 51.7 | 5.4 | 4.3 | 5.366531 544 | 0.3 | 96.4 | 1 | 1.5 | 5.823908 741 | 0.2 | 100 | 0 | 0.00 3 | 8.5228 78745 |
| sotalol | 1 | 111.4 | 3.953114 809 | 14.6 | 65.3 | 4.7 | 193. 3 | 3.713768 146 | 37.8 | 52 | 4.8 | 7013.9 | 2.154040 43 | 2955.8 | 5.6 | 7 | 14.6 9 | 4.8329 78204 |
| sparfloxacin | 1 | 22.1 | 4.655607 726 | 0.5 | 81.5 | 0.3 | 88.8 | 4.051587 034 | 17.7 | 50.3 | 7.6 | 2555 | 2.592609 096 | 1356.8 | 0.8 | 2.2 | 1.76 6 | 5.7530 09301 |
| sunitinib | 1 | 1.2 | 5.920818 754 | 0.2 | 60.5 | 7.7 | 33.4 | 4.476253 533 | 3.1 | 61.5 | 5.6 | 16.5 | 4.782516 056 | 1.9 | 66.4 | 6.1 | 0.01 3 | 7.8860 56648 |
| telbivudine | 0 | 422.7 | 3.373967 752 | 66.9 | 17.4 | 2.2 | 713. 9 | 3.146362 618 | 93 | 22.1 | 2.2 | 1095.2 | 2.960506 565 | 366.9 | 15.1 | 5 | 19.7 2 | 4.7050 93089 |
| terfenadine | 1 | 0.05 | 7.301029 996 | 0.004 | 84.6 | 0.4 | 0.93 | 6.031517 051 | 0.07 | 98 | 1.2 | 2 | 5.698970 004 | 0.2 | 99 | 1 | 0.00 9 | 8.0457 57491 |
| terodiline | 1 | 0.65 | 6.187086 643 | 0.07 | 82.3 | 2.4 | 4.8 | 5.318758 763 | 1 | 95.4 | 3.2 | 7.4 | 5.130768 28 | 1.2 | 100 | 0 | 0.14 5 | 6.8386 31998 |
| thioridazine | 1 | 0.5 | 6.301029 996 | 0.06 | 98.6 | 0.4 | 3.5 | 5.455931 956 | 0.3 | 82.9 | 3.8 | 1.4 | 5.853871 964 | 0.1 | 99.3 | 0.3 | 0.98 | 6.0087 73924 |
| verapamil | 0 | 0.25 | 6.602059 991 | 0.03 | 78.7 | 1.9 | 0.2 | 6.698970 004 | 0.02 | 87.6 | 3.4 | 32.5 | 4.488116 639 | 4.2 | 80.4 | 4.8 | 0.08 8 | 7.0555 17328 |
| voriconazole | 1 | 490.9 | 3.309006 968 | 96 | 28.9 | 4.9 | 414. 2 | 3.382789 905 | 43 | 40.9 | 2.9 | 1550.5 | 2.809528 229 | 450.9 | 15.8 | 6.3 | 7.56 3 | 5.1213 059 |

### APPENDIX B

[image]

### Nav15 Activity Prediction

Can we use molecular structures, related properties and similarities to distinguish between Nav1.5 active and inactive compounds?
desired accuracy +90%

“‘Database functionality’”
# (pip install chembl_webresource_client)
“‘RDKit and sklearn imports and related functions”’

In :

“‘Test of function”’
df=pd.DataFrame(list(MolecularProperties([‘CCC’,‘C1CCOC1(=N)’,‘OCO’])))
df

Out

| | CSP3 | LogP | MW | NAro mRing | NHAt | NHA cc | NHD on | NOCount | NR ing | NR otB | . | fr_sulfide | fr_sulfonamd | fr_s |
|---|---|---|---|---|---|---|---|---|---|---|---|---|---|---|
| 0 | 1.00 | 1.41630 | 44.062600 | 0 | 0 | 3 | 0 | 0 | 0 | 0 | ⋮ | 0 | 0 | 0 |
| 1 | 0.75 | 0.77407 | 85.052764 | 0 | 2 | 6 | 1 | 2 | 1 | 0 | ⋮ | 0 | 0 | 0 |
| 2 | 1.00 | 1.07150 | 48.021129 | 0 | 2 | 3 | 2 | 2 | 0 | 0 | ⋮ | 0 | 0 | 0 |

3 rows × 96 columns

In :

“‘Miscelaneous’”
from IPython.display import Image
Data acquisition

In :

# REC
REC_ID=‘CHEMBL1980’
# Get compound record...
target = targets.get(REC_ID)

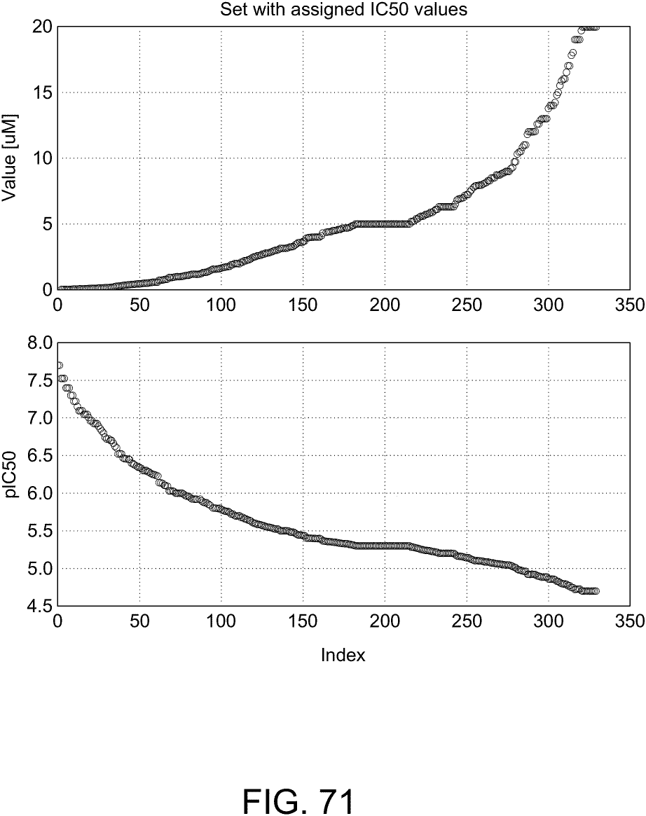

Number of entries before filtering: 695 Number of entries after filtering: 240 (see FIG. 71)

In :

Out

| | |
|---|---|
| 376 | 1 |
| 394 | 1 |
| 173 | 1 |
| 435 | 1 |
| 378 | 1 |
| 459 | 1 |
| 379 | 1 |
| 433 | 1 |
| 418 | 1 |
| 391 | 1 |
| 434 | 1 |
| 417 | 1 |
| 432 | 1 |
| 375 | 1 |
| 436 | 1 |
| ... | |
| 11 | 1 |
| 496 | 1 |
| 580 | 1 |
| 340 | 1 |
| 451 | 1 |
| 492 | 0 |
| 622 | 0 |
| 89 | 0 |
| 351 | 0 |
| 352 | 0 |
| 452 | 0 |
| 475 | 0 |
| 520 | 0 |
| 557 | 0 |
| 602 | 0 |

Name: activity, Length: 240, dtype: int64 In [17]:

The data reveals several plateaus e.g. at 10000 nM. These might be experimental artefacts. The data spans six orders of magnitude. In this figures compounds without a value (the inactive set) is omitted.

In :

Get structures’
print “Database connection:”, compounds.status()
structures=compounds.get(list(REC_bioact.parent_cmpd_chemblid))
#Get the compound structures from the ChEMBL database
a=[i for i in structures if type(i)!=int]
REC_cmpds=pd.DataFrame(a)[[‘chemblld’,‘smiles’]]
REC_cmpds=REC_cmpds.dropna()
print ‘Found structures of %d compounds’ %(len(REC_cmpds))
REC_cmps_properties=pd.DataFrame(list(MolecularProperties(REC_cmpds.smiles)))
dfl=AddMolProp(REC_cmpds)
df1.count()
REC_data=pd.merge(REC_bioact.rename(columns={“parent_cmpd_chemblid’:‘chemblId’}),df
1,on=‘chemblId’)
REC_data=REC_data[REC_data.NHAt>10]
print ‘Found structures of %d compounds’ %(len(REC_data))

Database connection: True

Found structures of 240 compounds Found structures of 240 compounds In [19]:

REC _data[‘activity’] Out[20]:

| | | |
|---|---|---|
| | 90 | 1 |
| | 74 | 1 |
| | 2 | 1 |
| | 37 | 1 |
| | 232 | 0 |
| | 64 | 1 |
| | 4 | 1 |
| | 235 | 0 |
| | 127 | 1 |
| | 231 | 0 |
| | 154 | 1 |
| | 136 | 1 |
| | 207 | 1 |
| | 236 | 0 |
| | 118 | 1 |
| | ... | |
| | 133 | 1 |
| | 199 | 1 |
| | 96 | 1 |
| | 193 | 1 |
| | 128 | 1 |
| | 224 | 1 |
| | 87 | 1 |
| 119 | 1 | |
| 169 | 1 | |
| 40 | 1 | |
| 84 | 1 | |
| 89 | 1 | |
| 13 | 1 | |
| 165 | 1 | |
| 60 | 1 | |

Name: activity, Length: 100, dtype: int64 In [20]:

#del decoys
‘“Decoys”’
‘“Filtering with pandas is a PITA”’
‘Filter out the entries with empthy smiles strings’ ‘Filter out smiles with two or more
molecules’
‘check for smiles entries that contain multiple molecules’ ‘Data Transformation’
Doublicates 0
952

Build training, test and validation set
Found 92 actives and 860 inactive compounds. 952 CHEMBL569085 1

| | |
|---|---|
| CHEMBL123454 | 1 |
| CHEMBL111622 | 1 |
| CHEMBL335768 | 1 |
| CHEMBL458520 | 1 |

dtype: int64 In [25]:

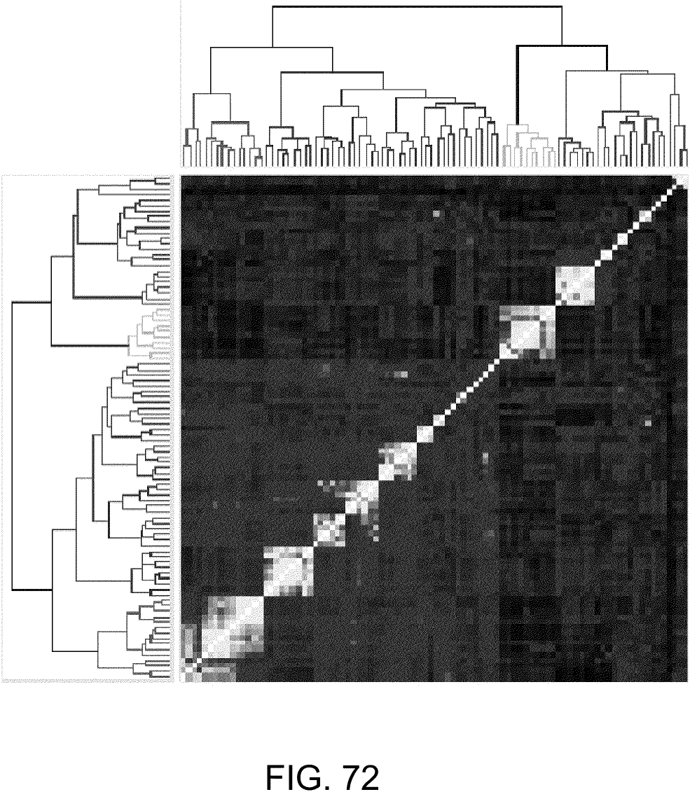

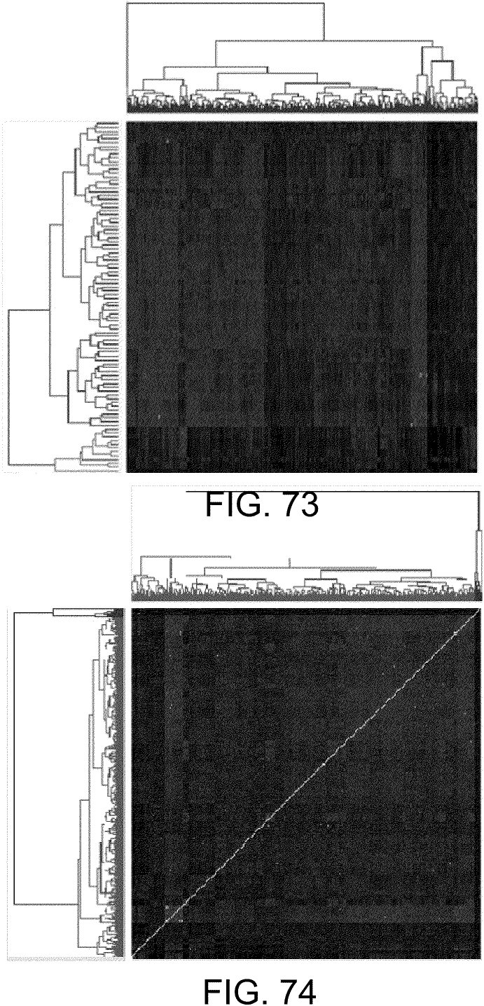

(see FIGS. 72-74)

### Exploratory Data Analysis

Application of PCA to reduce set redudancy

Heatmap of the randomly selected active (left) and inactive (top) compounds, based on Dice similarity score:

$$S D i c e = 2 p + q + d$$

Where
P number of positive in both,
Q number of positive in A but not B,
d number of positive in B but not A . Note that aggreements are weighted twice

### Results

The similarity of the inactive compounds is much higher than expected, if only the REC inactive records in CHEMBLDB are used.
therefore ~800 decoys were added randomly selected from CHEMBLDB
the dissimilarity is OK In [28]:

Properties = [‘CSP3’,‘LogP’,‘MW’,‘NAromRing’,\
‘NHAt’,‘NOCount’,‘NRings’,‘NRotB’,‘TPSA’,
‘fNHAcc’,‘fNHDon’]
pd.scatter_matrix(train[[‘value’,‘logval’]+properties],figsize=(20,20))
plt.show()

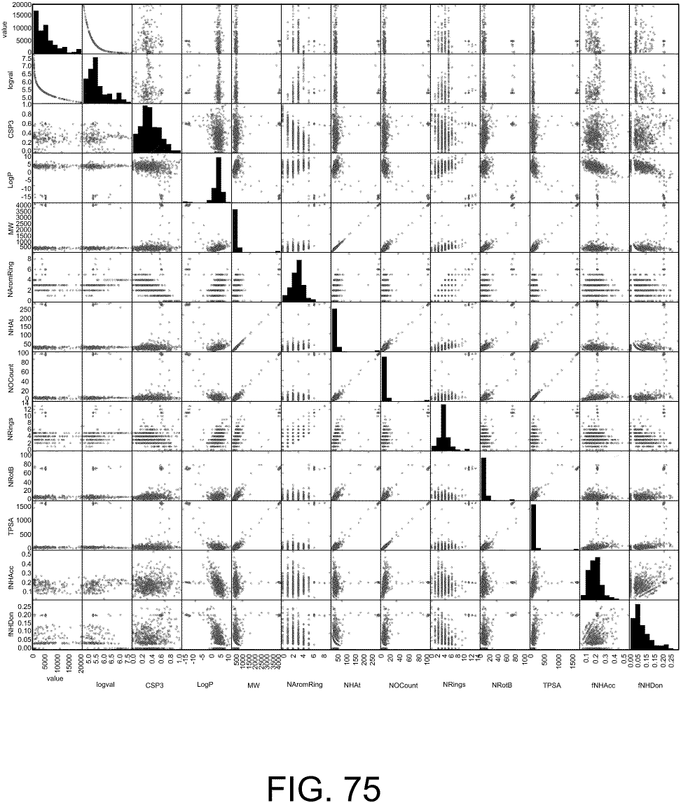

(see FIG. 75)

Draw.MolsToGridImage([Chem.MolFromSmiles(x) for x in
act.smiles.head(9)],subImgSize=(200,200))

Out:

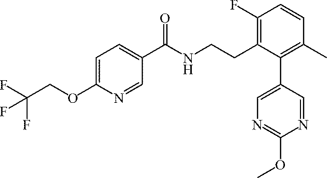 [SMILES: COc1ncc(-c2c(C)ccc(F)c2CCNC(=O)c2ccc(OCC(F)(F)F)nc2)cn1]

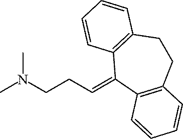 [SMILES: CN(C)CCC=C1c2ccccc2CCc2ccccc21]

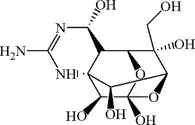

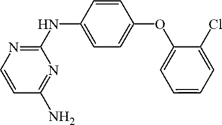 [SMILES: Nc1ccnc(Nc2ccc(Oc3ccccc3Cl)cc2)n1]

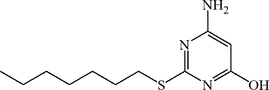 [SMILES: CCCCCCCSc1nc(N)cc(O)n1]

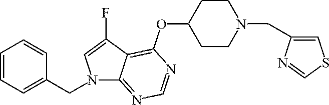 [SMILES: Fc1cn(Cc2ccccc2)c2ncnc(OC3CCN(Cc4cscn4)CC3)c12]

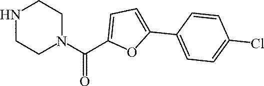 [SMILES: O=C(c1ccc(-c2ccc(Cl)cc2)o1)N1CCNCC1]

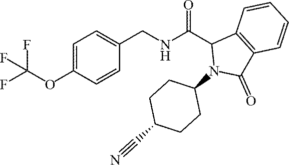 [SMILES: N#C[C@H]1CC[C@H](N2C(=O)c3ccccc3C2C(=O)NCc2ccc(OC(F)(F)F)cc2)CC1]

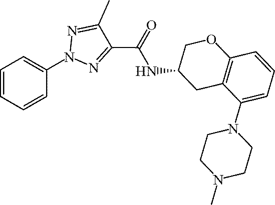 [SMILES: Cc1nn(-c2ccccc2)nc1C(=O)N[C@@H]1COc2cccc(N3CCN(C)CC3)c2C1]

In :

Draw.MolsToGridImage([Chem.MolFromSmiles(x) for x in
inact.smiles.head(9)],subImgSize=(200,200))

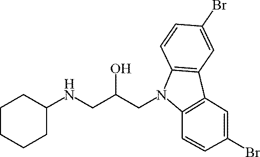 [SMILES: OC(CNC1CCCCC1)Cn1c2ccc(Br)cc2c2cc(Br)ccc21]

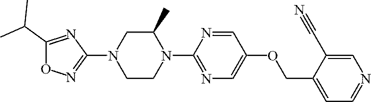 [SMILES: CC(C)c1nc(N2CCN(c3ncc(OCc4ccncc4C#N)cn3)[C@H](C)C2)no1]

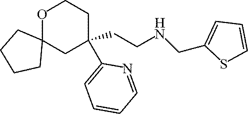 [SMILES: c1ccc([C@]2(CCNCc3cccs3)CCOC3(CCCC3)C2)nc1]

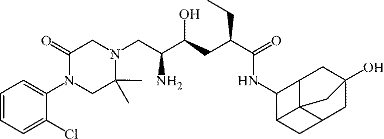 [SMILES: CC[C@H](C[C@H](O)[C@@H](N)CN1CC(=O)N(c2ccccc2Cl)CC1(C)C)C(=O)NC1C2CC3CC4(O)CC1C32C4]

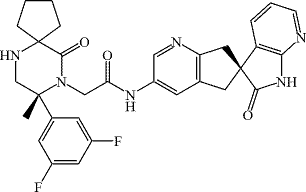 [SMILES: C[C@@]1(c2cc(F)cc(F)c2)CNC2(CCCC2)C(=O)N1CC(=O)Nc1cnc2c(c1)C[C@@]1(C2)C(=O)Nc2ncccc21]

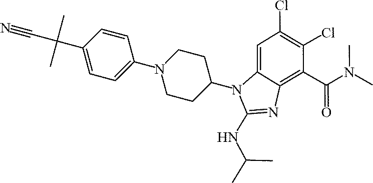 [SMILES: CC(C)Nc1nc2c(C(=O)N(C)C)c(Cl)c(Cl)cc2n1C1CCN(c2ccc(C(C)(C)C#N)cc2)CC1]

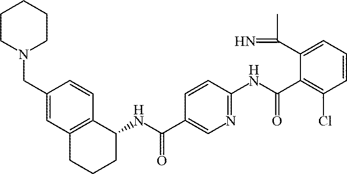 [SMILES: CC(=N)c1cccc(Cl)c1C(=O)Nc1ccc(C(=O)N[C@@H]2CCCc3cc(CN4CCCCC4)ccc32)cn1]

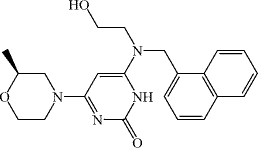 [SMILES: C[C@H]1CN(c2cc(N(CCO)Cc3cccc4ccccc34)[nH]c(=O)n2)CCO1]

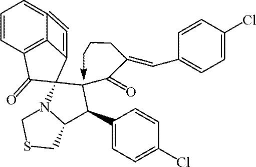

In :

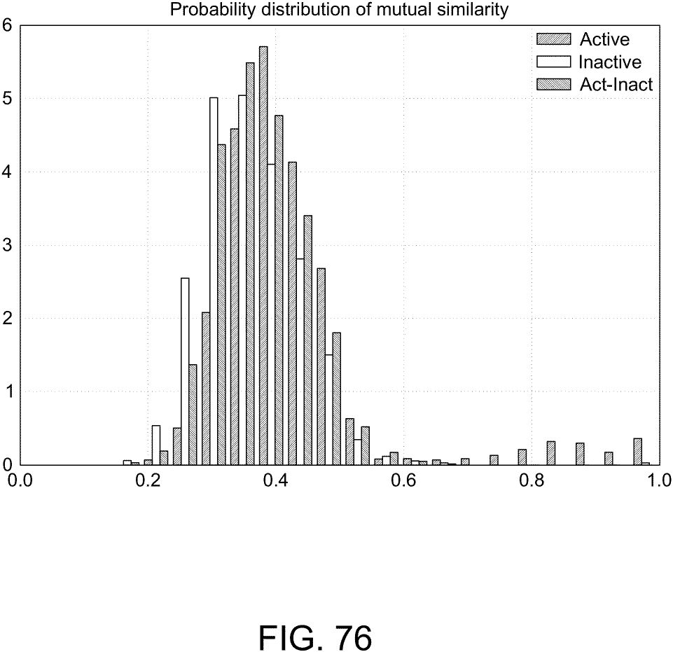

(see FIG. 76)

The similarity distribution with the actives and the inactive sets do overlap as shown in the figure. The plot reveals, there are significantly more similar compounds with similarity scores above 0.6 in the actives set. What is the actual fraction of very similar compounds? We should add a filtering step before generating training, test and validation sets

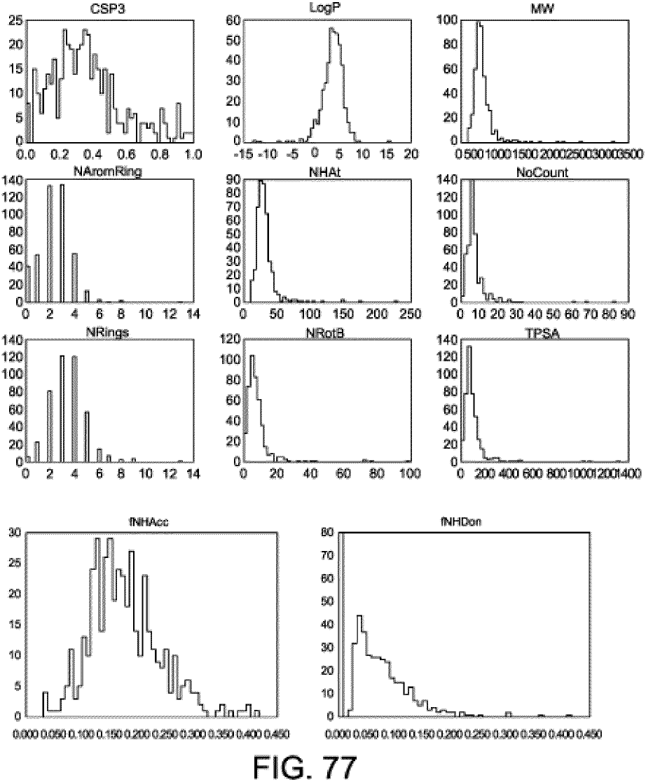

(see FIG. 77)

In :

for i in properties:
pit.hist(train[train.activity==0][i].values,alpha=0.5,color=‘b’,normed=True)
plt.hist(train[train.activity==1][i].values,alpha=0.5,color=‘r’,normed=True)
train.boxplot(by=‘activity’)
plt.title(i) pit.show()
#plt.hist(train(train.activity==1])

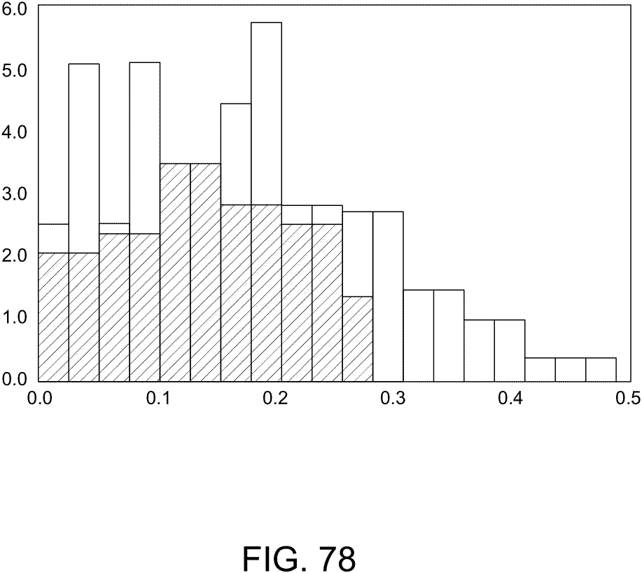

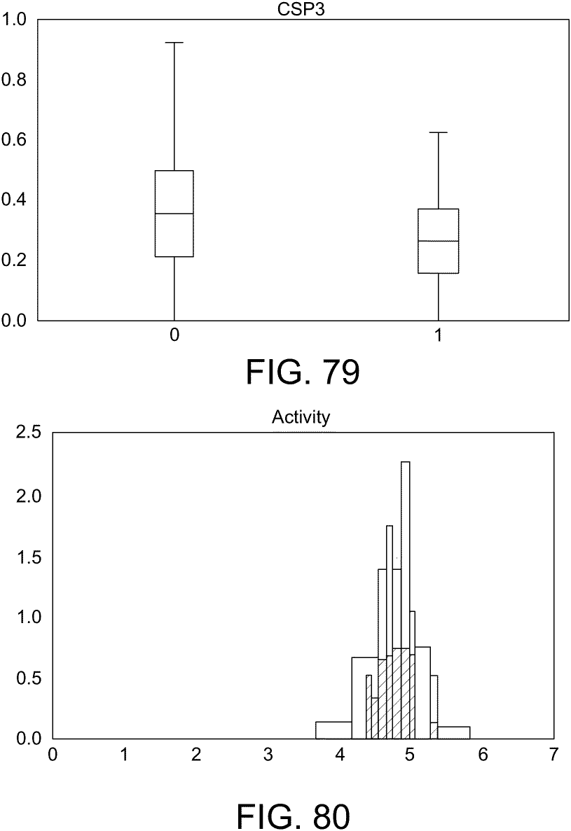

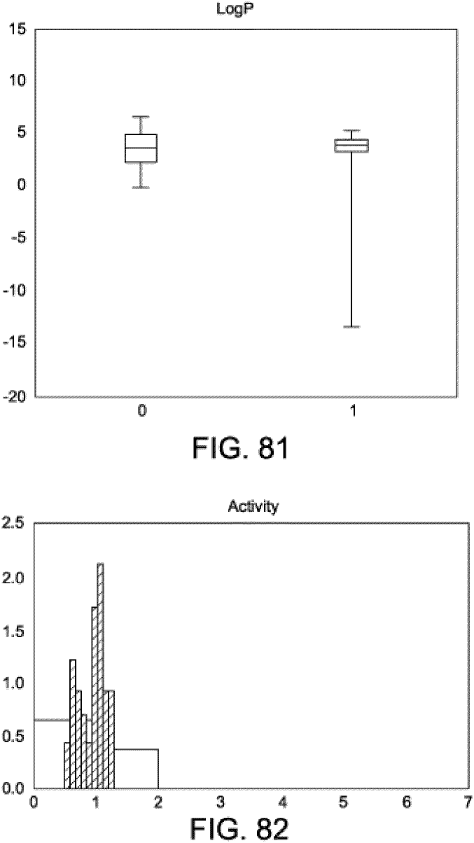

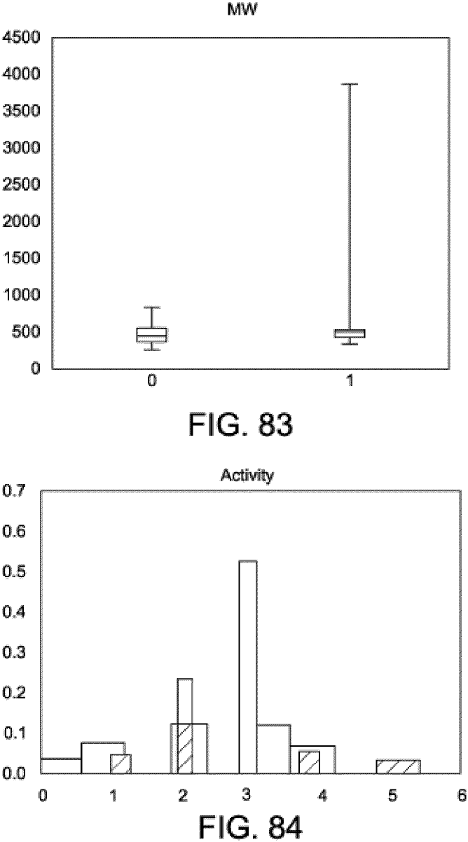

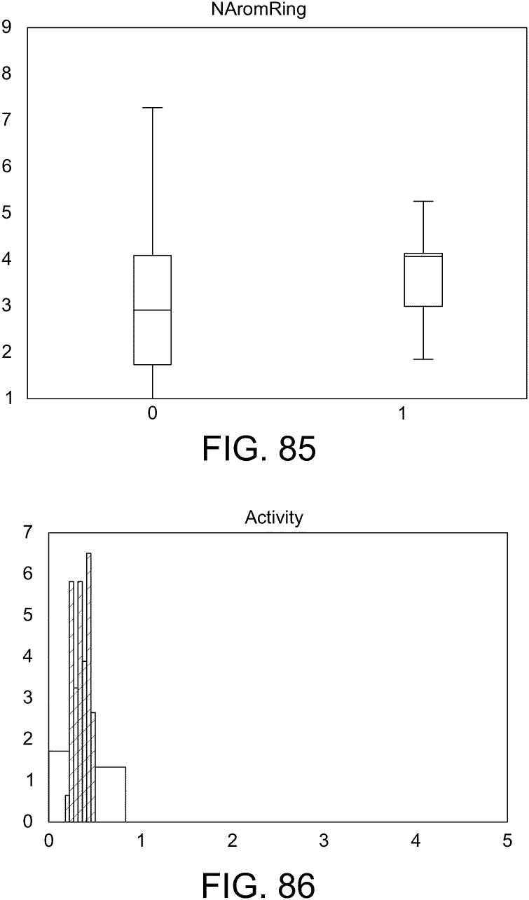

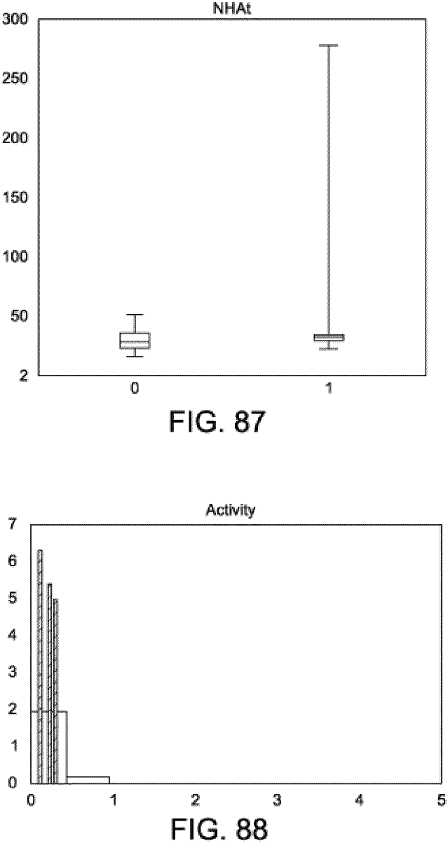

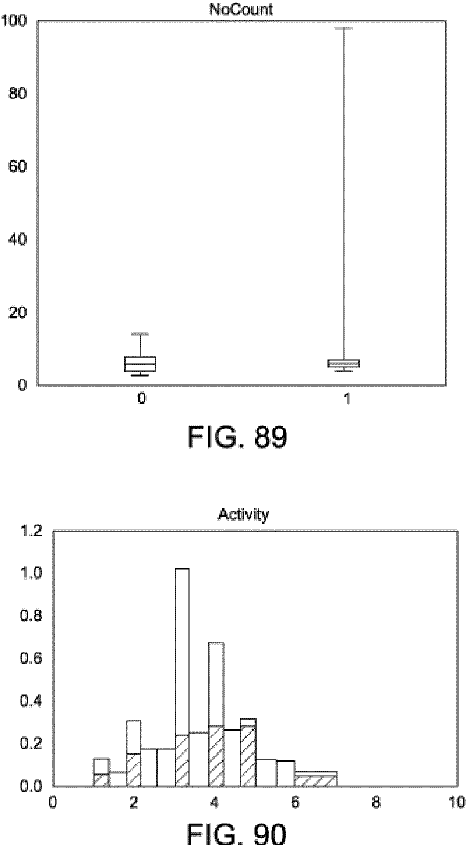

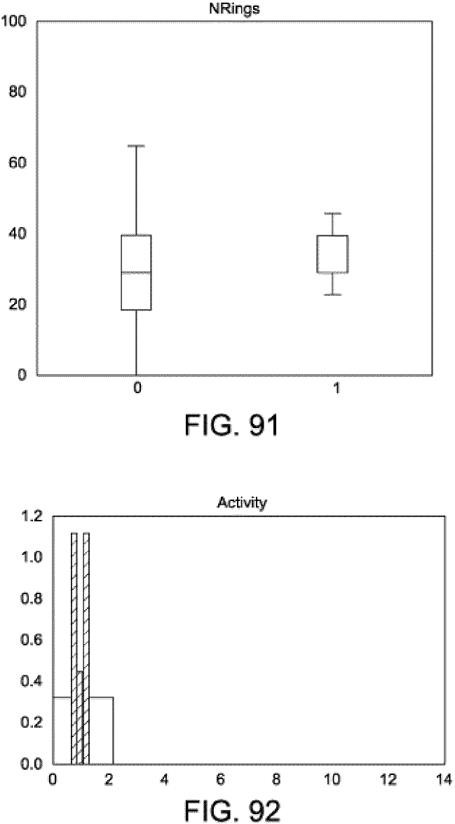

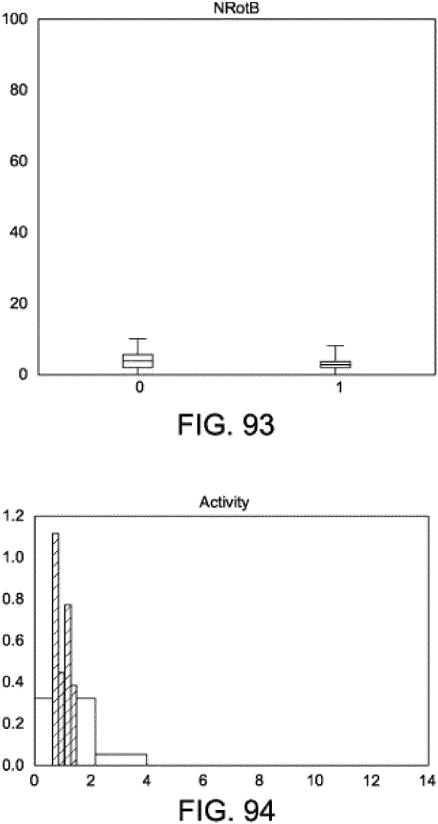

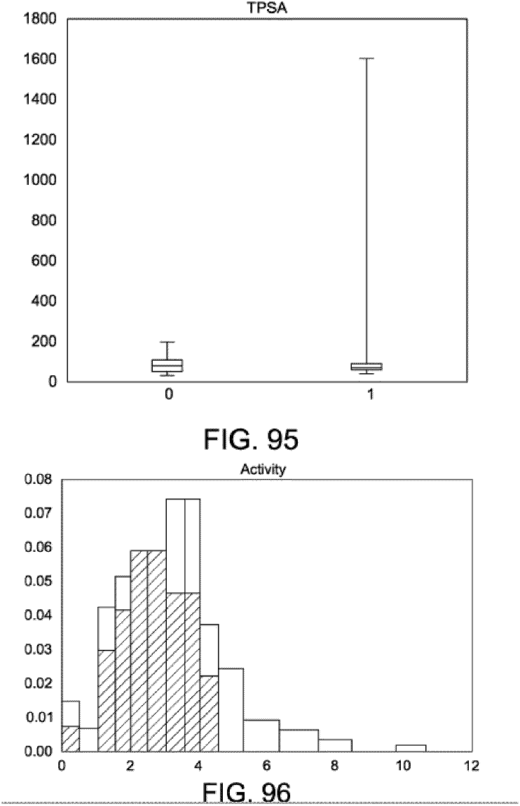

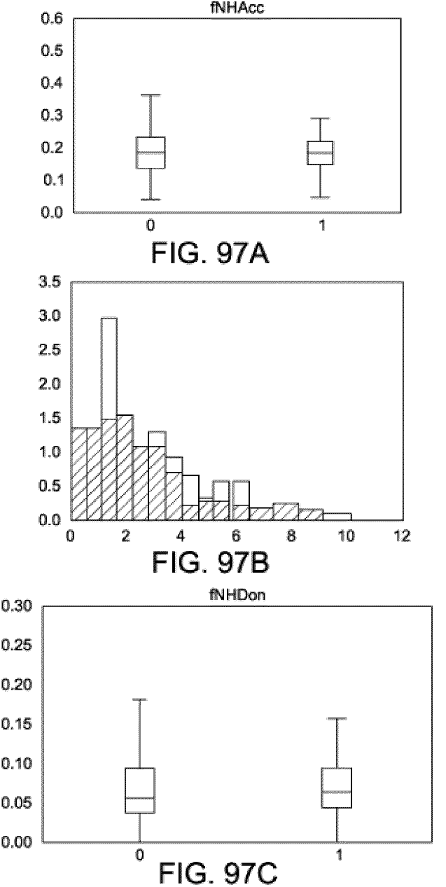

(see FIGS. 78-97)

### Activity Bar-Graph

In :

### Chemical Groups Based Predictions

In :

list(Data.index) Out[36]:
[‘fr_C_O’, ‘fr_bicyclic’, ‘fr_unbrch_alkane’, ‘fr_C_O_noCOO’, ‘fr_Al_OH’,
‘fr_Al_OH_noTert’, ‘fr_methoxy’, ‘fr_amide’, ‘fr_COO’, ‘fr_COO2’,
‘fr_allylic_oxid’, ‘fr_Ar_OH’, ‘fr_Al_COO’, ‘fr_ester’, ‘fr_phenol’,
‘fr_phenol_noOrthoHbond’, ‘fr_Ar_NH’,
‘fr_Nhpyrrole’, ‘fr_ketone’, ‘fr_imidazole’, ‘fr_sulfonamd’, ‘fr_ketone_Topliss’,
‘fr_thiazole’, ‘fr_guanido’, ‘fr_benzene’, ‘fr_NH0’,
‘fr_ArN’, ‘fr_aryl_methyl’, ‘fr_pyridine’, ‘fr_Ar_N’, ‘fr_aniline’, ‘fr_alkyl _halide’,
‘fr_halogen’]

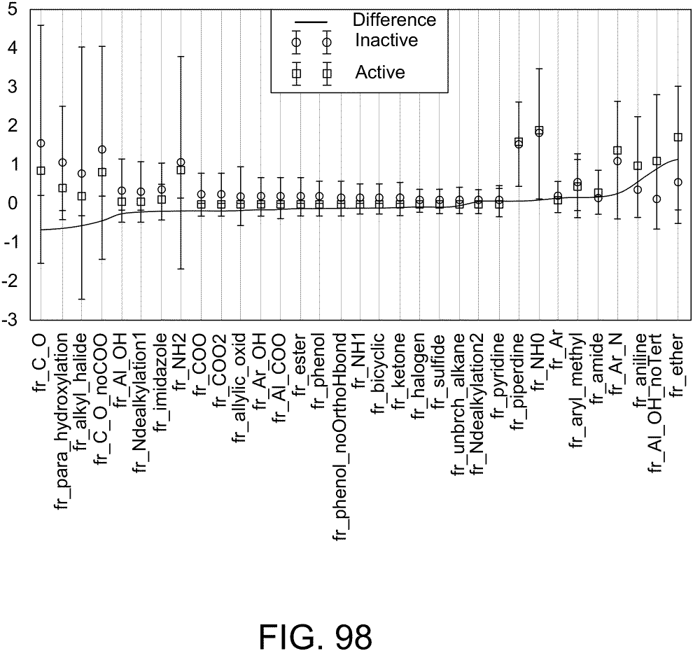

In [37]: (see FIG. 98)

The analysis of the mean of the counts of chemical groups shows that the chemical moieties correlate strongly in both sets active and inactive. A high number of a particular moiety in one group usually means a higher number in the other group too. For all groups the means are within the mutual standard deviations. Therefore, we cannot conclude that the occurrence of a certain moiety has special explanatory power, in the first place. However, we should look at the p-values. Also interesting would be to look at combinations.

In :

### NULL Models

In :

[‘CSP3’, ‘LogP’, ‘MW’, ‘NAromRing’, ‘NHAt’, ‘NOCount’, ‘NRings’, ‘NRotB’, ‘TPSA’,
‘fNHAcc’, ‘fNHDon’] 11 2048

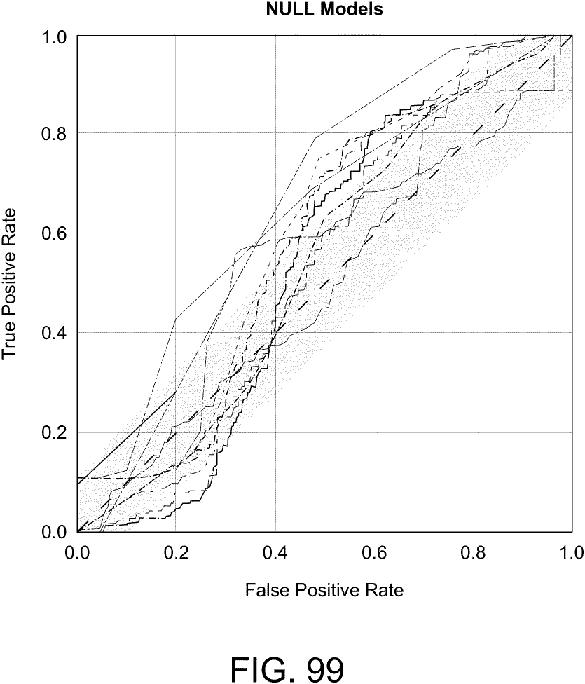

In [40]: (see FIG. 99)

X auroc

| | | | |
|---|---|---|---|
| 0 | CSP3 0.651163 | | |
| 1 | LogP 0.590469 | | |
| 3 9 | NAromRing 0.5854305 fNHAcc 0.557681 | NOCount 0.580426 8 | TPSA 0.575317 |
| 2 | MW 0.543939 | | |
| 4 | NHAt 0.542970 | | |
| 7 | NRotB 0.538055 | | |
| 6 | NRings 0.523908 | | |
| 10 | fNHDon 0.506272 11 | | |

The shape of the ROCs shows that the deviation of the active compounds is lower as compared to the decoys with a similar mean as seen in the

histograms. Can these variables be called weak predictors? Are these properties sufficient? In fact, there is no predictive power in the variables. In [41]:

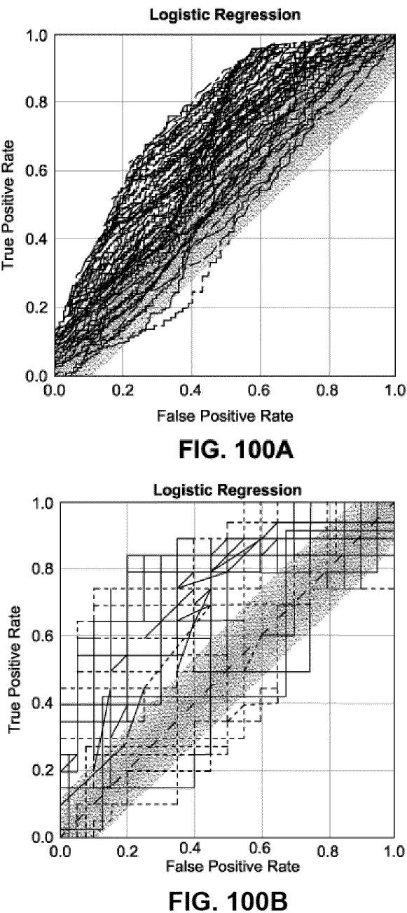

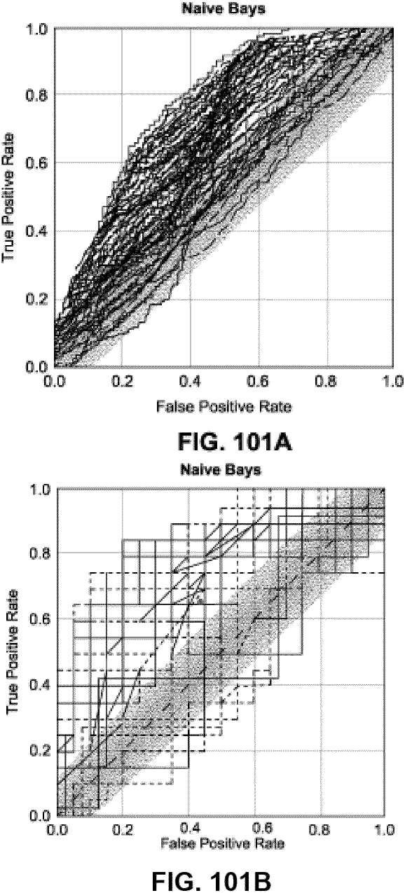

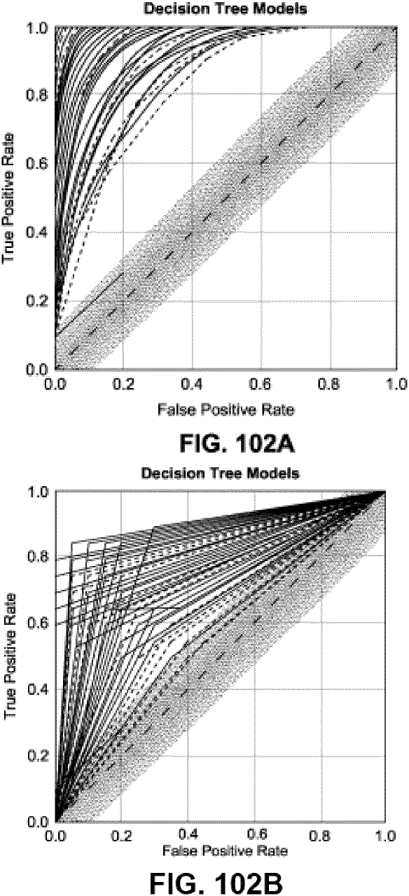

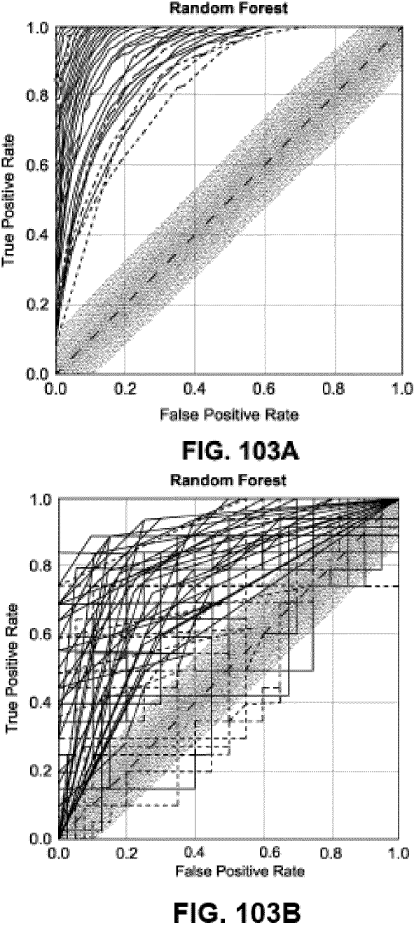

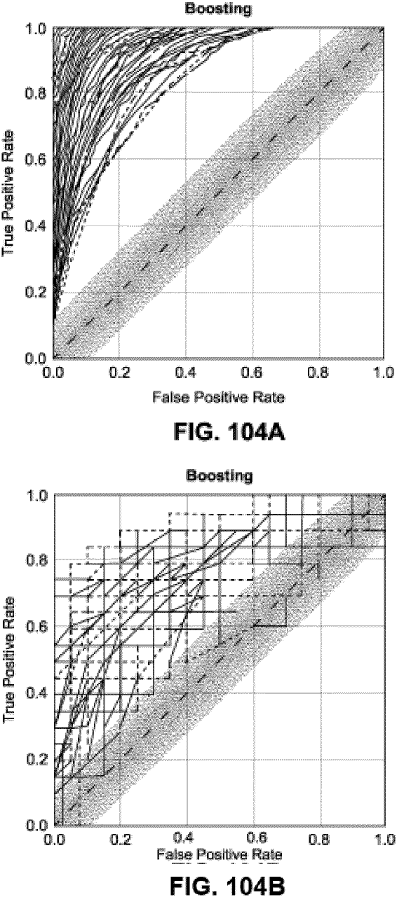

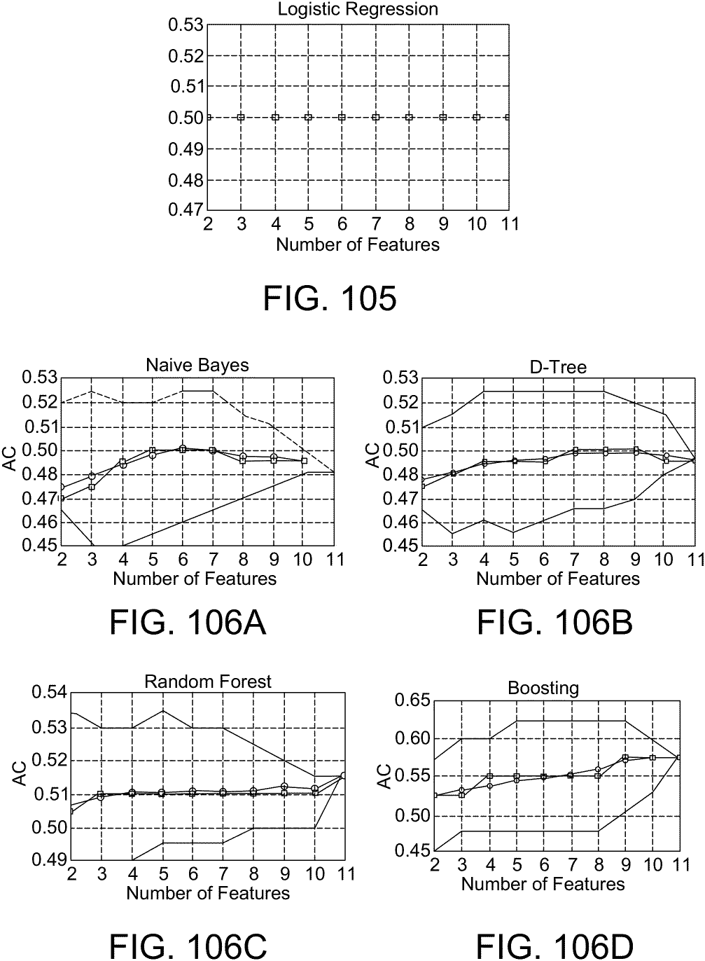

Model building Logistic Regression In [42]: (see FIGS. 100-106) 2035

### Naive Bayes

“Naive Bayes methods are a set of supervised learning algorithms based on applying Bayes’ theorem with the “naive” assumption of independence between every pair of features. Naive Bayes learners and classifiers can be extremely fast compared to more sophisticated methods. [...] [Although] naive Bayes is known as a decent classifier, it is known to be a bad estimator, so the probability outputs from predict_proba are not to be taken too seriously.” scikit-learn 0.15.2 documentation

In :

Decision Tree

In :

Random Forest

In :

Boosting

In :

### Number of Features and Prediction Accuracy

Type Markdown and LaTeX:

α
2

### Select Best Models

In :

LR_model=LR_models_test[[‘model’,‘AC’]].sort(‘AC’,ascending=False).model.values[0]
NB_model=NB_models_test[[‘model’,‘AC’]].sort(‘AC’,ascending=False).model.values[0]
DT_model=DT_models_test[[‘model’,‘AC’]].sort(‘AC’,ascending=False).model.values[0]
RF_model=RF_models_test[[‘model’,‘AC’]].sort(‘AC’,ascending=False).model.values[0]
BO_model=BO_models_test[[‘model’,‘AC’]].sort(‘AC’,ascending=False).model.values[0]

The finally selected Boosting model contains only 4 variables (NAromRing, fr Al COO, fNHDon and TPSA). It is interesting that the models often use different variables.

In :

A=EvalModels(BestModels_byAC,train,labels=BestModels_byAC.Family,plot=True,l
inewidth=2)
<matplotlib.text.Text at 0x67ca7d90>

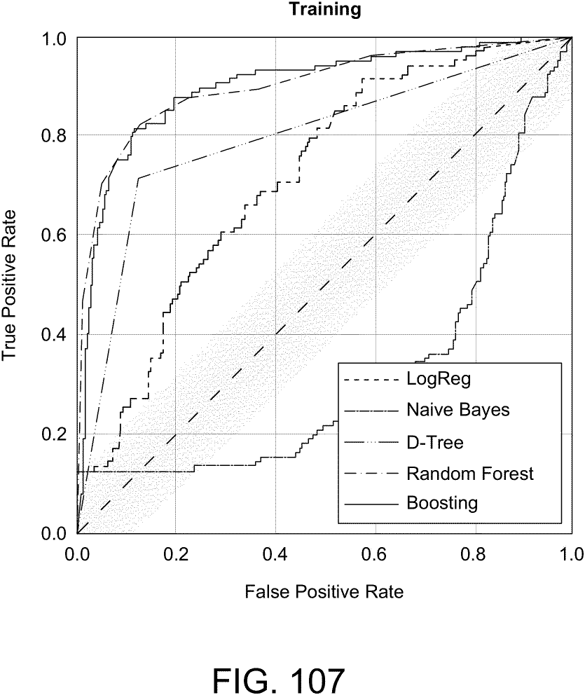

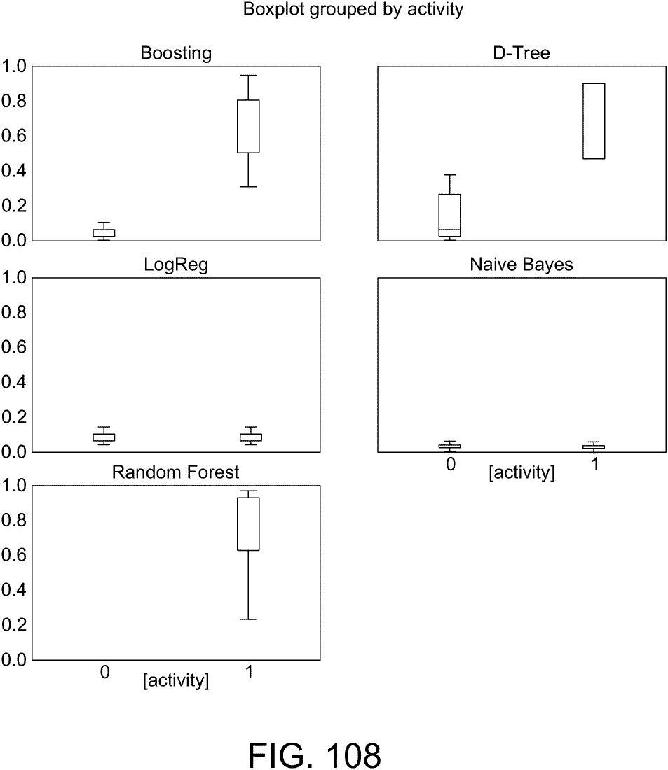

(see FIG. 107)

In :

A.sort(‘AC’,ascending=False
)

Out[51]

| | Family | AC | Sensitivit | Specificit | TP | TN | FP | FN |
|---|---|---|---|---|---|---|---|---|
| 2 | D-Tree | 0.990909 | 0.982143 | 1.000000 | 1.000000 | 0.981818 | 0.000000 | 0.018182 |
| 4 | Boosting | 0.927273 | 0.873016 | 1.000000 | 1.000000 | 0.854545 | 0.000000 | 0.145455 |
| 3 | Random Forest | 0.879880 | 0.808222 | 0.994950 | 0.996124 | 0.763636 | 0.003876 | 0.236364 |
| 1 | Naive Bayes | 0.729968 | 0.821867 | 0.678892 | 0.587209 | 0.872727 | 0.412791 | 0.127273 |
| 0 | LogReg | 0.500000 | 0.500000 | NaN | 1.000000 | 0.000000 | 0.000000 | 1.000000 |

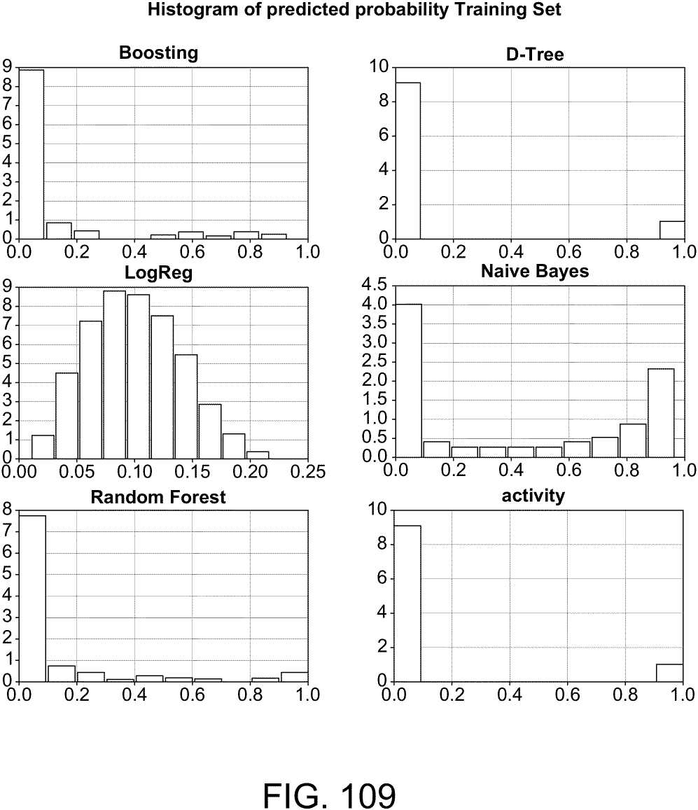

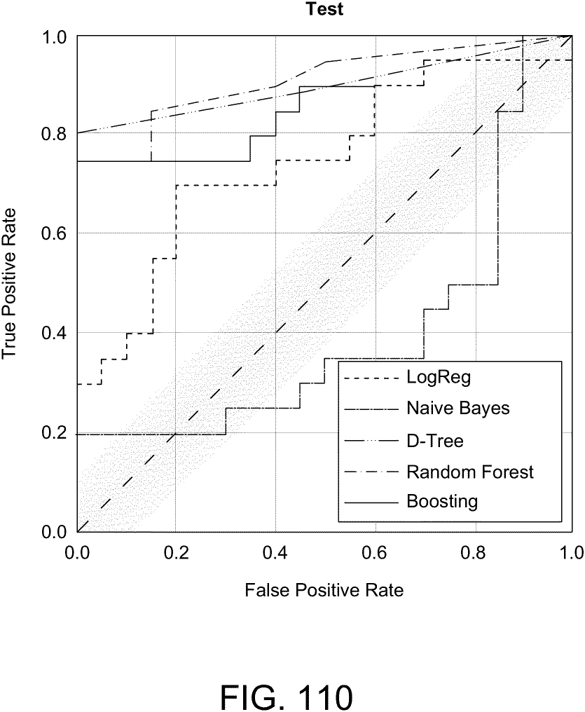

In : (see FIGS. 108-110)

In :

B.sort(‘AC’,ascending=False
)

Out[54]

| | Family | AC | Sensitivity | Specificity | TP | TN | FP | FN |
|---|---|---|---|---|---|---|---|---|
| 1 | Naive Bayes | 0.725 | 0.764706 | 0.695652 | 0.6 5 | 0.8 0 | 0.3 5 | 0.2 0 |
| 2 | D-Tree | 0.725 | 0.645161 | 1.000000 | 1.0 0 | 0.4 5 | 0.0 0 | 0.5 5 |
| 3 | Random Forest | 0.675 | 0.606061 | 1.000000 | 1.0 0 | 0.3 5 | 0.0 0 | 0.6 5 |
| 4 | Boosting | 0.625 | 0.571429 | 1.000000 | 1.0 | 0.2 | 0.0 | 0.7 |
| | | | | | 0 | 5 | 0 | 5 |
| 0 | LogReg | 0.500 | 0.500000 | NaN | 1.0 0 | 0.0 0 | 0.0 0 | 1.0 0 |

In :

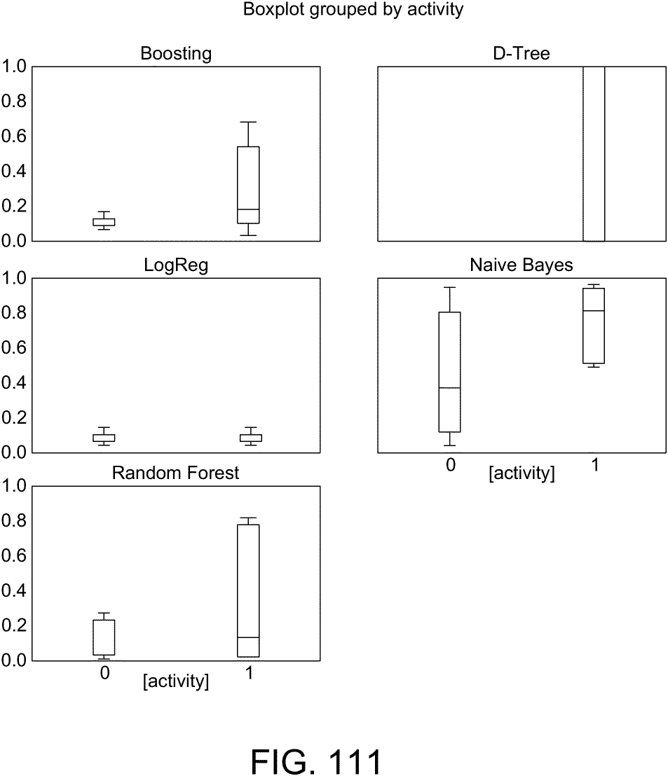

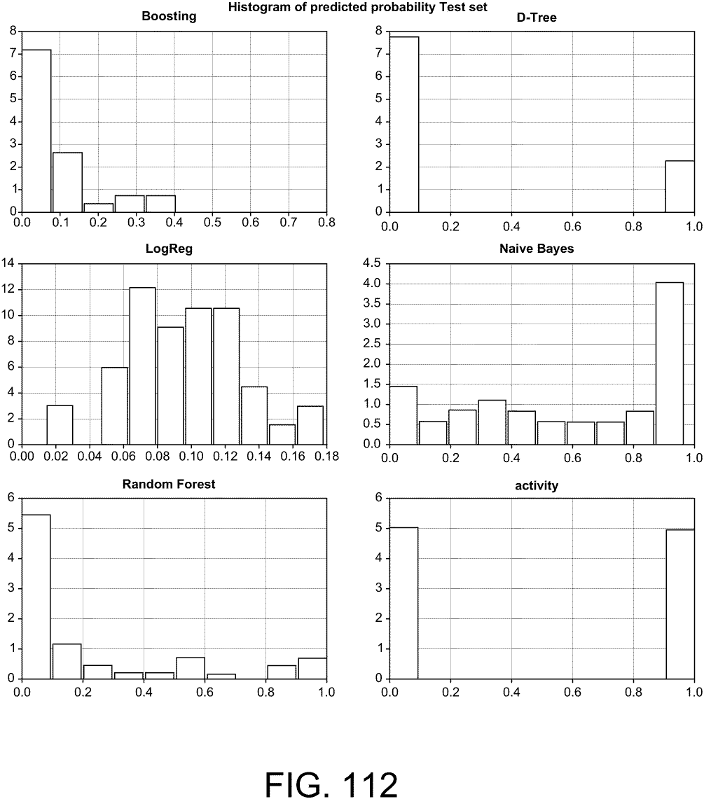

of predicted probabilities\nTestset′) plt.show() (see FIGS. 111 and 112)

In :

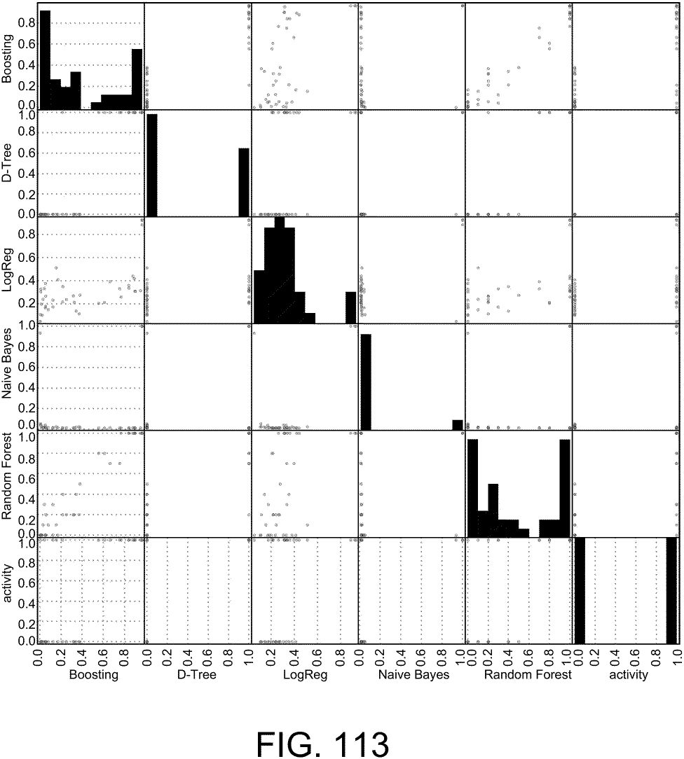

pd.scatter_matrix(Result_test,figsize=(10,10)) plt.show() (see FIG. 113)

### Error Estimation

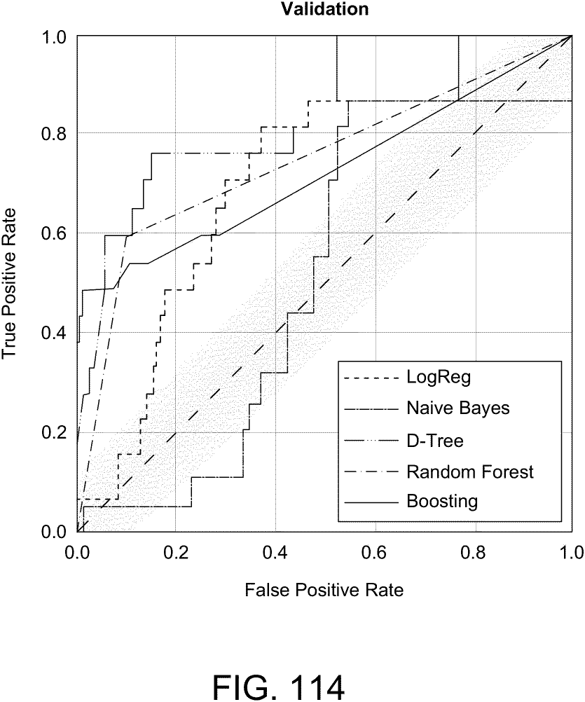

In : (see FIG. 114)

In :

C.sort(‘AC’,ascending=False
)

Out[58]

| | Family | AC | Sensitivity | Specificity | TP | TN | FP | FN |
|---|---|---|---|---|---|---|---|---|
| 2 | D-Tree | 0.771060 | 0.717195 | 0.860454 | 0.895062 | 0.647059 | 0.104938 | 0.352941 |
| 1 | Naive Bayes | 0.736020 | 0.900246 | 0.667353 | 0.530864 | 0.941176 | 0.469136 | 0.058824 |
| 3 | Random Forest | 0.659495 | 0.598875 | 0.912248 | 0.966049 | 0.352941 | 0.033951 | 0.647059 |
| 4 | Boosting | 0.645516 | 0.585457 | 0.989615 | 0.996914 | 0.294118 | 0.003086 | 0.705882 |
| 0 | LogReg | 0.500000 | 0.500000 | NaN | 1.000000 | 0.000000 | 0.000000 | 1.000000 |

In :

Result_valid={}
0 324
1 17
dtype: int64

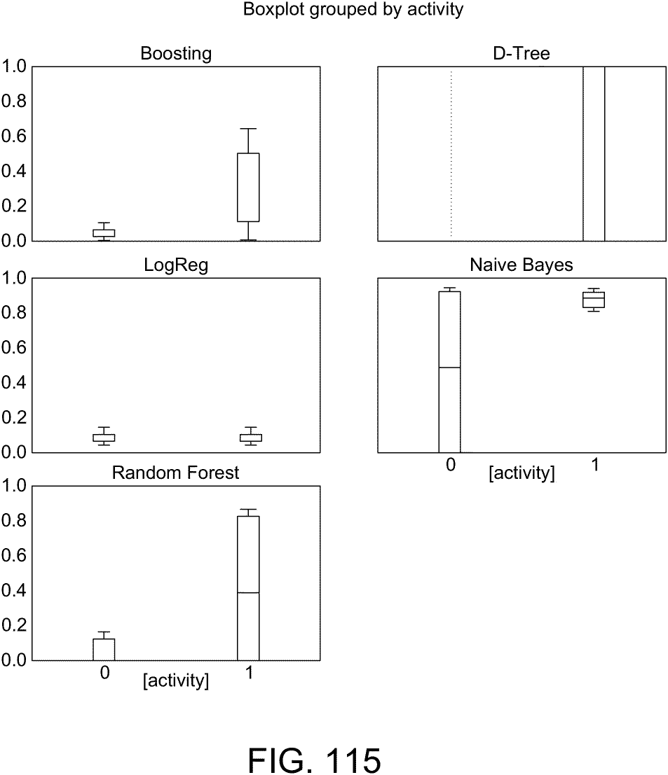

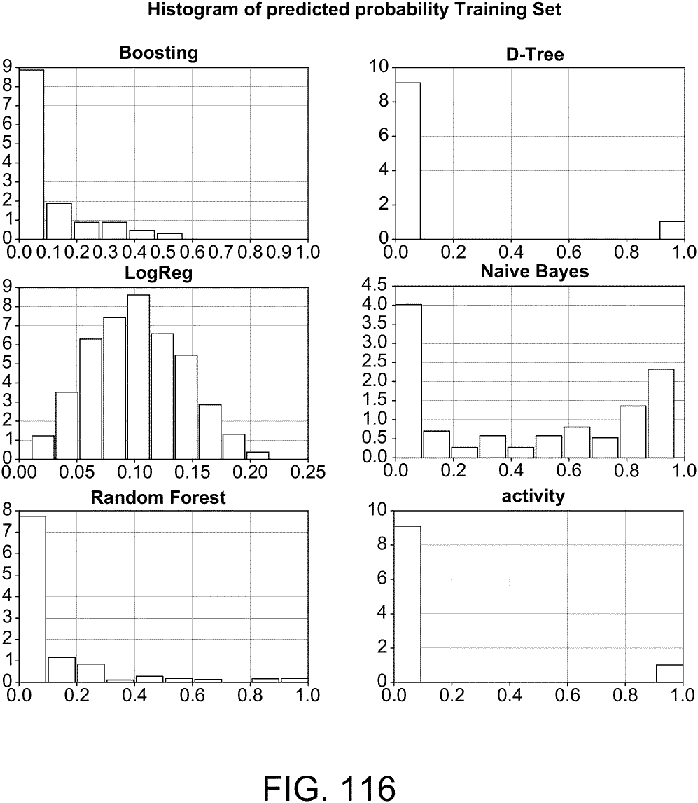

(see FIGS. 115 and 116)

Model Predictions Interpretation

In :

The classification accuracy of the models is between 0.55 for the LR model and and 0.75 for the Naive Bayes model. The true negative rate of the Naive Bayes model is best with 0.88. The FP rate of the RF and LR model are smallest. The RF model has a very high FN rate, although the accuracy is pretty high. Alltogether, the NB model seems to be the most robust model with a sensitivity of ~0.85 and specificity of ~0.7. However, the ROC shows that the RF model can perform much better. No literature data on machine learning in Nav1.5 available. The test set of experimental pIC50 is to be generated by Achlys Inc cell lines.
How is the classification done with model.predict()? Sensitivity TP/(TP+FN)

Specificity

TN/(FP+TN)
Accuracy (TP+TN)/(TP+FP+FN+TN)

### Quantitative Prediction

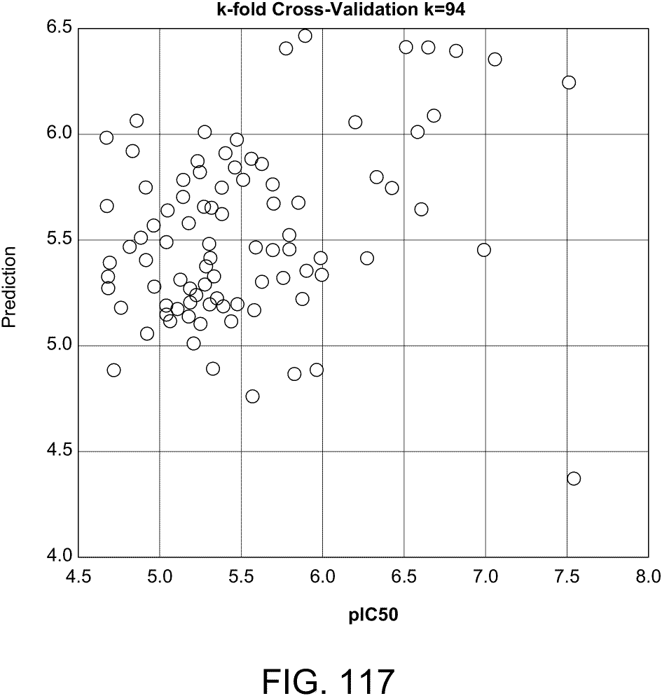

In :
94
Correlation coefficient: 0.296106199119
(see FIG. 117)

In :

The Brown set data points were used as a validation set.

### APPENDIX C

[image]

### Cav12 Activity Prediction

Can we use molecular structures, related properties and similiarities to distinguish between REC active and inactive compounds?
desired accuracy +90%

“‘RDKit and sklearn imports and related functions’”

import rdkit
from rdkit import Chem
from rdkit.Chem import AllChem
from rdkit.Chem import Draw
from rdkit.Chem.Draw import IPythonConsole
from sklearn.metrics import
roc_curve,confusion_matrix,roc_auc_score

Descriptors

‘NHAcc’ :d.NumHAcceptors(m),\ ‘MW’:d.ExactMolWt(m),\
‘NHAt’ :d.HeavyAtomCount(m),\
‘NRotB’ :d.NumRotatableBonds(m),\ ‘TPSA’ :d.TPSA(m),\
‘CSP3’ :d.FractionCSP3(m),\ ‘NRings’:d.RingCount(m),\
‘NOCount’:L.NOCount(m),\ ‘NAromRing’:L.NumAromaticRings(m),\
‘fr_Al_COO’:d.fr_Al_COO(m),\ ‘fr_aniline’:d.fr_aniline(m),\
‘fr_nitro’:d.fr_nitro(m),\‘fr_Al_OH’:d.fr_Al_OH(m),\
‘fr_aryl_methyl’:d.fr_aryl_methyl(m),\
‘fr_nitro_arom’:d.fr_nitro_arom(m),\
‘fr_Al_OH_noTert’:d.fr_Al_OH_noTert(m),\ ‘fr_azide’:d.fr_azide(m),\
‘fr_nitro_arom_nonortho’:d.fr_nitro_arom_nonortho(m),\
‘fr_ArN’:d.fr_ArN(m),\
‘fr_azo’:d.fr_azo(m),\ ‘fr_nitroso’:d.fr_nitroso(m),\
‘fr_Ar_COO’:d.fr_Ar_COO(m),\ ‘fr_barbitur’:d.fr_barbitur(m),\
‘fr_oxazole’:d.fr_oxazole(m),\ ‘fr_Ar_N’:d.fr_Ar_N(m),\
‘fr_benzene’:d.fr_benzene(m),\ ‘fr_oxime’:d.fr_oxime(m),\
‘fr_Ar_NH’:d.fr_Ar_NH(m),\
‘fr_benzodiazepine’:d.fr_benzodiazepine(m),\
‘fr_para_hydroxylation’:d.fr_para_hydroxylation(m),\
‘fr_Ar_OH’:d.fr_Ar_OH(m),\ ‘fr_bicyclic’:d.fr_bicyclic(m),\
‘fr_phenol’:d.fr_phenol(m),\
‘fr_COO’:d.fr_COO(m),\ ‘fr_diazo’:d.fr_diazo(m),\
‘fr_phenol_noOrthoHbond’:d.fr_phenol_noOrthoHbond(m),\
‘fr_COO2’:d.fr_COO2(m),\
‘fr_dihydropyridine’:d.fr_dihydropyridine(m),\
‘fr_phos_acid’:d.fr_phos_acid(m),\
‘fr_C_O’:d.fr_C_O(m),\ ‘fr_epoxide’:d.fr_epoxide(m),\
‘fr_phos_ester’:d.fr_phos_ester(m),\
‘fr_C_O_noCOO’:d.fr_C_O_noCOO(m),\ ‘fr_ester’:d.fr_ester(m),\
‘fr_piperdine’:d.fr_piperdine(m),\ ‘fr_C_S’:d.fr_C_S(m),\
‘fr_ether’:d.fr_ether(m),\ ‘fr_piperzine’:d.fr_piperzine(m),\
‘fr_HOCCN’:d.fr_HOCCN(m),\ ‘fr_furan’:d.fr_furan(m),\
‘fr_priamide’:d.fr_priamide(m),\ ‘fr_Imine’:d.fr_Imine(m),\
‘fr_guanido’:d.fr_guanido(m),\ ‘fr_prisulfonamd’:d.fr_prisulfonamd(m),\
‘fr_NH0’:d.fr_NH0(m),\ ‘fr_halogen’:d.fr_halogen(m),\
‘fr_pyridine’:d.fr_pyridine(m),\ ‘fr_NH1’:d.fr_NH1(m),\
‘fr_hdrzine’:d.fr_hdrzine(m),\ ‘fr_quatN’:d.fr_quatN(m),\
‘fr_NH2’:d.fr_NH2(m),\ ‘fr_hdrzone’:d.fr_hdrzone(m),\
‘fr_sulfide’:d.fr_sulfide(m),\ ‘fr_N_O’:d.fr_N_O(m),\
‘fr_imidazole’:d.fr_imidazole(m),\ ‘fr_sulfonamd’:d.fr_sulfonamd(m),\
‘fr_Ndealkylation1’:d.fr_Ndealkylation1(m),\ ‘fr_imide’:d.fr_imide(m),\
‘fr_sulfone’:d.fr_sulfone(m),\
‘fr_Ndealkylation2’:d.fr_Ndealkylation2(m),\
‘fr_isocyan’:d.fr_isocyan(m),\
‘fr_term_acetylene’.d.fr_term_acetylene(m),\
‘fr_Nhpyrrole’:d.fr_Nhpyrrole(m),\
‘fr_isothiocyan’:d.fr_isothiocyan(m),\ ‘fr_tetrazole’:d.fr_tetrazole(m),\
‘fr_SH’:d.fr_SH(m),\ ‘fr_ketone’:d.fr_ketone(m),\
‘fr_thiazole’:d.fr_thiazole(m),\ ‘fr_aldehyde’:d.fr_aldehyde(m),\
‘fr_ketone_Topliss’:d.fr_ketone_Topliss(m),\
‘fr_thiocyan’:d.fr_thiocyan(m),\
‘fr_alkyl_carbamate’:d.fr_alkyl_carbamate(m),\
‘fr_lactam’:d.fr_lactam(m),\ ‘fr_thiophene’:d.fr_thiophene(m),\
‘fr_alkyl_halide’:d.fr_alkyl_halide(m),\
‘fr_lactone’:d.fr_lactone(m),\
‘fr_unbrch_alkane’:d.fr_unbrch_alkane(m),\
‘fr_allylic_oxid’:d.fr_allylic_oxid(m),\ ‘fr_methoxy’:d.fr_methoxy(m),\
‘fr_urea’:d.fr_urea(m),\ ‘fr_amide’:d.fr_amide(m),\
‘fr_morpholine’.d.fr_morpholine(m)\ ‘fr_amidine’:d.fr_amidine(m),\
‘fr_nitrile’:d.fr_nitrile(m),
}
return out

“‘Test of function’”
df=pd.DataFrame(list(MolecularProperties([‘CCC’,‘C1CCOC1(=N)’,‘O
CO’]))) df

Out:

| | CSP3 | LogP | MW | NAromRing | NHAcc | NHAt | NHDon | NO Count | NRings |
|---|---|---|---|---|---|---|---|---|---|
| 0 | 1.00 | 1.41630 | 44.062600 | 0 | 0 | 3 | 0 | 0 | 0 |
| 1 | 0.75 | 0.77407 | 85.052764 | 0 | 2 | 6 | 1 | 2 | 1 |
| 2 | 1.00 | -1.07150 | 48.021129 | 0 | 2 | 3 | 2 | 2 | 0 |

| | NRotB | fr_sulfide | fr_sulfonamd | fr_sulfone | fr_term_acetylene | fr_tetrazole | fr_thiazole | fr_thiocyan | fr_thiophene | |
|---|---|---|---|---|---|---|---|---|---|---|
| 0 | 0 | 0 | 0 | 0 | 0 | 0 | 0 | 0 | 0 | 0 |
| 1 | 0 | 0 | 0 | 0 | 0 | 0 | 0 | 0 | 0 | 0 |
| 2 | 0 | 0 | 0 | 0 | 0 | 0 | 0 | 0 | 0 | 0 |

3 rows × 96 columns

In :

Number of entries before filtering: 254 Number of entries after filtering: 27

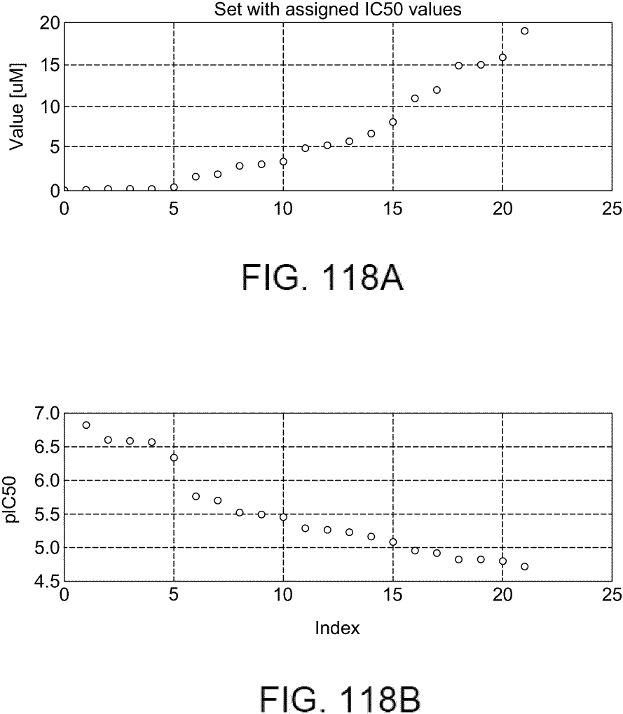

(see FIGS. 118A and 118B)

The data reveals several plateaus e.g. at 10000 nM and 30000 nM. These might be experimental artifacts. The data spans six orders of magnitude. In this figures compounds without a value (the inactive set) is omitted.

In :

‘Get structures’
print “Database connection:”, compounds.status()
structures=compounds.get(list(REC_bioact.parent_cmpd_chemblid))
#Get the compound structures from the ChEMBL database

Database connection: True

Found structures of 27 compounds In [15]:

#del decoys
“‘Decoys’”

“‘Filtering with pandas is a PITA’”
‘Filter out the entries with empthy smiles strings’
‘check for smiles entries that contain multiple molecules’
print len(REC_data)
‘Data Transformation’ Doublicates 0
879

Found 16 actives and 863 inactive compounds. 879

CHEMBL585802 1
CHEMBL470713 1
CHEMBL282299 1
CHEMBL412607 1
CHEMBL98558 1
dtype: int64
In [17]:

### Exploratory Data Analysis

In :

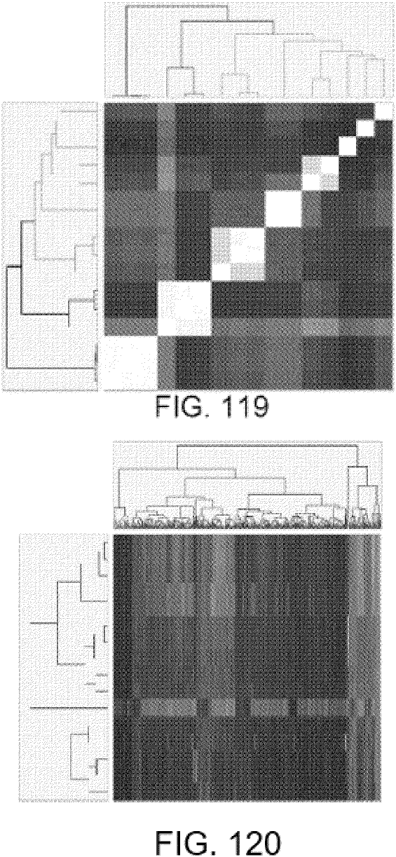

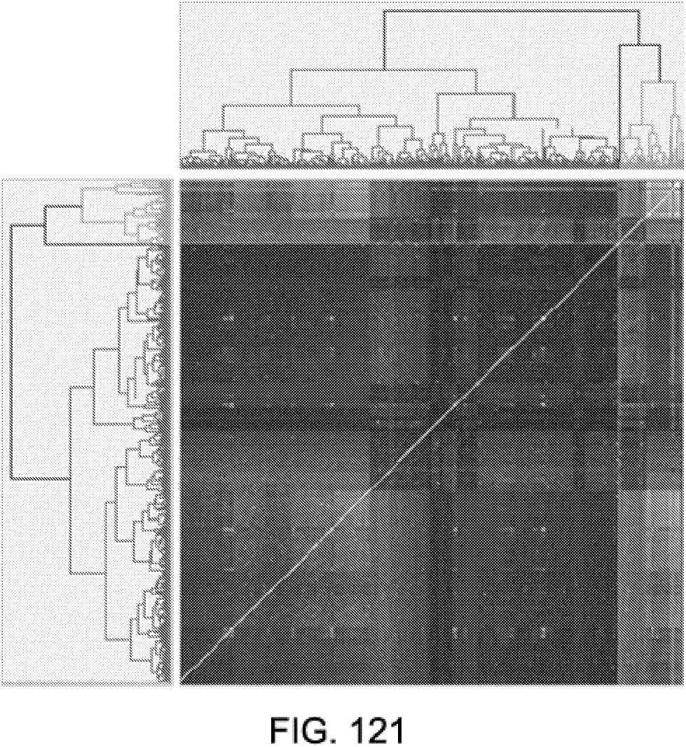

(see FIGS. 119-121)

Heatmap of the randomly selected active (left) and inactive (top) compounds, based on Dice similarity score:

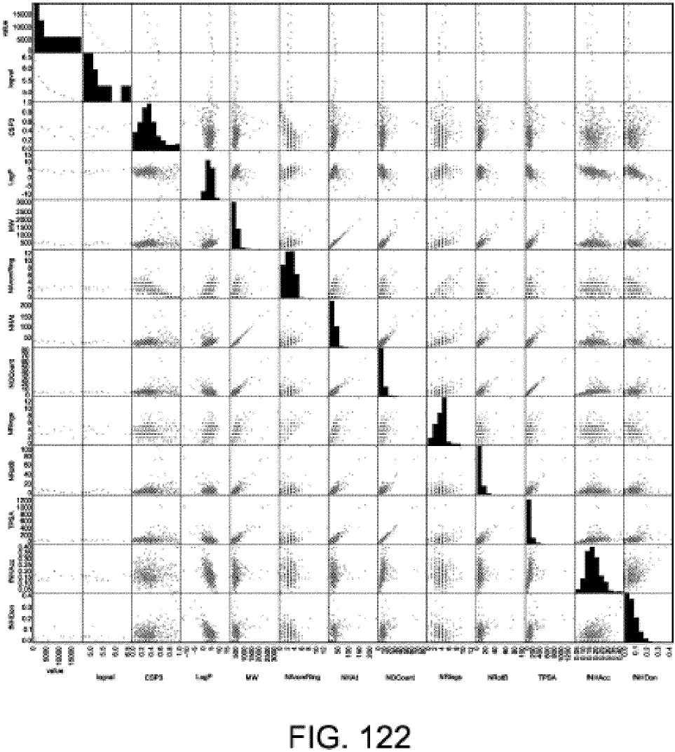

S Dice= 2p+q+d

Where
P number of positive in both,
Q number of positive in A but not B
(see FIG. 122)

d number of positive in B but not A

### Results

The similarity of the inactive compounds is much higher than expected, if only the REC inactive records in CHEMBLDB are used.
therefore ~800 decoys were added randomly selected from CHEMBLDB
the dissimilarity is OK In [20]:

properties = [‘CSP3’,‘LogP’,‘MW,’NAromRing’,\
‘NHAt’,‘NOCount’,‘NRings’,‘NRotB’,‘TPSA’,
‘fNHAcc’,‘fNHDon’]
pd.scatter_matrix(train[[‘value’,‘logval’]+properties],figsize=(20,20))
plt.show()

In :

Draw.MolsToGridlmage([Chem.MolFromSmiles(x) for x in act.smiles.head(9)],sublmgSize=(200,200))

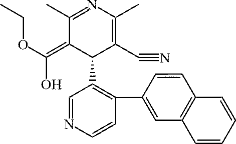 [SMILES: CCO/C(O)=C1\C(C)=NC(C)=C(C#N)[C@@H]1c1cnccc1-c1ccc2ccccc2c1]

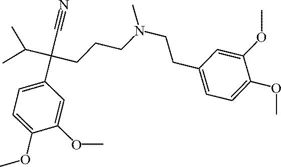

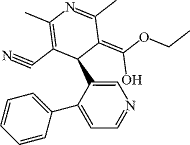 [SMILES: CCO/C(O)=C1\C(C)=NC(C)=C(C#N)[C@@H]1c1cnccc1-c1ccccc1]

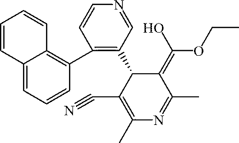 [SMILES: CCO/C(O)=C1\C(C)=NC(C)=C(C#N)[C@@H]1c1cnccc1-c1cccc2ccccc12]

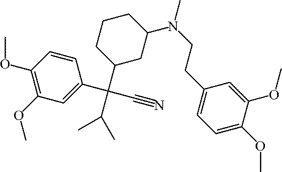 [SMILES: COc1ccc(CCN(C)C2CCCC(C(C#N)(c3ccc(OC)c(OC)c3)C(C)C)C2)cc1OC]

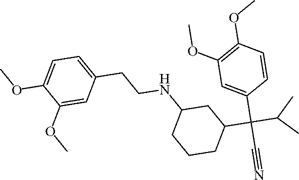 [SMILES: COc1ccc(CCNC2CCCC(C(C#N)(c3ccc(OC)c(OC)c3)C(C)C)C2)cc1OC]

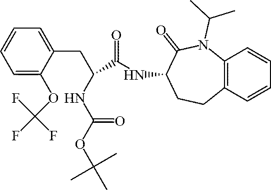 [SMILES: CC(C)N1C(=O)[C@@H](NC(=O)[C@@H](Cc2ccccc2OC(F)(F)F)NC(=O)OC(C)(C)C)CCc2ccccc21]

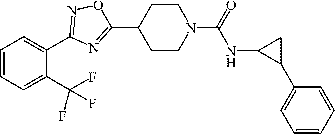 [SMILES: O=C(NC1CC1c1ccccc1)N1CCC(c2nc(-c3ccccc3C(F)(F)F)no2)CC1]

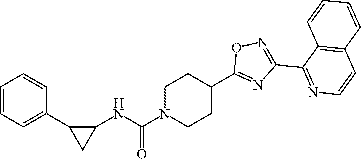 [SMILES: O=C(NC1CC1c1ccccc1)N1CCC(c2nc(-c3nccc4ccccc34)no2)CC1]

In :

Draw.MolsToGridlmage([Chem.MolFromSmiles(x) for x in inact.smiles.head(9)],sublmgSize=(200,200))

Out:

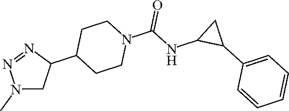 [SMILES: CN1CC(C2CCN(C(=O)NC3CC3c3ccccc3)CC2)N=N1]

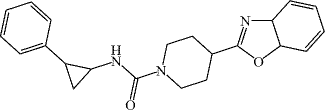 [SMILES: O=C(NC1CC1c1ccccc1)N1CCC(C2=NC3C=CC=CC3O2)CC1]

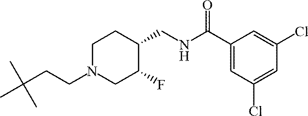 [SMILES: CC(C)(C)CCN1CC[C@H](CNC(=O)c2cc(Cl)cc(Cl)c2)[C@H](F)C1]

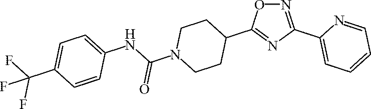 [SMILES: O=C(Nc1ccc(C(F)(F)F)cc1)N1CCC(c2nc(-c3ccccn3)no2)CC1]

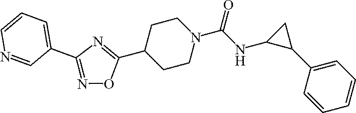 [SMILES: O=C(NC1CC1c1ccccc1)N1CCC(c2nc(-c3cccnc3)no2)CC1]

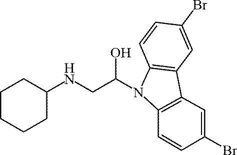 [SMILES: OC(CNC1CCCCC1)n1c2ccc(Br)cc2c2cc(Br)ccc21]

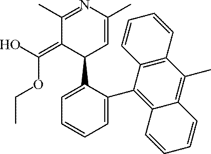 [SMILES: CCO/C(O)=C1/C(C)=NC(C)=C[C@H]1c1ccccc1-c1c2ccccc2c(C)c2ccccc12]

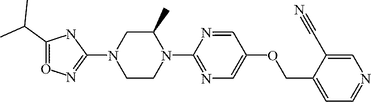

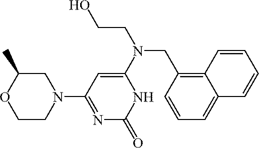 [SMILES: C[C@H]1CN(c2cc(N(CCO)Cc3cccc4ccccc34)[nH]c(=O)n2)CCO1]

In :

The similarity distribution with the actives and the inactive sets do overlap as shown in the figure. The plot reveals, there are a view more similar compounds with similarity scores above 0.8 in the actives set. The inactive set has a view compounds wich are rather dissimilar from the rest. However, the fraction of both have only minor impact on the modeling.

In :

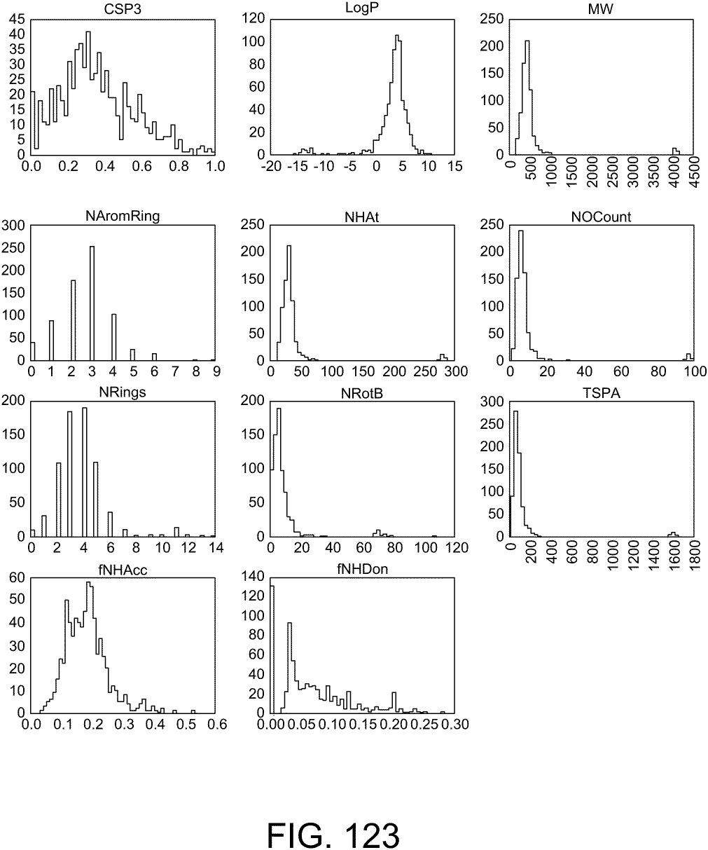

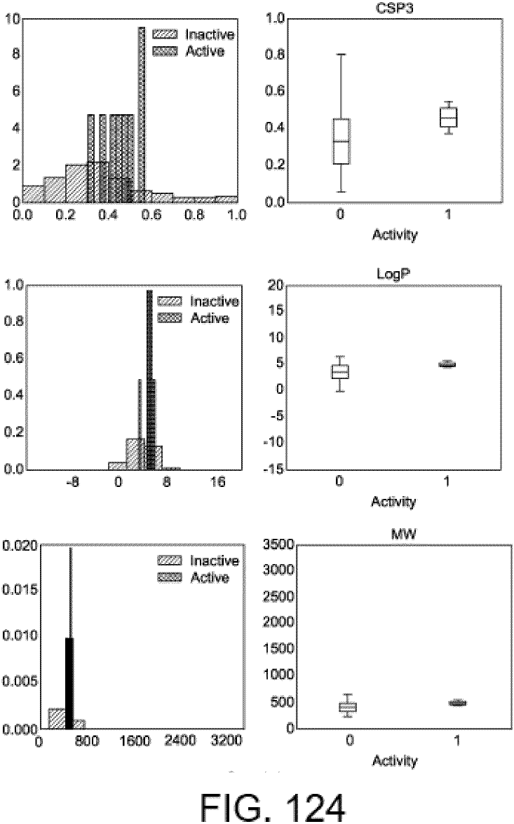

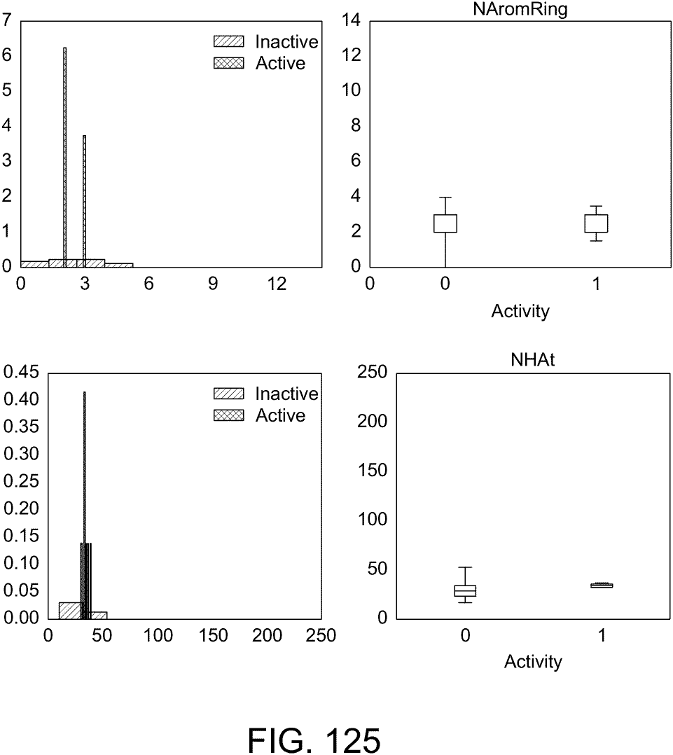

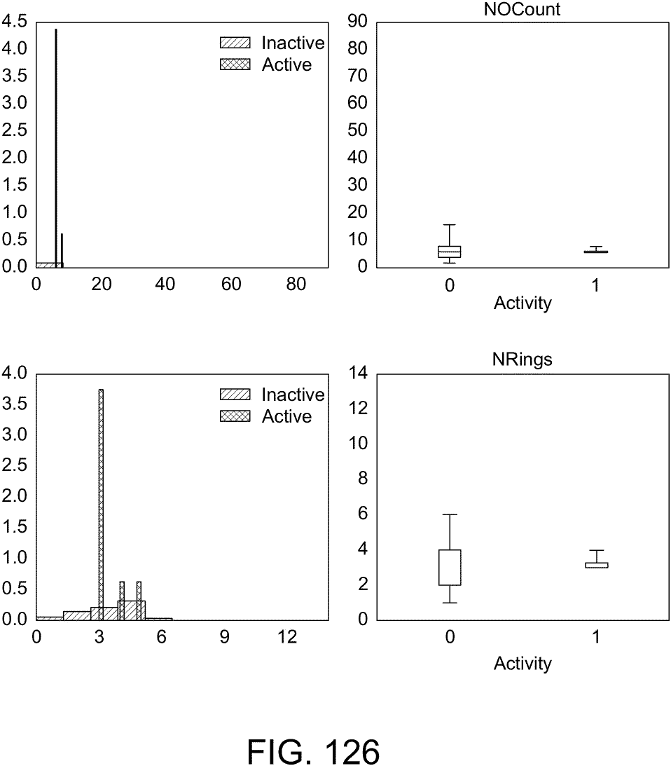

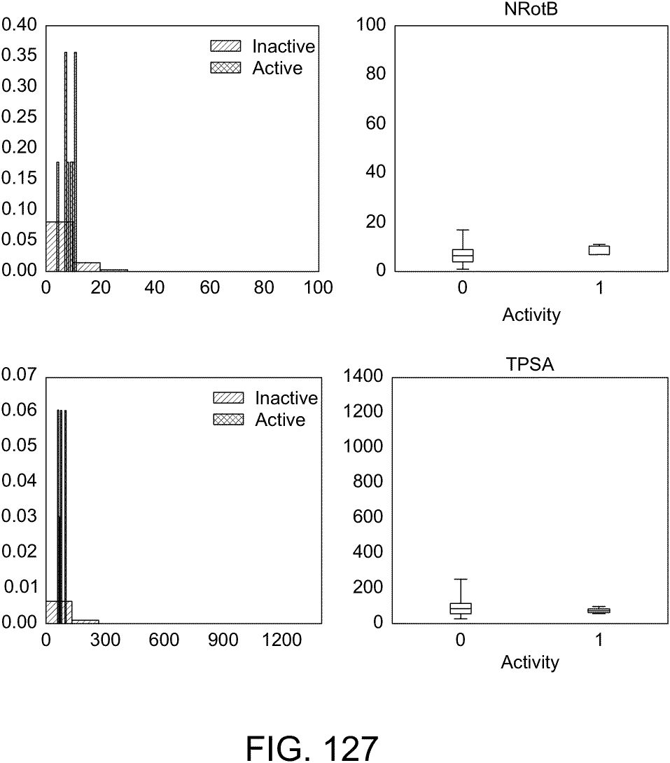

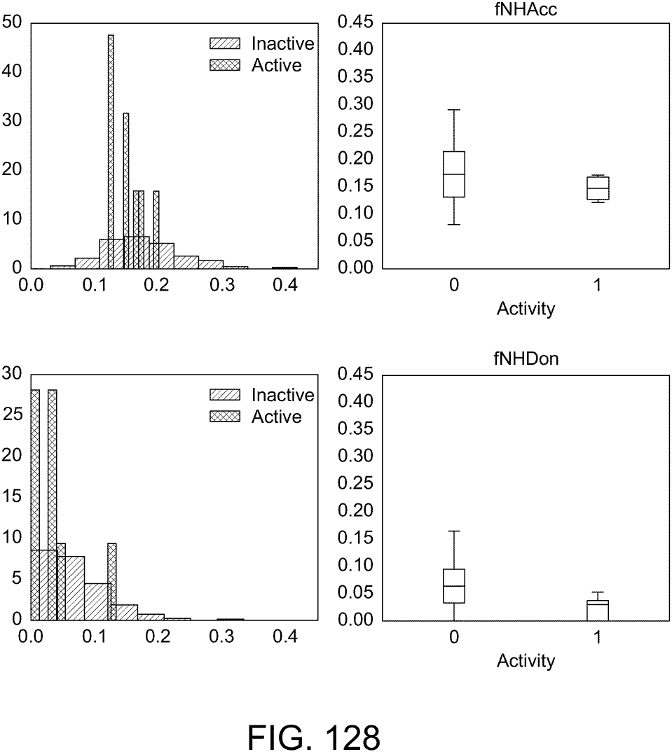

(see FIGS. 123-128)

In :

### Chemical Groups Based Predictions

In :

Means=train.filter(regex=‘fr_|activity’).groupby(‘activity’).mean(
).T Means[‘dMean’]=Means[1 ]-Means[0]
Stds=train.filter(regex=‘fr_|activity’).groupby(‘activity’).std().T
Means=Means.rename(columns={0:‘Mean-0’,1:‘Mean-1’})
Stds=Stds.rename(columns={0:‘STD-0’,1:‘STD-1’})
Data=Means.join(Stds)
Data=Data.sort(‘dMean’)
Data=Data[np.abs(Data.dMean)>0.05]

In :

list(Data.index)

Out:

[‘fr_bicyclic’, ‘fr_unbrch_alkane’, ‘fr_Ar_N’,
‘fr_C_O’,
‘fr_NH1’,
‘fr_COO’, ‘fr_COO2’, ‘fr_ketone’, ‘fr_Ar_OH’, ‘fr_Al_COO’,
‘fr_phenol’,
‘fr_C_O_noCOO’, ‘fr_phenol_noOrthoHbond’,
‘fr_ketone_Topliss’, ‘fr_NH2’,
‘fr_sulfide’, ‘fr_aryl_methyl’, ‘fr_Al_OH’, ‘fr_Ndealkylation1’,
‘fr_priamide’, ‘fr_pyridine’, ‘fr_ArN’, ‘fr_sulfonamd’, ‘fr_NH0’,
‘fr_piperzine’, ‘fr_Imine’, ‘fr_urea’, ‘fr_Ndealkylation2’, ‘fr_thiazole’,
‘fr_alkyl_carbamate’, ‘fr_piperdine’, ‘fr_ester’, ‘fr_allylic_oxid’,
‘fr_nitrile’, ‘fr_benzene’,
‘fr_para_hydroxylation’, ‘fr_halogen’,
‘fr_ether’, ‘fr_alkyl_halide’, ‘fr_methoxy’]

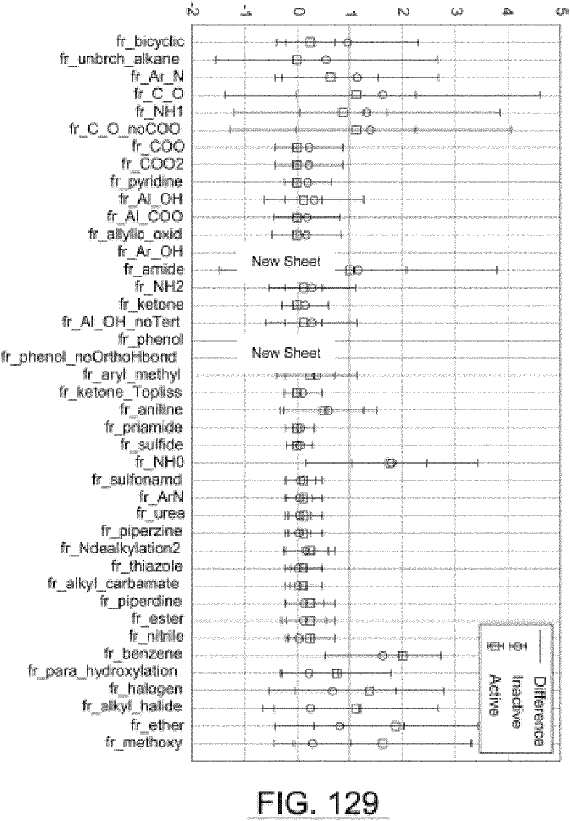

In : (see FIG. 129)

The analysis of the mean of the counts of chemical groups shows that the chemical moieties correlate strongly in both sets active and inactive. A high number of a particular moiety in one group usually means a higher number in the other group too. For all groups the means are within the mutual standard deviations. Therefore, we cannot conclude that the occurrence of a certain moiety has special explanatory power, in the first place. However, we should look at the p-values. Also interesting would be to look at combinations.

In :

### NULL Models

In :

print features
print properties
X_names = features#[‘TPSA’, ‘MW’, ‘fNHAcc’, ‘fNHDon’,‘LogP’,
‘NRings’, ‘NRotB’, ‘NHAcc’, ‘NHDon’]
y_name = ‘activity’
print len(X_names), pow(2,len(X_names))
[‘CSP3’, ‘LogP’, ‘MW, ‘NAromRing’, ‘NHAt’, ‘NOCount’,
‘NRings’, ‘NRotB’, ‘TPSA’, ‘fNHAcc’, ‘fNHDon’]
[‘CSP3’, ‘LogP’, ‘MW, ‘NAromRing’, ‘NHAt’, ‘NOCount’,
‘NRings’, ‘NRotB’, ‘TPSA’, ‘fNHAcc’, ‘fNHDon’] 11 2048

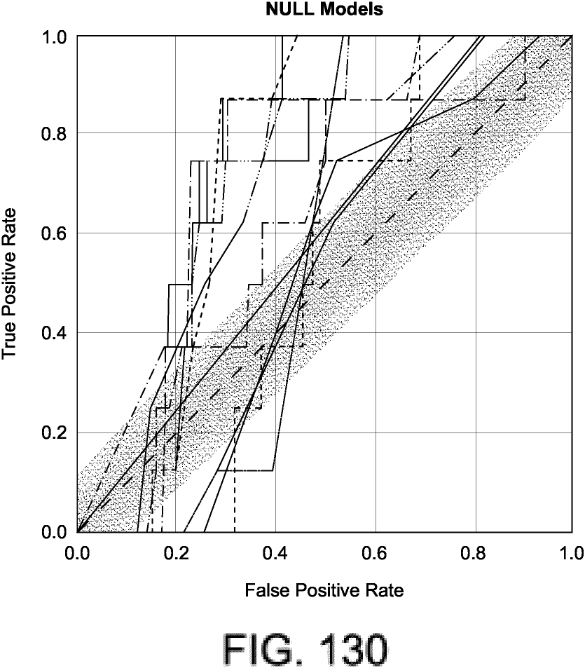

In : (see FIG. 130)

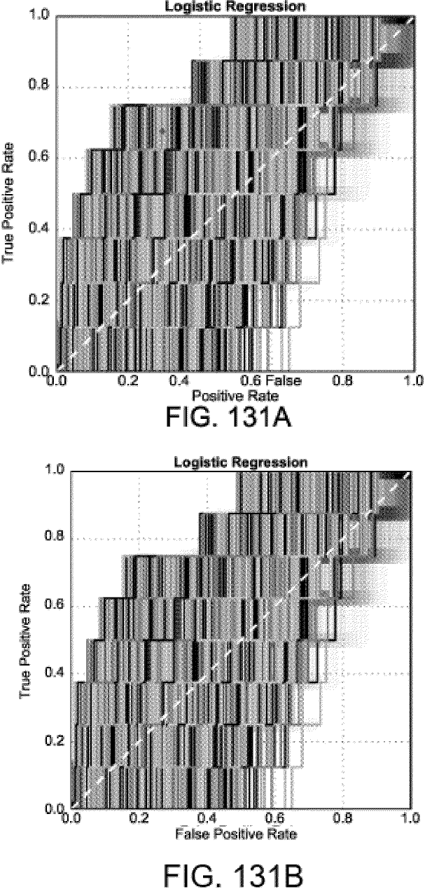

In : (see FIGS. 131A and 131B)

X_names=NullModels.X[:12]

### Naive Bayes

“Naive Bayes methods are a set of supervised learning algorithms based on applying Bayes’ theorem with the “naive” assumption of independence between every pair of features. Naive Bayes learners and classifiers can be extremely fast compared to more sophisticated methods. [...] [Although] naive Bayes is known as a decent classifier, it is known to be a bad estimator, so the probability outputs from predict_proba are not to be taken too seriously.” scikit-learn 0.15.2 documentation

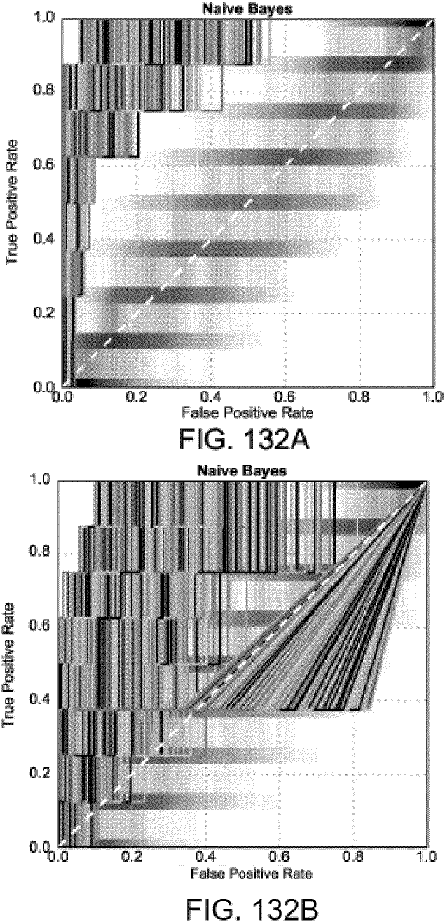

In : (see FIGS. 132A and 132B)

models={}

### Decision Tree

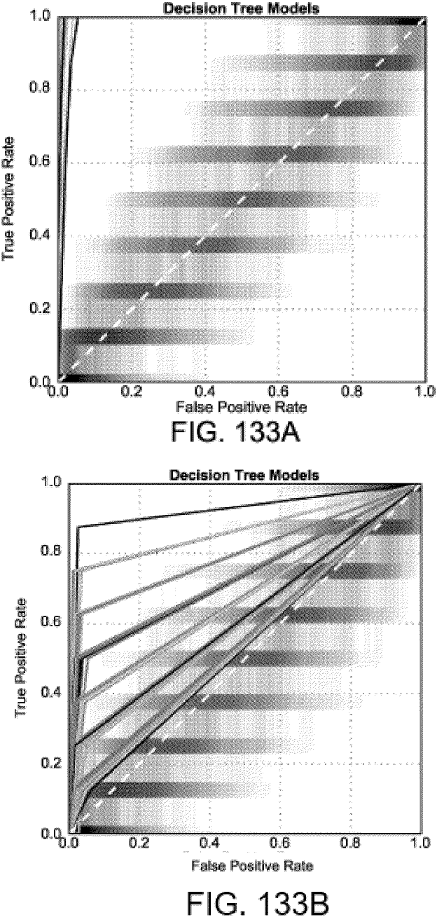

In : (see FIGS. 133A and 133B)

### Random Forest

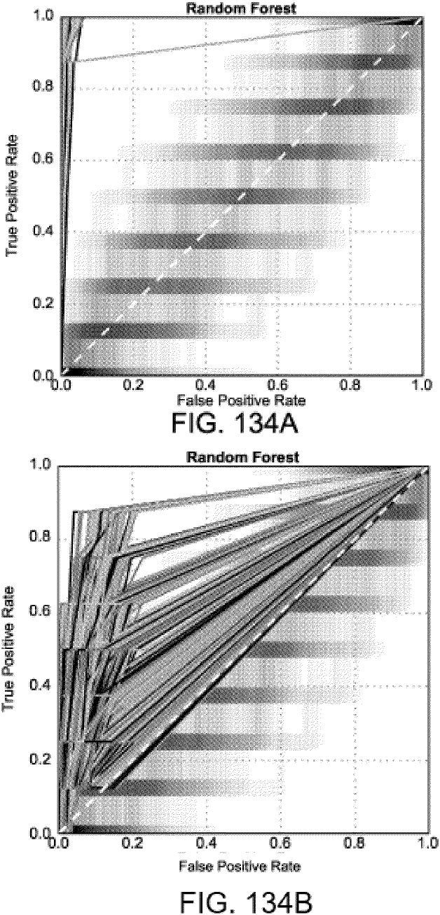

In : (see FIGS. 134A and 134B)

<matplotlib.text.Text at 0×29f45910>

### Boosting

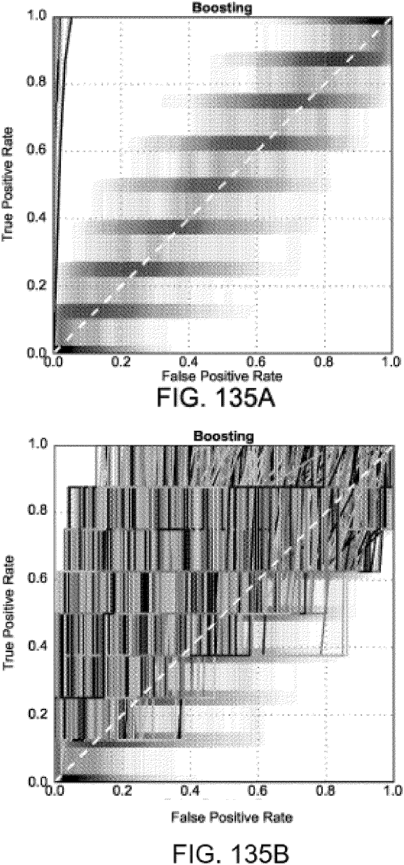

In : (see FIGS. 135A and 135B)

random.seed(34345)

### Number of Features and Prediction Accuracy

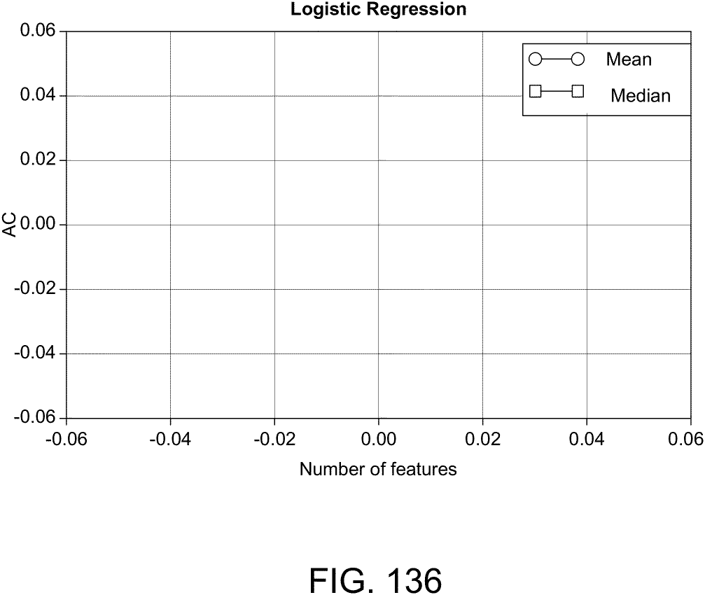

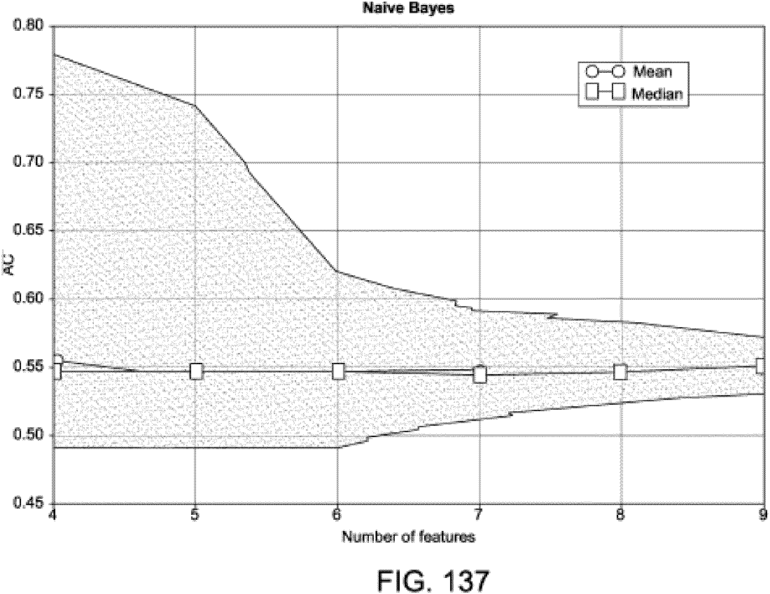

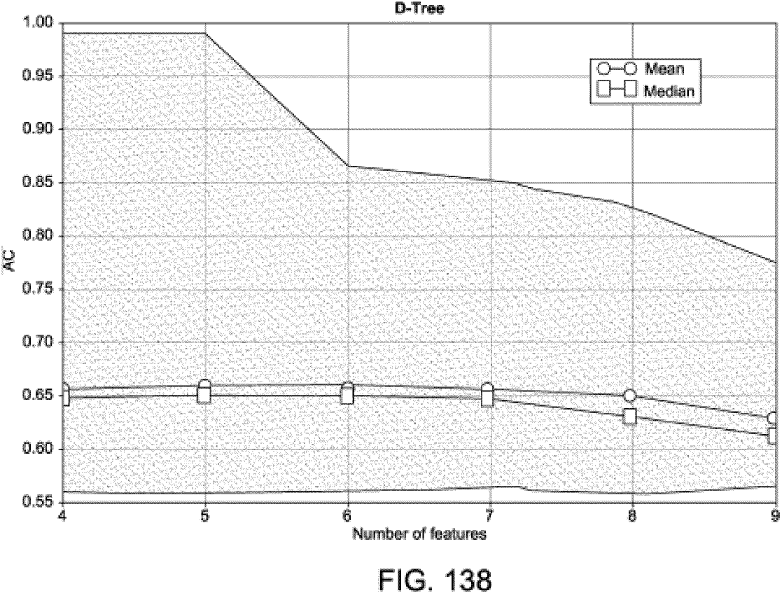

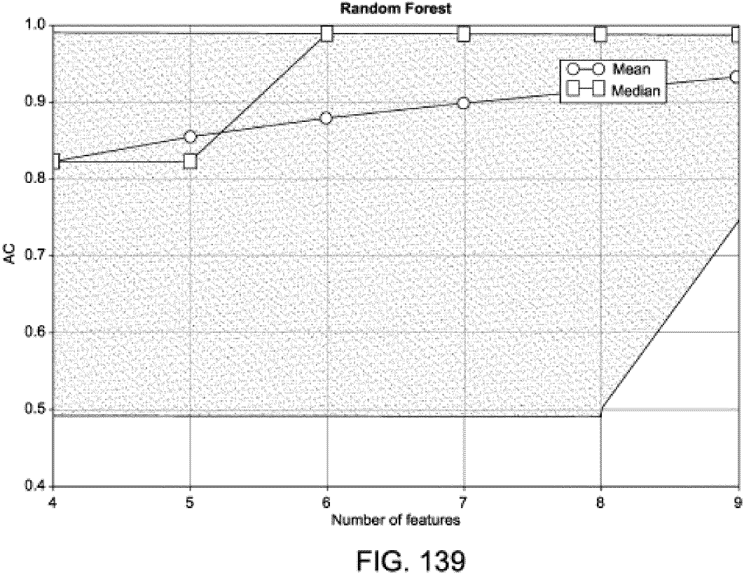

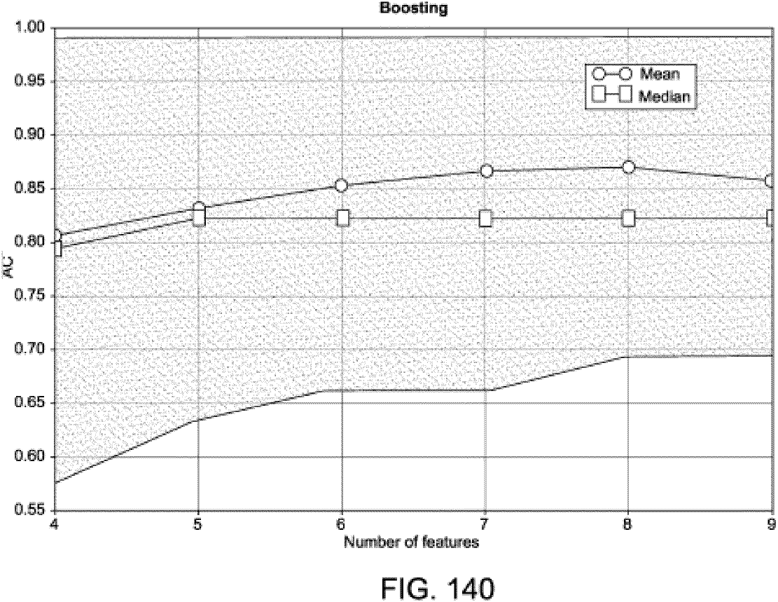

In : (see FIGS. 136-140)

Type Markdown and LaTeX:

α
2

### Select Best Models

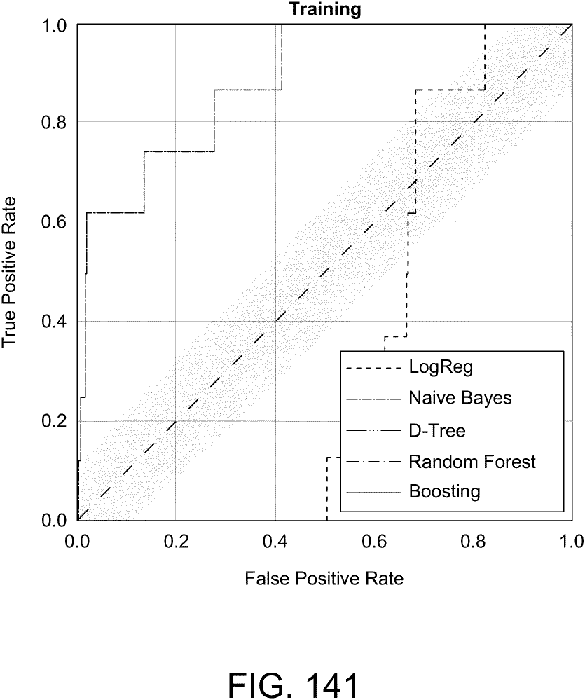

In : (see FIG. 141)

In :

A.sort(‘
AC’,ascending=False)

Out[43]

| | Family | AC | Sensitivity | Specificity | TP | TN | FP | FN |
|---|---|---|---|---|---|---|---|---|
| 2 | D-Tree | 1.000000 | 1.000000 | 1.000000 | 1.000000 | 1.000000 | 0.000000 | 0.000000 |
| 4 | Boosting | 1.000000 | 1.000000 | 1.000000 | 1.000000 | 1.000000 | 0.000000 | 0.000000 |
| 3 | Random Forest | 0.998843 | 1.000000 | 0.997691 | 0.997685 | 1.000000 | 0.002315 | 0.000000 |
| 1 | Naive Baves | 0.704820 | 0.629937 | 0.983418 | 0.992974 | 0.416667 | 0.007026 | 0.583333 |
| 0 | LoqReq | NaN | NaN | \|NaN | 0.981777 | NaN | 0.018223 | NaN |

In :

Result_train={}
for name,model in
zip(BestModels_byAC.Family,BestModels_byAC.model): X= model.X
Result_train[name]=model.predict_proba(train[X]).T[1]
Result_train=pd.DataFrame(Result_train)
Result_train.index=train[[‘activity’]].index
Result_train=Result_train.join(train[[‘activity’]])
Result_train.boxplot(by=‘activity’,figsize=(10,10)) plt.show()
Result_train.hist(figsize=(10,10),normed=True,bins=10)
plt.suptitle(‘Histogram of predicted probabilities\nTrainig set’)
plt.show()

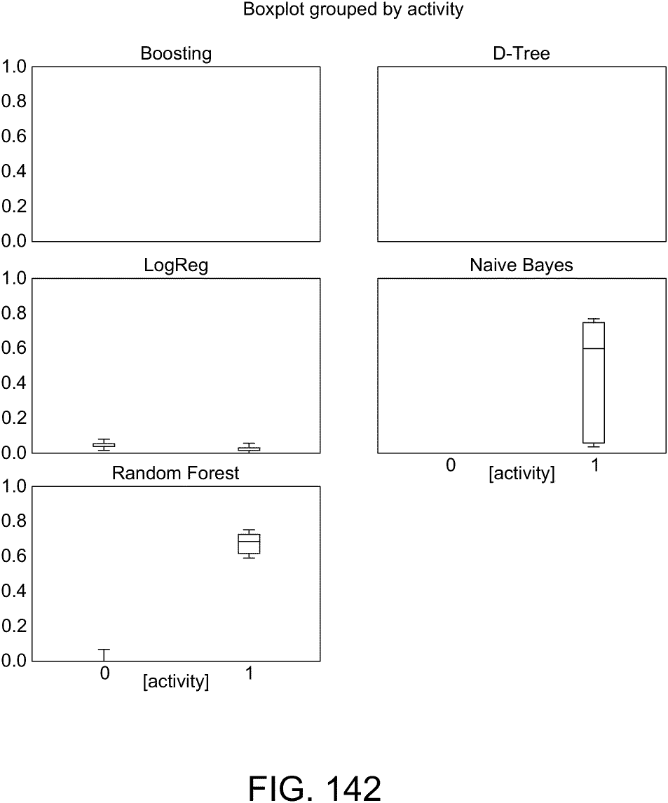

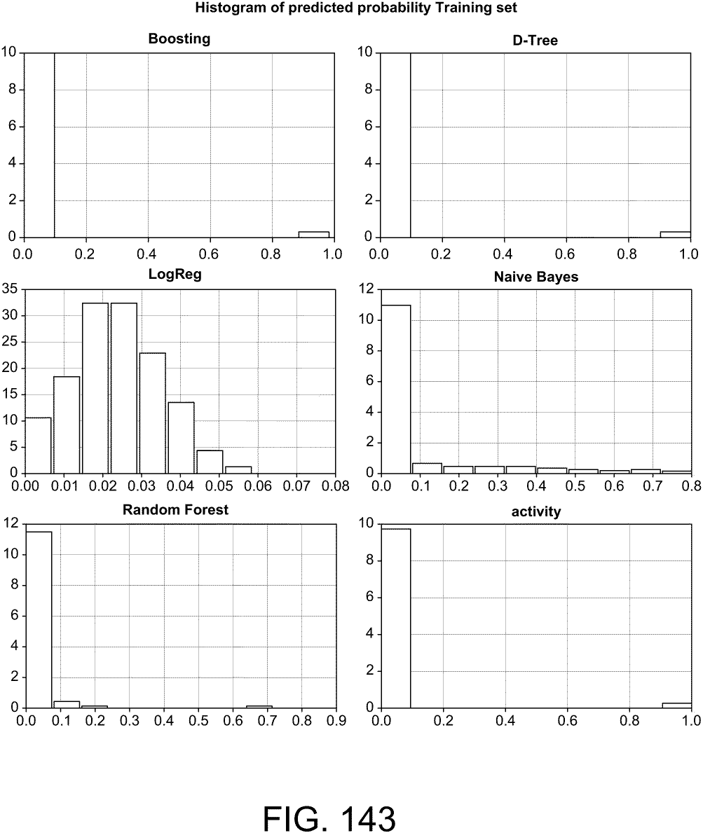

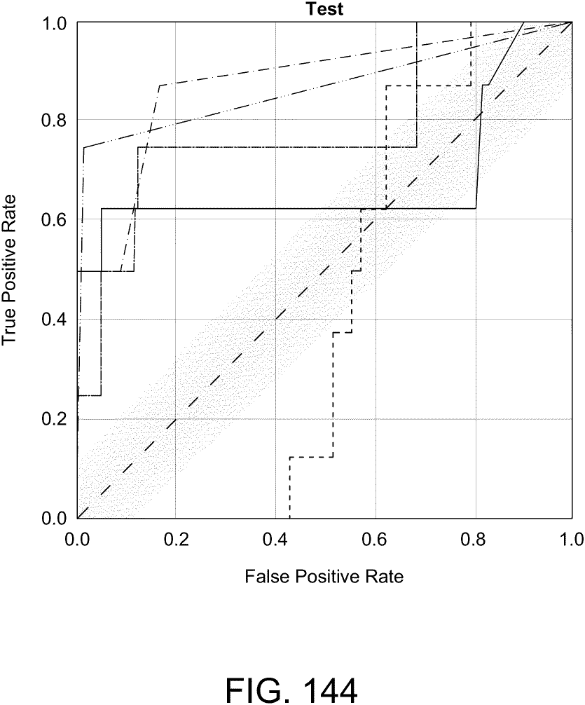

(see FIGS. 142-144)

In :

In :

Out

| | Family | AC | Sensitivity | Specificity | TP | TN | FP | FN |
|---|---|---|---|---|---|---|---|---|
| 3 | Random | 0.995413 | 1.000000 | 0.990909 | 0.990826 | 1.000000 | 0.009174 | 0.000000 |
| 2 | D-Tree | 0.994279 | 1.000000 | 0.988688 | 0.988558 | 1.000000 | 0.011442 | 0.000000 |
| 4 | Boosting | 0.994279 | 1.000000 | 0.988688 | 0.988558 | 1.000000 | 0.011442 | 0.000000 |
| 1 | Naive | 0.781095 | 0.698047 | 0.984091 | 0.990762 | 0.571429 | 0.009238 | 0.428571 |
| 0 | LogReg | NaN | NaN | NaN | 0.981818 | NaN | 0.018182 | NaN |

Type Markdown and LaTeX:
α
2

In :

Result_ test={}
for name,model in
zip(BestModels_byAC.Family,BestModels_byAC.model): X=
model.X
Result_test[name]=model.predict_proba(test[X]).T[1]
Result_test=pd.DataFrame(Result_test)
Result_test.index=test[[‘activity’]].index
Result_test=Result_test.join(test[[‘activity’]])
Result_test.boxplot(by=‘activity’,figsize=(10,10)) plt.show()
Result_test.hist(figsize=(10,10),normed=True,bins=10)
plt.suptitle(‘Histogram of predicted probabilities\nTestset’) plt.show()

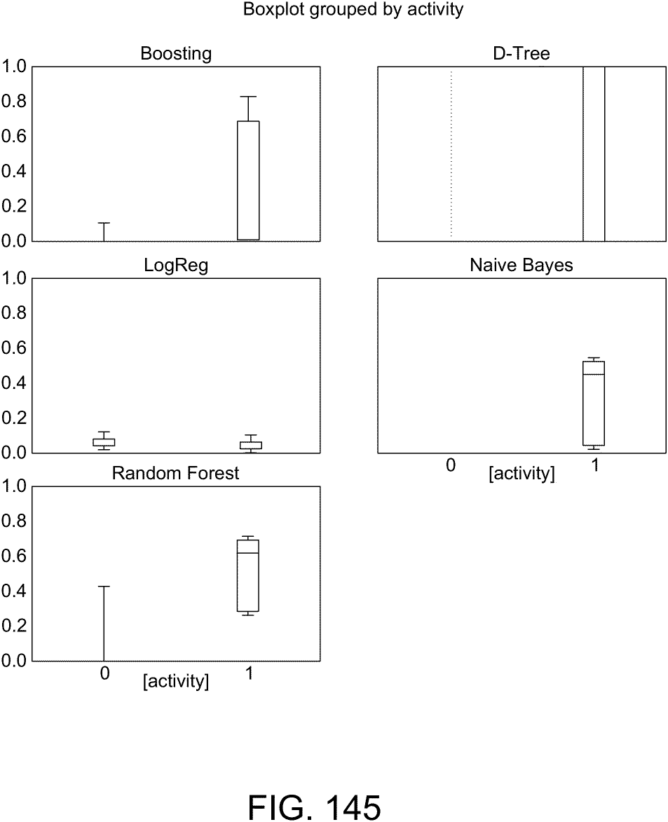

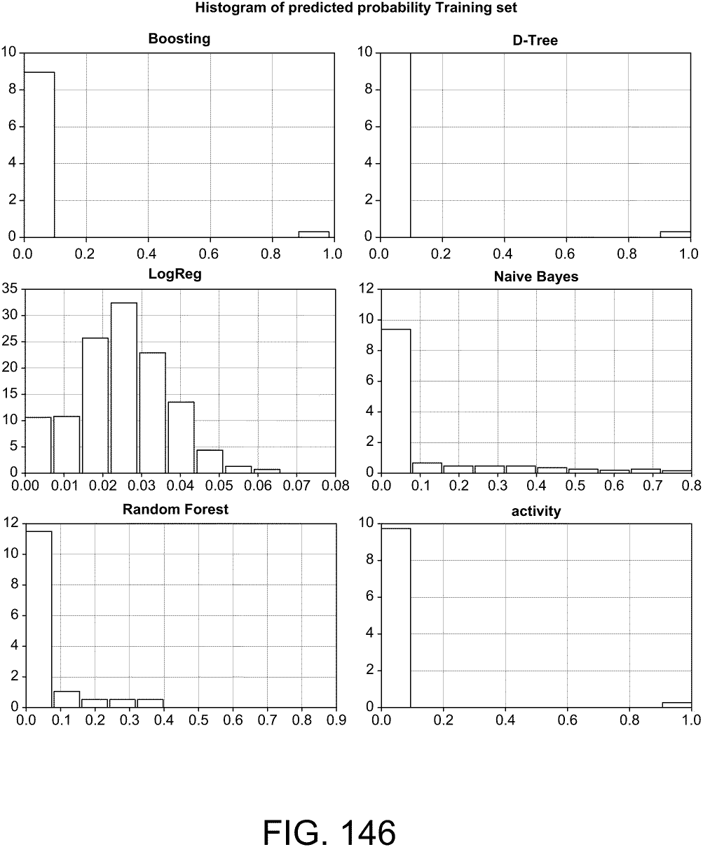

(see FIGS. 145 and 146)

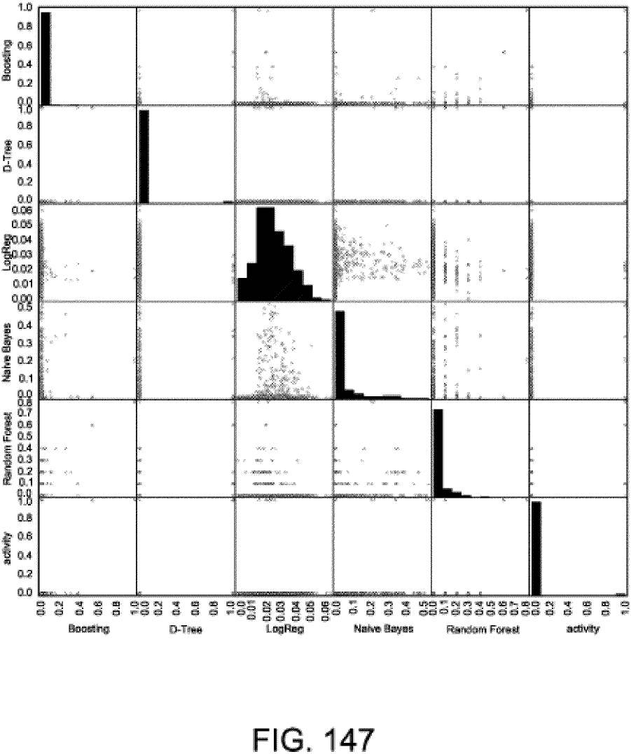

In : (see FIG. 147)

### Error Estimation

In :

The classification accuracy of the models is between 0.55 for the LR model and and 0.75 for the Naive Bayes model. The true negative rate of the Naive Bayes model is best with 0.88. The FP rate of the RF and LR model are smallest. The RF model has a very high FN rate, although the accuracy is pretty high. Alltogether, the NB model seems to be the most robust model with a sensitivity of ~0.85 and specificity of ~0.7. However, the ROC shows that the RF model can perform much better.
How is the classification done with model.predict()? Sensitivity
TPI(TP+FN)

Specificity

TNI(FP+TN)

Accuracy (TP+TN)/(TP+FP+FN+TN)

### Quantitative Prediction

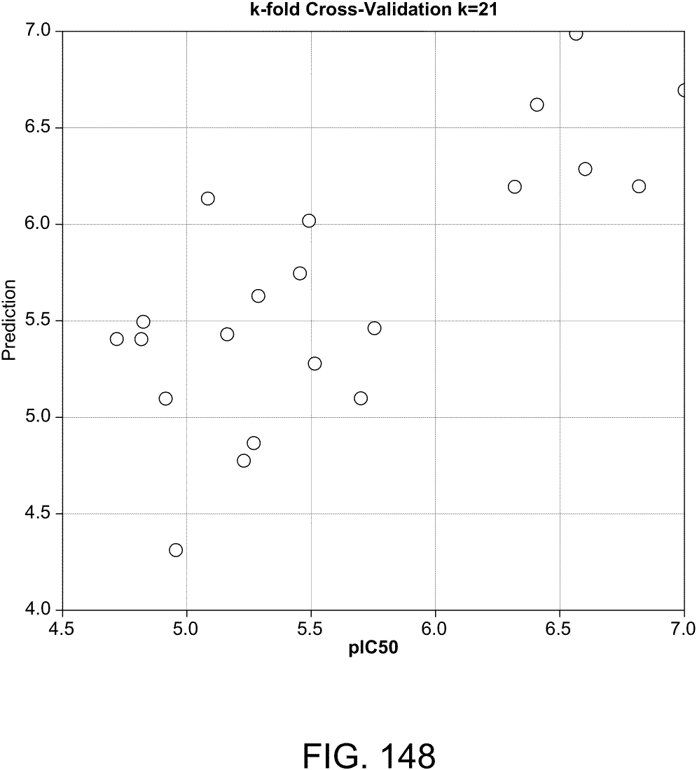

In [ ]: (see FIG. 148)

What is claimed is:

1. A computer-implemented method of predicting cardiotoxicity of molecular parameters of a compound, the method including:
by a computer, providing as input to a machine learning algorithm the molecular parameters of the compound,
the molecular parameters including at least structural information about the compound,
the machine learning algorithm having been trained using respective molecular parameters of compounds known to have cardiotoxicity and of compounds known not to have cardiotoxicity; and
by the computer, receiving as output from the machine learning algorithm a representation of the predicted cardiotoxicity of each molecular parameter of at least a subset of the molecular parameters of the compound.

2. The method of claim 1, wherein the representation of the predicted cardiotoxicity includes, for each molecular parameter of at least the subset of the molecular parameters of the compound, a numerical value representing the predicted cardiotoxicity of that molecular parameter.

3. The method of claim 1, further comprising redesigning the compound so as not to include at least one of the molecular parameters of at least the subset.

4. The method of claim 3, further comprising:
by the computer, providing as input to the machine learning algorithm the molecular parameters of the redesigned compound; and
by the computer, receiving as output from the machine learning algorithm a representation of the predicted cardiotoxicity of each molecular parameter of at least a subset of the molecular parameters of the redesigned compound.

5. The method of claim 1, wherein the representation includes a value representative of a prediction that the molecular parameter of at least the subset will cause the compound to block two or more cardiac ion protein channels.

6. The method of claim 4, wherein the two or more cardiac ion protein channels are selected from the group consisting of: sodium ion channel proteins, calcium ion channel proteins, and potassium ion channel proteins.

7. The method of claim 6, wherein the potassium ion channel protein is HERG1, wherein the sodium ion channel protein is hNaᵥ1.5, or wherein the calcium channel protein is hCaᵥ1.2.

8. The method of claim 1, further comprising:
by the computer, providing as input to the machine learning algorithm, respective molecular parameters of a plurality of compounds of which the previously recited compound is a member;
by the computer, receiving as output from the machine learning algorithm a representation of the predicted cardiotoxicity of each molecular parameter of at least a subset of the molecular parameters of each of the compounds of the plurality of compounds; and
by the computer, selecting a compound of the plurality of compounds based on the predicted cardiotoxicity of each molecular parameter of at least a subset of the molecular parameters of each of the compounds of the plurality of compounds.

9. The method of claim 1, wherein the compounds known to have cardiotoxicity and the compounds known not to have cardiotoxicity are selected based on a statistical analysis of the molecular parameters of those compounds.

10. The method of claim 1, wherein the machine learning algorithm is selected from the group consisting of: a naive Bayes model, a naive Bayes bitvectors model, a decision tree model, a random forest model, a LogReg model, and a boosting model.

11. The method of claim 1, wherein the machine learning algorithm comprises a XGBoost algorithm.

12. The method of claim 1, wherein the molecular parameters further include one or more of physical information about the compound, and chemical information about the compound.

13. A computer system for predicting cardiotoxicity of molecular parameters of a compound, the computer system including:
a processor; and
at least one computer-readable medium storing:
the molecular parameters of the compound, the molecular parameters including at least structural information about the compound;
a machine learning algorithm having been trained using respective molecular parameters of compounds known to have cardiotoxicity and of compounds known not to have cardiotoxicity; and
instructions for causing the processor to perform steps including:
providing as input to the machine learning algorithm the molecular parameters of the compound; and
receiving as output from the machine learning algorithm a representation of the predicted cardiotoxicity of each molecular parameter of at least a subset of the molecular parameters of the compound.

14. The system of claim 13, wherein the representation of the predicted cardiotoxicity includes, for each molecular parameter of at least a subset of the molecular parameters of the compound, a numerical value representing the predicted cardiotoxicity of that molecular parameter.

15. The system of claim 13, the at least one computer-readable medium further storing instructions for causing the processor to redesign the compound so as not to include at least one of the molecular parameters of at least the subset.

16. The system of claim 15, the at least one computer-readable medium further storing instructions for causing the processor to:
provide as input to the machine learning algorithm the molecular parameters of the redesigned compound; and
receive as output from the machine learning algorithm a representation of the predicted cardiotoxicity of each molecular parameter of at least a subset of the molecular parameters of the redesigned compound.

17. The system of claim 16, wherein the representation includes a value representative of a prediction that the molecular parameter of at least the subset will cause the compound to block two or more cardiac ion protein channels.

18. The system of claim 17, wherein the two or more cardiac ion protein channels are selected from the group consisting of: sodium ion channel proteins, calcium ion channel proteins, and potassium ion channel proteins.

19. The system of claim 18, wherein the potassium ion channel protein is HERG1, wherein the sodium ion channel protein is hNav1.5, or wherein the calcium channel protein is hCaᵥ1.2.

20. The system of claim 13, the at least one computer-readable medium further storing instructions for causing the processor to:
provide as input to the machine learning algorithm respective molecular parameters of a plurality of compounds of which the previously recited compound is a member;
receive as output from the machine learning algorithm a representation of the predicted cardiotoxicity of each molecular parameter of at least a subset of the molecular parameters of each of the compounds of the plurality of compounds; and
select a compound of the plurality of compounds based on the predicted cardiotoxicity of each molecular parameter of at least a subset of the molecular parameters of each of the compounds of the plurality of compounds.

21. The system of claim 13, wherein the compounds known to have cardiotoxicity and the compounds known not to have cardiotoxicity are selected based on a statistical analysis of the molecular parameters of those compounds.

22. The system of claim 13, wherein the machine learning algorithm is selected from the group consisting of: a naive Bayes model, a naive Bayes bitvectors model, a decision tree model, a random forest model, a LogReg model, and a boosting model.

23. The system of claim 13, wherein the machine learning algorithm comprises a XGBoost algorithm.

24. The system of claim 13, wherein the molecular parameters are selected from the group consisting of: structural information about the compound, physical information about the compound, and chemical information about the compound.

25. At least one computer-readable medium for use in predicting cardiotoxicity of molecular parameters of a compound, the at least one computer-readable medium storing:
the molecular parameters of the compound, the molecular parameters including at least structural information about the compound;
a machine learning algorithm having been trained using respective molecular parameters of compounds known to have cardiotoxicity and of compounds known not to have cardiotoxicity; and
instructions for causing a processor to perform steps including:
providing as input to the machine learning algorithm the molecular parameters of the compound; and
receiving as output from the machine learning algorithm a representation of the predicted cardiotoxicity of each molecular parameter of at least a subset of the molecular parameters of the compound.
